(19) United States
(12) Patent Application Publication
(10) Pub. No.: US 2026/0035750 A1
(43) Pub. Date: Feb. 5, 2026

(54) TRANSCRIPTIONAL REGULATORS OF THE TEAD FAMILY

(71) Applicant: Sanofi, Paris (FR)

(72) Inventor: Emmanuel SPANAKIS, Paris (FR)

(21) Appl. No.: 18/849,391

(22) PCT Filed: Mar. 22, 2023
(86) PCT No.: PCT/EP2023/057332

§ 371 Date: Sep. 20, 2024

(30) Foreign Application Priority Data
Mar. 23, 2022 (EP) 22315071.5

(51) Int. Cl. C12Q 1/6886

(52) U.S. Cl. CPC C12Q 1/6886; C12Q 2600/106; C12Q 2600/112; C12Q 2600/158

(57) ABSTRACT

The present disclosure relates to transcriptional signatures obtained on a set of genes comprising any of 220 to 249 of a set of genes (1) and any of 210 to 233 of a set of genes (2) from a TEAD-500 signature and its use for determining the TEAD-activity of a cancer. The transcriptional signatures are useful in a variety of applications, including, predicting the likelihood that a subject will respond to a TEAD-pathway inhibitor treatment, selecting patients for clinical trials, assessing efficacy of TEAD-pathway inhibitor molecules, and prognosing survival, response to, and benefit from anti-TEAD pathway treatments.

### CROSS REFERENCE TO RELATED APPLICATIONS

This application is a U.S. national stage application under 35 U.S.C. § 371 of International Application No. PCT/EP2023/057332, filed internationally on Mar. 22, 2023, which claims priority to EP Application Serial No. 22315071.5, filed Mar. 23, 2022, the disclosures of which are herein incorporated by reference in their entirety.

### BACKGROUND OF THE DISCLOSURE

In normal tissues, the transcription co-factors YAP1 and WWTR1 bind to transcription factors of the TEAD family (TEAD1, TEAD2, TEAD3, and TEAD4), collectively referred to as “TEAD,” to form an active protein complex. This complex recognizes and binds to a specific sequence motif in the promoters of target genes and initiates or inhibits their transcription. Cell proliferation, survival, plasticity, and migration necessary for normal biological processes such as organ growth and wound healing are therefore regulated by TEAD (Totaro A, et al. Nat Cell Biol. 2018; 20 (8): 888-899. doi: 10.1038/s41556-018-0142-z). In intact adult mammalian tissues, YAP1 and WWTR1 are generally phosphorylated by kinases of the Hippo pathway, and thereby retained in the cytoplasm and degraded. In this case, there is no active transcription complex in the nucleus (Totaro A, et al. Nat Cell Biol 2018. August; 20 (8): 888-899).

Over the past few years, YAP1, WWTR1, their TEAD partners, and their upstream regulators—notably the tumor suppressor pathway known as Hippo pathway—present increasing interest in cancer research (Zhang N et al. Dev Cell. 2010 Jul. 20; 19(1):27-38. doi: 10.1016/j.devcel.2010.06.015; Lu L, et al. Proc Natl Acad Sci USA. 2010; 107(4):1437-42. doi: 10.1073/pnas.0911427107; Nishio M, et al. Proc Natl Acad Sci USA. 2016; 113(1):E71-80. doi: 10.1073/pnas.1517188113; Liu-Chittenden Y, et al., Genes Dev. 2012; 26(12):1300-5. doi: 10.1101/gad.192856.112). Mutations or physiological dysregulation of either YAP1, WWTR1, or any of their upstream regulators, may result in insufficient phosphorylation and degradation of these proteins. Without phosphorylation and degradation, the transcription co-factors enter the nucleus, bind to TEAD, and initiate oncogenic transcription. Increased activity of either component of the complex may increase, or decrease, the transcription of downstream effectors (TEAD-dependent transcription). Some of the modulated genes are direct targets of TEAD bearing TEAD-binding motifs (Zanconato, F. et al. Nat Cell Biol 2015:17:1218-1227). Others are modulated indirectly. Thus, the Hippo-YAP1/WWTR1-TEAD pathway (hereinafter “TEAD pathway”) may directly trigger tumorigenesis or render existing tumors resistant to targeted treatments (Reggiani F et al. Biochim Biophys Acta Rev Cancer. 2020: 1873 (1): 188341. doi: 10.1016/j.bbcan.2020.188341; Kurppa K J et al. Cancer Cell. 2020; 37 (1): 104-122.e12. doi: 10.1016/j.ccell.2019.12.006).

For example, mutations and deletions of Hippo genes such as LATS2 and NF2 account for a large proportion of malignant mesothelioma tumors (Sekido Y et al. Cancers (Basel). 2018; 10 (4): 90. doi: 10.3390/cancers10040090). YAP1 is found focally amplified, and over-expressed at both RNA and protein levels, in several types of tumors, notably in cervical cancers (Zanconato F et al. Cancer Cell. 2016; 29 (6): 783-803. doi: 10.1016/j.ccell.2016.05.005). Activation of TEAD-dependent transcription may give tumor cells a mesenchymal stem cell-like phenotype since notorious stem-cell transcription factors like SOX2, OCT3/4, NANOG and MYC are also controlled by TEAD (Bora-Singhal N et al. Stem Cells. 2015; 33 (6): 1705-18. doi: 10.1002/stem. 1993).

To measure the transcriptional activity of such complexes, estimate the proportion of cases where TEAD-dependent transcription may be responsible for tumor development or evolution, and evaluate the target engagement and pharmacodynamics of novel TEAD-pathway inhibitors, we have identified a generic as well as several tissue-specific transcriptional signatures comprising genes that are upregulated (positive effectors) or downregulated (negative effectors) when a YAP1/WWTR1-TEAD complex (“TEAD complex”) is activated. Inversely, the positive effectors are downregulated, and the negative effectors are upregulated, when the complex is inhibited.

The generic signature, referred to herein as the “TEAD-500” gene list, is derived from the top 500 differentially expressed genes with positive and negative downstream effectors at about equal proportions. More precisely, this TEAD-500 genes list comprises from 482 genes down to about 90% of these genes. The generic signature is robustly applicable to all types of cancer. It robustly measures TEAD activity, classifies samples, and predicts their response to TEAD-pathway modulation. The TEAD-500 may be used to compute a TEAD-score as detailed thereafter.

Additional signatures, produced by discriminant function analysis (SPSS statistics, IBM, Armonk, New York), were identified and are applicable to specific types of cancer (indications) and serve to classify TEAD-active versus TEAD-inactive tumors, as these are defined by TEAD-500, with accuracy approaching or very frequently reaching 100%. To a specific, shorter, signature is associated a specific type of cancer. A shorter signature comprises an optimal discriminant function and occasionally a minimal discriminant function. A discriminant function comprises a set of genes and a constant coefficient of the discriminant function. To each gene of the set of gene is associated a coefficient. For a given signature, a minimal discriminant function comprises the genes that are essential for 100% correct classification. Any missing piece of data will introduce some degree of inaccuracy. For a given signature, an optimal discriminant function uses more predictors but will still achieve 100% correct classification of patients when some data are missing. The additional signatures, minimal and optimal discriminant functions, can be used, according to one method, to compute a discriminant score (S), as detailed thereafter. The optimal discriminant function may alternately be used, according to another method, to compute a discriminant score (DS), as detailed thereafter.

The identified signatures may be utilized in a variety of clinical and research-related utilities, including, but not limited to, monitoring the activity of the TEAD complex and evaluating the pharmacodynamics of novel inhibitors of the TEAD pathway in vitro or in vivo, to diagnose tumors of which the development or evolution is attributable to activation of the TEAD complex, to recruit patients with active TEAD to clinical trials designed to evaluate TEAD pathway inhibitors, to prognose survival, response to, and benefit from an anti-TEAD pathway treatment, alone or in combination with other treatments, to estimate the proportion of cases within cohorts, for each cancer indication or subtype, where TEAD-dependent transcription may be responsible for tumor development or evolution to treatment resistance, and to measure TEAD-dependent transcription in any tissue, under any condition, pathological or not, in the laboratory or in the clinic.

### ABBREVIATIONS

ACC Adrenocortical carcinoma
BID Twice a day
BLCA Bladder Urothelial Carcinoma
BRCA Breast invasive carcinoma
CCLE Cancer Cell Line Encyclopedia
CESC Cervical squamous cell carcinoma and endocervical adenocarcinoma
CHOL Cholangiocarcinoma
CMS1 Consensus molecular subtypes 1 of colorectal cancer
CMS2 Consensus molecular subtypes 2 of colorectal cancer
CMS3 Consensus molecular subtypes 3 of colorectal cancer
CMS4 Consensus molecular subtypes 4 of colorectal cancer
COAD Colon adenocarcinoma
ddeR Relative TEAD activity score based on effector ranks (difference from control)
deR Absolute TEAD activity score based on effector ranks
dgR Absolute TEAD activity score based on whole-transcriptome ranks
DLBC Lymphoid Neoplasm Diffuse Large B-cell Lymphoma
DMSO Dimethyl sulfoxide
DN Dominant negative
ESCA Esophageal carcinoma
GBM Glioblastoma multiforme
GEO Gene Expression Omnibus
GTEx Genotype Tissue Expression Project
HNSC Head and Neck squamous cell carcinoma
HuGO Human Genome Organization
KICH Kidney Chromophobe
KIRC Kidney renal clear cell carcinoma
KIRP Kidney renal papillary cell carcinoma
LGG Brain Lower Grade Glioma
LIHC Liver hepatocellular carcinoma
LUAD Lung adenocarcinoma
LUSC Lung squamous cell carcinoma
MESO Mesothelioma
NA Not applicable
NCBI National Center for Biotechnology Information
OV Ovarian serous cystadenocarcinoma
PAAD Pancreatic adenocarcinoma
PCPG Pheochromocytoma and Paraganglioma
PRAD Prostate adenocarcinoma
QD Once daily
READ Rectum adenocarcinoma
RT-qPCR Reverse transcription-quantitative polymerase chain reaction
SARC Sarcoma
SCID Severe combined immunodeficiency
SKCM Skin Cutaneous Melanoma
STAD Stomach adenocarcinoma
TCGA The Cancer Genome Atlas
TGCT Testicular Germ Cell Tumors
THCA Thyroid carcinoma
THYM Thymoma
UCEC Uterine Corpus Endometrial Carcinoma
UCS Uterine Carcinosarcoma

### BRIEF DESCRIPTION OF THE DRAWINGS

FIG. 1. Comparison of TEAD-500 deR and dgR scores in experimental samples (N=1176).

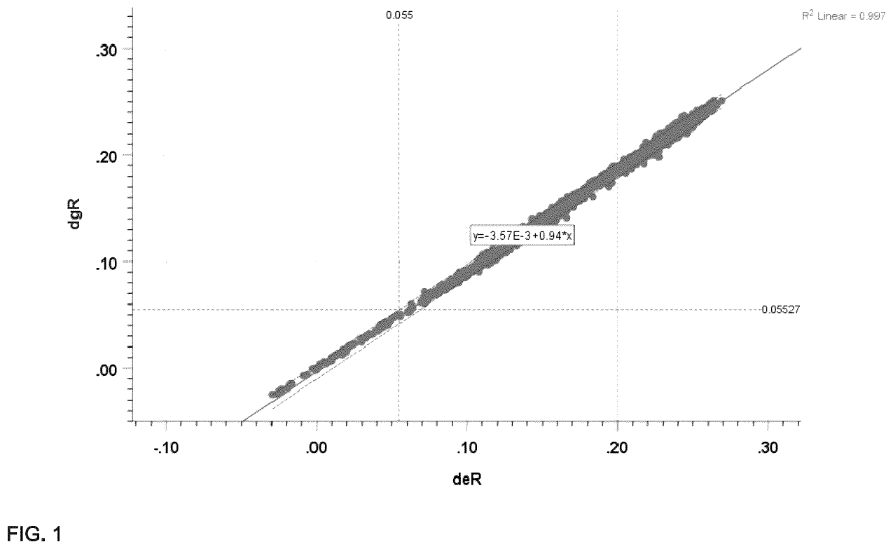

FIG. 2. Robustness of the TEAD-500 scores. Two TEAD-500 sub-lists, each with 10% of the genes randomly removed, resulted in very similar TEAD-activity scores for 1112 experimental samples from our collection.

FIGS. 3A-3C. Dose-dependent regression of the TEAD-500 score (A) and inhibition of the CCN1 marker of TEAD activity, at RNA (FIG. 3B) and protein (C) level, by a benchmark chemical inhibitor of TEAD (K-975) in mesothelioma NCI-H226 cells grown in vitro.

FIGS. 4A-4B. Validation of the TEAD-500 signature in vivo. MSTO-211H cells transfected with a doxycycline-inducible TEAD1-DN construct were implanted in SCID mice. When TEAD1-DN transcription was induced, the xenografts began to regress (A) while a significant fall of the TEAD-500 score was observed within 96 h (B).

FIGS. 5A-5B. SCID mice implanted with malignant mesothelioma parental NCI-H226 cells were treated with 200 mg/kg BID of the benchmark TEAD-inhibitor K-975 for 17 consecutive days (bottom grey line-square dots). The dose of 100 mg/kg BID was still active with almost tumor stasis; dotted line), while the dose of 30 mg/kg BID was inactive (upper light grey line with black dots). (A). Approximately 30-40% inhibition of the YAP1/TEAD-dependent transcription in 24 h (darker lines) is sufficient for tumor regression in this model (B).

FIG. 6. The TEAD-500 score distribution moves towards higher values in tumors (TCGA primary tumors) compared to normal tissues (GTEx or TCGA tumor-adjacent normal tissues). Primary tumors tend to score higher than adjacent normal tissues. Recurrent and metastatic tumors score higher than primary tumors. Therefore, YAP1/TEAD-dependent transcription increases with tumorigenesis and tumor progression. Cell lines (CCLE) attain much higher scores, and mesothelioma cell lines are among the top scorers.

FIGS. 7A-7B. TEAD-500 score distribution means in cervical cancers (TCGA CESC Cohort) with YAP1 amplification compared to homologous tumors with diploid YAP1 (A) and in malignant mesothelioma tumors (TCGA MESO cohort) compared to all other TCGA primary tumors (B).

FIG. 8. Overall survival of malignant mesothelioma patients (TCGA MESO cohort) with tumors scoring high or low for TEAD-dependent transcription as measured by TEAD-500.

FIG. 9. Overall survival of patients with all TCGA cancer types pooled, in relation to the level of TEAD-dependent transcription as scored by TEAD-500.

FIG. 10. Optimal discrimination of mesothelioma samples with active or inactive TEAD-pathway.

FIG. 11. Minimal discrimination of mesothelioma samples with active or inactive TEAD-pathway.

FIG. 12. Comparison of optimal and minimal discriminant scores.

### SUMMARY

Provided herein are gene signatures (e.g., panels of biomarkers) useful in a variety of clinical and diagnostic applications, including, but not limited to, determining whether a subject will respond to particular treatments, monitoring the activity of the TEAD complex, evaluating the pharmacodynamics of inhibitors of the TEAD pathway in vitro or in vivo, diagnosing tumors of which the development or evolution is attributable to activation of the TEAD complex, recruiting patients with active TEAD to clinical trials designed to evaluate TEAD pathway inhibitors, prognosing survival, response to, and benefit from an anti-TEAD pathway treatment, alone or in combination with other treatments, estimating the proportion of cases within cohorts where TEAD-dependent transcription may be responsible for tumor development or evolution to treatment resistance, and to measure TEAD-dependent transcription in any tissue, under any condition, pathological or not, in the laboratory or in the clinic.

To arrive at the gene signatures, the activity of TEAD-complexes in single tissue-samples of various kinds was evaluated by observing the relative expression of the top-500 most significant differentially expressed genes that are influenced by reported experimental modulations of the TEAD-complex. The TEAD-500 signature derives from the top-500 most significant differentially expressed genes by dropping out the redundant or not relevant gene.

We herein refer to this panel as “TEAD-500,” which actually consists of about 482 genes. Up to 10% of the genes may be randomly removed from the TEAD-500 without significantly compromising the accuracy of the results.

The present disclosure relates to a panel of nucleic acid biomarkers having N biomarkers, wherein N is an integer of at least 160 and equal to or less than about 482, and wherein the N biomarkers comprise nucleic acid capable of detecting expression levels of any 80 to about 249 of genes of a set of genes (1) (the positive effector genes) and any of 80 to about 233 of genes of a set of genes (2) (the negative effector genes).

A transcriptional signature described herein may be obtained by measure of expression levels of genes of a set of genes comprising any of 220 to 249 of the genes of a set of genes (1) (positive effector genes) and any of 210 to 233 of genes of a set of genes (2) (negative effector genes) as listed below. Such transcriptional signatures, or related sets of genes, are named herein “TEAD-signature”.

Embodiment 1. The 249 positive effector genes in TEAD-500 (or set of genes (1)): AASS, ABAT, ACAT2, ADAMTS1, ADM, ADRB2, AMOT, ANXA3, ARHGAP11A, ARHGDIB, AURKB, AVPI1, AXL, AZIN1, B4GALT4, BCAT1, BIRC5, BTG3, C4BPB, CAP2, CAV1, CAVIN1, CCBE1, CCDC80, CCN1, CCN2, CDC25A, CDC6, CDCA3, CDCA4, CDCA5, CDCA8, CDH4, CDK2, CDK6, CDV3, CENPA, CENPI, CENPM, CENPN, CHRNB1, CHST13, CKS2, CLDN1, CLIC3, CNN3, COBL, COL8A1, COTL1, CPA4, CRIM1, CRY1, CTH, CXCL1, CYTH3, DAPK1, DCLRE1B, DDAH1, DHCR7, DHFR, DIAPH3, DKK1, DLL1, DONSON, DUSP14, DUT, EBP, EIF2AK3, EMG1, EPHA2, EPS8L2, ESM1, ETS1, EXO1, EXOSC2, F3, FAHD2A, FAM83D, FANCA, FAT4, FDPS, FEN1, FMR1, FST, FSTL1, FSTL3, GADD45A, GADD45B, GINS1, GPC6, GPR176, GPRC5A, GPRC5B, GRAMD2B, HASPIN, HEG1, HEXB, HPS5, HSPB11, IDI1, IGFBP7, IKBIP, IL6, ITGB2, JDP2, JPH2, KPNA2, KRT8, KRT80, LCA5, LHFPL6, LMCD1, LMNB2, LRP8, LRRFIP2, LSM5, LYPD6, LYRM1, MAD2L1, MAP6D1, MATN2, MATN3, MCM10, MCM2, MCM5, MDC1, METRNL, MICB, MID1, MRPL33, MSRB3, MVD, MXRA7, NCAPD3, NEDD4, NEDD4L, NEK2, NEXN, NFIB, NNMT, NOC3L, NTN4, NUAK1, NUAK2, NUDCD1, NUP107, NUP37, OGFRL1, OLFML3, OLR1, OXCT1, PAK2, PCBD1, PCNA, PDLIM2, PDZD2, PEPD, PHLPP1, PKMYT1, PKP2, PKP4, PLCE1, PLEKHA7, PLK2, PLOD2, PPIH, PRPS1, PRPS2, PRSS23, PSG2, PSG6, PSG7, PSG9, PVR, PXMP2, QDPR, QKI, RAB11FIP1, RAB32, RACGAP1, RBM24, RBMS2, RCN2, RFC4, RND3, RNF144B, ROR1, RPS24, SCD5, SCML1, SDC2, SEC14L1, SGK1, SGMS2, SGTB, SH3RF1, SHCBP1, SKP2, SLC25A23, SLC25A3, SLC38A5, SLC3A2, SLC7A1, SLC7A5, SMPD4, SNAPC1, SNX24, SORT1, SPAG1, SPATA5, STK3, STX11, STXBP6, SUSD2, SUV39H1, SYDE2, TACC3, TAGLN, TEAD1, TEAD4, TENT5B, TGM2, THBS1, TK1, TMEM139, TMEM160, TNFAIP3, TNFRSF12A, TNNC1, TPM1, TPX2, TRIP13, TSPAN2, TTF2, TUBB6, TUFT1, TYMS, UAP1, UBE2C, UGCG, UHRF1, VKORC1L1, WWC1, WWC2, YAP1, ZBED2, ZDHHC18, ZNF488, ZNF704, and

Embodiment 2. The 233 negative effector genes in TEAD-500 (set of genes (2)): AASDH, ABCA1, ABCC5, ABI3BP, ABLIM3, ACADVL, ACOT11, ACOX2, ACSL5, ADAM28, AGL, AGPAT4, ALDH3A2, ANKRD12, ANKRD22, ANKRD29, ANKRD42, ANTXR2, APBB3, ARAP3, ARHGEF2, ASF1A, ATP7A, ATXN1, BCL11B, BHLHE41, BMF, CA2, CASP1, CBR3, CCNG2, CDC42EP4, CDK1, CEBPB, CELSR3, CLCN3, CLDN4, COL6A1, COL6A2, CPE, CRABP2, CROT, CSRNP2, CSTA, CTNNBIP1, CTSB, CTSK, CXXC5, CYP1B1, CYP27C1, DDR1, DEDD2, DHX32, DIAPH2, DSC2, DSG3, DUSP6, DYNC2LI1, ELN, EPS8L3, ERAP2, FAM102A, FAM117B, FAM83B, FAM89B, FERMT1, FKBP2, FOS, FTH1, FXYD3, GDPD1, GOLGA5, GOLPH3L, GPNMB, GPRC5C, GRB10, GSN, HAS3, HBP1, HDAC1, HDHD2, HEY1, HOXA5, IFI44, IGSF3, IGSF9, INTS3, IRAK2, IRF9, IRX5, ITGA2, KCNMA1, KCNMB3, KCNN4, KIFAP3, KLF10, KLF13, KLHL3, KLK11, KRCC1, KRIT1, KRTDAP, LMTK3, LRP10, LTBP4, LXN, LYPD3, MALL, MANSC1, MAPK13, MARCKSL1, MFSD1, MFSD5, MGST2, MGST3, MLLT11, MLPH, MMP13, MSX2, MTMR11, MTMR9, MTSS1, MYO1A, NAGK, NAPEPLD, NCOA3, NFIL3, NPAS2, NRIP1, OAS1, OAS2, OASL, OFD1, OSBPL7, OTUB2, OVOL1, PAG1, PAK1, PCDHB2, PCDHB9, PCGF3, PCMTD2, PERP, PHF21A, PIK3C2B, PIK3R1, PIK3R2, PIK3R3, PIP4P2, PJA2, PKIA, PLA2G4C, PNRC1, PPP1R11, PRRX2, PTPRE, PYGB, RAC2, RALGPS1, RAPGEFL1, RBM23, RBM45, RBM47, RBP1, REEP6, RGL2, RGS17, RHOC, S100A14, SAMD9, SEC14L2, SECISBP2, SH3PXD2A, SH3TC1, SHROOM2, SLC14A1, SLC1A2, SLC30A9, SLC35C1, SLC37A2, SLC39A11, SLFN5, SLITRK6, SLK, SMOC1, SNCG, SP1, SPIRE2, SPRY4, SQSTM1, SRD5A3, SSPN, STMN3, STX1A, TBX3, TCF25, TDO2, TET2, TFF1, TLR3, TMC4, TMC7, TMEM140, TMEM144, TMEM45B, TP53INP1, TP63, TPD52L1, TRAPPC6B, TRIB1, TRIB2, TRIM13, TRIM31, TRIM38, TRIOBP, TRIP11, TSC22D1, TSPAN1, TTC17, TTLL3, TUBB3, UBC, ULK1, VAMP8, VGF, VPS52, VSNL1, WDR13, ZCWPW1, ZNF292, ZNF467, ZNF75D, ZSWIM7.

Note that some HuGO approved gene symbols, including the classical YAP1-activity markers CCN1 (previously CYR61) and CCN2 (previously CTGF) have not yet been implemented in The Cancer Genome Atlas (TCGA) but have already been adopted in other datasets.

In some embodiments, a panel is encompassed comprising nucleic acid capable of detecting N genes, wherein N is an integer of at least 160 and equal to or less than about 482, and wherein the N genes comprise any 80 to 249 of the following genes of a set of genes (1) (positive effector genes): AASS, ABAT, ACAT2, ADAMTS1, ADM, ADRB2, AMOT, ANXA3, ARHGAP11A, ARHGDIB, AURKB, AVPI1, AXL, AZIN1, B4GALT4, BCAT1, BIRC5, BTG3, C4BPB, CAP2, CAV1, CAVIN1, CCBE1, CCDC80, CCN1, CCN2, CDC25A, CDC6, CDCA3, CDCA4, CDCA5, CDCA8, CDH4, CDK2, CDK6, CDV3, CENPA, CENPI, CENPM, CENPN, CHRNB1, CHST13, CKS2, CLDN1, CLIC3, CNN3, COBL, COL8A1, COTL1, CPA4, CRIM1, CRY1, CTH, CXCL1, CYTH3, DAPK1, DCLRE1B, DDAH1, DHCR7, DHFR, DIAPH3, DKK1, DLL1, DONSON, DUSP14, DUT, EBP, EIF2AK3, EMG1, EPHA2, EPS8L2, ESM1, ETS1, EXO1, EXOSC2, F3, FAHD2A, FAM83D, FANCA, FAT4, FDPS, FEN1, FMR1, FST, FSTL1, FSTL3, GADD45A, GADD45B, GINS1, GPC6, GPR176, GPRC5A, GPRC5B, GRAMD2B, HASPIN, HEG1, HEXB, HPS5, HSPB11, IDI1, IGFBP7, IKBIP, IL6, ITGB2, JDP2, JPH2, KPNA2, KRT8, KRT80, LCA5, LHFPL6, LMCD1, LMNB2, LRP8, LRRFIP2, LSM5, LYPD6, LYRM1, MAD2L1, MAP6D1, MATN2, MATN3, MCM10, MCM2, MCM5, MDC1, METRNL, MICB, MID1, MRPL33, MSRB3, MVD, MXRA7, NCAPD3, NEDD4, NEDD4L, NEK2, NEXN, NFIB, NNMT, NOC3L, NTN4, NUAK1, NUAK2, NUDCD1, NUP107, NUP37, OGFRL1, OLFML3, OLR1, OXCT1, PAK2, PCBD1, PCNA, PDLIM2, PDZD2, PEPD, PHLPP1, PKMYT1, PKP2, PKP4, PLCE1, PLEKHA7, PLK2, PLOD2, PPIH, PRPS1, PRPS2, PRSS23, PSG2, PSG6, PSG7, PSG9, PVR, PXMP2, QDPR, QKI, RAB11FIP1, RAB32, RACGAP1, RBM24, RBMS2, RCN2, RFC4, RND3, RNF144B, ROR1, RPS24, SCD5, SCML1, SDC2, SEC14L1, SGK1, SGMS2, SGTB, SH3RF1, SHCBP1, SKP2, SLC25A23, SLC25A3, SLC38A5, SLC3A2, SLC7A1, SLC7A5, SMPD4, SNAPC1, SNX24, SORT1, SPAG1, SPATA5, STK3, STX11, STXBP6, SUSD2, SUV39H1, SYDE2, TACC3, TAGLN, TEAD1, TEAD4, TENT5B, TGM2, THBS1, TK1, TMEM139, TMEM160, TNFAIP3, TNFRSF12A, TNNC1, TPM1, TPX2, TRIP13, TSPAN2, TTF2, TUBB6, TUFT1, TYMS, UAP1, UBE2C, UGCG, UHRF1, VKORC1L1, WWC1, WWC2, YAP1, ZBED2, ZDHHC18, ZNF488, and ZNF704; and

the N genes comprise any 80 to 233 of the following genes of a set of genes (2) (negative effector genes): AASDH, ABCA1, ABCC5, ABI3BP, ABLIM3, ACADVL, ACOT11, ACOX2, ACSL5, ADAM28, AGL, AGPAT4, ALDH3A2, ANKRD12, ANKRD22, ANKRD29, ANKRD42, ANTXR2, APBB3, ARAP3, ARHGEF2, ASF1A, ATP7A, ATXN1, BCL11B, BHLHE41, BMF, CA2, CASP1, CBR3, CCNG2, CDC42EP4, CDK1, CEBPB, CELSR3, CLCN3, CLDN4, COL6A1, COL6A2, CPE, CRABP2, CROT, CSRNP2, CSTA, CTNNBIP1, CTSB, CTSK, CXXC5, CYP1B1, CYP27C1, DDR1, DEDD2, DHX32, DIAPH2, DSC2, DSG3, DUSP6, DYNC2LI1, ELN, EPS8L3, ERAP2, FAM102A, FAM117B, FAM83B, FAM89B, FERMT1, FKBP2, FOS, FTH1, FXYD3, GDPD1, GOLGA5, GOLPH3L, GPNMB, GPRC5C, GRB10, GSN, HAS3, HBP1, HDAC1, HDHD2, HEY1, HOXA5, IFI44, IGSF3, IGSF9, INTS3, IRAK2, IRF9, IRX5, ITGA2, KCNMA1, KCNMB3, KCNN4, KIFAP3, KLF10, KLF13, KLHL3, KLK11, KRCC1, KRIT1, KRTDAP, LMTK3, LRP10, LTBP4, LXN, LYPD3, MALL, MANSC1, MAPK13, MARCKSL1, MFSD1, MFSD5, MGST2, MGST3, MLLT11, MLPH, MMP13, MSX2, MTMR11, MTMR9, MTSS1, MYO1A, NAGK, NAPEPLD, NCOA3, NFIL3, NPAS2, NRIP1, OAS1, OAS2, OASL, OFD1, OSBPL7, OTUB2, OVOL1, PAG1, PAK1, PCDHB2, PCDHB9, PCGF3, PCMTD2, PERP, PHF21A, PIK3C2B, PIK3R1, PIK3R2, PIK3R3, PIP4P2, PJA2, PKIA, PLA2G4C, PNRC1, PPP1R11, PRRX2, PTPRE, PYGB, RAC2, RALGPS1, RAPGEFL1, RBM23, RBM45, RBM47, RBP1, REEP6, RGL2, RGS17, RHOC, S100A14, SAMD9, SEC14L2, SECISBP2, SH3PXD2A, SH3TC1, SHROOM2, SLC14A1, SLC1A2, SLC30A9, SLC35C1, SLC37A2, SLC39A11, SLFN5, SLITRK6, SLK, SMOC1, SNCG, SP1, SPIRE2, SPRY4, SQSTM1, SRD5A3, SSPN, STMN3, STX1A, TBX3, TCF25, TDO2, TET2, TFF1, TLR3, TMC4, TMC7, TMEM140, TMEM144, TMEM45B, TP53INP1, TP63, TPD52L1, TRAPPC6B, TRIB1, TRIB2, TRIM13, TRIM31, TRIM38, TRIOBP, TRIP11, TSC22D1, TSPAN1, TTC17, TTLL3, TUBB3, UBC, ULK1, VAMP8, VGF, VPS52, VSNL1, WDR13, ZCWPW1, ZNF292, ZNF467, ZNF75D, and ZSWIM7.

In some embodiments, N is an integer of 482, and wherein the 482 genes comprise the following 249 positive effector genes: AASS, ABAT, ACAT2, ADAMTS1, ADM, ADRB2, AMOT, ANXA3, ARHGAP11A, ARHGDIB, AURKB, AVPI1, AXL, AZIN1, B4GALT4, BCAT1, BIRC5, BTG3, C4BPB, CAP2, CAV1, CAVIN1, CCBE1, CCDC80, CCN1, CCN2, CDC25A, CDC6, CDCA3, CDCA4, CDCA5, CDCA8, CDH4, CDK2, CDK6, CDV3, CENPA, CENPI, CENPM, CENPN, CHRNB1, CHST13, CKS2, CLDN1, CLIC3, CNN3, COBL, COL8A1, COTL1, CPA4, CRIM1, CRY1, CTH, CXCL1, CYTH3, DAPK1, DCLRE1B, DDAH1, DHCR7, DHFR, DIAPH3, DKK1, DLL1, DONSON, DUSP14, DUT, EBP, EIF2AK3, EMG1, EPHA2, EPS8L2, ESM1, ETS1, EXO1, EXOSC2, F3, FAHD2A, FAM83D, FANCA, FAT4, FDPS, FEN1, FMR1, FST, FSTL1, FSTL3, GADD45A, GADD45B, GINS1, GPC6, GPR176, GPRC5A, GPRC5B, GRAMD2B, HASPIN, HEG1, HEXB, HPS5, HSPB11, IDI1, IGFBP7, IKBIP, IL6, ITGB2, JDP2, JPH2, KPNA2, KRT8, KRT80, LCA5, LHFPL6, LMCD1, LMNB2, LRP8, LRRFIP2, LSM5, LYPD6, LYRM1, MAD2L1, MAP6D1, MATN2, MATN3, MCM10, MCM2, MCM5, MDC1, METRNL, MICB, MID1, MRPL33, MSRB3, MVD, MXRA7, NCAPD3, NEDD4, NEDD4L, NEK2, NEXN, NFIB, NNMT, NOC3L, NTN4, NUAK1, NUAK2, NUDCD1, NUP107, NUP37, OGFRL1, OLFML3, OLR1, OXCT1, PAK2, PCBD1, PCNA, PDLIM2, PDZD2, PEPD, PHLPP1, PKMYT1, PKP2, PKP4, PLCE1, PLEKHA7, PLK2, PLOD2, PPIH, PRPS1, PRPS2, PRSS23, PSG2, PSG6, PSG7, PSG9, PVR, PXMP2, QDPR, QKI, RAB11FIP1, RAB32, RACGAP1, RBM24, RBMS2, RCN2, RFC4, RND3, RNF144B, ROR1, RPS24, SCD5, SCML1, SDC2, SEC14L1, SGK1, SGMS2, SGTB, SH3RF1, SHCBP1, SKP2, SLC25A23, SLC25A3, SLC38A5, SLC3A2, SLC7A1, SLC7A5, SMPD4, SNAPC1, SNX24, SORT1, SPAG1, SPATA5, STK3, STX11, STXBP6, SUSD2, SUV39H1, SYDE2, TACC3, TAGLN, TEAD1, TEAD4, TENT5B, TGM2, THBS1, TK1, TMEM139, TMEM160, TNFAIP3, TNFRSF12A, TNNC1, TPM1, TPX2, TRIP13, TSPAN2, TTF2, TUBB6, TUFT1, TYMS, UAP1, UBE2C, UGCG, UHRF1, VKORC1L1, WWC1, WWC2, YAP1, ZBED2, ZDHHC18, ZNF488, and ZNF704.

In some embodiments, N is an integer of 482, and wherein the 482 genes comprise the following 233 negative effector genes: AASDH, ABCA1, ABCC5, ABI3BP, ABLIM3, ACADVL, ACOT11, ACOX2, ACSL5, ADAM28, AGL, AGPAT4, ALDH3A2, ANKRD12, ANKRD22, ANKRD29, ANKRD42, ANTXR2, APBB3, ARAP3, ARHGEF2, ASF1A, ATP7A, ATXN1, BCL11B, BHLHE41, BMF, CA2, CASP1, CBR3, CCNG2, CDC42EP4, CDK1, CEBPB, CELSR3, CLCN3, CLDN4, COL6A1, COL6A2, CPE, CRABP2, CROT, CSRNP2, CSTA, CTNNBIP1, CTSB, CTSK, CXXC5, CYP1B1, CYP27C1, DDR1, DEDD2, DHX32, DIAPH2, DSC2, DSG3, DUSP6, DYNC2LI1, ELN, EPS8L3, ERAP2, FAM102A, FAM117B, FAM83B, FAM89B, FERMT1, FKBP2, FOS, FTH1, FXYD3, GDPD1, GOLGA5, GOLPH3L, GPNMB, GPRC5C, GRB10, GSN, HAS3, HBP1, HDAC1, HDHD2, HEY1, HOXA5, IFI44, IGSF3, IGSF9, INTS3, IRAK2, IRF9, IRX5, ITGA2, KCNMA1, KCNMB3, KCNN4, KIFAP3, KLF10, KLF13, KLHL3, KLK11, KRCC1, KRIT1, KRTDAP, LMTK3, LRP10, LTBP4, LXN, LYPD3, MALL, MANSC1, MAPK13, MARCKSL1, MFSD1, MFSD5, MGST2, MGST3, MLLT11, MLPH, MMP13, MSX2, MTMR11, MTMR9, MTSS1, MYO1A, NAGK, NAPEPLD, NCOA3, NFIL3, NPAS2, NRIP1, OAS1, OAS2, OASL, OFD1, OSBPL7, OTUB2, OVOL1, PAG1, PAK1, PCDHB2, PCDHB9, PCGF3, PCMTD2, PERP, PHF21A, PIK3C2B, PIK3R1, PIK3R2, PIK3R3, PIP4P2, PJA2, PKIA, PLA2G4C, PNRC1, PPP1R11, PRRX2, PTPRE, PYGB, RAC2, RALGPS1, RAPGEFL1, RBM23, RBM45, RBM47, RBP1, REEP6, RGL2, RGS17, RHOC, S100A14, SAMD9, SEC14L2, SECISBP2, SH3PXD2A, SH3TC1, SHROOM2, SLC14A1, SLC1A2, SLC30A9, SLC35C1, SLC37A2, SLC39A11, SLFN5, SLITRK6, SLK, SMOC1, SNCG, SP1, SPIRE2, SPRY4, SQSTM1, SRD5A3, SSPN, STMN3, STX1A, TBX3, TCF25, TDO2, TET2, TFF1, TLR3, TMC4, TMC7, TMEM140, TMEM144, TMEM45B, TP53INP1, TP63, TPD52L1, TRAPPC6B, TRIB1, TRIB2, TRIM13, TRIM31, TRIM38, TRIOBP, TRIP11, TSC22D1, TSPAN1, TTC17, TTLL3, TUBB3, UBC, ULK1, VAMP8, VGF, VPS52, VSNL1, WDR13, ZCWPW1, ZNF292, ZNF467, ZNF75D, and ZSWIM7.

In some embodiments, a panel of the disclosure may be used for measuring TEAD-dependent transcription levels of genes in a sample from a subject with or suspected of having cancer comprising: isolating a biological sample from the subject and detecting the expression level of each of the genes of the panel.

In some embodiments, a panel of the disclosure may be used for characterizing a subject as having a TEAD-active or TEAD-inactive cancer comprising: isolating a biological sample from a subject with or suspected of having cancer and detecting the expression level of each of the genes of the panel, wherein the subject has a TEAD-active cancer if the dgR or deR is greater than about 0.055.

In some embodiments, a panel of the disclosure may be used for predicting whether a subject will respond positively to a TEAD-pathway inhibitor comprising: isolating a biological sample from a subject with or suspected of having cancer and detecting the expression level of each of the genes of the panel, wherein the subject is likely to respond positively to a TEAD-pathway inhibitor if the dgR or deR is greater than about 0.055.

In some embodiments, the present disclosure relates to a transcriptional signature (“TEAD-signature”) obtained by measure of expression levels of genes of a set of genes comprising any of 220 to 249 of genes of a set of genes (1) (the positive effector genes) and any of 210 to 233 of genes of a set of genes (2) (the negative effector genes).

A transcriptional signature (“TEAD-signature”) may be used for determining the TEAD-activity of a cancer. The transcriptional signatures may be useful in a variety of applications, including, predicting the likelihood that a subject will respond to a TEAD-pathway inhibitor treatment, selecting patients for clinical trials, assessing efficacy of TEAD-pathway inhibitor molecules, and prognosing survival, response to, and benefit from anti-TEAD pathway treatments.

In some embodiments, the present disclosure relates to a use of a transcriptional signature for measuring the TEAD-activity of a cancer in a subject in need thereof, the transcriptional signature being obtained by measure of expression levels of genes of a set of genes in a biological sample of a tumor said cancer, the set of genes comprising any of 220 to 249 of genes of a set of genes (1) (the positive effector genes) and any of 210 to 233 of genes of a set of genes (2) (the negative effector genes).

In some embodiments, the present disclosure relates to a use of a transcriptional signature for characterizing the TEAD-activity of a cancer in a subject in need thereof, the transcriptional signature being obtained by measure of expression levels of genes of a set of genes in a biological sample of a tumor said cancer, the set of genes comprising any of 220 to 249 of genes of a set of genes (1) (the positive effector genes) and any of 210 to 233 of genes of a set of genes (2) (the negative effector genes).

A transcriptional signature (“TEAD-signature”) according to the disclosure may be used to determine a TEAD score.

In some embodiments, a TEAD score is calculated according to either of the following methods:

### Method 1: Calculate the dgR Score as Follows:

1. For each gene tested (e.g., a full transcriptome), convert the gene's expression level into a fractional rank, which according to the definitions herein, is the rank of the gene (i.e., where the expression level falls within the list of all the genes tested, where the gene with the lowest expression level is given the rank 1, the next highest level of expression a rank 2, and so on until the highest expressing gene is given the highest rank; equal expression levels are given the average rank, e.g. two genes with 0 expression are given the rank 1.5) divided by the number of genes tested.
2. Isolate the genes of the set of genes (1) (positive effectors genes) and compute their mean fractional rank (MFR-positive)
3. Isolate the genes of the set of genes (2) (negative effectors genes) and compute their mean fractional rank (MFR-negative)
4. Compute the dgR score as MFR-positive-MFR-negative

### Method 2: Calculate the deR Score as Follows:

1. Isolate the genes of the set of genes (1) and (2) (positive and negative effectors genes) in the TEAD-500 panel.
2. For each gene tested, convert the gene's expression level into a fractional rank, which according to the definitions herein, is the rank of the gene (i.e., where the expression level falls within the list of all the genes tested, where the gene with the lowest expression level is given the rank 1, the next highest level of expression a rank 2, and so on until the highest expressing gene is given the highest rank; equal expression levels are given the average rank, e.g. two genes with 0 expression are given the rank 1.5) divided by the number of genes tested.
3. Isolate the positive effectors and compute their mean fractional rank (MFR-positive)
4. Isolate the negative effectors and compute their mean fractional rank (MFR-negative)
5. Compute the deR score as MFR-positive-MFR-negative

The dgR and deR scores can be used to determine if a subject has a TEAD-active or TEAD-inactive cancer. If the dgR or deR score is greater than about 0.055, then the cancer is TEAD-active. If the dgR or deR score is less than or equal to about 0.055, then the cancer is TEAD-inactive.

In some embodiments, the set of genes of a transcriptional signature (“TEAD-signature”) may comprise from 430 to 482 gene.

In some embodiments, the set of genes of a transcriptional signature (“TEAD-signature”) may comprise 430, 431, 432, 433, 434, 436, 437, or 482 genes.

In some embodiments, the cancer may be a cancer comprising a solid tumor.

In some embodiments, the solid tumor may be in the lung, colon, ovary, cervix, uterus, peritoneum, testicles, penis, tongue, lymph node, pancreas bone, breast, prostate, soft tissue, connective tissue, kidney, liver, brain, thyroid, or skin.

In some embodiments, the cancer comprising a solid tumor may be selected from the group consisting of adrenocortical carcinoma, bladder urothelial carcinoma, breast invasive carcinoma, cervical squamous cell carcinoma and endocervical adenocarcinoma, cholangiocarcinoma, consensus molecular subtypes 1 of colorectal cancer, consensus molecular subtypes 2 of colorectal cancer, consensus molecular subtypes 3 of colorectal cancer, consensus molecular subtypes 4 of colorectal cancer, colon adenocarcinoma, lymphoid neoplasm diffuse large b-cell lymphoma, esophageal carcinoma, glioblastoma multiforme, head and neck squamous cell carcinoma, kidney renal clear cell carcinoma, kidney renal papillary cell carcinoma, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, and uterine carcinosarcoma.

In some embodiments, a use of a transcriptional signature (“TEAD signature”) may comprise:
a) measuring, in a transcriptome obtained from said biological sample, the expression levels of genes,
b) for each gene of said set of genes, converting the gene's expression level obtained at step a) into a fractional rank by dividing the rank of said gene by the number of genes from said transcriptome,
c) isolating from the fractional ranks obtained at step b) the fractional rank obtained for the genes of the set of genes (1) and computing their mean fractional rank (MFR-positive),
d) isolating from the fractional ranks obtained at step b) the fractional rank obtained for the genes of the set of genes (2) and computing their mean fractional rank (MFR-negative), and
e) computing a dgR score as MFR-positive-MFR-negative;
or
a) measuring, in said biological sample, the expression level of each gene of said set of genes,
b) for each gene of said set of genes, converting the gene's expression level obtained at step a) into a fractional rank by dividing the rank of said gene by the number of genes from said set of genes,
c) isolating from the fractional ranks obtained at step b) the fractional rank obtained for the genes of the set of genes (1) and computing their mean fractional rank (MFR-positive),
d) isolating from the fractional ranks obtained at step b) the fractional rank obtained for the genes of the set of genes (2) and computing their mean fractional rank (MFR-negative), and
e) computing a deR score as MFR-positive-MFR-negative;
wherein when the dgR or the deR score is greater than about 0.055, then the cancer is TEAD-active, and when the dgR or deR score is less than or equal to about 0.055, then the cancer is TEAD-inactive;

In some embodiments, a transcriptional signature may be reduced to a shorter signature. Those shorter signature are named thereafter “short TEAD signature”.

A “short TEAD signature” is obtained by measure of expression levels of genes of a reduced number of genes and is specific to a given cancer.

In some embodiments, the disclosure relates to a use of a transcriptional signature for measuring the TEAD-activity of a cancer in a subject in need thereof, the transcriptional signature being obtained by measure of expression levels of genes of a set of genes in a biological sample of a tumor of said cancer, the set of genes being selected from the group consisting of sets a) to mmmm) of Table 1 below (“short TEAD signature”).

In some embodiments, the disclosure relates to a use of a transcriptional signature for characterizing the TEAD-activity of a cancer in a subject in need thereof, the transcriptional signature being obtained by measure of expression levels of genes of a set of genes in a biological sample of a tumor of said cancer, the set of genes being selected from the group consisting of sets a) to mmmm) of Table 1 below (“short TEAD signature”).

In some embodiments, the disclosure relates to a panel of nucleic acid biomarkers comprising nucleic acid capable of detecting expression levels of each gene within any one of the following sets of genes a) through mmmm):

TABLE 1
Sets of genes (“short TEAD signature”) for specific cancer indications

| # | Sets of genes for specific cancer indications |
|---|---|
| a) | a) AASS, ARHGDIB, ASF1A, CA2, CBR3, CCDC80, CLIC3, COL8A1, CPA4, CPE, |
| | CYP27C1, DSG3, ESM1, F3, FXYD3, GADD45A, GSN, HOXA5, IGSF9, IRAK2, IRF9, |
| | KLF10, KLF13, KLHL3, KRT8, LCA5, LYRM1, MICB, MTMR9, OGFRL1, PIK3C2B, PKP2, |
| | PNRC1, RBP1, SEC14L1, SGTB, SH3TC1, SORT1, STX11, STX1A, SYDE2, TEAD4, |
| | TFF1, TMC7, TMEM139, TP63, TSPAN1, TUBB6, ULK1, |
| b) | b) ULK1, TMC7, LCA5, ZSWIM7, PNRC1, |
| c) | c) ACOX2, ADAMTS1, AMOT, AURKB, AVPI1, CA2, CCNG2, CDH4, DONSON, DSC2, |
| | EPS8L2, FXYD3, GPRC5C, HDAC1, HDHD2, HEY1, IGSF3, KCNMA1, KLHL3, KRCC1, |
| | KRT8, KRT80, LCA5, LTBP4, MAPK13, MGST2, MLPH, NFIL3, NRIP1, NUAK2, PIK3R3, |
| | PKP2, PNRC1, PSG2, RAPGEFL1, RBP1, RGL2, RND3, RNF144B, S100A14, SEC14L1, |
| | SH3RF1, SHCBP1, SLC1A2, SLC25A3, STX1A, TTF2, TUFT1, TYMS, VAMP8, ZNF75D, |
| d) | d) ABAT, ACAT2, ADM, ADRB2, ANXA3, ARAP3, BCAT1, CAV1, CCN1, CCN2, |
| | CDC42EP4, CDK1, COTL1, CPA4, CSRNP2, CTSK, CXCL1, CXXC5, DIAPH3, |
| | DONSON, EXO1, FAHD2A, FOS, FSTL1, FTH1, GOLPH3L, GPR176, GPRC5B, IRF9, |
| | KIFAP3, KRT80, KRTDAP, LMCD1, LMTK3, MALL, MDC1, MGST2, MLPH, MMP13, |
| | NUP107, OLFML3, PAK1, PAK2, PCDHB9, PDLIM2, PERP, PIK3R3, PIP4P2, PKP4, |
| | PTPRE, RAPGEFL1, RBMS2, RGL2, RHOC, SHCBP1, SLC38A5, SLC3A2, SMOC1, |
| | SMPD4, SNAPC1, SNCG, SPATA5, STXBP6, THBS1, TNNC1, TPD52L1, TPM1, |
| | ZCWPW1, ZNF488, |
| e) | e) AASDH, ABCC5, ADM, BCL11B, BMF, C4BPB, CAVIN1, CCBE1, CCDC80, CDCA8, |
| | CDV3, COTL1, CSRNP2, CTSK, DAPK1, DIAPH3, ESM1, EXO1, GPC6, HEY1, IRF9, |
| | JDP2, KIFAP3, KRT80, LXN, MGST2, MXRA7, NCAPD3, NEDD4, NEK2, NTN4, |
| | PIK3C2B, PIK3R3, PKP4, PPIH, PSG2, RAPGEFL1, RBM24, RGL2, RNF144B, |
| | SLC25A3, SLC35C1, SLC38A5, SLC39A11, SNCG, STX11, SYDE2, TDO2, TENT5B, |
| | TET2, TPM1, VKORC1L1, ZCWPW1, |
| f) | f) DIAPH3, MGST2, RGL2, KIFAP3, IRF9, CTSK, SNCG, ACAT2, JDP2, RBM24, COTL1, |
| | PKP4, C4BPB, CDCA8, MXRA7, SLC38A5, SLC35C1, CDV3, RNF144B, STX11, ADM, |
| | PSG2, GPC6, BCL11B, VKORC1L1, RAPGEFL1, SLC25A3, ABCC5, TDO2, SLC39A11, |
| | CCDC80, CAVIN1, EXO1, NEK2, TET2, LXN, CCBE1, ZCWPW1, PPIH, PIK3R3, |
| | AASDH, KRT80, NEDD4, NCAPD3, |
| g) | g) ABAT, ARAP3, ARHGDIB, BCL11B, BMF, CAV1, CCN2, CDC25A, CDK2, CDK6, |
| | CLDN4, CLIC3, COL8A1, DDR1, DHFR, DHX32, DKK1, DUSP6, EXOSC2, FAHD2A, |
| | FAM102A, FDPS, FST, GADD45A, GINS1, GSN, JPH2, KLF13, KRCC1, LMCD1, LRP10, |
| | LYPD6, MAP6D1, MCM2, MCM5, MSX2, MTMR11, MTMR9, NCOA3, NEXN, OAS1, |
| | OLFML3, PCMTD2, PIK3C2B, PIP4P2, PKIA, PLEKHA7, PSG6, RAB11FIP1, RBP1, |
| | ROR1, SCD5, SECISBP2, SKP2, SNAPC1, TRIOBP, TSPAN1, TUFT1, WDR13, ZBED2, |
| h) | h) JPH2, SKP2, DUSP6, OLFML3, TSPAN1, CDK2, NEXN, DDR1, OAS1, GPC6, DKK1, |
| | GADD45A, KRCC1, MALL, GSN, FST, ARAP3, MTMR11, EXOSC2, CAV1, PSG6, |
| | SECISBP2, NCOA3, |
| i) | i) ADM, ATXN1, AURKB, BHLHE41, BMF, CDH4, CELSR3, CTNNBIP1, CYTH3, DHCR7, |
| | EBP, GINS1, GPRC5C, GSN, HPS5, JPH2, LHFPL6, LRP8, LYRM1, MRPL33, |
| | NAPEPLD, NEK2, OAS1, PAG1, PCDHB2, PCGF3, QDPR, RAPGEFL1, RBMS2, ROR1, |
| | S100A14, SEC14L1, SLC37A2, SUV39H1, TEAD4, TP53INP1, |
| j) | j) ADAM28, CRABP2, ELN, ERAP2, EXOSC2, FAM83D, ITGB2, MARCKSL1, MFSD1, |
| | MGST2, MLLT11, NUAK2, PLA2G4C, RND3, SHCBP1, SLK, SMOC1, SMPD4, TMC7, |
| | TRIM31, ULK1, VPS52, WWC2, ZNF292, ZNF467, |
| k) | k) VPS52, SHCBP1, EXOSC2, MGST2, |
| l) | l) AGL, ATXN1, CCDC80, CDCA8, CENPI, CLDN4, CRY1, EPS8L3, EXO1, FXYD3, |
| | GPC6, GPRC5C, IGSF9, IRF9, ITGA2, KLK11, KRT8, LHFPL6, MAP6D1, MYO1A, |
| | NCAPD3, NTN4, PAG1, PCDHB2, PLEKHA7, PSG9, PTPRE, PYGB, RAPGEFL1, |
| | RBMS2, RBP1, RGS17, RHOC, RND3, SLC14A1, SLC35C1, SLC38A5, SSPN, TFF1, |
| | TK1, TMC4, TMEM45B, TRIM38, TRIP13, UGCG, VPS52, WWC1, |
| m) | m) AASDH, ACADVL, BCAT1, BMF, CAP2, CCBE1, CDH4, DUSP14, EMG1, EPHA2, |
| | ESM1, HEXB, HOXA5, IGSF9, MCM5, PDLIM2, PEPD, PKP4, PNRC1, RCN2, S100A14, |
| | SDC2, SLC30A9, SSPN, TFF1, TMEM140, TMEM45B, UBC, ZDHHC18, |
| n) | n) TMEM45B, TFF1, SSPN, S100A14, EPHA2, SLC30A9, HOXA5, BCAT1, |
| o) | o) AURKB, CENPI, CENPM, CLDN4, DDR1, DLL1, EPS8L3, FDPS, FEN1, KRT8, |
| | PRPS2, RAB32, SLC1A2, TSPAN2, |
| p) | p) CLDN4, EPS8L3, CENPI, DDR1, AURKB, CENPM, |
| q) | q) ADRB2, BHLHE41, CAVIN1, CBR3, CCN2, CHST13, CTSB, CXXC5, DDR1, GPRC5A, |
| | GRAMD2B, IFI44, IGFBP7, IGSF9, IRF9, ITGA2, KLF10, LTBP4, MATN3, MLLT11, |
| | MSX2, NPAS2, PCDHB2, PIK3R1, PRPS2, ROR1, RPS24, S100A14, SHCBP1, |
| | SLC30A9, SLC35C1, SLC3A2, SLK, SNCG, TEAD1, TMC7, TSPAN1, TTLL3, WWC1, |
| q1) | q1) DDR1, |
| r) | r) ATXN1, CKS2, CLCN3, COBL, DSC2, EIF2AK3, INTS3, IRAK2, NFIB, OTUB2, RBM23, |
| | RBM24, RHOC, SECISBP2, SLC7A1, SLFN5, SUV39H1, THBS1, TNFRSF12A, |
| | TPD52L1, TSPAN1, TSPAN2, TTLL3, UHRF1, WDR13, |
| s) | s) THBS1, RBM24, TNFRSF12A, IRAK2, SLC7A1, OTUB2, TSPAN2, COBL, |
| t) | t) ANKRD42, ANXA3, AXL, BCAT1, BCL11B, CASP1, CDC25A, CDH4, CDV3, CENPN, |
| | CHST13, CRIM1, CXCL1, CXXC5, CYTH3, DUT, ELN, ERAP2, ESM1, HASPIN, IRX5, |
| | MCM2, NEDD4L, NEXN, NFIL3, NRIP1, NUAK2, OLR1, PKP2, PLA2G4C, PLK2, PSG6, |
| | RBP1, REEP6, RGS17, SGTB, SLC25A3, SLFN5, SPATA5, SUV39H1, TMEM144, |
| | TPD52L1, TRAPPC6B, TSPAN1, TYMS, UHRF1, VGF, VSNL1, WWC1, YAP1, ZNF704, |
| u) | u) CHST13, PLK2, CENPN, ANKRD42, AXL, PSG6, CDC25A, IRX5, NRIP1, OLR1, |
| | TYMS, CTH, HASPIN, BCL11B, NEXN, ELN, CXCL1, TRAPPC6B, SLFN5, PLA2G4C, |
| | CASP1, NUAK2, NEDD4L, YAP1, ANXA3, TSPAN1, MLLT11, SLC25A3, SGTB, CENPM, |
| | CRIM1, ESM1, CDH4, PKP2, UHRF1, |
| v) | v) AASS, ACSL5, AMOT, ARHGEF2, BCAT1, BHLHE41, CA2, CAV1, CCN2, CCNG2, |
| | CDCA3, CELSR3, CENPI, CLCN3, CLDN1, COTL1, CYP27C1, DCLRE1B, ERAP2, |
| | FAM102A, FAM89B, FDPS, GADD45B, GINS1, GPRC5B, HASPIN, HEXB, IFI44, IGSF3, |
| | IRAK2, KRCC1, KRT8, LMCD1, MCM10, MGST2, MLLT11, MTMR11, NFIL3, NUAK1, |
| | OAS1, OFD1, PAK1, PCDHB9, PCGF3, PHLPP1, PIK3C2B, PIP4P2, PKP2, PLK2, |
| | PSG6, RAB32, RACGAP1, RALGPS1, RBM45, ROR1, SHROOM2, SP1, SRD5A3, |
| | STK3, TACC3, TMC4, TRIM31, TUBB6, VPS52, VSNL1, |
| w) | w) CENPI, IFI44, SP1, AMOT, ROR1, BCAT1, IRAK2, KRT8, TRIM31, RBM45, VPS52, |
| | PHLPP1, TMC4, BHLHE41, HASPIN, TRIOBP, MCM10, CAV1, CCN2, CLCN3, |
| | CYP27C1, MTMR11, VSNL1, IGSF3, SHROOM2, CLDN1, AASS, DCLRE1B, LMCD1, |
| | GADD45B, NFIL3, TACC3, CA2, PCDHB9, ACSL5, NUAK1, PIP4P2, PCGF3, FAM89B, |
| | PKP2, PAK1, TUBB6, FDPS, |
| x) | x) ACAT2, ANKRD22, ANKRD29, ANTXR2, ANXA3, B4GALT4, BHLHE41, CA2, CAV1, |
| | CCBE1, CDCA3, CENPN, CNN3, CRIM1, CTNNBIP1, CYTH3, DDAH1, DSC2, EIF2AK3, |
| | ERAP2, F3, FAM117B, FEN1, GOLGA5, GSN, HSPB11, IKBIP, LRRFIP2, LSM5, |
| | MANSC1, METRNL, MGST2, NEDD4, NUAK1, NUAK2, PCBD1, PEPD, PHF21A, |
| | PHLPP1, PIK3R1, PLCE1, PSG2, RFC4, SCD5, SGTB, SLC38A5, SPRY4, SQSTM1, |
| | STX1A, SUSD2, TMC4, TSPAN1, ZNF704, |
| y) | y) ACSL5, AMOT, ANXA3, ARHGAP11A, ARHGEF2, AXL, CASP1, CDC25A, CELSR3, |
| | CENPN, COL8A1, CXXC5, DONSON, FAHD2A, FAM83B, FERMT1, FSTL1, GPC6, |
| | GPRC5C, HASPIN, HBP1, HDAC1, IFI44, IGFBP7, KRTDAP, LRP8, MANSC1, MAPK13, |
| | MATN2, MICB, MID1, MXRA7, NEDD4, NEK2, NRIP1, NUP107, PAG1, PIK3C2B, |
| | PIK3R3, PIP4P2, QDPR, RACGAP1, RBP1, RCN2, RGS17, SECISBP2, SLC25A3, |
| | SLC39A11, SRD5A3, ULK1, VKORC1L1, ZNF488, |
| z) | z) HASPIN, MATN2, CXXC5, CELSR3, RBP1, |
| aa) | aa) ACAT2, APBB3, ATXN1, AURKB, BCAT1, BIRC5, CDC42EP4, CDC6, CDCA3, |
| | CDCA8, CDK1, CENPA, CLDN4, CRIM1, CYTH3, DKK1, EPS8L2, F3, FOS, GPC6, |
| | GPRC5C, HAS3, KRT8, LSM5, MGST2, MTMR11, MXRA7, NEDD4L, OLR1, PHF21A, |
| | QKI, RBM23, RBM24, RNF144B, SDC2, SKP2, SLC38A5, SNAPC1, SSPN, TFF1, |
| | TMEM139, TMEM140, TPX2, TSPAN1, UBE2C, ZCWPW1, |
| bb) | bb) ABLIM3, AURKB, BCAT1, BHLHE41, CDCA3, CDCA8, CDH4, CEBPB, COL6A1, |
| | COL6A2, CRABP2, CSTA, CTSK, CXCL1, DAPK1, DDR1, ESM1, EXO1, FAM89B, |
| | FANCA, FSTL1, FXYD3, GINS1, GOLPH3L, ITGA2, KCNMA1, LRP8, LRRFIP2, LTBP4, |
| | MAPK13, MTSS1, MYO1A, NAPEPLD, OLFML3, PAK1, PERP, PLOD2, PSG2, RBM23, |
| | RFC4, SECISBP2, SHCBP1, SLC25A23, SLC25A3, SLC37A2, SNCG, SPATA5, |
| | TENT5B, TRIM31, TTF2, VAMP8, WDR13, |
| cc) | cc) CENPA, WDR13, PERP, MTSS1, BHLHE41, DAPK1, CRABP2, GOLPH3L, ABLIM3, |
| | RBM23, COL6A1, MYO1A, CDCA8, COL6A2, MAPK13, CDH4, BCAT1, PLOD2, |
| | SPATA5, RFC4, NAPEPLD, PAK1, SLC25A23, OLFML3, TTF2, PIK3R2, CDCA3, |
| | KCNMA1, GRB10, SGMS2, |
| dd) | dd) ADAMTS1, ARAP3, BTG3, CCN2, CDCA8, CLCN3, CSRNP2, CTSB, EBP, ERAP2, |
| | ESM1, EXO1, FAM83B, FAM83D, FAM89B, FAT4, GOLGA5, GPRC5B, HAS3, IRAK2, |
| | IRF9, IRX5, KCNMA1, KCNMB3, KIFAP3, LMCD1, LMNB2, MCM10, MICB, MSX2, |
| | NAPEPLD, NEDD4L, NEK2, NEXN, PCMTD2, PHLPP1, PIK3C2B, PPIH, PRPS2, PYGB, |
| | RBM24, SCML1, SEC14L2, SGMS2, SKP2, SLC7A5, SNAPC1, SNCG, SNX24, |
| | SQSTM1, SUSD2, TACC3, TBX3, TENT5B, TFF1, TNFAIP3, TP53INP1, TPX2, |
| | TRAPPC6B, VGF, WDR13, WWC1, ZNF467, |
| ee) | ee) EXO1, FAM83B, SUSD2, CTSB, NEXN, TNFAIP3, PIK3C2B, FAM83D, TPX2, TBX3, |
| | PPIH, NEK2, MICB, CLCN3, PHLPP1, ADAMTS1, LMCD1, GOLGA5, WDR13, SCML1, |
| | SNCG, NEDD4L, SEC14L2, BTG3, CCN2, SQSTM1, IRF9, FAT4, TP53INP1, IRAK2, |
| | RBM24, LMNB2, CSRNP2, TRAPPC6B, ZNF467, SKP2, SGMS2, TENT5B, PCMTD2, |
| | SNX24, FAM89B, HAS3, KCNMB3, |
| ff) | ff) ACADVL, CAV1, CBR3, CDCA8, CEBPB, CPA4, DONSON, EPS8L2, FAM89B, |
| | FANCA, FAT4, GPRC5B, GSN, HASPIN, IGFBP7, KRIT1, MAD2L1, MFSD1, MICB, |
| | MLLT11, NAPEPLD, NCAPD3, NEDD4L, OLR1, PEPD, PIK3R2, PLEKHA7, QDPR, |
| | SH3PXD2A, SKP2, SLC7A5, SMPD4, TMEM140, TMEM160, TRIM38, UHRF1, |
| | ZDHHC18, ZNF467, |
| gg) | gg) AASS, ABLIM3, ACADVL, ARHGAP11A, B4GALT4, CAP2, CBR3, CCDC80, CDCA5, |
| | CDV3, COBL, CPE, DAPK1, DHCR7, DUT, ERAP2, EXO1, FAT4, KLK11, KRIT1, |
| | LMCD1, LYPD6, MCM10, MLLT11, MMP13, NTN4, PAK1, PNRC1, QKI, RHOC, SLC3A2, |
| | SLK, SUSD2, SUV39H1, TACC3, TLR3, UBE2C, VKORC1L1, ZNF292, |
| hh) | hh) MCM10, CDCA5, MLLT11, |
| ii) | ii) ABAT, ABI3BP, ACADVL, ARHGAP11A, ATXN1, BCL11B, BTG3, CCBE1, CCN2, |
| | CDC25A, CDC42EP4, CDCA4, COL6A1, COL8A1, CTH, CYTH3, DDR1, DEDD2, |
| | DIAPH2, DLL1, DONSON, DUSP6, DYNC2LI1, ELN, EMG1, ERAP2, ESM1, EXOSC2, |
| | FANCA, FAT4, FEN1, FOS, FST, FSTL3, GINS1, GSN, HBP1, HDAC1, HEY1, IGSF3, |
| | IGSF9, IRX5, KCNMB3, KPNA2, KRCC1, KRIT1, KRTDAP, LHFPL6, LMTK3, MAD2L1, |
| | MFSD1, MLLT11, MYO1A, NCAPD3, NEDD4L, NFIB, NPAS2, PAG1, PCDHB2, |
| | PIK3C2B, PIK3R2, PLCE1, PRPS1, PRPS2, PTPRE, PYGB, QDPR, RACGAP1, RFC4, |
| | RHOC, RNF144B, SH3PXD2A, SHCBP1, SLC25A3, SLC35C1, SLC39A11, SLC3A2, |
| | SMPD4, STX11, TAGLN, TLR3, TMEM144, TP63, TRAPPC6B, TRIB2, TRIM31, |
| | TRIOBP, TRIP13, TSC22D1, VAMP8, VGF, ZSWIM7, |
| jj) | jj) TRIP13, SLC35C1, DYNC2LI1, TP63, MLLT11, RNF144B, MAD2L1, GSN, TAGLN, |
| | CCBE1, FST, DEDD2, DUSP6, CYTH3, TRIM31, GINS1, DCLRE1B, KRCC1, |
| | CDC42EP4, COL6A1, KRTDAP, MFSD1, DONSON, FOS, AASS, SMPD4, TRAPPC6B, |
| | PIK3R2, ESM1, NFIB, TSC22D1, CTH, RACGAP1, NCAPD3, ACADVL, PHF21A, PAG1, |
| kk) | kk) ADRB2, ARHGEF2, DUSP6, EXO1, GPRC5B, GSN, IL6, MAD2L1, NAGK, OLR1, |
| | PERP, PLK2, PSG7, PXMP2, RBMS2, RBP1, SCML1, SH3RF1, TP53INP1, TPD52L1, |
| | TSPAN2, ZCWPW1, |
| ll) | ll) MAD2L1, IL6, PSG7, ZCWPW1, NAGK, DUSP6, OLR1, PLK2, EXO1, PERP, |
| | TP53INP1, ADRB2, SCML1, ARHGEF2, |
| mm) | mm) ACOT11, ADAM28, ADM, ANKRD42, ANXA3, ARHGEF2, ATP7A, AXL, BHLHE41, |
| | CASP1, CAV1, CCBE1, CDC25A, CELSR3, CLIC3, COTL1, CYP1B1, CYTH3, DHFR, |
| | DHX32, DIAPH3, DSG3, DUSP14, DUT, DYNC2LI1, EXO1, FEN1, FST, GPR176, |
| | GRB10, HBP1, HOXA5, IGFBP7, IGSF3, INTS3, KCNMB3, KIFAP3, KLF13, KRTDAP, |
| | MGST2, MLPH, NEDD4, NEK2, NUAK2, PAK1, PRSS23, RAB32, RBM47, RBP1, |
| | SCML1, SEC14L1, SGK1, SH3PXD2A, SLC37A2, SNCG, SP1, STXBP6, TACC3, |
| | TMEM144, TMEM45B, TTF2, VGF, WDR13, WWC2, ZNF488, |
| nn) | nn) ACAT2, ACOT11, AGL, ANTXR2, AXL, B4GALT4, CAVIN1, CCN2, CCNG2, |
| | CDC25A, CDH4, CENPA, CHRNB1, COBL, CPA4, CTNNBIP1, DDR1, DHFR, DONSON, |
| | EBP, EPS8L2, EXO1, FAHD2A, FAM83D, FEN1, GOLGA5, GRAMD2B, IFI44, INTS3, |
| | IRAK2, IRX5, KCNMB3, KRCC1, KRT8, KRTDAP, MATN2, MRPL33, NAGK, NEXN, |
| | NUP37, OAS2, OTUB2, PAK2, PERP, PKIA, PKMYT1, PLK2, PNRC1, PRRX2, RBM45, |
| | RBP1, REEP6, SLITRK6, SMPD4, SNX24, STXBP6, TK1, TMC4, TMC7, TRIM13, |
| | TSPAN1, UAP1, VPS52, WWC1, YAP1, |
| oo) | oo) ACOT11, EXO1, AXL, CHRNB1, KRTDAP, CTNNBIP1, PSG2, TNFRSF12A, CPA4, |
| | PDLIM2, FEN1, VPS52, FAM83D, MDC1, EBP, NAPEPLD, TK1, EPS8L2, NEXN, |
| | CENPA, COBL, TNFAIP3, DHFR, ANTXR2, NUP37, |
| pp) | pp) ATP7A, ATXN1, CCBE1, CCN2, CDCA4, CDK6, COL8A1, CRABP2, CRIM1, CSTA, |
| | CTH, CTSK, DCLRE1B, DHX32, EPS8L3, ERAP2, F3, FAT4, GPR176, HBP1, HDHD2, |
| | HEY1, IGSF3, IGSF9, IKBIP, JPH2, KCNMA1, KCNMB3, KLHL3, KRIT1, LRP10, |
| | LRRFIP2, LYRM1, MAPK13, METRNL, MSX2, NCOA3, NEXN, NOC3L, NUP37, OAS1, |
| | OLFML3, PAK2, PDLIM2, PEPD, PJA2, PKP4, PLA2G4C, PLK2, PSG6, PVR, RALGPS1, |
| | RBM23, RBM24, RFC4, RGS17, RHOC, RNF144B, ROR1, S100A14, SMPD4, SSPN, |
| | TDO2, TLR3, TMC4, TMC7, TMEM139, TMEM45B, TP63, TRIM38, TRIP11, TSPAN1, |
| | TSPAN2, TTLL3, ZDHHC18, ZNF488, |
| qq) | qq) CDC25A, CCBE1, TSPAN1, TMC4, ZNF488, CTSK, CCN2, HEY1, TMC7, LYRM1, |
| | ATXN1, FAT4, DHX32, IGSF3, PDLIM2, KRIT1, DCLRE1B, CRIM1, PVR, NCOA3, |
| | OLFML3, GSN, PLK2, RHOC, GPR176, KLHL3, |
| rr) | rr) AGL, AGPAT4, ATXN1, AURKB, BMF, CBR3, CCNG2, CDC6, CDK6, CDV3, CENPN, |
| | COBL, CRABP2, CTH, DDR1, DEDD2, DIAPH3, DKK1, ETS1, EXO1, GADD45B, |
| | GPRC5C, GRB10, JPH2, KRIT1, LYPD3, MALL, MATN3, MCM10, MLPH, MVD, NEXN, |
| | NFIL3, OASL, OXCT1, PCDHB9, PCGF3, PERP, PKIA, PLOD2, PRPS1, RAPGEFL1, |
| | RBM45, RBP1, SPIRE2, STMN3, SUSD2, TET2, TRIB1, TRIP13, VAMP8, |
| ss) | ss) AASS, ABAT, ACSL5, AGPAT4, ANKRD22, ARHGEF2, AXL, BIRC5, C4BPB, CAV1, |
| | CAVIN1, CCDC80, CCN1, CENPA, CHRNB1, CHST13, CLDN4, CTSB, DDAH1, DHCR7, |
| | EPS8L2, FMR1, FST, GPNMB, HEY1, IGSF9, IKBIP, ITGA2, ITGB2, KRIT1, LCA5, |
| | LMCD1, LXN, MALL, MAPK13, MFSD5, MSRB3, NNMT, OVOL1, PCDHB2, PCDHB9, |
| | PERP, PKMYT1, PLA2G4C, PLEKHA7, PLOD2, PRPS2, PVR, PYGB, RAB32, RAC2, |
| | SGTB, SHCBP1, SMOC1, SPATA5, SPIRE2, SPRY4, STMN3, TMEM144, TNNC1, |
| | TP63, TRIM31, TTF2, TYMS, UGCG, ZCWPW1, ZNF488, |
| tt) | tt) GPNMB, NNMT, CTSB, TMEM139, KRIT1, ZCWPW1, MSRB3, ZNF488, CCN1, |
| uu) | uu) CLDN4, DEDD2, GPNMB, HAS3, MATN3, MMP13, MSRB3, PDZD2, PKP2, |
| | RAPGEFL1, RBP1, TMC4, TP63, TPD52L1, UAP1, |
| vv) | vv) ACOX2, CCNG2, CDCA4, CDH4, CDV3, CENPM, CENPN, CLCN3, COL6A1, |
| | COL8A1, CSTA, CTSB, CYP27C1, DAPK1, DCLRE1B, DKK1, DUSP6, EBP, FAM83B, |
| | HPS5, IGSF9, IL6, ITGA2, LMNB2, MAD2L1, MSX2, NEDD4L, NNMT, OTUB2, OXCT1, |
| | PAK2, PCBD1, PIK3R1, PKP2, PKP4, PLCE1, RBM23, RCN2, RFC4, RGS17, S100A14, |
| | SAMD9, SCML1, SEC14L2, SH3RF1, SLC25A23, SLC38A5, SNCG, TMC4, TMEM139, |
| | TNNC1, TP53INP1, TPD52L1, TRIP11, TRIP13, TUBB6, UGCG, VAMP8, WDR13, |
| | WWC1, ZBED2, ZNF467, ZNF704, |
| ww) | ww) IL6, SHROOM2, SHCBP1, DCLRE1B, CDV3, SAMD9, IGSF9, OTUB2, COL6A1, |
| | TPD52L1, WDR13, RBM23, SMOC1, CCNG2, TNNC1, FDPS, LMNB2, CCBE1, CTSB, |
| | ZNF704, PKP2, WWC1, CRY1, SH3RF1, CYP27C1, SLC25A23, CENPN, |
| xx) | xx) ABAT, ADAMTS1, AMOT, ANXA3, ARAP3, ATXN1, CAP2, CDC25A, CEBPB, |
| | CENPM, CLIC3, COL6A2, CROT, CTH, DAPK1, DCLRE1B, DDAH1, DLL1, DSC2, |
| | EIF2AK3, ESM1, ETS1, EXO1, FAHD2A, FAM102A, FKBP2, GADD45B, GRAMD2B, |
| | HDHD2, IGSF3, JPH2, KRT8, LMTK3, LRP10, LRP8, MAP6D1, MCM5, MFSD5, MID1, |
| | MLLT11, MRPL33, NCAPD3, NFIB, NUAK1, OAS1, OAS2, PDZD2, PIK3R3, PKIA, |
| | PKMYT1, PLA2G4C, PLK2, PPP1R11, PSG6, PSG7, PTPRE, PYGB, ROR1, SECISBP2, |
| | SH3PXD2A, SLC25A3, SLC30A9, SLC3A2, SNAPC1, STK3, STX1A, TAGLN, TDO2, |
| | TMEM144, TMEM45B, TP53INP1, TPX2, TRIB2, TRIP13, VPS52, ZBED2, |
| yy) | yy) RAPGEFL1, JPH2, STX1A, PKIA, LRP8, TAGLN, TMEM144, SNAPC1, CTH, QKI, |
| | LMTK3, PIK3R3, KRT8, ANXA3, PSG6, ESM1, SH3PXD2A, OAS2, IGSF3, SLC3A2, |
| | CROT, MFSD5, CENPM, EIF2AK3, MRPL33, ROR1, |
| zz) | zz) AASS, ACOX2, ADAMTS1, BCAT1, CEBPB, CHRNB1, FXYD3, IL6, IRX5, LYPD3, |
| | MALL, NUAK2, PAK1, PCNA, PHF21A, PYGB, RACGAP1, RFC4, ROR1, RPS24, |
| | SMOC1, SORT1, SUSD2, TEAD4, TMC4, TPM1, |
| aaa) | aaa) ACOX2, TPM1, PHF21A, IRX5, BCAT1, SORT1, CHRNB1, FXYD3, PYGB, PAK1, |
| | IL6, |
| bbb) | bbb) CA2, CAP2, CCDC80, CDC25A, CDCA3, CDCA4, DDAH1, DLL1, DUSP6, ELN, |
| | FEN1, IRF9, ITGA2, KRT80, LMCD1, MAPK13, MCM2, MFSD5, MLLT11, NRIP1, OFD1, |
| | OSBPL7, PCNA, PHF21A, PIK3R3, PLK2, PRRX2, PTPRE, QKI, RAPGEFL1, RBMS2, |
| | SH3TC1, SHCBP1, SLC30A9, SLC7A1, SNX24, TMC4, TMEM45B, UGCG, ULK1, |
| | WDR13, |
| ccc) | ccc) CCBE1, CCNG2, CHST13, DHCR7, FERMT1, FMR1, GRB10, HBP1, HPS5, IFI44, |
| | IRAK2, KCNMB3, LYPD3, MCM2, MSX2, NNMT, PCBD1, PKP2, PLA2G4C, PSG9, |
| | REEP6, STK3, TEAD4, TMC4, TUFT1, WWC1, |
| ddd) | ddd) CCBE1, HBP1, CHST13, MSX2, PKP2, PCBD1, |
| eee) | eee) AASS, ARAP3, BTG3, C4BPB, CDK2, CNN3, COBL, COTL1, DCLRE1B, DSG3, |
| | DYNC2LI1, HOXA5, HPS5, KLF13, KRTDAP, LHFPL6, MCM10, MFSD1, OFD1, |
| | OSBPL7, OTUB2, PAK1, PIK3R2, QKI, RBM45, RCN2, SGK1, SHCBP1, SLFN5, SNX24, |
| | SPIRE2, STK3, TMC7, TMEM144, |
| fff) | fff) SHCBP1, PIK3R2, MFSD1, QKI, SNX24, SPIRE2, AASS, DSG3, |
| ggg) | ggg) ABAT, ABI3BP, AMOT, B4GALT4, BCL11B, CDC25A, CDCA4, CHRNB1, CLDN4, |
| | DCLRE1B, DLL1, EBP, EXO1, FSTL1, FSTL3, IL6, IRF9, KCNMB3, MCM10, MLPH, |
| | NOC3L, NUP37, PDZD2, PSG9, RAB32, RND3, SH3RF1, SLC25A23, SMOC1, SYDE2, |
| | TGM2, TP63, TYMS, ZNF488, |
| hhh) | hhh) CDC25A, RAB32, RND3, ZNF488, SH3RF1, NOC3L, AMOT, CDCA4, DCLRE1B, |
| | FSTL3, |
| iii) | iii) ACAT2, ANKRD12, CCBE1, CCN1, CDC25A, CDK1, CDK2, COBL, CROT, CYP27C1, |
| | EPHA2, F3, HOXA5, ITGB2, KPNA2, LRP10, LRRFIP2, MLLT11, OVOL1, PIP4P2, PLK2, |
| | PNRC1, PRRX2, PSG9, REEP6, SGMS2, SGTB, SH3TC1, SNAPC1, SPAG1, TNNC1, |
| | TRIP11, TSPAN1, WWC2, |
| jjj) | jjj) CDC25A, PLK2, SGTB, MLLT11, TSPAN1, CCBE1, COBL, SNAPC1, |
| kkk) | kkk) ABCA1, ACADVL, ACOT11, AGL, AVPI1, CCBE1, CLDN4, CPE, CRIM1, CROT, |
| | DDR1, EPS8L2, FAM117B, FXYD3, GADD45B, GRAMD2B, GSN, HASPIN, JDP2, |
| | KCNMB3, KRT8, MATN2, MDC1, MFSD5, MLLT11, MMP13, PAG1, PCNA, PHLPP1, |
| | PIK3R3, PSG2, PSG6, PVR, RFC4, SGTB, SLC25A23, SLC3A2, SNCG, TACC3, |
| | TENT5B, TRIM13, VSNL1, WWC2, |
| kkk1) | kkk1) CCBE1, |
| lll) | lll) ANTXR2, CLDN1, GPRC5A, GPRC5C, MALL, PVR, TEAD1, TMC4, TRIP13, |
| | ZCWPW1, |
| mmm) | mmm) EPS8L3, PLOD2, |
| nnn) | nnn) CDC25A, DCLRE1B, DHFR, FOS, HASPIN, JDP2, LMCD1, MID1, MXRA7, |
| | NEDD4L, SCML1, SH3PXD2A, TDO2, TMEM140, ZSWIM7, |
| ooo) | ooo) HASPIN, NEDD4L, SCML1, |
| ppp) | ppp) AURKB, CCBE1, COL6A2, DIAPH3, HDHD2, LSM5, MDC1, PLOD2, PPIH, SGK1, |
| | TMC4, TRIP13, VSNL1, |
| ppp1) | ppp1) TRIP13, |
| qqq) | qqq) ADRB2, AURKB, CDK6, TEAD4, VAMP8, |
| qqq1) | qqq1) ADRB2, |
| rrr) | rrr) ABCC5, AGL, ASF1A, BIRC5, CENPN, CLCN3, COL6A1, COL6A2, DDAH1, |
| | DONSON, ERAP2, FAM117B, FEN1, FMR1, GADD45A, GPC6, GRAMD2B, HOXA5, |
| | IGSF3, KRTDAP, MAPK13, NCAPD3, NEDD4L, NEXN, OAS2, OFD1, PAK2, PHF21A, |
| | QKI, RAC2, RAPGEFL1, RNF144B, SGK1, SLC38A5, SPRY4, TNNC1, TSPAN1, TUFT1, |
| sss) | sss) ACAT2, ADM, AVPI1, AZIN1, BIRC5, CCN1, CDC6, CDK6, CEBPB, CENPA, CENPI, |
| | COL6A2, CSTA, CXCL1, DIAPH2, DYNC2LI1, EMG1, FAM83D, FST, GOLGA5, HASPIN, |
| | HEG1, HEY1, HPS5, ITGA2, ITGB2, KRT80, LRP10, LTBP4, LYPD6, MATN2, MCM10, |
| | MCM5, MICB, MSRB3, NFIL3, NUAK1, OLR1, PJA2, PNRC1, PRSS23, PSG9, PTPRE, |
| | ROR1, SEC14L1, SHCBP1, SHROOM2, SLC1A2, SLC37A2, SLFN5, TDO2, TMEM140, |
| | TNFRSF12A, TPX2, TRIM13, TRIM38, |
| ttt) | ttt) CDCA5, CDK6, CSTA, CXXC5, DYNC2LI1, EIF2AK3, EMG1, EPS8L3, EXOSC2, |
| | FKBP2, HSPB11, IRF9, ITGA2, LTBP4, MATN2, METRNL, MMP13, MRPL33, OLR1, |
| | PCMTD2, PHF21A, PRSS23, PTPRE, RND3, SHCBP1, SHROOM2, SLC37A2, SLC3A2, |
| | SLK, SNAPC1, TBX3, TRIB1, UBE2C, UHRF1, |
| uuu) | uuu) AGPAT4, AZIN1, BHLHE41, CDCA8, COBL, CSRNP2, CTNNBIP1, FAM83D, |
| | FANCA, FAT4, FEN1, GOLPH3L, IL6, INTS3, JDP2, KLF10, KRTDAP, LMTK3, LXN, |
| | MANSC1, MARCKSL1, MRPL33, MXRA7, NAPEPLD, NCAPD3, NNMT, NOC3L, |
| | NUAK2, OAS1, PAK1, PCBD1, PEPD, PRSS23, PSG2, PSG6, PSG7, PYGB, RGL2, |
| | SGK1, SNCG, SPATA5, SRD5A3, STX1A, SUSD2, TCF25, THBS1, TP53INP1, |
| | TPD52L1, ZDHHC18, |
| vvv) | vvv) CDCA8, LXN, INTS3, PSG2, ZDHHC18, PEPD, MARCKSL1, PRSS23, PSG7, |
| | PSG6, MXRA7, KRTDAP, SPATA5, JDP2, IL6, PCBD1, KLF10, AZIN1, SGK1, NCAPD3, |
| | RCN2, CSRNP2, TP53INP1, FOS, |
| www) | www) AASDH, ANKRD42, ANTXR2, ASF1A, BTG3, CCN1, CDC42EP4, CDH4, CDK2, |
| | CELSR3, CENPA, CENPN, CNN3, COL8A1, CTSB, CXCL1, CYP1B1, DDAH1, DIAPH2, |
| | DLL1, DUT, DYNC2LI1, ESM1, FAM83D, FXYD3, GPR176, LMTK3, LRRFIP2, MCM10, |
| | MMP13, MYO1A, NCAPD3, NEDD4, NEXN, NTN4, NUP37, OLFML3, OVOL1, PAG1, |
| | PCBD1, PCDHB9, PDZD2, PHLPP1, PKMYT1, PLEKHA7, PRPS2, PSG9, RAB11FIP1, |
| | RBMS2, RFC4, SHCBP1, SLC1A2, SPAG1, STK3, TK1, TNNC1, TRIP13, TTC17, |
| | UHRF1, WWC1, WWC2, ZDHHC18, ZNF292, ZNF75D, |
| xxx) | xxx) MCM10, CCN1, DUT, NUP37, WWC2, CELSR3, TRIP13, DIAPH2, ASF1A, PCBD1, |
| | RFC4, LMTK3, TNNC1, ZNF292, OVOL1, ESM1, PAG1, COL8A1, ZNF75D, NEDD4, |
| | PCDHB9, DYNC2LI1, CNN3, OLFML3, |
| yyy) | yyy) AASDH, ANKRD29, ANXA3, BCAT1, C4BPB, CLDN4, CLIC3, COTL1, CSTA, |
| | CXXC5, CYP27C1, DSC2, FOS, HASPIN, IGSF3, KLF13, LRP10, MALL, MDC1, MGST2, |
| | MID1, MTMR11, MTSS1, NPAS2, NUAK1, PIK3C2B, PKP2, PRPS1, PSG6, PSG7, |
| | PSG9, RAPGEFL1, SCML1, SEC14L1, SH3TC1, SKP2, SLFN5, SNAPC1, TMEM139, |
| | TPD52L1, TSPAN1, VAMP8, WWC2, |
| zzz) | zzz) ABCA1, ALDH3A2, ARHGAP11A, ARHGEF2, BIRC5, CAVIN1, CCDC80, CDCA8, |
| | CDK2, CLDN4, COL8A1, CYP1B1, DHX32, DIAPH3, ESM1, FAM83D, FMR1, GOLPH3L, |
| | GPNMB, HBP1, HDAC1, JDP2, JPH2, KLHL3, KPNA2, LRP10, MDC1, MSRB3, MTSS1, |
| | MVD, MXRA7, NNMT, NUAK2, OVOL1, PCDHB2, PEPD, PHF21A, PIK3C2B, PKP4, |
| | PLOD2, PSG2, PXMP2, PYGB, RAC2, RAPGEFL1, SCML1, SGTB, SPAG1, TAGLN, |
| | TK1, TLR3, TMEM160, TNNC1, TRIP13, TSPAN1, TTF2, TTLL3, TYMS, UHRF1, VGF, |
| | VKORC1L1, VSNL1, ZCWPW1, |
| aaaa) | aaaa) TSPAN1, CLDN4, LYPD6, MSRB3, PHF21A, SCML1, TTF2, PIK3C2B, GOLPH3L, |
| | VKORC1L1, MVD, JDP2, CAVIN1, PYGB, RAPGEFL1, CCDC80, COL8A1, MDC1, |
| | ZCWPW1, CYP1B1, DHX32, RFC4, CDCA8, ESM1, OVOL1, |
| bbbb) | bbbb) ACSL5, ANKRD22, ATP7A, AXL, BCL11B, BIRC5, BMF, CCBE1, CCDC80, |
| | CDCA3, CNN3, COL6A1, COL6A2, CTNNBIP1, CTSB, DHX32, DKK1, DSC2, DUSP6, |
| | DYNC2LI1, FAM102A, FAM83B, FSTL1, FXYD3, GRAMD2B, GSN, HEXB, IRX5, |
| | KCNMB3, KLK11, LRP8, LSM5, MALL, MAP6D1, MCM2, METRNL, MFSD1, MGST3, |
| | MLPH, MMP13, MRPL33, MSX2, NEDD4L, NFIL3, NNMT, NPAS2, NRIP1, OAS2, OLR1, |
| | OTUB2, PCMTD2, PCNA, PIK3R2, PSG2, RAB11FIP1, RACGAP1, RAPGEFL1, RBMS2, |
| | RFC4, RNF144B, S100A14, SAMD9, SCD5, SDC2, SEC14L2, SHCBP1, SLC1A2, |
| | SLC3A2, SLC7A1, SLFN5, SMPD4, SPATA5, SPIRE2, SQSTM1, TACC3, TDO2, TMC4, |
| | TNNC1, TRIB1, TRIB2, TSPAN1, UAP1, UBE2C, UHRF1, VSNL1, WDR13, ZNF704, |
| | ZSWIM7, |
| cccc) | cccc) IRX5, TSPAN1, NPAS2, GSN, |
| dddd) | dddd) ACOT11, ACSL5, ANKRD42, ANTXR2, ARAP3, BCAT1, CDC25A, CDV3, |
| | CELSR3, COL6A1, COL8A1, CPA4, CPE, CTSK, DAPK1, DONSON, DSG3, DUSP14, |
| | EBP, FKBP2, FSTL1, FXYD3, GDPD1, GPRC5C, HASPIN, KCNMA1, KRT80, LRRFIP2, |
| | LYPD6, MDC1, MXRA7, NUDCD1, PLA2G4C, PNRC1, PSG7, PSG9, RAPGEFL1, |
| | RBM23, RCN2, RGL2, SAMD9, SEC14L1, SH3RF1, SKP2, SLC14A1, SNAPC1, TACC3, |
| | TDO2, TEAD1, TNFAIP3, TPM1, TRIB1, TRIP11, UBE2C, UGCG, VAMP8, VSNL1, |
| | WDR13, |
| eeee) | eeee) HASPIN, PIK3C2B, CPA4, UGCG, PSG7, VAMP8, VSNL1, WDR13, FKBP2, |
| | MDC1, ACOT11, KRT80, |
| ffff) | ffff) ARHGAP11A, AVPI1, CDCA4, CDCA8, CDK2, CLDN4, COTL1, CXCL1, CYP27C1, |
| | DHFR, DUSP6, EBP, FAT4, GPC6, GPR176, GSN, HEXB, IGSF3, KLF10, LMTK3, |
| | MCM10, MICB, MLLT11, NFIL3, NRIP1, PCDHB2, PCGF3, PKIA, PSG7, RAB32, |
| | RBM45, RCN2, S100A14, SH3PXD2A, SKP2, SPAG1, SPATA5, SSPN, STK3, STXBP6, |
| | TNFRSF12A, TRIB1, TRIM13, TRIM31, TRIM38, TYMS, ZNF75D, |
| gggg) | gggg) TFF1, GPR176, NRIP1, PCDHB2, SKP2, CLDN4, CDCA4, STXBP6, DIAPH3, |
| hhhh) | hhhh) ABAT, ARHGAP11A, ARHGDIB, ATXN1, AURKB, B4GALT4, BHLHE41, CAP2, |
| | CCN2, CCNG2, CLDN1, CRIM1, CSTA, CTSB, DCLRE1B, DIAPH2, DSC2, FAHD2A, |
| | GRB10, JDP2, KLF10, LRP10,, LRP8, LRRFIP2, MDC1, MMP13, MTMR9, NEDD4, |
| | NFIL3, NPAS2, NRIP1, OSBPL7, PAK2, PCGF3, PKP2, PPP1R11, PRSS23, PSG9, |
| | RBP1,, RCN2, RHOC, SH3TC1, SLC25A23, SLC25A3, SLC30A9, SLFN5, SLITRK6, |
| | SPAG1, STX1A, TACC3, TEAD4, TMEM160, TMEM45B, TNFRSF12A, TRAPPC6B, |
| | TRIM13, TRIOBP, TRIP13, TUBB6, UAP1, |
| iiii) | iiii) FAHD2A, PCGF3, TMEM45B, PKP2, SLFN5, TRIOBP, MMP13, LRP10, CCN2, |
| | KLF10, NEDD4, RBP1, MDC1, DIAPH2, IGSF3, TACC3, SLC25A3, SLITRK6, CTSB, |
| | AURKB, SLC25A23, DCLRE1B, TRIM13, PRSS23, SLC30A9, GDPD1, NFIL3, CSTA, |
| | PSG9, CRIM1, BHLHE41, |
| jjjj) | jjjj) CTSB, SHROOM2, TNNC1, ZCWPW1, |
| kkkk) | kkkk) ADAM28, ANXA3, AURKB, CAVIN1, CCN2, CDCA3, CDCA5, CENPI, CENPN, |
| | CTSK, DONSON, FKBP2, FSTL1, GPR176, GPRC5C, GSN, HEG1, IFI44, IRX5, ITGB2, |
| | KIFAP3, MALL, MCM10, MICB, MLPH, MYO1A, NUAK1, PPP1R11, PSG6, PXMP2, |
| | SGK1, SH3TC1, SKP2, SLC1A2, SLC39A11, SLITRK6, SLK, TK1, TMC7, TPM1, TPX2, |
| | TRIM38, TRIOBP, TRIP13, TSPAN2, TTC17, ZNF75D, |
| llll) | llll) AASDH, ABCC5, ANKRD29, ASF1A, AXL, CDK1, CKS2, CLDN1, CLDN4, CLIC3, |
| | CPA4, CTH, DONSON, EIF2AK3, FOS, FXYD3, GADD45B, HBP1, IFI44, MID1, NTN4, |
| | PCDHB2, PHLPP1, PPP1R11, RGS17, SKP2, SLC3A2, SMOC1, SPRY4, STX1A, |
| | SUSD2, TTC17, TYMS, UBC, VKORC1L1, VPS52, ZBED2, or |
| mmmm) | mmmm) FXYD3, SKP2, SMOC1, DONSON, CTH, VPS52, CPA4, SLC3A2. |

In some embodiments, a set of genes for a “short TEAD signature” may be associated to a cancer selected from: Adrenocortical carcinoma (ACC) tumor; Bladder Urothelial Carcinoma (BLCA) tumor; Breast invasive carcinoma (BRCA); BRCA basal tumor; BRCA non-basal tumor; Cervical squamous cell carcinoma or endocervical adenocarcinoma (CESC) tumor; Cholangiocarcinoma (CHOL) tumor; Colon adenocarcinoma (COAD) tumor; COAD CMS1 tumor; COAD CMS2 tumor; COAD CMS4 tumor; Lymphoid Neoplasm Diffuse Large B-cell Lymphoma (DLBC) tumor; Esophageal carcinoma (ESCA) tumor; Glioblastoma multiforme (GBM) tumor; Head or Neck squamous cell carcinoma (HNSC) tumor; Kidney Chromophobe (KICH) tumor; Kidney renal clear cell carcinoma (KIRC) tumor; Kidney renal papillary cell carcinoma (KIRP) tumor; Brain Lower Grade Glioma (LGG) tumor; Liver hepatocellular carcinoma (LIHC) tumor; LIHC S1 tumor; LIHC S2 tumor; LIHC S3 tumor; Lung adenocarcinoma (LUAD) tumor; LUAD proximal-inflammatory tumor; LUAD proximal-proliferative tumor; Lung squamous cell carcinoma (LUSC) tumor; LUSC basal tumor; LUSC classical tumor; LUSC primitive tumor; LUSC secretory tumor; Malignant mesothelioma (MESO) tumor; Ovarian serous cystadenocarcinoma (OV) tumor; OV differentiated tumor; for OV immune-reactive tumor; OV mesenchymal tumor; OV proliferative tumor; Pancreatic adenocarcinoma (PAAD) tumor; Rectum adenocarcinoma (READ) tumor; READ CMS1 tumor; READ CMS2 tumor; READ CMS4 tumor; READ unclassifiable tumor; Sarcoma (SARC) tumor; Skin Cutaneous Melanoma (SKCM) tumor; SKCM immune tumor; SKCM keratin tumor; SKCM MITF-low tumor; Stomach adenocarcinoma (STAD) tumor; STAD MSI tumor; STAD MSS_EMT tumor; STAD MSS_TP53 tumor; STAD MSS_TP53+ tumor; Testicular Germ Cell Tumor (TGCT) tumor; Thyroid carcinoma (THYM) tumor; Uterine Corpus Endometrial Carcinoma (UCEC) tumor; and Uterine Carcinosarcoma (UCS) tumor.

The “short TEAD signature” may be used in methods for computing a discriminant score (S) (method 3) or (DS) (method 4).

### Method 3: Determinate a Discriminant Score (S).

To reduce the dimensionality of testing, subsets of the most informative genes in the TEAD-500 panel that are applicable to specific cancer types were determined. These are panels of fewer genes that predict the status of the TEAD-complex (active or inactive) in samples from a given cancer indication or subtype.

To determine TEAD-active or TEAD-inactive status of a sample, the fractional ranks of the genes in the optimal or minimal sets are multiplied by the corresponding coefficients in parenthesis. The products are summed to give a discriminant score (S). The discriminant score (S) is compared to the centroids of the TEAD-active (A) and TEAD-inactive (I) groups, which are provided for each of the corresponding cancer indication or subtype. If(S) is closer to (A) than it is to (I), then, the sample is classified as TEAD-active. Otherwise, the sample is classified as TEAD-inactive.

For example, expression levels are determined, and the tested gene's fractional rank is calculated and multiplied by the corresponding coefficient in parentheses. The products are summed to give a discriminant score (S). One then essentially applies the following calculation: max(S,A)−min(S,A)<max(S,I)−min(S,I) as follows: compare (S) to the centroid for the TEAD-active (A) and choose the maximum. Thus, if (S) is 5 and (A) is −268.9, one would choose 5. Then compare the (S) and (A) and choose the minimum. Thus, if (S) is 5 and (A) is −268.9, one would choose-268.9. Then compare (S) and (I) and choose the max. Thus, if (S) is 5 and (I) is 22.1, one would choose 22.1. Then compare (S) and (I) and choose the min, thus, if (S) is 5 and (I) is 22.1, choose 5. Then ask if 5 minus negative 268.9 is less than 22.1 minus 5 (i.e., is 5−(−268.9)<22.1−5). If yes, the cancer is TEAD-active. If no, the cancer is TEAD-inactive. In this hypothetical example, the cancer is TEAD-inactive because 273.9 is not less than 17.1.

As an alternative method for scoring the discriminant functions, one can consider the mean between the provided centroid for TEAD-active (A) and TEAD-inactive (I), e.g., the mean between (0.5 and −5.7)=−2.6. If the calculated score is >−2.6, then the sample may be considered TEAD inactive. If not, it may be considered TEAD-active.

The following Embodiments 3-59 provide alternative panels that can be used in the methods and uses described herein, where the method/use is specific to particular cancer types. For most embodiments there are two useful panels—an “optimal discriminant function” and “minimal discriminant function.” The term “optimal” should not be construed to be better than the minimal.

TABLE 2
Optimal and minimal discriminant predictors of sets of genes for specific cancer indications

| # | Sets of genes for specific cancer indications |
|---|---|
| | Embodiment 3. Optimal and minimal discriminant predictors, with their |
| | unstandardized coefficients, and group centroids for Adrenocortical |
| | carcinoma (ACC) tumors: |
| a) | Set a): Optimal discriminant function: AASS (56.8) + ARHGDIB (138.6) + ASF1A (−15.7) + |
| | CA2 (−17.6) + CBR3 (−51.2) + CCDC80 (110.0) + CLIC3 (−268.7) + COL8A1 (−24.6) + CPA4 |
| | (15.1) + CPE (−62.7) + CYP27C1 (1089.0) + DSG3 (326.8) + ESM1 (−79.8) + F3 (−64.7) + |
| | FXYD3 (−58.5) + GADD45A (−18.0) + GSN (388.7) + HOXA5 (−19.4) + IGSF9 (206.3) + |
| | IRAK2 (−105.3) + IRF9 (152.3) + KLF10 (53.6) + KLF13 (−5.8) + KLHL3 (−15.6) + KRT8 (5.8) + |
| | LCA5 (−285.3) + LYRM1 (−18.5) + MICB (−42.2) + MTMR9 (−61.9) + OGFRL1 (−176.3) + |
| | PIK3C2B (−82.7) + PKP2 (−43.5) + PNRC1 (943.5) + RBP1 (94.4) + SEC14L1 (263.8) + |
| | SGTB (−32.5) + SH3TC1 (21.2) + SORT1 (51.3) + STX11 (124.3) + STX1A (−52.5) + SYDE2 |
| | (−283.9) + TEAD4 (−24.3) + TFF1 (−136.8) + TMC7 (−19.7) + TMEM139 (29.3) + TP63 |
| | (−206.9) + TSPAN1 (56.6) + TUBB6 (81.4) + ULK1 (518.7) − 1967.8 |
| | Optimal function group centroids: TEAD-inactive (I) 22.1; TEAD-active (A) −268.9 |
| b) | Set b): Minimal discriminant function: ULK1 (15.4) + TMC7 (−13.4) + LCA5 (−10.4) + |
| | ZSWIM7 (3.8) + PNRC1 (16.0) − 26.0 |
| | Minimal function group centroids: TEAD-inactive (I) 0.5; TEAD-active (A) −5.7 |
| | Embodiment 4. Optimal and minimal discriminant predictors, with their |
| | unstandardized coefficients, and group centroids for Bladder Urothelial |
| | Carcinoma (BLCA) tumors: |
| c) | Set c): Optimal discriminant function: ACOX2 (2.2) + ADAMTS1 (−1.5) + AMOT (−3.0) + |
| | AURKB (−2.1) + AVPI1 (−1.5) + CA2 (1.6) + CCNG2 (1.3) + CDH4 (−8.1) + DONSON (−1.8) + |
| | DSC2 (1.6) + EPS8L2 (−1.8) + FXYD3 (4.7) + GPRC5C (1.8) + HDAC1 (6.9) + HDHD2 |
| | (1.9) + HEY1 (.8) + IGSF3 (−2.3) + KCNMA1 (2.5) + KLHL3 (−4.0) + KRCC1 (−1.6) + KRT8 |
| | (−2.0) + KRT80 (−1.3) + LCA5 (−4.3) + LTBP4 (1.0) + MAPK13 (−3.6) + MGST2 (7.8) + MLPH |
| | (−.7) + NFIL3 (2.7) + NRIP1 (3.9) + NUAK2 (−3.1) + PIK3R3 (1.2) + PKP2 (−1.6) + PNRC1 |
| | (−4.2) + PSG2 (5.0) + RAPGEFL1 (2.3) + RBP1 (2.2) + RGL2 (3.6) + RND3 (1.2) + |
| | RNF144B (1.1) + S100A14 (1.5) + SEC14L1 (−2.3) + SH3RF1 (1.9) + SHCBP1 (−3.7) + |
| | SLC1A2 (8.4) + SLC25A3 (−27.7) + STX1A (2.3) + TTF2 (3.6) + TUFT1 (2.3) + TYMS (2.4) + |
| | VAMP8 (5.6) + ZNF75D (5.7) + .9 |
| | Optimal function group centroids: TEAD-inactive (I) 0.8; TEAD-active (A) −5.0 |
| | Minimal discriminant function: NA |
| | Embodiment 5. Optimal and minimal discriminant predictors, with their |
| | unstandardized coefficients, and group centroids for Breast invasive |
| | carcinoma (BRCA) tumors: |
| d) | Set d): Optimal discriminant function: ABAT (.9) + ACAT2 (.6) + ADM (.6) + ADRB2 (−1.4) + |
| | ANXA3 (.6) + ARAP3 (−2.2) + BCAT1 (1.9) + CAV1 (1.5) + CCN1 (2.7) + CCN2 (−5.6) + |
| | CDC42EP4 (1.1) + CDK1 (−1.5) + COTL1 (2.3) + CPA4 (−1.7) + CSRNP2 (1.4) + CTSK |
| | (1.7) + CXCL1 (.9) + CXXC5 (−1.5) + DIAPH3 (2.4) + DONSON (1.4) + EXO1 (2.3) + |
| | FAHD2A (1.5) + FOS (−1.3) + FSTL1 (4.3) + FTH1 (88.1) + GOLPH3L (1.5) + GPR176 (1.9) + |
| | GPRC5B (.9) + IRF9 (−1.3) + KIFAP3 (−3.6) + KRT80 (.6) + KRTDAP (−.9) + LMCD1 |
| | (−1.0) + LMTK3 (.7) + MALL (−.6) + MDC1 (1.5) + MGST2 (−3.4) + MLPH (−2.4) + MMP13 |
| | (−.5) + NUP107 (1.2) + OLFML3 (−1.3) + PAK1 (−.7) + PAK2 (−2.2) + PCDHB9 (−2.1) + PDLIM2 |
| | (1.9) + PERP (−1.0) + PIK3R3 (−1.0) + PIP4P2 (−.9) + PKP4 (2.8) + PTPRE (−1.3) + |
| | RAPGEFL1 (.7) + RBMS2 (−2.9) + RGL2 (−4.3) + RHOC (3.6) + SHCBP1 (−2.2) + SLC38A5 |
| | (−1.2) + SLC3A2 (2.3) + SMOC1 (1.4) + SMPD4 (2.1) + SNAPC1 (1.4) + SNCG (−.7) + |
| | SPATA5 (3.1) + STXBP6 (−2.1) + THBS1 (2.4) + TNNC1 (1.9) + TPD52L1 (−.7) + TPM1 |
| | (2.5) + ZCWPW1 (−2.0) + ZNF488 (−4.8) − 93.0 |
| | Optimal function group centroids: TEAD-inactive (I) −0.4; TEAD-active (A) 5.4 |
| | Minimal discriminant function: NA |
| | Embodiment 6. Optimal and minimal discriminant predictors, with their unstandardized |
| | coefficients, and group centroids for BRCA basal tumors: |
| e) | Set e): Optimal discriminant function: AASDH (5.1) + ABCC5 (−4.1) + ADM (1.4) + BCL11B |
| | (−4.5) + BMF (−1.6) + C4BPB (6.3) + CAVIN1 (6.4) + CCBE1 (−9.2) + CCDC80 (−5.3) + |
| | CDCA8 (4.7) + CDV3 (7.4) + COTL1 (11.5) + CSRNP2 (3.3) + CTSK (10.4) + DAPK1 (3.4) + |
| | DIAPH3 (3.7) + ESM1 (2.2) + EXO1 (5.6) + GPC6 (4.8) + HEY1 (−1.8) + IRF9 (−6.0) + |
| | JDP2 (5.1) + KIFAP3 (−4.9) + KRT80 (2.3) + LXN (−1.5) + MGST2 (−3.0) + MXRA7 (5.5) + |
| | NCAPD3 (3.6) + NEDD4 (6.0) + NEK2 (−4.7) + NTN4 (−1.6) + PIK3C2B (2.5) + PIK3R3 |
| | (−3.3) + PKP4 (10.0) + PPIH (8.7) + PSG2 (172.5) + RAPGEFL1 (2.5) + RBM24 (4.6) + RGL2 |
| | (−7.1) + RNF144B (4.6) + SLC25A3 (31.9) + SLC35C1 (−3.3) + SLC38A5 (−4.3) + SLC39A11 |
| | (−3.7) + SNCG (−1.1) + STX11 (11.6) + SYDE2 (−4.3) + TDO2 (−4.1) + TENT5B (1.3) + TET2 |
| | (−7.7) + TPM1 (3.8) + VKORC1L1 (−5.4) + ZCWPW1 (−4.2) − 69.5 |
| | Optimal function group centroids: TEAD−inactive (I) −2.1; TEAD-active (A) 5.4 |
| f) | Set f): Minimal discriminant function: DIAPH3 (3.9) + MGST2 (−3.5) + RGL2 (−7.2) + KIFAP3 |
| | (−3.6) + IRF9 (−5.4) + CTSK (9.4) + SNCG (−1.4) + ACAT2 (0.9) + JDP2 (3.8) + RBM24 (3.4) + |
| | COTL1 (9.0) + PKP4 (7.3) + C4BPB (5.8) + CDCA8 (4.9) + MXRA7 (4.5) + SLC38A5 |
| | (−3.7) + SLC35C1 (−3.0) + CDV3 (8.9) + RNF144B (3.9) + STX11 (10.1) + ADM (1.2) + PSG2 |
| | (157.5) + GPC6 (3.4) + BCL11B (−4.7) + VKORC1L1 (−4.4) + RAPGEFL1 (1.8) + SLC25A3 |
| | (20.4) + ABCC5 (−3.4) + TDO2 (−3.7) + SLC39A11 (−3.6) + CCDC80 (−3.9) + CAVIN1 (5.0) + |
| | EXO1 (4.2) + NEK2 (−4.3) + TET2 (−5.2) + LXN (−1.9) + CCBE1 (−9.6) + ZCWPW1 (−2.9) + |
| | PPIH (8.2) + PIK3R3 (−2.3) + AASDH (4.1) + KRT80 (2.1) + NEDD4 (3.6) + NCAPD3 |
| | (2.3) − 47.3 |
| | Minimal function group centroids: TEAD-inactive (I) −1.8; TEAD-active (A) 4.6 |
| | Embodiment 7. Optimal and minimal discriminant predictors, with their |
| | unstandardized coefficients, and group centroids for BRCA non-basal |
| | tumors: |
| g) | Set g): Optimal discriminant function: ABAT (1.3) + ARAP3 (−4.2) + ARHGDIB (−6.5) + |
| | BCL11B (3.4) + BMF (1.5) + CAV1 (3.1) + CCN2 (−3.0) + CDC25A (3.2) + CDK2 (2.2) + |
| | CDK6 (2.8) + CLDN4 (−6.4) + CLIC3 (1.4) + COL8A1 (2.8) + DDR1 (−3.8) + DHFR (−2.9) + |
| | DHX32 (1.4) + DKK1 (−1.1) + DUSP6 (−1.3) + EXOSC2 (4.2) + FAHD2A (2.5) + FAM102A |
| | (3.4) + FDPS (−4.5) + FST (−1.8) + GADD45A (2.4) + GINS1 (1.9) + GSN (−8.7) + JPH2 |
| | (8.4) + KLF13 (1.9) + KRCC1 (2.4) + LMCD1 (−1.7) + LRP10 (−3.7) + LYPD6 (.7) + MAP6D1 |
| | (−1.7) + MCM2 (−3.3) + MCM5 (2.5) + MSX2 (−.9) + MTMR11 (1.5) + MTMR9 (−3.9) + |
| | NCOA3 (4.2) + NEXN (−3.3) + OAS1 (−.9) + OLFML3 (3.6) + PCMTD2 (−3.0) + PIK3C2B |
| | (−2.2) + PIP4P2 (−1.3) + PKIA (−.8) + PLEKHA7 (2.3) + PSG6 (40.7) + RAB11FIP1 (−1.1) + |
| | RBP1 (.6) + ROR1 (−3.3) + SCD5 (1.2) + SECISBP2 (−3.6) + SKP2 (2.2) + SNAPC1 (−2.0) + |
| | TRIOBP (−2.0) + TSPAN1 (−1.5) + TUFT1 (1.7) + WDR13 (−2.7) + ZBED2 (−6.4) + 24.5 |
| | Optimal function group centroids: TEAD-inactive (I) −0.1; TEAD-active (A) 13.6 |
| h) | Set h): Minimal discriminant function: JPH2 (10.6) + SKP2 (3.4) + DUSP6 (−1.8) + OLFML3 |
| | (4.2) + TSPAN1 (−1.1) + CDK2 (2.5) + NEXN (−4.4) + DDR1 (−4.1) + OAS1 (−0.8) + GPC6 |
| | (1.8) + DKK1 (−1.5) + GADD45A (2.1) + KRCC1 (2.3) + MALL (1.7) + GSN (−5.7) + FST |
| | (−1.3) + ARAP3 (−3.7) + MTMR11 (2.0) + EXOSC2 (4.2) + CAV1 (1.9) + PSG6 (54.4) + |
| | SECISBP2 (−2.9) + NCOA3 (2.3) − 1.1 |
| | Minimal function group centroids: TEAD-inactive (I) 0.0; TEAD-active (A) 9.3 |
| | Embodiment 8. Optimal and minimal discriminant predictors, with their |
| | unstandardized coefficients, and group centroids for Cervical squamous cell |
| | carcinoma and endocervical adenocarcinoma (CESC) tumors: |
| i) | Set i): Optimal discriminant function: ADM (−1.3) + ATXN1 (2.5) + AURKB (−2.5) + BHLHE41 |
| | (1.5) + BMF (2.2) + CDH4 (−2.4) + CELSR3 (2.6) + CTNNBIP1 (3.5) + CYTH3 (−2.4) + |
| | DHCR7 (−1.1) + EBP (−2.8) + GINS1 (−3.5) + GPRC5C (2.4) + GSN (−4.9) + HPS5 (−3.8) + |
| | JPH2 (−5.0) + LHFPL6 (1.3) + LRP8 (2.9) + LYRM1 (2.4) + MRPL33 (−6.2) + NAPEPLD |
| | (5.4) + NEK2 (−5.7) + OAS1 (2.1) + PAG1 (5.4) + PCDHB2 (4.0) + PCGF3 (2.6) + QDPR |
| | (−1.9) + RAPGEFL1 (2.6) + RBMS2 (−2.5) + ROR1 (7.4) + S100A14 (3.3) + SEC14L1 (−3.1) + |
| | SLC37A2 (3.7) + SUV39H1 (4.2) + TEAD4 (−2.5) + TP53INP1 (−1.8) + 7.7 |
| | Optimal function group centroids: TEAD-inactive (I) 1.1; TEAD-active (A) −4.1 |
| | Minimal discriminant function: NA |
| | Embodiment 9. Optimal and minimal discriminant predictors, with their |
| | unstandardized coefficients, and group centroids for Cholangiocarcinoma |
| | (CHOL) tumors: |
| j) | Set j): Optimal discriminant function: ADAM28 (25.7) + CRABP2 (−4.1) + ELN (−51.1) + |
| | ERAP2 (−7.3) + EXOSC2 (156.3) + FAM83D (21.0) + ITGB2 (28.2) + MARCKSL1 (18.7) + |
| | MFSD1 (48.7) + MGST2 (−483.6) + MLLT11 (15.9) + NUAK2 (−36.9) + PLA2G4C (−15.4) + |
| | RND3 (−37.2) + SHCBP1 (78.9) + SLK (−144.9) + SMOC1 (22.8) + SMPD4 (231.1) + TMC7 |
| | (117.9) + TRIM31 (24.6) + ULK1 (−160.3) + VPS52 (−258.5) + WWC2 (238.6) + ZNF292 |
| | (−174.9) + ZNF467 (53.3) + 486.0 |
| | Optimal function group centroids: TEAD-inactive (I) −12.7; TEAD-active (A) 178.4 |
| k) | Set k): Minimal discriminant function: VPS52 (20.8) + SHCBP1 (−5.3) + EXOSC2 (−10.5) + |
| | MGST2 (9.4) − 17.7 |
| | Minimal function group centroids: TEAD-inactive (I) 0.4; TEAD-active (A) −5.7 |
| | Embodiment 10. Optimal and minimal discriminant predictors, with their |
| | unstandardized coefficients, and group centroids for Colon adenocarcinoma |
| | (COAD) tumors: |
| l) | Set l): Optimal discriminant function: AGL (−2.6) + ATXN1 (2.3) + CCDC80 (−3.2) + CDCA8 |
| | (4.0) + CENPI (−5.4) + CLDN4 (−11.5) + CRY1 (−3.0) + EPS8L3 (2.8) + EXO1 (−4.5) + FXYD3 |
| | (8.8) + GPC6 (4.4) + GPRC5C (3.0) + IGSF9 (−1.2) + IRF9 (1.9) + ITGA2 (1.8) + KLK11 |
| | (1.0) + KRT8 (−14.1) + LHFPL6 (1.9) + MAP6D1 (4.5) + MYO1A (1.8) + NCAPD3 (3.4) + |
| | NTN4 (−1.6) + PAG1 (−2.2) + PCDHB2 (−1.9) + PLEKHA7 (3.7) + PSG9 (8.0) + PTPRE |
| | (−2.8) + PYGB (9.4) + RAPGEFL1 (1.8) + RBMS2 (−2.6) + RBP1 (1.0) + RGS17 (−9.7) + |
| | RHOC (−15.0) + RND3 (−1.8) + SLC14A1 (−.9) + SLC35C1 (−4.6) + SLC38A5 (−.9) + SSPN |
| | (2.5) + TFF1 (2.3) + TK1 (−3.5) + TMC4 (3.3) + TMEM45B (3.4) + TRIM38 (5.3) + TRIP13 |
| | (3.3) + UGCG (4.1) + VPS52 (−3.7) + WWC1 (−2.9) + 11.5 |
| | Optimal function group centroids: TEAD-inactive (I) 0.3; TEAD-active (A) −7.0 |
| | Minimal discriminant function: NA |
| | Embodiment 11. Optimal and minimal discriminant predictors, with their |
| | unstandardized coefficients, and group centroids for COAD CMS1 tumors: |
| m) | Set m): Optimal discriminant function: AASDH (−94.5) + ACADVL (−272.0) + BCAT1 (−85.9) + |
| | BMF (−14.2) + CAP2 (42.4) + CCBE1 (−447.0) + CDH4 (−404.0) + DUSP14 (6.8) + EMG1 |
| | (10.8) + EPHA2 (−191.6) + ESM1 (54.9) + HEXB (−49.2) + HOXA5 (−74.4) + IGSF9 (−101.4) + |
| | MCM5 (71.4) + PDLIM2 (−23.0) + PEPD (31.0) + PKP4 (103.0) + PNRC1 (51.3) + RCN2 |
| | (45.2) + S100A14 (337.1) + SDC2 (−29.2) + SLC30A9 (−223.9) + SSPN (207.3) + TFF1 |
| | (127.9) + TMEM140 (64.0) + TMEM45B (331.8) + UBC (−958.1) + ZDHHC18 (−68.7) + 678.4 |
| | Optimal function group centroids: TEAD-inactive (I) 16.4; TEAD-active (A) −157.4 |
| n) | Set n): Minimal discriminant function: TMEM45B (20.3) + TFF1 (8.0) + SSPN (16.0) + |
| | S100A14 (18.4) + EPHA2 (−11.6) + SLC30A9 (−11.1) + HOXA5 (−3.8) + BCAT1 (−6.5) - 25.8 |
| | Minimal function group centroids: TEAD-inactive (I) 1.0; TEAD-active (A) −9.9 |
| | Embodiment 12. Optimal and minimal discriminant predictors, with their |
| | unstandardized coefficients, and group centroids for COAD CMS2 tumors: |
| o) | Set o): Optimal discriminant function: AURKB (5.9) + CENPI (−20.7) + CENPM (10.3) + |
| | CLDN4 (233.5) + DDR1 (34.6) + DLL1 (−4.4) + EPS8L3 (8.2) + FDPS (10.0) + FEN1 (−10.7) + |
| | KRT8 (157.8) + PRPS2 (13.6) + RAB32 (−7.3) + SLC1A2 (−89.9) + TSPAN2 (−5.7) − 432.7 |
| | Optimal function group centroids: TEAD-inactive (I) 0.6; TEAD-active (A) −12.4 |
| p) | Set p): Minimal discriminant function: CLDN4 (196.9) + EPS8L3 (8.0) + CENPI (−12.0) + |
| | DDR1 (27.1) + AURKB (4.8) + CENPM (4.7) − 228.6 |
| | Minimal function group centroids: TEAD-inactive (I) 0.4; TEAD-active (A) −8.0 |
| | Embodiment 13. Optimal and minimal discriminant predictors, with their |
| | unstandardized coefficients, and group centroids for COAD CMS4 tumors: |
| q) | Set q): Optimal discriminant function: ADRB2 (338.8) + BHLHE41 (144.4) + CAVIN1 |
| | (−342.7) + CBR3 (51.3) + CCN2 (169.4) + CHST13 (203.2) + CTSB (−518.2) + CXXC5 (78.7) + |
| | DDR1 (235.4) + GPRC5A (328.9) + GRAMD2B (−24.8) + IFI44 (−143.8) + IGFBP7 (919.9) + |
| | IGSF9 (24.9) + IRF9 (301.8) + ITGA2 (56.8) + KLF10 (−76.7) + LTBP4 (−89.3) + MATN3 |
| | (−10.3) + MLLT11 (−152.5) + MSX2 (−20.3) + NPAS2 (−107.8) + PCDHB2 (45.0) + PIK3R1 |
| | (−58.0) + PRPS2 (−11.9) + ROR1 (84.9) + RPS24 (7816.7) + S100A14 (−109.2) + SHCBP1 |
| | (16.8) + SLC30A9 (−28.5) + SLC35C1 (−60.3) + SLC3A2 (103.8) + SLK (35.4) + SNCG |
| | (25.6) + TEAD1 (84.6) + TMC7 (−109.6) + TSPAN1 (21.9) + TTLL3 (50.9) + WWC1 (−109.0) − |
| | 8644.2 |
| | Optimal function group centroids: TEAD-inactive (I) 8.4; TEAD-active (A) −535.2 |
| q1) | Set q1): Minimal discriminant function: DDR1 (24.6) − 21.6 |
| | Minimal function group centroids: TEAD-inactive (I) 0.2; TEAD-active (A) −9.6 |
| | Embodiment 14. Optimal and minimal discriminant predictors, with their |
| | unstandardized coefficients, and group centroids for Lymphoid Neoplasm |
| | Diffuse Large B-cell Lymphoma (DLBC) tumors: |
| r) | Set r): Optimal discriminant function: ATXN1 (−11.3) + CKS2 (−131.5) + CLCN3 (−116.8) + |
| | COBL (−53.5) + DSC2 (28.5) + EIF2AK3 (16.3) + INTS3 (46.4) + IRAK2 (−132.2) + NFIB |
| | (211.9) + OTUB2 (−361.2) + RBM23 (71.7) + RBM24 (438.0) + RHOC (−207.9) + SECISBP2 |
| | (137.2) + SLC7A1 (206.8) + SLFN5 (54.8) + SUV39H1 (221.3) + THBS1 (195.5) + |
| | TNFRSF12A (282.3) + TPD52L1 (58.2) + TSPAN1 (−10.0) + TSPAN2 (103.8) + TTLL3 |
| | (−18.9) + UHRF1 (−164.0) + WDR13 (−124.1) − 162.0 |
| | Optimal function group centroids: TEAD-inactive (I) −54.6; TEAD-active (A) 64.5 |
| s) | Set s): Minimal discriminant function: THBS1 (8.2) + RBM24 (24.6) + TNFRSF12A (18.1) + |
| | IRAK2 (−11.5) + SLC7A1 (14.4) + OTUB2 (−20.2) + TSPAN2 (5.8) + COBL (−5.0) − 17.8 |
| | Minimal function group centroids: TEAD-inactive (I) −3.5; TEAD-active (A) 4.2 |
| | Embodiment 15. Optimal and minimal discriminant predictors, with their |
| | unstandardized coefficients, and group centroids for Esophageal carcinoma |
| | (ESCA) tumors: |
| t) | Set t): Optimal discriminant function: ANKRD42 (−9.5) + ANXA3 (−2.2) + AXL (−4.7) + BCAT1 |
| | (2.2) + BCL11B (7.8) + CASP1 (2.4) + CDC25A (−7.0) + CDH4 (8.9) + CDV3 (13.8) + |
| | CENPN (−6.8) + CHST13 (−4.5) + CRIM1 (−5.1) + CXCL1 (−1.6) + CXXC5 (1.9) + CYTH3 |
| | (6.4) + DUT (−3.4) + ELN (2.7) + ERAP2 (.9) + ESM1 (2.6) + HASPIN (−9.5) + IRX5 (−1.8) + |
| | MCM2 (−3.6) + NEDD4L (−5.5) + NEXN (−3.4) + NFIL3 (3.3) + NRIP1 (1.6) + NUAK2 (−2.4) + |
| | OLR1 (−3.6) + PKP2 (5.1) + PLA2G4C (13.9) + PLK2 (−3.6) + PSG6 (−45.1) + RBP1 (−1.5) + |
| | REEP6 (−3.3) + RGS17 (5.6) + SGTB (−13.0) + SLC25A3 (47.4) + SLFN5 (2.1) + SPATA5 |
| | (7.9) + SUV39H1 (5.4) + TMEM144 (2.8) + TPD52L1 (2.9) + TRAPPC6B (−5.1) + TSPAN1 |
| | (1.8) + TYMS (6.3) + UHRF1 (−2.8) + VGF (3.8) + VSNL1 (2.6) + WWC1 (−3.8) + YAP1 (7.1) + |
| | ZNF704 (−4.4) − 52.4 |
| | Optimal function group centroids: TEAD-inactive (I) 0.8; TEAD-active (A) −10.1 |
| u) | Set u): Minimal discriminant function: CHST13 (4.5) + PLK2 (1.8) + CENPN (2.9) + |
| | ANKRD42 (9.4) + AXL (3.2) + PSG6 (27.9) + CDC25A (4.9) + IRX5 (2.9) + NRIP1 (−2.8) + |
| | OLR1 (2.7) + TYMS (−4.0) + CTH (−2.8) + HASPIN (6.0) + BCL11B (−6.5) + NEXN (2.6) + |
| | ELN (−2.2) + CXCL1 (1.4) + TRAPPC6B (4.9) + SLFN5 (−2.2) + PLA2G4C (−10.9) + CASP1 |
| | (−1.2) + NUAK2 (1.8) + NEDD4L (2.8) + YAP1 (−4.8) + ANXA3 (2.0) + TSPAN1 (−1.7) + |
| | MLLT11 (−1.5) + SLC25A3 (−27.6) + SGTB (5.9) + CENPM (2.1) + CRIM1 (4.0) + ESM1 |
| | (−1.8) + CDH4 (−3.4) + PKP2 (−1.6) + UHRF1 (2.0) + 19.6 |
| | Minimal function group centroids: TEAD-inactive (I) −0.6; TEAD-active (A) 7.4 |
| | Embodiment 16. Optimal and minimal discriminant predictors, with their |
| | unstandardized coefficients, and group centroids for Glioblastoma |
| | multiforme (GBM) tumors: |
| v) | Set v): Optimal discriminant function: AASS (−17.4) + ACSL5 (8.9) + AMOT (−8.9) + |
| | ARHGEF2 (5.9) + BCAT1 (−4.6) + BHLHE41 (13.4) + CA2 (−9.9) + CAV1 (−4.2) + CCN2 |
| | (8.6) + CCNG2 (−8.3) + CDCA3 (−4.4) + CELSR3 (−4.5) + CENPI (−21.0) + CLCN3 (17.9) + |
| | CLDN1 (−9.1) + COTL1 (−27.9) + CYP27C1 (16.0) + DCLRE1B (−18.4) + ERAP2 (2.6) + |
| | FAM102A (6.9) + FAM89B (−26.0) + FDPS (10.7) + GADD45B (−14.5) + GINS1 (7.3) + |
| | GPRC5B (−12.7) + HASPIN (34.3) + HEXB (−6.6) + IFI44 (8.1) + IGSF3 (8.4) + IRAK2 (8.8) + |
| | KRCC1 (6.6) + KRT8 (−17.1) + LMCD1 (−3.3) + MCM10 (−18.3) + MGST2 (11.9) + MLLT11 |
| | (4.7) + MTMR11 (4.8) + NFIL3 (14.6) + NUAK1 (−3.7) + OAS1 (3.1) + OFD1 (−3.2) + PAK1 |
| | (8.6) + PCDHB9 (5.5) + PCGF3 (8.4) + PHLPP1 (−16.4) + PIK3C2B (−2.8) + PIP4P2 (20.4) + |
| | PKP2 (−7.7) + PLK2 (−3.4) + PSG6 (71.4) + RAB32 (−5.2) + RACGAP1 (−3.8) + RALGPS1 |
| | (4.2) + RBM45 (16.4) + ROR1 (−14.1) + SHROOM2 (7.1) + SP1 (25.6) + SRD5A3 (4.0) + |
| | STK3 (−9.0) + TACC3 (10.8) + TMC4 (5.7) + TRIM31 (−118.7) + TUBB6 (5.8) + VPS52 |
| | (32.8) + VSNL1 (3.8) − 37.2 |
| | Optimal function group centroids: TEAD-inactive (I) 1.7; TEAD-active (A) −9.7 |
| w) | Set w): Minimal discriminant function: CENPI (−13.1) + IFI44 (7.0) + SP1 (18.1) + AMOT |
| | (−4.3) + ROR1 (−8.1) + BCAT1 (−2.6) + IRAK2 (8.5) + KRT8 (−9.7) + TRIM31 (−60.7) + RBM45 |
| | (11.1) + VPS52 (20.5) + PHLPP1 (−10.6) + TMC4 (9.1) + BHLHE41 (7.0) + HASPIN (20.1) + |
| | TRIOBP (−3.4) + MCM10 (−14.0) + CAV1 (−4.2) + CCN2 (6.1) + CLCN3 (11.6) + CYP27C1 |
| | (6.3) + MTMR11 (3.7) + VSNL1 (2.1) + IGSF3 (3.3) + SHROOM2 (4.8) + CLDN1 (−7.2) + |
| | AASS (−11.4) + DCLRE1B (−9.8) + LMCD1 (−3.3) + GADD45B (−7.3) + NFIL3 (8.1) + TACC3 |
| | (4.6) + CA2 (−9.4) + PCDHB9 (3.1) + ACSL5 (7.0) + NUAK1 (−4.5) + PIP4P2 (6.6) + PCGF3 |
| | (4.8) + FAM89B (−9.2) + PKP2 (−3.7) + PAK1 (4.8) + TUBB6 (2.5) + FDPS (9.7) − 38.8 |
| | Minimal function group centroids: TEAD-inactive (I) 1.1; TEAD-active (A) −6.0 |
| | Embodiment 17. Optimal and minimal discriminant predictors, with their |
| | unstandardized coefficients, and group centroids for Head and Neck |
| | squamous cell carcinoma (HNSC) tumors: |
| x) | Set x): Optimal discriminant function: ACAT2 (−.9) + ANKRD22 (1.1) + ANKRD29 (1.6) + |
| | ANTXR2 (1.5) + ANXA3 (−1.3) + B4GALT4 (−1.4) + BHLHE41 (−1.5) + CA2 (1.7) + CAV1 |
| | (1.5) + CCBE1 (−6.0) + CDCA3 (−1.2) + CENPN (−2.3) + CNN3 (−1.7) + CRIM1 (−2.0) + |
| | CTNNBIP1 (3.7) + CYTH3 (−3.1) + DDAH1 (−2.6) + DSC2 (1.8) + EIF2AK3 (2.5) + ERAP2 |
| | (.6) + F3 (1.2) + FAM117B (2.5) + FEN1 (4.4) + GOLGA5 (4.2) + GSN (10.2) + HSPB11 |
| | (3.0) + IKBIP (−2.9) + LRRFIP2 (−2.9) + LSM5 (−2.3) + MANSC1 (2.2) + METRNL (2.1) + |
| | MGST2 (3.8) + NEDD4 (−1.8) + NUAK1 (1.5) + NUAK2 (−1.5) + PCBD1 (−2.6) + PEPD (4.1) |
| | + PHF21A (2.8) + PHLPP1 (−3.5) + PIK3R1 (−1.2) + PLCE1 (2.5) + PSG2 (−20.8) + RFC4 |
| | (−1.7) + SCD5 (−1.1) + SGTB (6.6) + SLC38A5 (−1.2) + SPRY4 (1.4) + SQSTM1 (3.9) + |
| | STX1A (2.9) + SUSD2 (1.4) + TMC4 (1.2) + TSPAN1 (1.0) + ZNF704 (2.5) − 26.8 |
| | Optimal function group centroids: TEAD-inactive (I) 0.5; TEAD-active (A) −4.4 |
| | Minimal discriminant function: NA |
| | Embodiment 18. Optimal and minimal discriminant predictors, with their |
| | unstandardized coefficients, and group centroids for Kidney Chromophobe |
| | (KICH) tumors: |
| y) | Set y): Optimal discriminant function: ACSL5 (63.3) + AMOT (−44.2) + ANXA3 (56.4) + |
| | ARHGAP11A (−311.3) + ARHGEF2 (−53.4) + AXL (19.5) + CASP1 (58.2) + CDC25A (277.7) + |
| | CELSR3 (183.5) + CENPN (12.3) + COL8A1 (−47.5) + CXXC5 (−254.0) + DONSON (17.3) + |
| | FAHD2A (64.5) + FAM83B (54.2) + FERMT1 (51.2) + FSTL1 (27.0) + GPC6 (−7.5) + |
| | GPRC5C (−113.1) + HASPIN (412.1) + HBP1 (53.3) + HDAC1 (63.6) + IFI44 (−57.8) + |
| | IGFBP7 (−773.8) + KRTDAP (−89.9) + LRP8 (100.1) + MANSC1 (−63.4) + MAPK13 (12.7) + |
| | MATN2 (19.9) + MICB (66.3) + MID1 (68.8) + MXRA7 (−107.4) + NEDD4 (−10.9) + NEK2 |
| | (173.9) + NRIP1 (−52.6) + NUP107 (223.2) + PAG1 (41.4) + PIK3C2B (24.4) + PIK3R3 |
| | (87.7) + PIP4P2 (−134.7) + QDPR (39.0) + RACGAP1 (6.3) + RBP1 (32.5) + RCN2 (67.2) + |
| | RGS17 (111.4) + SECISBP2 (−136.2) + SLC25A3 (592.6) + SLC39A11 (−62.2) + SRD5A3 |
| | (−18.5) + ULK1 (30.8) + VKORC1L1 (−29.6) + ZNF488 (129.8) + 289.3 |
| | Optimal function group centroids: TEAD-inactive (I) −9.7; TEAD-active (A) 433.0 |
| z) | Set z): Minimal discriminant function: HASPIN (33.2) + MATN2 (4.1) + CXXC5 (−12.2) + |
| | CELSR3 (10.5) + RBP1 (2.5) + 4.2 |
| | Minimal function group centroids: TEAD-inactive (I) −0.3; TEAD-active (A) 13.6 |
| | Embodiment 19. Optimal and minimal discriminant predictors, with their |
| | unstandardized coefficients, and group centroids for Kidney renal clear cell |
| | carcinoma (KIRC) tumors: |
| aa) | Set aa): Optimal discriminant function: ACAT2 (3.3) + APBB3 (−1.8) + ATXN1 (−3.3) + |
| | AURKB (4.1) + BCAT1 (2.9) + BIRC5 (3.8) + CDC42EP4 (−3.7) + CDC6 (7.7) + CDCA3 |
| | (4.6) + CDCA8 (3.1) + CDK1 (−7.7) + CENPA (9.4) + CLDN4 (1.5) + CRIM1 (9.3) + CYTH3 + |
| | (4.7) + DKK1 (−2.1) + EPS8L2 (5.0) + F3 (2.1) + FOS (2.0) + GPC6 (.8) + GPRC5C (−3.8) |
| | HAS3 (2.7) + KRT8 (10.3) + LSM5 (3.7) + MGST2 (−6.2) + MTMR11 (2.8) + MXRA7 (1.5) + |
| | NEDD4L (1.2) + OLR1 (.8) + PHF21A (−6.1) + QKI (−4.2) + RBM23 (1.8) + RBM24 (2.5) + |
| | RNF144B (3.2) + SDC2 (−2.5) + SKP2 (5.7) + SLC38A5 (.9) + SNAPC1 (1.9) + SSPN |
| | (−3.4) + TFF1 (2.2) + TMEM139 (−1.6) + TMEM140 (−3.6) + TPX2 (3.8) + TSPAN1 (−.8) + |
| | UBE2C (−4.6) + ZCWPW1 (−4.3) − 17.8 |
| | Optimal function group centroids: TEAD-inactive (I) −0.3; TEAD-active (A) 10.6 |
| | Minimal discriminant function: NA |
| | Embodiment 20. Optimal and minimal discriminant predictors, with their |
| | unstandardized coefficients, and group centroids for Kidney renal papillary |
| | cell carcinoma (KIRP) tumors: |
| bb) | Set bb): Optimal discriminant function: ABLIM3 (−2.3) + AURKB (−4.2) + BCAT1 (6.4) + |
| | BHLHE41 (−4.4) + CDCA3 (8.0) + CDCA8 (10.0) + CDH4 (3.3) + CEBPB (2.9) + COL6A1 |
| | (−6.0) + COL6A2 (1.7) + CRABP2 (−2.5) + CSTA (2.5) + CTSK (2.5) + CXCL1 (−1.7) + |
| | DAPK1 (3.7) + DDR1 (5.6) + ESM1 (1.2) + EXO1 (9.8) + FAM89B (−10.0) + FANCA (−4.1) + |
| | FSTL1 (5.5) + FXYD3 (−1.8) + GINS1 (5.3) + GOLPH3L (−3.1) + ITGA2 (3.4) + KCNMA1 |
| | (−1.7) + LRP8 (−3.2) + LRRFIP2 (5.5) + LTBP4 (2.2) + MAPK13 (−1.7) + MTSS1 (−6.9) + |
| | MYO1A (−9.4) + NAPEPLD (−6.2) + OLFML3 (3.3) + PAK1 (5.3) + PERP (−5.6) + PLOD2 |
| | (−3.2) + PSG2 (40.2) + RBM23 (4.6) + RFC4 (5.2) + SECISBP2 (3.7) + SHCBP1 (−3.7) + |
| | SLC25A23 (11.5) + SLC25A3 (−40.9) + SLC37A2 (−3.5) + SNCG (.8) + SPATA5 (14.0) + |
| | TENT5B (3.0) + TRIM31 (4.5) + TTF2 (9.8) + VAMP8 (52.4) + WDR13 (−13.1) − 20.0 |
| | Optimal function group centroids: TEAD-inactive (I) −0.3; TEAD-active (A) 16.4 |
| cc) | Set cc): Minimal discriminant function: CENPA (3.3) + WDR13 (−10.5) + PERP (−3.6) + |
| | MTSS1 (−4.8) + BHLHE41 (−4.5) + DAPK1 (3.8) + CRABP2 (−1.8) + GOLPH3L (−7.8) + |
| | ABLIM3 (−2.2) + RBM23 (3.3) + COL6A1 (−8.3) + MYO1A (−7.4) + CDCA8 (5.3) + COL6A2 |
| | (2.7) + MAPK13 (−2.1) + CDH4 (2.5) + BCAT1 (4.8) + PLOD2 (−2.9) + SPATA5 (10.6) + |
| | RFC4 (4.4) + NAPEPLD (−5.3) + PAK1 (3.4) + SLC25A23 (10.7) + OLFML3 (2.5) + TTF2 |
| | (8.3) + PIK3R2 (−5.2) + CDCA3 (5.5) + KCNMA1 (−1.4) + GRB10 (2.4) + SGMS2 (2.1) + |
| | 11.3 |
| | Minimal function group centroids: TEAD-inactive (I) −0.2; TEAD-active (A) 11.6 |
| | Embodiment 21. Optimal and minimal discriminant predictors, with their |
| | unstandardized coefficients, and group centroids for Brain Lower Grade |
| | Glioma (LGG) tumors: |
| dd) | Set dd): Optimal discriminant function: ADAMTS1 (1.5) + ARAP3 (−3.2) + BTG3 (−2.3) + |
| | CCN2 (1.9) + CDCA8 (4.2) + CLCN3 (−3.3) + CSRNP2 (−4.0) + CTSB (−16.8) + EBP (−2.8) + |
| | ERAP2 (−.8) + ESM1 (1.2) + EXO1 (13.5) + FAM83B (31.6) + FAM83D (3.7) + FAM89B |
| | (−8.4) + FAT4 (4.6) + GOLGA5 (−9.5) + GPRC5B (12.4) + HAS3 (7.2) + IRAK2 (−2.9) + IRF9 |
| | (2.5) + IRX5 (−3.7) + KCNMA1 (1.5) + KCNMB3 (−4.8) + KIFAP3 (−5.6) + LMCD1 (3.6) + |
| | LMNB2 (8.6) + MCM10 (−4.1) + MICB (−3.9) + MSX2 (1.0) + NAPEPLD (−3.8) + NEDD4L |
| | (−1.6) + NEK2 (3.6) + NEXN (4.6) + PCMTD2 (−5.6) + PHLPP1 (10.3) + PIK3C2B (−3.2) + |
| | PPIH (−4.9) + PRPS2 (1.5) + PYGB (−10.0) + RBM24 (4.4) + SCML1 (2.5) + SEC14L2 |
| | (−1.5) + SGMS2 (−7.3) + SKP2 (4.4) + SLC7A5 (4.1) + SNAPC1 (3.9) + SNCG (1.7) + SNX24 |
| | (6.8) + SQSTM1 (−13.3) + SUSD2 (2.7) + TACC3 (−2.3) + TBX3 (−5.3) + TENT5B (2.8) + |
| | TFF1 (5.0) + TNFAIP3 (4.2) + TP53INP1 (−2.6) + TPX2 (−3.4) + TRAPPC6B (6.4) + VGF |
| | (−1.1) + WDR13 (−11.0) + WWC1 (−2.0) + ZNF467 (3.4) + 38.4 |
| | Optimal function group centroids: TEAD-inactive (I) −0.2; TEAD-active (A) 11.3 |
| ee) | Set ee): Minimal discriminant function: EXO1 (12.2) + FAM83B (35.4) + SUSD2 (2.4) + |
| | CTSB (−14.7) + NEXN (4.1) + TNFAIP3 (4.1) + PIK3C2B (−3.1) + FAM83D (4.4) + TPX2 |
| | (−5.7) + TBX3 (−5.8) + PPIH (−4.4) + NEK2 (4.1) + MICB (−3.3) + CLCN3 (−2.6) + PHLPP1 |
| | (10.0) + ADAMTS1 (1.7) + LMCD1 (3.4) + GOLGA5 (−5.8) + WDR13 (−12.9) + SCML1 (2.4) + |
| | SNCG (1.1) + NEDD4L (−2.2) + SEC14L2 (−2.2) + BTG3 (−2.2) + CCN2 (2.7) + SQSTM1 |
| | (−13.1) + IRF9 (4.3) + FAT4 (4.9) + TP53INP1 (−2.5) + IRAK2 (−2.8) + RBM24 (3.8) + LMNB2 |
| | (6.1) + CSRNP2 (−4.7) + TRAPPC6B (3.6) + ZNF467 (2.5) + SKP2 (3.4) + SGMS2 (−5.9) + |
| | TENT5B (2.7) + PCMTD2 (−4.5) + SNX24 (4.3) + FAM89B (−5.6) + HAS3 (3.8) + KCNMB3 |
| | (−4.0) + 34.3 |
| | Minimal function group centroids: TEAD-inactive (I) −0.2; TEAD-active (A) 9.6 |
| | Embodiment 22. Optimal and minimal discriminant predictors, with their |
| | unstandardized coefficients, and group centroids for Liver hepatocellular |
| | carcinoma (LIHC) tumors: |
| ff) | Set ff): Optimal discriminant function: ACADVL (−17.1) + CAV1 (2.4) + CBR3 (−2.3) + |
| | CDCA8 (2.7) + CEBPB (6.3) + CPA4 (3.0) + DONSON (5.0) + EPS8L2 (−5.3) + FAM89B |
| | (−4.0) + FANCA (4.1) + FAT4 (3.2) + GPRC5B (1.4) + GSN (−3.4) + HASPIN (7.2) + IGFBP7 |
| | (15.9) + KRIT1 (−3.2) + MAD2L1 (3.6) + MFSD1 (−4.5) + MICB (−2.5) + MLLT11 (1.9) + |
| | NAPEPLD (−2.7) + NCAPD3 (3.3) + NEDD4L (−1.1) + OLR1 (1.1) + PEPD (−9.0) + PIK3R2 |
| | (−4.3) + PLEKHA7 (−2.0) + QDPR (3.9) + SH3PXD2A (−4.3) + SKP2 (2.7) + SLC7A5 (1.0) + |
| | SMPD4 (3.4) + TMEM140 (−3.5) + TMEM160 (−1.9) + TRIM38 (−2.6) + UHRF1 (−5.2) + |
| | ZDHHC18 (2.5) + ZNF467 (1.8) + 13.4 |
| | Optimal function group centroids: TEAD-inactive (I) −0.5; TEAD-active (A) 5.1 |
| | Minimal discriminant function: NA |
| | Embodiment 23. Optimal and minimal discriminant predictors, with their |
| | unstandardized coefficients, and group centroids for LIHC S1 tumors: |
| gg) | Set gg): Optimal discriminant function: AASS (24.7) + ABLIM3 (−88.7) + ACADVL (−216.5) + |
| | ARHGAP11A (124.1) + B4GALT4 (16.6) + CAP2 (−35.2) + CBR3 (−18.8) + CCDC80 (29.7) + |
| | CDCA5 (−225.2) + CDV3 (33.3) + COBL (82.5) + CPE (13.5) + DAPK1 (−14.9) + DHCR7 |
| | (138.5) + DUT (−18.1) + ERAP2 (−7.0) + EXO1 (76.1) + FAT4 (−58.9) + KLK11 (22.3) + |
| | KRIT1 (−22.4) + LMCD1 (34.6) + LYPD6 (30.7) + MCM10 (255.9) + MLLT11 (72.9) + |
| | MMP13 (−84.4) + NTN4 (7.4) + PAK1 (−89.5) + PNRC1 (110.9) + QKI (47.0) + RHOC (370.8) + |
| | SLC3A2 (67.1) + SLK (114.8) + SUSD2 (−14.4) + SUV39H1 (136.5) + TACC3 (−32.7) + |
| | TLR3 (18.1) + UBE2C (17.3) + VKORC1L1 (−250.2) + ZNF292 (−35.7) − 370.2 |
| | Optimal function group centroids: TEAD-inactive (I) −2.7; TEAD-active (A) 205.4 |
| hh) | Set hh): Minimal discriminant function: MCM10 (25.7) + CDCA5 (−8.7) + MLLT11 (4.4) − 0.8 |
| | Minimal function group centroids: TEAD-inactive (I) −0.1; TEAD-active (A) 9.3 |
| | Embodiment 24. Optimal and minimal discriminant predictors, with their |
| | unstandardized coefficients, and group centroids for LIHC S2 tumors: |
| ii) | Set ii): Optimal discriminant function: ABAT (39.1) + ABI3BP (63.9) + ACADVL (523.7) + |
| | ARHGAP11A (−24.1) + ATXN1 (87.8) + BCL11B (−36.8) + BTG3 (−73.6) + CCBE1 (−164.8) + |
| | CCN2 (−109.2) + CDC25A (−81.8) + CDC42EP4 (230.9) + CDCA4 (120.2) + COL6A1 |
| | (208.4) + COL8A1 (94.6) + CTH (52.1) + CYTH3 (192.5) + DDR1 (8.1) + DEDD2 (211.1) + |
| | DIAPH2 (42.8) + DLL1 (21.5) + DONSON (−160.1) + DUSP6 (188.9) + DYNC2LI1 (106.5) + |
| | ELN (−45.2) + EMG1 (52.1) + ERAP2 (34.1) + ESM1 (43.5) + EXOSC2 (−51.0) + FANCA |
| | (−30.7) + FAT4 (−53.2) + FEN1 (−57.3) + FOS (−103.2) + FST (−61.6) + FSTL3 (−5.1) + GINS1 |
| | (253.9) + GSN (235.3) + HBP1 (−77.8) + HDAC1 (255.9) + HEY1 (16.8) + IGSF3 (15.6) + |
| | IGSF9 (−16.5) + IRX5 (51.8) + KCNMB3 (53.5) + KPNA2 (47.5) + KRCC1 (107.4) + KRIT1 |
| | (21.0) + KRTDAP (156.7) + LHFPL6 (34.3) + LMTK3 (9.9) + MAD2L1 (−132.9) + MFSD1 |
| | (200.5) + MLLT11 (−81.9) + MYO1A (−18.2) + NCAPD3 (−146.1) + NEDD4L (81.7) + NFIB |
| | (−138.4) + NPAS2 (43.0) + PAG1 (−118.4) + PCDHB2 (51.2) + PIK3C2B (76.9) + PIK3R2 |
| | (32.0) + PLCE1 (−13.1) + PRPS1 (60.2) + PRPS2 (19.4) + PTPRE (31.6) + PYGB (−101.4) + |
| | QDPR (−76.3) + RACGAP1 (182.5) + RFC4 (−141.3) + RHOC (−109.4) + RNF144B |
| | (−127.8) + SH3PXD2A (37.2) + SHCBP1 (−64.1) + SLC25A3 (164.0) + SLC35C1 (177.8) + |
| | SLC39A11 (−10.2) + SLC3A2 (109.9) + SMPD4 (−236.4) + STX11 (94.5) + TAGLN (−229.4) + |
| | TLR3 (45.7) + TMEM144 (−45.5) + TP63 (370.4) + TRAPPC6B (−159.9) + TRIB2 (−10.0) + |
| | TRIM31 (85.0) + TRIOBP (33.0) + TRIP13 (−218.6) + TSC22D1 (−296.6) + VAMP8 (226.1) + |
| | VGF (−11.1) + ZSWIM7 (−15.0) − 1382.5 |
| | Optimal function group centroids: TEAD-inactive (I) 33.6; TEAD-active (A) −163.4 |
| jj) | Set jj): Minimal discriminant function: TRIP13 (−13.4) + SLC35C1 (10.5) + DYNC2LI1 (4.7) + |
| | TP63 (17.0) + MLLT11 (−5.3) + RNF144B (−6.0) + MAD2L1 (−9.2) + GSN (16.1) + TAGLN |
| | (−12.1) + CCBE1 (−6.6) + FST (−5.4) + DEDD2 (10.6) + DUSP6 (9.5) + CYTH3 (8.9) + |
| | TRIM31 (3.2) + GINS1 (11.4) + DCLRE1B (−6.8) + KRCC1 (7.9) + CDC42EP4 (9.3) + |
| | COL6A1 (8.4) + KRTDAP (7.7) + MFSD1 (7.7) + DONSON (−9.7) + FOS (−5.0) + AASS |
| | (2.7) + SMPD4 (−10.3) + TRAPPC6B (−5.9) + PIK3R2 (5.4) + ESM1 (2.5) + NFIB (−6.8) + |
| | TSC22D1 (−11.0) + CTH (3.6) + RACGAP1 (8.7) + NCAPD3 (−6.1) + ACADVL (24.7) + |
| | PHF21A (5.2) + PAG1 (−2.4) − 48.7 |
| | Minimal function group centroids: TEAD-inactive (I) 1.6; TEAD-active (A) −7.9 |
| | Embodiment 25. Optimal and minimal discriminant predictors, with their |
| | unstandardized coefficients, and group centroids for LIHC S3 tumors: |
| kk) | Set kk): Optimal discriminant function: ADRB2 (5.8) + ARHGEF2 (3.8) + DUSP6 (−4.9) + |
| | EXO1 (−6.1) + GPRC5B (3.1) + GSN (−4.5) + IL6 (10.4) + MAD2L1 (10.4) + NAGK (−7.1) + |
| | OLR1 (1.6) + PERP (4.0) + PLK2 (−6.5) + PSG7 (15.5) + PXMP2 (−23.1) + RBMS2 (7.7) + |
| | RBP1 (1.1) + SCML1 (−5.2) + SH3RF1 (5.1) + TP53INP1 (−4.7) + TPD52L1 (−2.2) + TSPAN2 |
| | (−14.7) + ZCWPW1 (−5.4) + 33.0 |
| | Optimal function group centroids: TEAD-inactive (I) −0.5; TEAD-active (A) 7.8 |
| ll) | Set ll): Minimal discriminant function: MAD2L1 (8.5) + IL6 (9.1) + PSG7 (14.1) + ZCWPW1 |
| | (−5.6) + NAGK (−7.7) + DUSP6 (−4.5) + OLR1 (1.4) + PLK2 (−5.7) + EXO1 (−5.2) + PERP |
| | (3.0) + TP53INP1 (−3.1) + ADRB2 (3.4) + SCML1 (−4.0) + ARHGEF2 (2.7) + 10.9 |
| | Minimal function group centroids: TEAD-inactive (I) −0.4; TEAD-active (A) 6.1 |
| | Embodiment 26. Optimal and minimal discriminant predictors, with their |
| | unstandardized coefficients, and group centroids for Lung adenocarcinoma |
| | (LUAD) tumors: |
| mm) | Set mm): Optimal discriminant function: ACOT11 (−2.0) + ADAM28 (2.5) + ADM (−.9) + |
| | ANKRD42 (4.9) + ANXA3 (1.0) + ARHGEF2 (1.3) + ATP7A (2.1) + AXL (1.9) + BHLHE41 |
| | (−2.3) + CASP1 (−2.1) + CAV1 (2.7) + CCBE1 (1.2) + CDC25A (6.9) + CELSR3 (−1.7) + |
| | CLIC3 (.9) + COTL1 (5.4) + CYP1B1 (1.0) + CYTH3 (1.4) + DHFR (1.3) + DHX32 (−1.1) + |
| | DIAPH3 (3.1) + DSG3 (−3.0) + DUSP14 (−1.4) + DUT (−2.4) + DYNC2LI1 (2.5) + EXO1 (5.5) + |
| | FEN1 (1.7) + FST (.8) + GPR176 (2.5) + GRB10 (1.2) + HBP1 (−1.8) + HOXA5 (−1.6) + |
| | IGFBP7 (−10.2) + IGSF3 (−2.6) + INTS3 (1.9) + KCNMB3 (2.1) + KIFAP3 (−2.3) + KLF13 |
| | (−2.7) + KRTDAP (5.0) + MGST2 (−3.4) + MLPH (−2.6) + NEDD4 (−1.5) + NEK2 (−1.7) + |
| | NUAK2 (2.1) + PAK1 (−1.8) + PRSS23 (−1.4) + RAB32 (3.9) + RBM47 (−2.1) + RBP1 (−1.5) + |
| | SCML1 (−2.3) + SEC14L1 (1.8) + SGK1 (1.5) + SH3PXD2A (1.9) + SLC37A2 (−2.0) + |
| | SNCG (−.6) + SP1 (−2.8) + STXBP6 (2.3) + TACC3 (−2.3) + TMEM144 (2.2) + TMEM45B |
| | (−1.2) + TTF2 (4.3) + VGF (−1.6) + WDR13 (2.9) + WWC2 (−3.1) + ZNF488 (7.3) + 6.1 |
| | Optimal function group centroids: TEAD-inactive (I) −0.4; TEAD-active (A) 5.8 |
| | Minimal discriminant function: NA |
| | Embodiment 27. Optimal and minimal discriminant predictors, with their |
| | unstandardized coefficients, and group centroids for LUAD proximal- |
| | inflammatory tumors: |
| nn) | Set nn): Optimal discriminant function: ACAT2 (7.3) + ACOT11 (−22.3) + AGL (−20.3) + |
| | ANTXR2 (−6.9) + AXL (20.8) + B4GALT4 (4.3) + CAVIN1 (−34.7) + CCN2 (−17.5) + CCNG2 |
| | (−10.5) + CDC25A (15.2) + CDH4 (18.0) + CENPA (8.3) + CHRNB1 (11.7) + COBL (14.4) + |
| | CPA4 (41.6) + CTNNBIP1 (−19.7) + DDR1 (14.3) + DHFR (6.4) + DONSON (−6.9) + EBP |
| | (8.9) + EPS8L2 (23.9) + EXO1 (58.4) + FAHD2A (−11.9) + FAM83D (−20.3) + FEN1 (17.5) + |
| | GOLGA5 (−10.0) + GRAMD2B (10.6) + IFI44 (7.9) + INTS3 (10.1) + IRAK2 (−2.9) + IRX5 |
| | (−7.9) + KCNMB3 (−18.2) + KRCC1 (−10.8) + KRT8 (−16.9) + KRTDAP (69.3) + MATN2 (6.4) + |
| | MRPL33 (48.3) + NAGK (−15.6) + NEXN (28.0) + NUP37 (−21.8) + OAS2 (−6.7) + OTUB2 |
| | (6.1) + PAK2 (37.8) + PERP (−10.9) + PKIA (−9.9) + PKMYT1 (14.5) + PLK2 (7.0) + PNRC1 |
| | (−11.2) + PRRX2 (20.6) + RBM45 (19.9) + RBP1 (−3.7) + REEP6 (−3.0) + SLITRK6 (−8.9) + |
| | SMPD4 (−31.1) + SNX24 (5.4) + STXBP6 (18.2) + TK1 (−24.5) + TMC4 (5.4) + TMC7 |
| | (−18.3) + TRIM13 (10.1) + TSPAN1 (−3.7) + UAP1 (−23.6) + VPS52 (−65.5) + WWC1 (−7.7) + |
| | YAP1 (24.7) + 57.7 |
| | Optimal function group centroids: TEAD-inactive (I) −3.6; TEAD-active (A) 29.8 |
| oo) | Set oo): Minimal discriminant function: ACOT11 (−4.1) + EXO1 (11.4) + AXL (6.7) + |
| | CHRNB1 (4.8) + KRTDAP (24.9) + CTNNBIP1 (−9.2) + PSG2 (93.7) + TNFRSF12A (9.4) + |
| | CPA4 (8.6) + PDLIM2 (5.6) + FEN1 (5.5) + VPS52 (−9.0) + FAM83D (−4.2) + MDC1 (3.1) + |
| | EBP (5.7) + NAPEPLD (4.3) + TK1 (−12.6) + EPS8L2 (4.8) + NEXN (5.3) + CENPA (7.5) + |
| | COBL (2.9) + TNFAIP3 (−3.1) + DHFR (5.0) + ANTXR2 (−3.8) + NUP37 (−4.4) − 11.0 |
| | Minimal function group centroids: TEAD-inactive (I) −0.8; TEAD-active (A) 7.0 |
| | Embodiment 28. Optimal and minimal discriminant predictors, with their |
| | unstandardized coefficients, and group centroids for LUAD proximal- |
| | proliferative tumors: |
| pp) | Set pp): Optimal discriminant function: ATP7A (−15.9) + ATXN1 (8.6) + CCBE1 (−24.2) + |
| | CCN2 (−5.6) + CDCA4 (−5.9) + CDK6 (−8.1) + COL8A1 (4.3) + CRABP2 (3.9) + CRIM1 |
| | (−14.0) + CSTA (2.4) + CTH (−3.0) + CTSK (5.5) + DCLRE1B (−21.6) + DHX32 (8.2) + EPS8L3 |
| | (−4.6) + ERAP2 (1.7) + F3 (−6.3) + FAT4 (−32.5) + GPR176 (−9.3) + HBP1 (12.9) + HDHD2 |
| | (2.7) + HEY1 (6.9) + IGSF3 (11.5) + IGSF9 (2.6) + IKBIP (−6.3) + JPH2 (−11.2) + KCNMA1 |
| | (8.1) + KCNMB3 (16.7) + KLHL3 (31.0) + KRIT1 (−14.2) + LRP10 (12.8) + LRRFIP2 (4.3) + |
| | LYRM1 (8.3) + MAPK13 (8.8) + METRNL (5.6) + MSX2 (−10.4) + NCOA3 (20.0) + NEXN |
| | (−5.7) + NOC3L (−4.4) + NUP37 (11.1) + OAS1 (−6.1) + OLFML3 (3.7) + PAK2 (−19.7) + |
| | PDLIM2 (−13.0) + PEPD (7.6) + PJA2 (3.5) + PKP4 (7.7) + PLA2G4C (−5.5) + PLK2 (−2.7) + |
| | PSG6 (13.3) + PVR (−9.8) + RALGPS1 (−10.1) + RBM23 (−10.8) + RBM24 (17.1) + RFC4 |
| | (5.2) + RGS17 (−14.8) + RHOC (44.5) + RNF144B (−3.1) + ROR1 (14.6) + S100A14 (−2.5) + |
| | SMPD4 (10.7) + SSPN (22.0) + TDO2 (3.9) + TLR3 (10.6) + TMC4 (10.9) + TMC7 (14.6) + |
| | TMEM139 (−5.2) + TMEM45B (6.3) + TP63 (−4.0) + TRIM38 (−13.7) + TRIP11 (6.8) + |
| | TSPAN1 (3.0) + TSPAN2 (5.3) + TTLL3 (−6.4) + ZDHHC18 (−9.6) + ZNF488 (−28.4) − 73.7 |
| | Optimal function group centroids: TEAD-inactive (I) 2.4; TEAD-active (A) −19.1 |
| qq) | Set qq): Minimal discriminant function: CDC25A (−3.3) + CCBE1 (−7.2) + TSPAN1 (2.4) + |
| | TMC4 (3.8) + ZNF488 (−6.2) + CTSK (5.8) + CCN2 (−3.3) + HEY1 (2.4) + TMC7 (4.2) + |
| | LYRM1 (4.4) + ATXN1 (4.3) + FAT4 (−9.3) + DHX32 (3.0) + IGSF3 (4.1) + PDLIM2 (−3.6) + |
| | KRIT1 (−6.4) + DCLRE1B (−7.6) + CRIM1 (−3.1) + PVR (−3.6) + NCOA3 (5.2) + OLFML3 |
| | (1.8) + GSN (−8.4) + PLK2 (−1.8) + RHOC (11.3) + GPR176 (−2.1) + KLHL3 (5.7) − 8.6 |
| | Minimal function group centroids: TEAD-inactive (I) 0.7; TEAD-active (A) −5.9 |
| | Embodiment 29. Optimal and minimal discriminant predictors, with their |
| | unstandardized coefficients, and group centroids for Lung squamous cell |
| | carcinoma (LUSC) tumors: |
| rr) | Set rr): Optimal discriminant function: AGL (−1.8) + AGPAT4 (3.3) + ATXN1 (3.1) + AURKB |
| | (−1.6) + BMF (1.6) + CBR3 (1.3) + CCNG2 (1.9) + CDC6 (−1.7) + CDK6 (−2.5) + CDV3 (3.4) + |
| | CENPN (−1.0) + COBL (−2.0) + CRABP2 (−1.3) + CTH (−1.7) + DDR1 (2.4) + DEDD2 |
| | (−2.6) + DIAPH3 (−3.4) + DKK1 (−1.1) + ETS1 (−2.5) + EXO1 (2.6) + GADD45B (−1.5) + |
| | GPRC5C (.8) + GRB10 (1.7) + JPH2 (−3.1) + KRIT1 (2.3) + LYPD3 (1.8) + MALL (1.8) + |
| | MATN3 (−1.1) + MCM10 (−4.1) + MLPH (1.1) + MVD (−1.9) + NEXN (3.1) + NFIL3 (1.6) + |
| | OASL (1.1) + OXCT1 (−2.2) + PCDHB9 (2.2) + PCGF3 (−2.0) + PERP (−10.2) + PKIA (1.6) + |
| | PLOD2 (−1.3) + PRPS1 (−3.0) + RAPGEFL1 (1.5) + RBM45 (4.8) + RBP1 (1.4) + SPIRE2 |
| | (−1.7) + STMN3 (2.0) + SUSD2 (−1.3) + TET2 (−2.3) + TRIB1 (1.7) + TRIP13 (2.6) + VAMP8 |
| | (9.3) − 3.7 |
| | Optimal function group centroids: TEAD-inactive (I) 0.5; TEAD-active (A) −3.5 |
| | Minimal discriminant function: NA |
| | Embodiment 30. Optimal and minimal discriminant predictors, with their |
| | unstandardized coefficients, and group centroids for LUSC basal tumors: |
| ss) | Set ss): Optimal discriminant function: AASS (26.7) + ABAT (−19.4) + ACSL5 (−40.8) + |
| | AGPAT4 (118.2) + ANKRD22 (−43.9) + ARHGEF2 (−28.5) + AXL (193.9) + BIRC5 (−36.2) + |
| | C4BPB (81.6) + CAV1 (14.2) + CAVIN1 (151.9) + CCDC80 (23.2) + CCN1 (−142.3) + |
| | CENPA (162.2) + CHRNB1 (88.4) + CHST13 (−299.2) + CLDN4 (138.7) + CTSB (1551.5) + |
| | DDAH1 (−32.5) + DHCR7 (−33.6) + EPS8L2 (−31.5) + FMR1 (−72.1) + FST (5.7) + GPNMB |
| | (430.0) + HEY1 (−11.6) + IGSF9 (−48.8) + IKBIP (−83.0) + ITGA2 (61.6) + ITGB2 (−101.3) + |
| | KRIT1 (132.9) + LCA5 (219.4) + LMCD1 (−41.3) + LXN (128.6) + MALL (−40.2) + MAPK13 |
| | (86.4) + MFSD5 (−120.9) + MSRB3 (−74.2) + NNMT (298.2) + OVOL1 (59.7) + PCDHB2 |
| | (−39.1) + PCDHB9 (43.7) + PERP (704.1) + PKMYT1 (95.7) + PLA2G4C (50.1) + PLEKHA7 |
| | (45.7) + PLOD2 (−17.1) + PRPS2 (−12.7) + PVR (27.8) + PYGB (−14.8) + RAB32 (−64.7) + |
| | RAC2 (123.8) + SGTB (135.8) + SHCBP1 (−53.8) + SMOC1 (32.5) + SPATA5 (−165.7) + |
| | SPIRE2 (161.1) + SPRY4 (−88.3) + STMN3 (−61.9) + TMEM144 (−24.2) + TNNC1 (−41.5) + |
| | TP63 (−112.3) + TRIM31 (−21.6) + TTF2 (−143.7) + TYMS (57.9) + UGCG (−155.9) + |
| | ZCWPW1 (−491.5) + ZNF488 (−302.2) − 2859.2 |
| | Optimal function group centroids: TEAD-inactive (I) 7.7; TEAD-active (A) −780.5 |
| tt) | Set tt): Minimal discriminant function: GPNMB (12.6) + NNMT (16.0) + CTSB (68.1) + |
| | TMEM139 (8.0) + KRIT1 (6.3) + ZCWPW1 (−18.3) + MSRB3 (−4.7) + ZNF488 (−5.2) + CCN1 |
| | (−4.7) − 88.6 |
| | Minimal function group centroids: TEAD-inactive (I) 0.2; TEAD-active (A) −20.5 |
| | Embodiment 31. Optimal and minimal discriminant predictors, with their |
| | unstandardized coefficients, and group centroids for LUSC classical tumors: |
| uu) | Set uu): Optimal discriminant function: CLDN4 (5.3) + DEDD2 (−4.4) + GPNMB (11.5) + |
| | HAS3 (−3.6) + MATN3 (−3.2) + MMP13 (2.8) + MSRB3 (−4.1) + PDZD2 (−3.8) + PKP2 (−1.9) + |
| | RAPGEFL1 (4.0) + RBP1 (1.9) + TMC4 (2.9) + TP63 (21.0) + TPD52L1 (3.4) + UAP1 |
| | (−3.3) − 35.9 |
| | Optimal function group centroids: TEAD-inactive (I) 0.4; TEAD-active (A) −4.0 |
| | Minimal discriminant function: NA |
| | Embodiment 32. Optimal and minimal discriminant predictors, with their |
| | unstandardized coefficients, and group centroids for LUSC primitive tumors: |
| vv) | Set vv): Optimal discriminant function: ACOX2 (−82.3) + CCNG2 (−216.8) + CDCA4 (−30.9) + |
| | CDH4 (−16.3) + CDV3 (−411.9) + CENPM (37.4) + CENPN (104.6) + CLCN3 (154.5) + |
| | COL6A1 (−54.2) + COL8A1 (55.5) + CSTA (−88.1) + CTSB (1473.3) + CYP27C1 (171.0) + |
| | DAPK1 (54.3) + DCLRE1B (237.4) + DKK1 (14.9) + DUSP6 (80.9) + EBP (−32.7) + FAM83B |
| | (16.5) + HPS5 (−19.1) + IGSF9 (−134.1) + IL6 (106.5) + ITGA2 (29.9) + LMNB2 (203.1) + |
| | MAD2L1 (7.9) + MSX2 (−104.7) + NEDD4L (23.0) + NNMT (−37.8) + OTUB2 (−218.3) + |
| | OXCT1 (15.9) + PAK2 (−58.0) + PCBD1 (−112.7) + PIK3R1 (−55.4) + PKP2 (68.0) + PKP4 |
| | (−97.8) + PLCE1 (8.8) + RBM23 (−283.7) + RCN2 (116.5) + RFC4 (43.8) + RGS17 (12.6) + |
| | S100A14 (26.0) + SAMD9 (−161.4) + SCML1 (37.0) + SEC14L2 (−67.9) + SH3RF1 (99.4) + |
| | SLC25A23 (122.8) + SLC38A5 (48.3) + SNCG (16.5) + TMC4 (−58.8) + TMEM139 (105.5) + |
| | TNNC1 (117.1) + TP53INP1 (−106.8) + TPD52L1 (−116.8) + TRIP11 (155.4) + TRIP13 |
| | (−16.3) + TUBB6 (−33.4) + UGCG (111.1) + VAMP8 (400.2) + WDR13 (−205.8) + WWC1 |
| | (−164.3) + ZBED2 (−53.2) + ZNF467 (8.7) + ZNF704 (77.3) − 1203.1 |
| | Optimal function group centroids: TEAD-inactive (I) −46.6; TEAD-active (A) 97.7 |
| ww) | Set ww): Minimal discriminant function: IL6 (7.1) + SHROOM2 (−2.6) + SHCBP1 (6.6) + |
| | DCLRE1B (14.1) + CDV3 (−17.8) + SAMD9 (−7.6) + IGSF9 (−5.5) + OTUB2 (−8.1) + COL6A1 |
| | (−3.5) + TPD52L1 (−6.8) + WDR13 (−11.7) + RBM23 (−16.7) + SMOC1 (2.1) + CCNG2 (−8.8) + |
| | TNNC1 (5.9) + FDPS (5.5) + LMNB2 (11.3) + CCBE1 (−2.4) + CTSB (38.3) + ZNF704 |
| | (4.0) + PKP2 (3.2) + WWWC1 (−6.5) + CRY1 (6.4) + SH3RF1 (5.6) + CYP27C1 (7.9) + |
| | SLC25A23 (3.3) + CENPN (3.0) − 16.0 |
| | Minimal function group centroids: TEAD-inactive (I) −2.3; TEAD-active (A) 4.9 |
| | Embodiment 33. Optimal and minimal discriminant predictors, with their |
| | unstandardized coefficients, and group centroids for LUSC secretory |
| | tumors: |
| xx) | Set xx): Optimal discriminant function: ABAT (−18.0) + ADAMTS1 (−21.3) + AMOT (−43.3) + |
| | ANXA3 (−96.9) + ARAP3 (−70.6) + ATXN1 (−81.8) + CAP2 (59.2) + CDC25A (−17.9) + |
| | CEBPB (−29.2) + CENPM (187.6) + CLIC3 (12.6) + COL6A2 (−84.9) + CROT (86.8) + CTH |
| | (177.8) + DAPK1 (72.9) + DCLRE1B (216.1) + DDAH1 (−51.0) + DLL1 (65.4) + DSC2 (15.3) + |
| | EIF2AK3 (−232.8) + ESM1 (98.5) + ETS1 (98.7) + EXO1 (12.1) + FAHD2A (179.3) + |
| | FAM102A (88.8) + FKBP2 (216.8) + GADD45B (35.4) + GRAMD2B (28.0) + HDHD2 (−70.4) + |
| | IGSF3 (−138.4) + JPH2 (734.5) + KRT8 (724.3) + LMTK3 (46.2) + LRP10 (−85.3) + LRP8 |
| | (−135.2) + MAP6D1 (50.8) + MCM5 (−72.6) + MFSD5 (−126.8) + MID1 (64.2) + MLLT11 |
| | (−3.8) + MRPL33 (165.7) + NCAPD3 (−24.3) + NFIB (11.5) + NUAK1 (20.7) + OAS1 (−23.8) + |
| | OAS2 (−130.8) + PDZD2 (114.8) + PIK3R3 (−124.3) + PKIA (−88.6) + PKMYT1 (−10.3) + |
| | PLA2G4C (−131.9) + PLK2 (99.2) + PPP1R11 (80.3) + PSG6 (−1089.7) + PSG7 (−1075.2) + |
| | PTPRE (140.0) + PYGB (−89.3) + ROR1 (142.0) + SECISBP2 (113.8) + SH3PXD2A |
| | (−50.5) + SLC25A3 (−382.8) + SLC30A9 (122.3) + SLC3A2 (286.7) + SNAPC1 (100.9) + |
| | STK3 (−51.7) + STX1A (−70.2) + TAGLN (−739.7) + TDO2 (41.1) + TMEM144 (−280.1) + |
| | TMEM45B (−35.8) + TP53INP1 (55.2) + TPX2 (66.2) + TRIB2 (35.9) + TRIP13 (−47.1) + |
| | VPS52 (−28.2) + ZBED2 (−8.5) − 187.9 |
| | Optimal function group centroids: TEAD-inactive (I) −30.2; TEAD-active (A) 162.9 |
| yy) | Set yy): Minimal discriminant function: RAPGEFL1 (4.3) + JPH2 (−34.3) + STX1A (7.4) + |
| | PKIA (4.4) + LRP8 (9.0) + TAGLN (44.2) + TMEM144 (16.3) + SNAPC1 (−5.5) + CTH (−5.0) + |
| | QKI (−2.0) + LMTK3 (−3.3) + PIK3R3 (4.4) + KRT8 (−26.4) + ANXA3 (6.0) + PSG6 (62.6) + |
| | ESM1 (−3.7) + SH3PXD2A (3.8) + OAS2 (5.7) + IGSF3 (4.6) + SLC3A2 (−18.3) + CROT |
| | (−4.3) + MFSD5 (7.0) + CENPM (−4.7) + EIF2AK3 (5.9) + MRPL33 (−9.4) + ROR1 (−5.5) − |
| | 11.1 |
| | Minimal function group centroids: TEAD-inactive (I) 1.2; TEAD-active (A) −6.6 |
| | Embodiment 34. Optimal and minimal discriminant predictors, with their |
| | unstandardized coefficients, and group centroids for Malignant |
| | mesothelioma (MESO) tumors: |
| zz) | Set zz): Optimal discriminant function: AASS (9.1) + ACOX2 (13.8) + ADAMTS1 (−2.6) + |
| | BCAT1 (−7.0) + CEBPB (−10.4) + CHRNB1 (22.6) + FXYD3 (8.6) + IL6 (−5.5) + IRX5 (10.3) + |
| | LYPD3 (9.9) + MALL (4.3) + NUAK2 (8.8) + PAK1 (−15.4) + PCNA (17.8) + PHF21A (18.3) + |
| | PYGB (9.6) + RACGAP1 (−9.6) + RFC4 (−14.3) + ROR1 (−4.3) + RPS24 (125.9) + SMOC1 |
| | (3.2) + SORT1 (8.0) + SUSD2 (−3.8) + TEAD4 (−7.5) + TMC4 (−4.9) + TPM1 (−12.0) − 124.5 |
| | Optimal function group centroids: TEAD-inactive (I) 7.7; TEAD-active (A) −4.5 |
| aaa) | Set aaa): Minimal discriminant function: ACOX2 (6.7) + TPM1 (−7.1) + PHF21A (11.3) + |
| | IRX5 (6.1) + BCAT1 (−3.5) + SORT1 (4.7) + CHRNB1 (8.3) + FXYD3 (2.4) + PYGB (7.4) + |
| | PAK1 (−6.0) + IL6 (−1.3) − 7.2 |
| | Minimal function group centroids: TEAD-inactive (I) 3.5; TEAD-active (A) −2.0 |
| | Embodiment 35. Optimal and minimal discriminant predictors, with their |
| | unstandardized coefficients, and group centroids for Ovarian serous |
| | cystadenocarcinoma (OV) tumors: |
| bbb) | Set bbb): Optimal discriminant function: CA2 (1.0) + CAP2 (−1.6) + CCDC80 (−1.2) + |
| | CDC25A (−5.0) + CDCA3 (−1.0) + CDCA4 (−1.7) + DDAH1 (−1.4) + DLL1 (−2.0) + DUSP6 |
| | (1.9) + ELN (1.3) + FEN1 (−4.2) + IRF9 (3.6) + ITGA2 (2.9) + KRT80 (−1.5) + LMCD1 (−1.6) + |
| | MAPK13 (2.2) + MCM2 (−3.3) + MFSD5 (2.6) + MLLT11 (1.0) + NRIP1 (−1.6) + OFD1 |
| | (2.5) + OSBPL7 (1.3) + PCNA (9.5) + PHF21A (4.1) + PIK3R3 (3.0) + PLK2 (−2.1) + PRRX2 |
| | (1.1) + PTPRE (2.4) + QKI (−1.7) + RAPGEFL1 (1.6) + RBMS2 (2.8) + SH3TC1 (−1.9) + |
| | SHCBP1 (−2.8) + SLC30A9 (2.6) + SLC7A1 (−1.0) + SNX24 (−2.5) + TMC4 (2.1) + |
| | TMEM45B (1.0) + UGCG (−3.0) + ULK1 (2.7) + WDR13 (2.4) − 13.6 |
| | Optimal function group centroids: TEAD-inactive (I) 1.0; TEAD-active (A) −2.4 |
| | Minimal discriminant function: NA |
| | Embodiment 36. Optimal and minimal discriminant predictors, with their |
| | unstandardized coefficients, and group centroids for OV differentiated |
| | tumors: |
| ccc) | Set ccc): Optimal discriminant function: CCBE1 (937.3) + CCNG2 (−10.3) + CHST13 (209.0) + |
| | DHCR7 (21.2) + FERMT1 (18.7) + FMR1 (−137.1) + GRB10 (117.7) + HBP1 (−284.3) + |
| | HPS5 (−30.7) + IFI44 (−5.4) + IRAK2 (62.3) + KCNMB3 (211.9) + LYPD3 (14.1) + MCM2 |
| | (85.2) + MSX2 (361.0) + NNMT (−20.3) + PCBD1 (1698.2) + PKP2 (−161.5) + PLA2G4C |
| | (−314.9) + PSG9 (−1855.8) + REEP6 (−50.2) + STK3 (142.3) + TEAD4 (45.2) + TMC4 (141.7) + |
| | TUFT1 (213.9) + WWC1 (−151.5) − 1648.7 |
| | Optimal function group centroids: TEAD-inactive (I) −18.2; TEAD-active (A) 173.0 |
| ddd) | Set ddd): Minimal discriminant function: CCBE1 (46.0) + HBP1 (−14.2) + CHST13 (19.9) + |
| | MSX2 (12.1) + PKP2 (−8.7) + PCBD1 (60.5) − 47.0 |
| | Minimal function group centroids: TEAD-inactive (I) −0.7; TEAD-active (A) 6.7 |
| | Embodiment 37. Optimal and minimal discriminant predictors, with their |
| | unstandardized coefficients, and group centroids for OV immune-reactive |
| | tumors: |
| eee) | Set eee): Optimal discriminant function: AASS (−148.4) + ARAP3 (82.9) + BTG3 (−33.2) + |
| | C4BPB (95.8) + CDK2 (82.1) + CNN3 (140.8) + COBL (−21.2) + COTL1 (23.5) + DCLRE1B |
| | (156.7) + DSG3 (−1387.4) + DYNC2LI1 (92.8) + HOXA5 (33.8) + HPS5 (26.5) + KLF13 |
| | (−44.0) + KRTDAP (35.5) + LHFPL6 (140.5) + MCM10 (−52.9) + MFSD1 (−233.7) + OFD1 |
| | (−169.1) + OSBPL7 (−83.5) + OTUB2 (−145.2) + PAK1 (40.1) + PIK3R2 (−250.4) + QKI (199.6) + |
| | RBM45 (113.8) + RCN2 (59.9) + SGK1 (45.1) + SHCBP1 (388.0) + SLFN5 (64.6) + |
| | SNX24 (202.6) + SPIRE2 (190.9) + STK3 (−64.6) + TMC7 (29.7) + TMEM144 (−58.1) − 333.5 |
| | Optimal function group centroids: TEAD-inactive (I) −29.1; TEAD-active (A) 91.4 |
| fff) | Set fff): Minimal discriminant function: SHCBP1 (13.5) + PIK3R2 (−13.5) + MFSD1 (−15.8) + |
| | QKI (12.0) + SNX24 (8.3) + SPIRE2 (6.7) + AASS (−16.7) + DSG3 (−61.6) + 7.5 |
| | Minimal function group centroids: TEAD-inactive (I) −1.2; TEAD-active (A) 3.6 |
| | Embodiment 38. Optimal and minimal discriminant predictors, with their |
| | unstandardized coefficients, and group centroids for OV mesenchymal |
| | tumors: |
| ggg) | Set ggg): Optimal discriminant function: ABAT (60.6) + ABI3BP (−31.1) + AMOT (−66.8) + |
| | B4GALT4 (28.1) + BCL11B (−179.7) + CDC25A (246.0) + CDCA4 (251.1) + CHRNB1 |
| | (−46.3) + CLDN4 (−285.3) + DCLRE1B (492.2) + DLL1 (37.3) + EBP (97.6) + EXO1 (−77.7) + |
| | FSTL1 (152.4) + FSTL3 (65.3) + IL6 (65.8) + IRF9 (−151.3) + KCNMB3 (179.9) + MCM10 |
| | (333.9) + MLPH (89.0) + NOC3L (−249.1) + NUP37 (−94.3) + PDZD2 (−29.8) + PSG9 (93.4) + |
| | RAB32 (400.6) + RND3 (204.4) + SH3RF1 (137.6) + SLC25A23 (218.6) + SMOC1 (31.0) + |
| | SYDE2 (−160.0) + TGM2 (53.5) + TP63 (−141.8) + TYMS (−19.0) + ZNF488 (132.7) − 900.9 |
| | Optimal function group centroids: TEAD-inactive (I) −73.7; TEAD-active (A) 68.5 |
| hhh) | Set hhh): Minimal discriminant function: CDC25A (13.7) + RAB32 (15.7) + RND3 (6.0) + |
| | ZNF488 (11.8) + SH3RF1 (8.6) + NOC3L (−11.9) + AMOT (−4.9) + CDCA4 (11.3) + |
| | DCLRE1B (13.0) + FSTL3 (4.3) − 31.8 |
| | Minimal function group centroids: TEAD-inactive (I) −2.7; TEAD-active (A) 2.5 |
| | Embodiment 39. Optimal and minimal discriminant predictors, with their |
| | unstandardized coefficients, and group centroids for OV proliferative tumors: |
| iii) | Set iii): Optimal discriminant function: ACAT2 (21.1) + ANKRD12 (106.6) + CCBE1 (−374.1) + |
| | CCN1 (74.0) + CDC25A (260.1) + CDK1 (109.0) + CDK2 (−23.5) + COBL (−170.1) + CROT |
| | (176.1) + CYP27C1 (50.4) + EPHA2 (−30.3) + F3 (−52.6) + HOXA5 (−55.0) + ITGB2 (18.1) + |
| | KPNA2 (169.1) + LRP10 (−203.7) + LRRFIP2 (64.3) + MLLT11 (−86.5) + OVOL1 (128.8) + |
| | PIP4P2 (160.2) + PLK2 (159.4) + PNRC1 (−356.5) + PRRX2 (31.3) + PSG9 (−280.8) + |
| | REEP6 (−97.4) + SGMS2 (133.0) + SGTB (605.9) + SH3TC1 (132.9) + SNAPC1 (−324.5) + |
| | SPAG1 (37.3) + TNNC1 (19.9) + TRIP11 (−57.9) + TSPAN1 (−87.9) + WWC2 (105.6) − 13.1 |
| | Optimal function group centroids: TEAD-inactive (I) −70.8; TEAD-active (A) 116.9 |
| jjj) | Set jjj): Minimal discriminant function: CDC25A (13.4) + PLK2 (6.1) + SGTB (19.5) + |
| | MLLT11 (−2.0) + TSPAN1 (−2.2) + CCBE1 (−9.3) + COBL (−4.7) + SNAPC1 (−7.5) − 4.2 |
| | Minimal function group centroids: TEAD-inactive (I) −1.8; TEAD-active (A) 3.0 |
| | Embodiment 40. Optimal and minimal discriminant predictors, with their |
| | unstandardized coefficients, and group centroids for Pancreatic |
| | adenocarcinoma (PAAD) tumors: |
| kkk) | Set kkk): Optimal discriminant function: ABCA1 (10.9) + ACADVL (19.2) + ACOT11 (−4.8) + |
| | AGL (6.7) + AVPI1 (4.1) + CCBE1 (−45.1) + CLDN4 (30.3) + CPE (7.1) + CRIM1 (−13.5) + |
| | CROT (4.5) + DDR1 (28.3) + EPS8L2 (6.4) + FAM117B (8.2) + FXYD3 (11.0) + GADD45B |
| | (−5.4) + GRAMD2B (2.4) + GSN (−20.7) + HASPIN (−35.3) + JDP2 (5.2) + KCNMB3 (−11.9) + |
| | KRT8 (−84.9) + MATN2 (3.6) + MDC1 (−6.6) + MFSD5 (4.1) + MLLT11 (5.6) + MMP13 |
| | (−1.5) + PAG1 (7.5) + PCNA (−5.9) + PHLPP1 (7.9) + PIK3R3 (−6.5) + PSG2 (−170.2) + PSG6 |
| | (288.1) + PVR (−3.1) + RFC4 (6.4) + SGTB (22.5) + SLC25A23 (−9.2) + SLC3A2 (−20.1) + |
| | SNCG (−2.0) + TACC3 (4.0) + TENT5B (8.0) + TRIM13 (4.2) + VSNL1 (−2.8) + WWC2 |
| | (−7.7) + 33.4 |
| | Optimal function group centroids: TEAD-inactive (I) 0.4; TEAD-active (A) −79.0 |
| kkk1) | Set kkk1): Minimal discriminant function: CCBE1 (44.3) − 1.3 |
| | Minimal function group centroids: TEAD-inactive (I) −0.1; TEAD-active (A) 14.6 |
| | Embodiment 41. Optimal and minimal discriminant predictors, with their |
| | unstandardized coefficients, and group centroids for Rectum |
| | adenocarcinoma (READ) tumors: |
| lll) | Set lll): Optimal discriminant function: ANTXR2 (−3.4) + CLDN1 (2.3) + GPRC5A (3.7) + |
| | GPRC5C (4.1) + MALL (6.2) + PVR (−3.7) + TEAD1 (2.8) + TMC4 (6.2) + TRIP13 (−4.9) + |
| | ZCWPW1 (3.9) − 12.8 |
| | Optimal function group centroids: TEAD-inactive (I) 0.3; TEAD-active (A) −3.8 |
| | Minimal discriminant function: NA |
| | Embodiment 42. Optimal and minimal discriminant predictors, with their |
| | unstandardized coefficients, and group centroids for READ CMS1 tumors: |
| mmm) | Set mmm): Optimal discriminant function: EPS8L3 (4259.1) + PLOD2 (−560.9) − 2818.3 |
| | Optimal function group centroids: TEAD-inactive (I) 558.0; TEAD-active (A) −558.0 |
| | Minimal discriminant function: NA |
| | Embodiment 43. Optimal and minimal discriminant predictors, with their |
| | unstandardized coefficients, and group centroids for READ CMS2 tumors: |
| nnn) | Set nnn): Optimal discriminant function: CDC25A (−121.8) + DCLRE1B (−184.1) + DHFR |
| | (43.5) + FOS (−37.2) + HASPIN (427.8) + JDP2 (120.9) + LMCD1 (18.9) + MID1 (−104.6) + |
| | MXRA7 (13.0) + NEDD4L (−353.4) + SCML1 (280.9) + SH3PXD2A (−107.1) + TDO2 (−17.9) + |
| | TMEM140 (19.4) + ZSWIM7 (39.4) − 4.6 |
| | Optimal function group centroids: TEAD-inactive (I) −9.6; TEAD-active (A) 67.5 |
| ooo) | Set ooo): Minimal discriminant function: HASPIN (21.0) + NEDD4L (−16.6) + SCML1 (8.7) − |
| | 2.5 |
| | Minimal function group centroids: TEAD-inactive (I) −0.5; TEAD-active (A) 3.6 |
| | Embodiment 44. Optimal and minimal discriminant predictors, with their |
| | unstandardized coefficients, and group centroids for READ CMS4 tumors: |
| ppp) | Set ppp): Optimal discriminant function: AURKB (−37.5) + CCBE1 (147.3) + COL6A2 |
| | (−673.4) + DIAPH3 (−321.3) + HDHD2 (134.8) + LSM5 (127.2) + MDC1 (164.3) + PLOD2 |
| | (−71.4) + PPIH (48.7) + SGK1 (101.2) + TMC4 (−74.7) + TRIP13 (313.0) + VSNL1 (−6.7) + |
| | 376.1 |
| | Optimal function group centroids: TEAD-inactive (I) −14.2; TEAD-active (A) 113.8 |
| ppp1) | Set ppp1): Minimal discriminant function: TRIP13 (11.9) − 5.5 |
| | Minimal function group centroids: TEAD-inactive (I) −0.4; TEAD-active (A) 3.2 |
| | Embodiment 45. Optimal and minimal discriminant predictors, with their |
| | unstandardized coefficients, and group centroids for READ unclassifiable |
| | tumors: |
| qqq) | Set qqq): Optimal discriminant function: ADRB2 (2553.4) + AURKB (60.5) + CDK6 (95.9) + |
| | TEAD4 (−33.9) + VAMP8 (−1534.3) + 1308.9 |
| | Optimal function group centroids: TEAD-inactive (I) −22.9; TEAD-active (A) 206.2 |
| qqq1) | Set qqq1): Minimal discriminant function: ADRB2 (97.2) − 4.3 |
| | Minimal function group centroids: TEAD-inactive (I) −0.7; TEAD-active (A) 6.1 |
| | Embodiment 46. Optimal and minimal discriminant predictors, with their |
| | unstandardized coefficients, and group centroids for Sarcoma (SARC) |
| | tumors: |
| rrr) | Set rrr): Optimal discriminant function: ABCC5 (2.9) + AGL (3.0) + ASF1A (3.3) + BIRC5 |
| | (−2.6) + CENPN (−4.9) + CLCN3 (2.8) + COL6A1 (26.4) + COL6A2 (17.5) + DDAH1 (−2.5) + |
| | DONSON (−5.7) + ERAP2 (−.6) + FAM117B (−2.9) + FEN1 (2.5) + FMR1 (2.7) + GADD45A |
| | (−4.5) + GPC6 (−1.8) + GRAMD2B (2.7) + HOXA5 (2.3) + IGSF3 (2.5) + KRTDAP (−2.5) + |
| | MAPK13 (1.8) + NCAPD3 (−6.4) + NEDD4L (.9) + NEXN (−1.2) + OAS2 (2.3) + OFD1 (3.0) + |
| | PAK2 (5.9) + PHF21A (6.7) + QKI (−5.3) + RAC2 (−1.5) + RAPGEFL1 (−3.1) + RNF144B |
| | (2.0) + SGK1 (−2.2) + SLC38A5 (−1.0) + SPRY4 (1.5) + TNNC1 (−1.8) + TSPAN1 (3.8) + |
| | TUFT1 (−2.8) − 40.9 |
| | Optimal function group centroids: TEAD-inactive (I) 2.9; TEAD-active (A) −2.3 |
| | Minimal discriminant function: NA |
| | Embodiment 47. Optimal and minimal discriminant predictors, with their |
| | unstandardized coefficients, and group centroids for Skin Cutaneous |
| | Melanoma (SKCM) tumors: |
| sss) | Set sss): Optimal discriminant function: ACAT2 (1.0) + ADM (.8) + AVPI1 (1.9) + AZIN1 |
| | (5.4) + BIRC5 (−3.3) + CCN1 (1.0) + CDC6 (1.9) + CDK6 (3.3) + CEBPB (−2.1) + CENPA |
| | (2.8) + CENPI (6.7) + COL6A2 (−2.9) + CSTA (−1.9) + CXCL1 (1.3) + DIAPH2 (2.0) + |
| | DYNC2LI1 (−3.8) + EMG1 (3.3) + FAM83D (3.9) + FST (1.2) + GOLGA5 (4.1) + HASPIN |
| | (3.7) + HEG1 (2.1) + HEY1 (−1.2) + HPS5 (−1.5) + ITGA2 (−1.7) + ITGB2 (2.1) + KRT80 |
| | (1.7) + LRP10 (−3.7) + LTBP4 (−2.1) + LYPD6 (−1.8) + MATN2 (.9) + MCM10 (3.9) + MCM5 |
| | (2.7) + MICB (1.7) + MSRB3 (1.6) + NFIL3 (1.1) + NUAK1 (1.9) + OLR1 (1.1) + PJA2 (−3.4) + |
| | PNRC1 (−2.6) + PRSS23 (3.0) + PSG9 (2.4) + PTPRE (−1.5) + ROR1 (−2.1) + SEC14L1 |
| | (2.5) + SHCBP1 (−2.6) + SHROOM2 (−1.0) + SLC1A2 (9.9) + SLC37A2 (−1.8) + SLFN5 |
| | (−1.8) + TDO2 (−2.0) + TMEM140 (−3.5) + TNFRSF12A (2.0) + TPX2 (−7.4) + TRIM13 (−2.2) + |
| | TRIM38 (−2.0) − 1.6 |
| | Optimal function group centroids: TEAD-inactive (I) −1.1; TEAD-active (A) 2.6 |
| | Minimal discriminant function: NA |
| | Embodiment 48. Optimal and minimal discriminant predictors, with their |
| | unstandardized coefficients, and group centroids for SKCM immune tumors: |
| ttt) | Set ttt): Optimal discriminant function: CDCA5 (−11.6) + CDK6 (−4.5) + CSTA (2.5) + CXXC5 |
| | (4.2) + DYNC2LI1 (9.3) + EIF2AK3 (−4.9) + EMG1 (−8.5) + EPS8L3 (−12.1) + EXOSC2 |
| | (−3.3) + FKBP2 (13.2) + HSPB11 (−5.9) + IRF9 (6.1) + ITGA2 (2.2) + LTBP4 (2.3) + MATN2 |
| | (−1.8) + METRNL (−2.2) + MMP13 (1.9) + MRPL33 (−5.8) + OLR1 (−2.3) + PCMTD2 (4.2) + |
| | PHF21A (11.1) + PRSS23 (−2.2) + PTPRE (2.5) + RND3 (−1.8) + SHCBP1 (3.9) + |
| | SHROOM2 (2.0) + SLC37A2 (3.2) + SLC3A2 (−12.0) + SLK (5.8) + SNAPC1 (−9.2) + TBX3 |
| | (2.5) + TRIB1 (4.6) + UBE2C (11.0) + UHRF1 (−3.0) − 7.5 |
| | Optimal function group centroids: TEAD-inactive (I) 1.5; TEAD-active (A) −3.6 |
| | Minimal discriminant function: NA |
| | Embodiment 49. Optimal and minimal discriminant predictors, with their |
| | unstandardized coefficients, and group centroids for SKCM keratin tumors: |
| uuu) | Set uuu): Optimal discriminant function: AGPAT4 (54.9) + AZIN1 (298.7) + BHLHE41 (9.8) + |
| | CDCA8 (176.8) + COBL (21.6) + CSRNP2 (−110.8) + CTNNBIP1 (26.8) + FAM83D |
| | (−40.3) + FANCA (121.2) + FAT4 (−29.7) + FEN1 (−21.6) + GOLPH3L (18.3) + IL6 (87.0) + |
| | INTS3 (−69.4) + JDP2 (49.9) + KLF10 (−39.7) + KRTDAP (−33.4) + LMTK3 (52.2) + LXN |
| | (−47.2) + MANSC1 (−23.8) + MARCKSL1 (−202.4) + MRPL33 (−63.3) + MXRA7 (−103.7) + |
| | NAPEPLD (−27.4) + NCAPD3 (78.3) + NNMT (−3.3) + NOC3L (−84.1) + NUAK2 (47.6) + |
| | OAS1 (−15.1) + PAK1 (−15.0) + PCBD1 (19.7) + PEPD (−159.4) + PRSS23 (48.1) + PSG2 |
| | (−1552.0) + PSG6 (794.8) + PSG7 (460.5) + PYGB (204.1) + RGL2 (−13.1) + SGK1 (−179.7) + |
| | SNCG (−35.3) + SPATA5 (39.3) + SRD5A3 (−22.0) + STX1A (−51.3) + SUSD2 (−10.5) + |
| | TCF25 (−191.5) + THBS1 (−51.7) + TP53INP1 (11.3) + TPD52L1 (−7.6) + ZDHHC18 (125.4) + |
| | 331.6 |
| | Optimal function group centroids: TEAD-inactive (I) −32.3; TEAD-active (A) 39.9 |
| vvv) | Set vvv): Minimal discriminant function: CDCA8 (26.2) + LXN (−4.7) + INTS3 (−7.0) + PSG2 |
| | (−260.2) + ZDHHC18 (13.7) + PEPD (−33.7) + MARCKSL1 (−22.7) + PRSS23 (7.3) + PSG7 |
| | (77.5) + PSG6 (92.2) + MXRA7 (−6.6) + KRTDAP (−4.4) + SPATA5 (16.4) + JDP2 (7.3) + |
| | IL6 (6.7) + PCBD1 (5.5) + KLF10 (−6.2) + AZIN1 (20.9) + SGK1 (−21.8) + NCAPD3 (11.5) + |
| | RCN2 (−9.6) + CSRNP2 (−8.8) + TP53INP1 (3.5) + FOS (3.4) + 36.2 |
| | Minimal function group centroids: TEAD-inactive (I) -3.9; TEAD-active (A) 4.8 |
| | Embodiment 50. Optimal and minimal discriminant predictors, with their |
| | unstandardized coefficients, and group centroids for SKCM MITF-low |
| | tumors: |
| www) | Set www): Optimal discriminant function: AASDH (68.8) + ANKRD42 (32.0) + ANTXR2 |
| | (−27.5) + ASF1A (−51.8) + BTG3 (6.0) + CCN1 (67.6) + CDC42EP4 (27.3) + CDH4 (−26.4) + |
| | CDK2 (−48.4) + CELSR3 (−119.5) + CENPA (170.0) + CENPN (−38.6) + CNN3 (144.2) + |
| | COL8A1 (66.9) + CTSB (243.0) + CXCL1 (−14.4) + CYP1B1 (−27.6) + DDAH1 (44.1) + |
| | DIAPH2 (167.0) + DLL1 (−139.7) + DUT (166.2) + DYNC2LI1 (−86.6) + ESM1 (29.1) + |
| | FAM83D (70.7) + FXYD3 (−10.4) + GPR176 (−73.4) + LMTK3 (−175.6) + LRRFIP2 (−29.0) + |
| | MCM10 (115.7) + MMP13 (−18.8) + MYO1A (−320.8) + NCAPD3 (14.9) + NEDD4 (136.5) + |
| | NEXN (92.6) + NTN4 (−35.2) + NUP37 (271.1) + OLFML3 (70.8) + OVOL1 (784.4) + PAG1 |
| | (31.6) + PCBD1 (−225.3) + PCDHB9 (57.1) + PDZD2 (−52.7) + PHLPP1 (−19.2) + PKMYT1 |
| | (13.3) + PLEKHA7 (27.5) + PRPS2 (−72.0) + PSG9 (305.8) + RAB11FIP1 (−31.3) + RBMS2 |
| | (120.9) + RFC4 (283.3) + SHCBP1 (−54.9) + SLC1A2 (215.6) + SPAG1 (21.1) + STK3 |
| | (114.0) + TK1 (−29.3) + TNNC1 (66.6) + TRIP13 (58.6) + TTC17 (151.5) + UHRF1 (−83.5) + |
| | WWC1 (−5.9) + WWC2 (274.4) + ZDHHC18 (−87.9) + ZNF292 (−359.4) + ZNF75D (148.4) − |
| | 950.0 |
| | Optimal function group centroids: TEAD-inactive (I) −52.4; TEAD-active (A) 98.6 |
| xxx) | Set xxx): Minimal discriminant function: MCM10 (16.2) + CCN1 (3.7) + DUT (6.0) + NUP37 |
| | (13.3) + WWC2 (18.0) + CELSR3 (−9.3) + TRIP13 (4.2) + DIAPH2 (6.0) + ASF1A (−4.3) + |
| | PCBD1 (−10.8) + RFC4 (14.5) + LMTK3 (−13.3) + TNNC1 (4.7) + ZNF292 (−14.8) + OVOL1 |
| | (37.8) + ESM1 (2.9) + PAG1 (4.7) + COL8A1 (3.1) + ZNF75D (11.8) + NEDD4 (6.3) + |
| | PCDHB9 (3.9) + DYNC2LI1 (−3.9) + CNN3 (5.2) + OLFML3 (2.4) − 38.0 |
| | Minimal function group centroids: TEAD-inactive (I) −2.8; TEAD-active (A) 5.3 |
| | Embodiment 51. Optimal and minimal discriminant predictors, with their |
| | unstandardized coefficients, and group centroids for Stomach |
| | adenocarcinoma (STAD) tumors: |
| yyy) | Set yyy): Optimal discriminant function: AASDH (−4.0) + ANKRD29 (2.5) + ANXA3 (−1.6) + |
| | BCAT1 (2.4) + C4BPB (1.7) + CLDN4 (−2.9) + CLIC3 (1.3) + COTL1 (5.1) + CSTA (−1.6) + |
| | CXXC5 (−1.3) + CYP27C1 (−11.8) + DSC2 (−1.9) + FOS (−2.3) + HASPIN (1.8) + IGSF3 |
| | (1.7) + KLF13 (1.9) + LRP10 (−3.2) + MALL (2.2) + MDC1 (2.7) + MGST2 (3.1) + MID1 (1.8) + |
| | MTMR11 (−1.7) + MTSS1 (1.2) + NPAS2 (−2.8) + NUAK1 (−1.9) + PIK3C2B (−3.5) + PKP2 |
| | (1.8) + PRPS1 (−2.3) + PSG6 (31.1) + PSG7 (59.7) + PSG9 (−15.7) + RAPGEFL1 (1.1) + |
| | SCML1 (−2.2) + SEC14L1 (3.5) + SH3TC1 (−1.3) + SKP2 (1.8) + SLFN5 (−1.5) + SNAPC1 |
| | (4.0) + TMEM139 (−1.9) + TPD52L1 (−1.3) + TSPAN1 (−2.1) + VAMP8 (9.5) + WWC2 (4.6) − |
| | 7.6 |
| | Optimal function group centroids: TEAD-inactive (I) −0.4; TEAD-active (A) 5.6 |
| | Minimal discriminant function: NA |
| | Embodiment 52. Optimal and minimal discriminant predictors, with their |
| | unstandardized coefficients, and group centroids for STAD MSI tumors: |
| zzz) | Set zzz): Optimal discriminant function: ABCA1 (−26.4) + ALDH3A2 (−76.0) + ARHGAP11A |
| | (40.3) + ARHGEF2 (24.0) + BIRC5 (−28.5) + CAVIN1 (212.6) + CCDC80 (−134.7) + CDCA8 |
| | (−195.6) + CDK2 (−183.6) + CLDN4 (304.8) + COL8A1 (114.0) + CYP1B1 (75.0) + DHX32 |
| | (42.8) + DIAPH3 (40.0) + ESM1 (−78.6) + FAM83D (97.1) + FMR1 (−29.1) + GOLPH3L |
| | (145.3) + GPNMB (28.0) + HBP1 (−48.9) + HDAC1 (317.2) + JDP2 (−49.3) + JPH2 (−56.6) + |
| | KLHL3 (27.4) + KPNA2 (77.3) + LRP10 (−22.7) + MDC1 (−116.9) + MSRB3 (−263.6) + |
| | MTSS1 (58.5) + MVD (106.8) + MXRA7 (35.8) + NNMT (−65.6) + NUAK2 (−31.9) + OVOL1 |
| | (48.9) + PCDHB2 (−8.2) + PEPD (28.3) + PHF21A (266.6) + PIK3C2B (150.0) + PKP4 |
| | (−104.5) + PLOD2 (−3.8) + PSG2 (−441.7) + PXMP2 (73.9) + PYGB (28.3) + RAC2 (−46.4) + |
| | RAPGEFL1 (−82.3) + SCML1 (115.6) + SGTB (22.2) + SPAG1 (19.8) + TAGLN (−43.5) + |
| | TK1 (160.3) + TLR3 (−34.7) + TMEM160 (−60.3) + TNNC1 (−16.8) + TRIP13 (70.8) + |
| | TSPAN1 (67.7) + TTF2 (−166.1) + TTLL3 (25.8) + TYMS (84.4) + UHRF1 (13.0) + VGF |
| | (5.8) + VKORC1L1 (−137.7) + VSNL1 (−10.7) + ZCWPW1 (72.5) − 868.2 |
| | Optimal function group centroids: TEAD-inactive (I) 18.7; TEAD-active (A) −126.8 |
| aaaa) | Set aaaa): Minimal discriminant function: TSPAN1 (3.8) + CLDN4 (17.7) + LYPD6 (−2.1) + |
| | MSRB3 (−14.2) + PHF21A (18.9) + SCML1 (7.1) + TTF2 (−11.3) + PIK3C2B (5.3) + |
| | GOLPH3L (10.0) + VKORC1L1 (−12.6) + MVD (11.4) + JDP2 (−4.8) + CAVIN1 (9.9) + PYGB |
| | (7.5) + RAPGEFL1 (−3.7) + CCDC80 (−7.9) + COL8A1 (5.1) + MDC1 (−6.9) + ZCWPW1 |
| | (13.7) + CYP1B1 (3.2) + DHX32 (5.0) + RFC4 (10.4) + CDCA8 (−5.5) + ESM1 (−2.4) + |
| | OVOL1 (2.1) − 44.8 |
| | Minimal function group centroids: TEAD-inactive (I) 1.3; TEAD-active (A) −8.5 |
| | Embodiment 53. Optimal and minimal discriminant predictors, with their |
| | unstandardized coefficients, and group centroids for STAD MSS_EMT |
| | tumors: |
| bbbb) | Set bbbb): Optimal discriminant function: ACSL5 (33.2) + ANKRD22 (−53.2) + ATP7A |
| | (−55.0) + AXL (127.0) + BCL11B (185.8) + BIRC5 (−71.9) + BMF (−44.2) + CCBE1 (−100.5) + |
| | CCDC80 (50.8) + CDCA3 (−38.4) + CNN3 (−467.2) + COL6A1 (−326.1) + COL6A2 (721.7) + |
| | CTNNBIP1 (−117.2) + CTSB (−844.5) + DHX32 (−27.0) + DKK1 (43.6) + DSC2 (−52.2) + |
| | DUSP6 (29.8) + DYNC2LI1 (−12.8) + FAM102A (78.3) + FAM83B (81.2) + FSTL1 (−671.0) + |
| | FXYD3 (72.4) + GRAMD2B (−84.8) + GSN (169.5) + HEXB (134.6) + IRX5 (−218.0) + |
| | KCNMB3 (−133.0) + KLK11 (29.0) + LRP8 (226.8) + LSM5 (47.4) + MALL (−26.1) + MAP6D1 |
| | (208.3) + MCM2 (−21.2) + METRNL (−44.8) + MFSD1 (75.8) + MGST3 (731.8) + MLPH |
| | (−37.9) + MMP13 (−98.1) + MRPL33 (86.2) + MSX2 (19.3) + NEDD4L (37.5) + NFIL3 (191.5) + |
| | NNMT (53.2) + NPAS2 (150.8) + NRIP1 (24.3) + OAS2 (−25.7) + OLR1 (18.1) + OTUB2 |
| | (−40.6) + PCMTD2 (−78.6) + PCNA (−71.8) + PIK3R2 (290.8) + PSG2 (1551.8) + RAB11FIP1 |
| | (116.6) + RACGAP1 (62.0) + RAPGEFL1 (−42.3) + RBMS2 (−170.3) + RFC4 (−195.9) + |
| | RNF144B (59.9) + S100A14 (−42.1) + SAMD9 (69.0) + SCD5 (52.4) + SDC2 (135.7) + |
| | SEC14L2 (28.0) + SHCBP1 (78.5) + SLC1A2 (33.1) + SLC3A2 (−134.7) + SLC7A1 (163.9) + |
| | SLFN5 (37.0) + SMPD4 (−260.5) + SPATA5 (−14.1) + SPIRE2 (18.6) + SQSTM1 (50.6) + |
| | TACC3 (13.2) + TDO2 (−14.8) + TMC4 (−24.5) + TNNC1 (35.4) + TRIB1 (172.3) + TRIB2 |
| | (−113.7) + TSPAN1 (76.8) + UAP1 (−21.6) + UBE2C (219.7) + UHRF1 (64.2) + VSNL1 (−86.0) + |
| | WDR13 (240.6) + ZNF704 (119.7) + ZSWIM7 (72.9) − 622.5 |
| | Optimal function group centroids: TEAD-inactive (I) 12.1; TEAD-active (A) −509.9 |
| cccc) | Set cccc): Minimal discriminant function: IRX5 (−5.1) + TSPAN1 (3.4) + NPAS2 (5.5) + GSN |
| | (13.6) − 18.7 |
| | Minimal function group centroids: TEAD-inactive (I) 0.2; TEAD-active (A) −7.7 |
| | Embodiment 54. Optimal and minimal discriminant predictors, with their |
| | unstandardized coefficients, and group centroids for STAD MSS_TP53- |
| | tumors: |
| dddd) | Set dddd): Optimal discriminant function: ACOT11 (125.2) + ACSL5 (21.5) + ANKRD42 |
| | (−76.2) + ANTXR2 (−68.8) + ARAP3 (47.6) + BCAT1 (31.6) + CDC25A (−121.3) + CDV3 (59.1) + |
| | CELSR3 (26.4) + COL6A1 (39.4) + COL8A1 (49.6) + CPA4 (536.3) + CPE (−65.9) + CTSK |
| | (−196.4) + DAPK1 (37.3) + DONSON (139.0) + DSG3 (8.9) + DUSP14 (26.5) + EBP (140.9) + |
| | FKBP2 (404.0) + FSTL1 (383.5) + FXYD3 (−45.8) + GDPD1 (−74.1) + GPRC5C (−19.9) + |
| | HASPIN (248.7) + KCNMA1 (99.4) + KRT80 (58.3) + LRRFIP2 (42.6) + LYPD6 (−23.8) + |
| | MDC1 (−17.9) + MXRA7 (71.1) + NUDCD1 (56.7) + PLA2G4C (−14.8) + PNRC1 (−47.9) + |
| | PSG7 (2345.6) + PSG9 (−516.2) + RAPGEFL1 (−21.7) + RBM23 (−111.3) + RCN2 (−240.0) + |
| | RGL2 (39.2) + SAMD9 (26.1) + SEC14L1 (−19.8) + SH3RF1 (35.4) + SKP2 (−14.7) + |
| | SLC14A1 (−28.5) + SNAPC1 (104.3) + TACC3 (69.1) + TDO2 (−77.0) + TEAD1 (−32.7) + |
| | TNFAIP3 (30.5) + TPM1 (−162.5) + TRIB1 (−55.9) + TRIP11 (94.4) + UBE2C (156.3) + |
| | UGCG (340.7) + VAMP8 (−542.1) + VSNL1 (−27.8) + WDR13 (−98.3) − 380.0 |
| | Optimal function group centroids: TEAD-inactive (I) −16.1; TEAD-active (A) 165.4 |
| eeee) | Set eeee): Minimal discriminant function: HASPIN (12.0) + PIK3C2B (−7.6) + CPA4 (19.3) |
| | + UGCG (9.9) + PSG7 (101.4) + VAMP8 (−11.3) + VSNL1 (−1.8) + WDR13 (−6.9) + FKBP2 |
| | (12.5) + MDC1 (4.9) + ACOT11 (2.4) + KRT80 (1.8) − 5.7 |
| | Minimal function group centroids: TEAD-inactive (I) −0.6; TEAD-active (A) 6.2 |
| | Embodiment 55. Optimal and minimal discriminant predictors, with their |
| | unstandardized coefficients, and group centroids for STAD MSS_TP53+ |
| | tumors: |
| ffff) | Set ffff): Optimal discriminant function: ARHGAP11A (48.8) + AVPI1 (16.2) + CDCA4 |
| | (−60.9) + CDCA8 (−33.0) + CDK2 (34.3) + CLDN4 (−52.8) + COTL1 (60.3) + CXCL1 (−6.3) + |
| | CYP27C1 (−288.9) + DHFR (10.1) + DUSP6 (−22.1) + EBP (36.7) + FAT4 (−59.1) + GPC6 |
| | (7.2) + GPR176 (100.6) + GSN (79.2) + HEXB (−24.3) + IGSF3 (−10.7) + KLF10 (7.6) + |
| | LMTK3 (10.8) + MCM10 (63.4) + MICB (6.7) + MLLT11 (16.0) + NFIL3 (−9.0) + NRIP1 |
| | (−20.4) + PCDHB2 (76.6) + PCGF3 (30.7) + PKIA (19.1) + PSG7 (1062.1) + RAB32 (−7.4) + |
| | RBM45 (−67.1) + RCN2 (−68.8) + S100A14 (−14.8) + SH3PXD2A (−10.9) + SKP2 (45.5) + |
| | SPAG1 (9.4) + SPATA5 (−55.1) + SSPN (5.8) + STK3 (10.9) + STXBP6 (−18.2) + |
| | TNFRSF12A (20.7) + TRIB1 (−35.1) + TRIM13 (17.3) + TRIM31 (−33.3) + TRIM38 (23.4) + |
| | TYMS (−18.3) + ZNF75D (49.8) − 13.3 |
| | Optimal function group centroids: TEAD-inactive (I) −5.0; TEAD-active (A) 82.7 |
| gggg) | Set gggg): Minimal discriminant function: TFF1 (2.8) + GPR176 (−11.1) + NRIP1 (7.4) + |
| | PCDHB2 (−4.8) + SKP2 (−5.0) + CLDN4 (5.5) + CDCA4 (4.9) + STXBP6 (3.8) + DIAPH3 |
| | (−4.3) − 8.5 |
| | Minimal function group centroids: TEAD-inactive (I) 0.4; TEAD-active (A) −7.0 |
| | Embodiment 56. Optimal and minimal discriminant predictors, with their |
| | unstandardized coefficients, and group centroids for Testicular Germ Cell |
| | Tumors (TGCT) tumors: |
| hhhh) | Set hhhh): Optimal discriminant function: ABAT (−7.3) + ARHGAP11A (−11.4) + |
| | ARHGDIB (11.9) + ATXN1 (−21.2) + AURKB (20.7) + B4GALT4 (−3.4) + BHLHE41 |
| | (11.3) + CAP2 (−4.8) + CCN2 (−6.0) + CCNG2 (−6.2) + CLDN1 (3.3) + CRIM1 (11.5) + |
| | CSTA (2.8) + CTSB (−42.4) + DCLRE1B (−18.4) + DIAPH2 (−8.6) + DSC2 (−6.7) + |
| | FAHD2A (−20.6) + GRB10 (−10.0) + JDP2 (−4.4) + KLF10 (18.8) + LRP10 (24.0) + |
| | LRP8 (−9.2) + LRRFIP2 (6.4) + MDC1 (37.8) + MMP13 (14.2) + MTMR9 (−7.0) + |
| | NEDD4 (27.5) + NFIL3 (−18.0) + NPAS2 (−6.6) + NRIP1 (14.0) + OSBPL7 (−10.7) + |
| | PAK2 (−9.1) + PCGF3 (6.1) + PKP2 (−19.5) + PPP1R11 (31.2) + PRSS23 (−9.9) + |
| | PSG9 (27.1) + RBP1 (14.3) + RCN2 (−11.4) + RHOC (−37.8) + SH3TC1 (15.9) + |
| | SLC25A23 (−14.5) + SLC25A3 (209.8) + SLC30A9 (20.3) + SLFN5 (9.2) + |
| | SLITRK6 (−11.7) + SPAG1 (8.1) + STX1A (14.6) + TACC3 (−43.7) + TEAD4 (−6.8) + |
| | TMEM160 (7.5) + TMEM45B (9.6) + TNFRSF12A (−5.9) + TRAPPC6B (7.9) + |
| | TRIM13 (17.2) + TRIOBP (−9.2) + TRIP13 (6.7) + TUBB6 (35.2) + UAP1 (−7.9) − |
| | 183.9 |
| | Optimal function group centroids: TEAD-inactive (I) 9.4; TEAD-active (A) −6.5 |
| iiii) | Set iiii): Minimal discriminant function: FAHD2A (−9.2) + PCGF3 (6.6) + TMEM45B (5.2) + |
| | PKP2 (−11.1) + SLFN5 (6.2) + TRIOBP (−5.0) + MMP13 (3.3) + LRP10 (13.0) + CCN2 (−5.7) + |
| | KLF10 (6.9) + NEDD4 (13.1) + RBP1 (5.8) + MDC1 (15.7) + DIAPH2 (−3.7) + IGSF3 |
| | (−4.9) + TACC3 (−20.6) + SLC25A3 (70.3) + SLITRK6 (−5.0) + CTSB (−18.2) + AURKB (12.4) + |
| | SLC25A23 (−6.4) + DCLRE1B (−9.9) + TRIM13 (7.3) + PRSS23 (−3.7) + SLC30A9 (5.6) + |
| | GDPD1 (−3.9) + NFIL3 (−4.1) + CSTA (2.0) + PSG9 (10.5) + CRIM1 (3.3) + BHLHE41 (2.2) − |
| | 54.9 |
| | Minimal function group centroids: TEAD-inactive (I) 4.2; TEAD-active (A) −2.9 |
| | Embodiment 57. Optimal and minimal discriminant predictors, with their |
| | unstandardized coefficients, and group centroids for Thyroid carcinoma |
| | (THYM) tumors: |
| jjjj) | Set jjjj): Optimal discriminant function: CTSB (8.1) + SHROOM2 (2.8) + TNNC1 (−6.9) + |
| | ZCWPW1 (7.4) − 9.8 |
| | Optimal function group centroids: TEAD-inactive (I) 0.1; TEAD-active (A) −2.7 |
| | Minimal discriminant function: NA |
| | Embodiment 58. Optimal and minimal discriminant predictors, with their |
| | unstandardized coefficients, and group centroids for Uterine Corpus |
| | Endometrial Carcinoma (UCEC) tumors: |
| kkkk) | Set kkkk): Optimal discriminant function: ADAM28 (.7) + ANXA3 (−2.2) + AURKB (2.2) + |
| | CAVIN1 (−1.4) + CCN2 (−1.5) + CDCA3 (−1.6) + CDCA5 (2.9) + CENPI (−5.9) + CENPN |
| | (−1.9) + CTSK (1.7) + DONSON (−3.2) + FKBP2 (−2.8) + FSTL1 (−3.3) + GPR176 (−1.8) + |
| | GPRC5C (1.2) + GSN (5.1) + HEG1 (−2.1) + IFI44 (1.7) + IRX5 (1.7) + ITGB2 (−1.1) + |
| | KIFAP3 (1.9) + MALL (−1.1) + MCM10 (−4.4) + MICB (−3.3) + MLPH (1.1) + MYO1A (2.4) + |
| | NUAK1 (−1.7) + PPP1R11 (3.1) + PSG6 (11.1) + PXMP2 (−1.9) + SGK1 (.9) + SH3TC1 (1.4) + |
| | SKP2 (−1.4) + SLC1A2 (4.3) + SLC39A11 (1.7) + SLITRK6 (.8) + SLK (2.1) + TK1 (−2.8) + |
| | TMC7 (4.5) + TPM1 (−2.8) + TPX2 (4.8) + TRIM38 (2.2) + TRIOBP (3.5) + TRIP13 (−1.9) + |
| | TSPAN2 (2.3) + TTC17 (−2.5) + ZNF75D (2.3) − .6 |
| | Optimal function group centroids: TEAD-inactive (I) 0.7; TEAD-active (A) −3.0 |
| | Minimal discriminant function: NA |
| | Embodiment 59. Optimal and minimal discriminant predictors, with their |
| | unstandardized coefficients, and group centroids for Uterine |
| | Carcinosarcoma (UCS) tumors: |
| llll) | Set llll): Optimal discriminant function: AASDH (89.6) + ABCC5 (−44.1) + ANKRD29 (−94.3) + |
| | ASF1A (−187.4) + AXL (112.4) + CDK1 (244.2) + CKS2 (−112.0) + CLDN1 (67.5) + CLDN4 |
| | (−49.1) + CLIC3 (−36.5) + CPA4 (186.7) + CTH (371.5) + DONSON (255.5) + EIF2AK3 |
| | (19.4) + FOS (−6.9) + FXYD3 (−87.6) + GADD45B (100.9) + HBP1 (50.0) + IFI44 (−25.4) + |
| | MID1 (−30.5) + NTN4 (−34.6) + PCDHB2 (21.7) + PHLPP1 (294.0) + PPP1R11 (48.6) + |
| | RGS17 (170.9) + SKP2 (288.8) + SLC3A2 (−512.6) + SMOC1 (−111.7) + SPRY4 (−96.8) + |
| | STX1A (−20.7) + SUSD2 (−35.1) + TTC17 (−48.3) + TYMS (−223.1) + UBC (−2396.5) + |
| | VKORC1L1 (18.2) + VPS52 (−205.7) + ZBED2 (−69.6) + 2724.7 |
| | Optimal function group centroids: TEAD-inactive (I) −112.4; TEAD-active (A) 101.1 |
| mmmm) | Set mmmm): Minimal discriminant function: FXYD3 (3.1) + SKP2 (−8.3) + SMOC1 (4.0) + |
| | DONSON (−6.0) + CTH (−7.9) + VPS52 (14.0) + CPA4 (−4.0) + SLC3A2 (14.5) − 16.0 |
| | Minimal function group centroids: TEAD-inactive (I) 2.7; TEAD-active (A) −2.4 |

In some embodiments, a panel may be used for characterizing a subject as having a TEAD-active or TEAD-inactive cancer or predicting whether a subject will respond positively to a TEAD-pathway inhibitor, the use may comprise:
determining the expression level of each tested gene;
determining the fractional rank for each gene;
multiplying the fractional rank for each gene by the corresponding coefficient shown in parenthesis below;
summing the products to give a discriminant score (S);
comparing the discriminant score (S) to the centroids of the TEAD-active (A) and TEAD-inactive (I) groups;
determining if (S) is closer to (A) than it is to (I) according to the formula max(S,A)−min(S,A)<max(S,I)−min(S,I); and
characterizing the sample as TEAD-active if (S) is closer to (A) than it is to (I), where the above embodiments (or sets) show the coefficients and centroids for each set a) to mmmm).

In some embodiments, a use of a “short TEAD signature” according to the disclosure may be such that when the cancer is a
Adrenocortical carcinoma (ACC) tumor, then the set of genes of the transcription signature is set of genes a) or b);
Bladder Urothelial Carcinoma (BLCA) tumor, then the set of genes of the transcription signature is set of genes c);
Breast invasive carcinoma (BRCA) tumor, then the set of genes of the transcription signature is set of genes d);
BRCA basal tumor, then the set of genes of the transcription signature is set of genes e) or f);
BRCA non-basal tumor, then the set of genes of the transcription signature is set of genes g) or h);
Cervical squamous cell carcinoma or endocervical adenocarcinoma (CESC) tumor, then the set of genes of the transcription signature is set of genes i);
Cholangiocarcinoma (CHOL) tumor, then the set of genes of the transcription signature is set of genes j) or k);
Colon adenocarcinoma (COAD) tumor, then the set of genes of the transcription signature is set of genes I);
COAD CMS1 tumor, then the set of genes of the transcription signature is set of genes m) or n);
COAD CMS2 tumor, then the set of genes of the transcription signature is set of genes o) or p);
COAD CMS4 tumor, then the set of genes of the transcription signature is set of genes q) or q1);
Lymphoid Neoplasm Diffuse Large B-cell Lymphoma (DLBC) tumor, then the set of genes of the transcription signature is set of genes r) or s);
Esophageal carcinoma (ESCA) tumor, then the set of genes of the transcription signature is set of genes t) or u);
Glioblastoma multiforme (GBM) tumor, then the set of genes of the transcription signature is set of genes v) or w);
Head or Neck squamous cell carcinoma (HNSC) tumor, then the set of genes of the transcription signature is set of genes x);
Kidney Chromophobe (KICH) tumor, then the set of genes of the transcription signature is set of genes y) or z);
Kidney renal clear cell carcinoma (KIRC) tumor, then the set of genes of the transcription signature is set of genes aa);
Kidney renal papillary cell carcinoma (KIRP) tumor, then the set of genes of the transcription signature is set of genes bb) or cc);
Brain Lower Grade Glioma (LGG) tumor, then the set of genes of the transcription signature is set of genes dd) or ee);
Liver hepatocellular carcinoma (LIHC) tumor, then the set of genes of the transcription signature is set of genes ff);
LIHC S1 tumor, then the set of genes of the transcription signature is set of genes gg) or hh);
LIHC S2 tumor, then the set of genes of the transcription signature is set of genes ii) or jj);
LIHC S3 tumor, then the set of genes of the transcription signature is set of genes kk) or II);
Lung adenocarcinoma (LUAD) tumor, then the set of genes of the transcription signature is set of genes mm);
LUAD proximal-inflammatory tumor, then the set of genes of the transcription signature is set of genes nn) or oo);
LUAD proximal-proliferative tumor, then the set of genes of the transcription signature is set of genes pp) or qq);
Lung squamous cell carcinoma (LUSC) tumor, then the set of genes of the transcription signature is set of genes rr);
LUSC basal tumor, then the set of genes of the transcription signature is set of genes ss) or tt);
LUSC classical tumor, then the set of genes of the transcription signature is set of genes uu);
LUSC primitive tumor, then the set of genes of the transcription signature is set of genes vv) or ww);
LUSC secretory tumor, then the set of genes of the transcription signature is set of genes xx) or yy);
Malignant mesothelioma (MESO) tumor, then the set of genes of the transcription signature is set of genes zz) or aaa);
Ovarian serous cystadenocarcinoma (OV) tumor, then the set of genes of the transcription signature is set of genes bbb);
OV differentiated tumor, then the set of genes of the transcription signature is set of genes ccc) or ddd);
OV immune-reactive tumor, then the set of genes of the transcription signature is set of genes eee) or fff);
OV mesenchymal tumor, then the set of genes of the transcription signature is set of genes ggg) or hhh);
OV proliferative tumor, then the set of genes of the transcription signature is set of genes iii) or jj);
Pancreatic adenocarcinoma (PAAD) tumor, then the set of genes of the transcription signature is set of genes kkk) or kkk1);
Rectum adenocarcinoma (READ) tumor, then the set of genes of the transcription signature is set of genes lll);
READ CMS1 tumor, then the set of genes of the transcription signature is set of genes mmm);
READ CMS2 tumor, then the set of genes of the transcription signature is set of genes nnn) or ooo);
READ CMS4 tumor, then the set of genes of the transcription signature is set of genes ppp) or ppp1);
READ unclassifiable tumor, then the set of genes of the transcription signature is set of genes qqq) or qqq1);
Sarcoma (SARC) tumor, then the set of genes of the transcription signature is set of genes rrr);
Skin Cutaneous Melanoma (SKCM) tumor, then the set of genes of the transcription signature is set of genes sss);
SKCM immune tumor, then the set of genes of the transcription signature is set of genes ttt);
SKCM keratin tumor, then the set of genes of the transcription signature is set of genes uuu) or vvv);
SKCM MITF-low tumor, then the set of genes of the transcription signature is set of genes www) or xxx);
Stomach adenocarcinoma (STAD) tumor, then the set of genes of the transcription signature is set of genes yyy);
STAD MSI tumor, then the set of genes of the transcription signature is set of genes zzz) or aaaa);
STAD MSS_EMT tumor, then the set of genes of the transcription signature is set of genes bbbb) or cccc);
STAD MSS_TP53-tumor, then the set of genes of the transcription signature is set of genes dddd) or eeee);
STAD MSS_TP53+ tumor, then the set of genes of the transcription signature is set of genes ffff) or gggg);
Testicular Germ Cell Tumor (TGCT) tumor, then the set of genes of the transcription signature is set of genes hhhh) or iiii);
Thyroid carcinoma (THYM) tumor, then the set of genes of the transcription signature is set of genes jjjj);
Uterine Corpus Endometrial Carcinoma (UCEC) tumor, then the set of genes of the transcription signature is set of genes kkkk); and
Uterine Carcinosarcoma (UCS) tumor, then the set of genes of the transcription signature is set of genes IIII) or mmmm).

In some embodiments, the use of “short TEAD signature” does not include the use of sets of genes q1), kkk1), ppp1) and qqq1).

In some embodiments, a use of as disclosed herein may comprise:
a) measuring, in a biological sample of a tumor of a cancer, the expression level of each gene of a set of genes of a “TEAD-signature”, i.e., any of 220 to 249 of genes of a set of genes (1) (positive effector genes) and any of 210 to 233 of genes of a set of genes (2) (negative effector genes),
b) for each gene of a set of genes shown in Table 1, converting the gene's expression level obtained at step a) into a fractional rank by dividing the rank of said gene by the number of genes of said set of genes of the “TEAD signature”;
c) multiplying each fractional rank obtained at step b) by a coefficient associated with each gene of said set of genes of Table 1, said coefficient for said gene and set of genes being shown in Table 2, to obtain a product for each gene;
d) summing the products obtained at step c) to obtain a discriminant score (S);
e) comparing the discriminant score (S) obtained at step d) to the centroids of the TEAD-active (A) and TEAD-inactive (I) groups shown in Table 2 for said set of genes of Table 1;
f) determining if (S) is closer to (A) than it is to (I) according to the formula max(S,A)−min(S,A)<max(S,I)−min(S,I); and
g) characterizing the sample as TEAD-active if (S) is closer to (A) than it is to (I).

In some embodiments, a use of as disclosed herein may be such that:
the cancer may be selected from the group consisting of Adrenocortical carcinoma (ACC) tumors; Bladder Urothelial Carcinoma (BLCA) tumors; Breast invasive carcinoma (BRCA) tumors; BRCA basal tumors; BRCA non-basal tumors; Cervical squamous cell carcinoma and endocervical adenocarcinoma (CESC) tumors; Cholangiocarcinoma (CHOL) tumors; Colon adenocarcinoma (COAD) tumors; COAD CMS1 tumors; COAD CMS4 tumors; Lymphoid Neoplasm Diffuse Large B-cell Lymphoma (DLBC) tumors; Esophageal carcinoma (ESCA) tumors; Glioblastoma multiforme (GBM) tumors; Head and Neck squamous cell carcinoma (HNSC) tumors; Kidney Chromophobe (KICH) tumors; Kidney renal clear cell carcinoma (KIRC) tumors; Kidney renal papillary cell carcinoma (KIRP) tumors; Brain Lower Grade Glioma (LGG) tumors; Liver hepatocellular carcinoma (LIHC) tumors; LIHC S1 tumors; LIHC S2 tumors; LIHC S3 tumors; Lung adenocarcinoma (LUAD) tumors; Lung squamous cell carcinoma (LUSC) tumors; LUSC basal tumors; LUSC classical tumors; LUSC primitive tumors; LUSC secretory tumors; Malignant mesothelioma (MESO) tumors; Pancreatic adenocarcinoma (PAAD) tumors; Rectum adenocarcinoma (READ) tumors; READ CMS1 tumors; READ CMS2 tumors; is READ CMS4 tumors; Sarcoma (SARC) tumors; Skin Cutaneous Melanoma (SKCM) tumors; SKCM immune tumors; SKCM keratin tumors; STAD MSI tumors; STAD MSS_EMT tumors; Testicular Germ Cell Tumors (TGCT) tumors; Uterine Corpus Endometrial Carcinoma (UCEC) tumors; Uterine Carcinosarcoma (UCS) tumors,
and
the use may comprise:
a) measuring, in a biological sample of a tumor of said cancer, the expression level of each gene of a set of genes associated with said tumor, the set of genes being selected from the group consisting of set a), set c), set d), set e), set g), set i), set j), set l), set m), set q), set r), t), set v), set x), set y), set aa), set bb), set dd), set ff), set gg), set ii), set kk), set mm), set rr), set ss), set uu), set vv), set xx), set zz), set kkk), set III), set mmm), set nnn), set ppp), set rrr), set sss), set ttt), set uuu), set zzz), set bbbb), set hhhh), set kkkk), and set IIII) of Table 1;
b) for each gene of said set of genes, converting the gene's expression level obtained at step a) into a fractional rank by dividing the rank of said gene by the number of genes of said set of genes;
c) multiplying each fractional rank obtained at step b) by a coefficient associated with each gene of said set of genes, said coefficient for said gene and set of genes being shown in Table 2, to obtain a product (Pgeneᵢ) for each gene;
d) determining a discriminant score (DS) of said set of genes, the discriminant score (DS) being the sum of the products Pgeneᵢ obtained at step c) plus the constant coefficient for said set of genes shown in Table 2,
e) comparing, for said set of genes, the discriminant score (DS) obtained at step d) with a threshold associated with said set of genes, the threshold being shown in Table 3, to determine if the cancer is TEAD-active or TEAD-inactive.

TABLE 3
Thresholds for calling the TEAD status of a tumor
‘active’ or ‘inactive’ per cohort (indication
or sub-indication) for the “short TEAD signature”.

| Cancer indication - | TEAD is active if the discriminant score (DS) is | |
|---|---|---|
| Set of genes | less than | greater than |
| ACC | −156.02 | NA |
| set a) | | |
| BLCA | −1.76 | NA |
| set c) | | |
| BRCA | NA | 2.95 |
| set d) | | |
| BRCA_basal | NA | 5.81 |
| set e) | | |
| BRCA_non-basal | NA | 8.1 |
| set g) | | |
| CESC | −2.15 | NA |
| set i) | | |
| CHOL | NA | 63.98 |
| set j) | | |
| COAD | −3.37 | NA |
| set l) | | |
| COAD_CMS1 | −116.35 | NA |
| set m) | | |
| COAD_CMS4 | −556.79 | NA |
| set q) | | |
| DLBC | NA | −132.6 |
| set r) | | |
| ESCA | −5.52 | NA |
| set t) | | |
| GBM | −7.19 | NA |
| set v) | | |
| HNSC | −2.65 | NA |
| set x) | | |
| KICH | NA | 393.41 |
| set y) | | |
| KIRC | NA | 3.09 |
| set aa) | | |
| KIRP | NA | 12.65 |
| set bb) | | |
| LGG | NA | 7.77 |
| set dd) | | |
| LIHC | NA. | 2.73 |
| set ff) | | |
| LIHC_S1 | NA | 118.11 |
| set gg) | | |
| LIHC_S2 | −60.84 | NA |
| set ii) | | |
| LIHC_S3 | NA | 4.88 |
| set kk) | | |
| LUAD | NA | 4.15 |
| set mm) | | |
| LUSC | −1.86 | NA |
| set rr) | | |
| LUSC_basal | −455.53 | NA |
| set ss) | | |
| LUSC_classical | −3.44 | NA |
| set uu) | | |
| LUSC_primitive | NA | 60.27 |
| set vv) | | |
| LUSC_secretory | NA | 80.4 |
| set xx) | | |
| MESO | 2.05 | NA |
| set zz) | | |
| PAAD | −43 | NA |
| set kkk) | | |
| READ | −1.78 | NA |
| set lll) | | |
| READ_CMS1 | −44.65 | NA |
| set mmm) | | |
| READ_CMS2 | NA | 89.22 |
| set nnn) | | |
| READ_CMS4 | NA | −10.47 |
| set ppp) | | |
| SARC | 0.5 | NA |
| set rrr) | | |
| SKCM | NA | −0.85 |
| set sss) | | |
| SKCM_immune | −3.41 | NA |
| set ttt) | | |
| SKCM_keratin | NA | −30.14 |
| set uuu) | | |
| STAD_MSI | −74.44 | NA |
| set zzz) | | |
| STAD_MSS_EMT | −340.92 | NA |
| set bbbb) | | |
| TGCT | 11.89 | NA |
| set hhhh) | | |
| UCEC | −0.67 | NA |
| set kkkk) | | |
| UCS | NA | −49.52 |
| set llll) | | |

NA: Not applicable

The above indicated use may be referred to as Method 4.

In some embodiments, the biological sample may be blood, plasma, serum, cerebral spinal fluid (CSF), a tissue, a cell, or a tumor biopsy.

In some embodiments, a use may be for characterizing a subject as having a TEAD-active or TEAD-inactive cancer

In some embodiments, a use may be for predicting whether a subject will respond positively to a TEAD-pathway inhibitor,

In some embodiments, the subject may be administered a TEAD-pathway inhibitor if the subject is characterized as having a TEAD-active cancer or if the subject is predicted to respond positively to a TEAD-pathway inhibitor.

In some embodiments, the disclosure relates to a method for characterizing the TEAD-activity of a cancer in a subject in need thereof, the method using a biological sample of a tumor of said cancer and comprising at least the steps of:
a) measuring, in a transcriptome obtained from said biological sample, the expression levels of genes,
b) for each gene of a set of genes of a “TEAD-signature”, i.e., any of 220 to 249 of genes of a set of genes (1) (positive effector genes) and any of 210 to 233 of genes of a set of genes (2) (negative effector genes), converting the gene's expression level obtained at step a) into a fractional rank by dividing the rank of said gene by the number of genes from said transcriptome,
c) isolating from the fractional ranks obtained at step b) the fractional rank obtained for the genes of the set of genes (1) and computing their mean fractional rank (MFR-positive),
d) isolating from the fractional ranks obtained at step b) the fractional rank obtained for the genes of the set of genes (2) and computing their mean fractional rank (MFR-negative), and
e) computing the dgR score as MFR-positive-MFR-negative;
or
a) measuring, in said biological sample, the expression level of each gene of a set of genes of a “TEAD-signature”, i.e., any of 220 to 249 of genes of a set of genes (1) (positive effector genes) and any of 210 to 233 of genes of a set of genes (2) (negative effector genes),
b) for each gene of said set of genes of a “TEAD signature”, converting the gene's expression level obtained at step a) into a fractional rank by dividing said rank of said gene by the number of genes from said set of genes,
c) isolating from the fractional ranks obtained at step b) the fractional rank obtained for the genes of the set of genes (1) and computing their mean fractional rank (MFR-positive),
d) isolating from the fractional ranks obtained at step b) the fractional rank obtained for the genes of the set of genes (2) and computing their mean fractional rank (MFR-negative),
e) computing the deR score as MFR-positive-MFR-negative;
wherein when the dgR or the deR score is greater than about 0.055, then the cancer is TEAD-active, when the dgR or deR score is less than or equal to about 0.055, then the cancer is TEAD-inactive.

In some embodiments, the disclosure relates a method for characterizing the TEAD-activity of a cancer in a subject in need thereof, the method using a biological sample of a tumor of said cancer and comprising at least the steps of:
a) measuring, in said biological sample, the expression level of each gene of a set of genes of a “TEAD-signature”, i.e., any of 220 to 249 of genes of a set of genes (1) (positive effector genes) and any of 210 to 233 of genes of a set of genes (2) (negative effector genes),
b) for each gene of a set of genes shown in Table 1, converting the gene's expression level obtained at step a) into a fractional rank by dividing the rank of said gene by the number of genes of said set of genes of the “TEAD signature”;
c) multiplying each fractional rank obtained at step b) by a coefficient associated with each gene of said set of genes of Table 1, said coefficient for said gene and set of genes being shown in Table 2, to obtain a product for each gene;
d) summing the products obtained at step c) to obtain a discriminant score (S);
e) comparing the discriminant score (S) obtained at step d) to the centroids of the TEAD-active (A) and TEAD-inactive (I) groups shown in Table 2 for said set of genes of Table 1;
f) determining if (S) is closer to (A) than it is to (I) according to the formula max(S,A)−min(S,A)<max(S,I)−min(S,I); and
g) characterizing the cancer as TEAD-active if (S) is closer to (A) than it is to (I).

In some embodiments, the disclosure relates to a method for characterizing the TEAD-activity of a cancer in a subject in need thereof, wherein
the cancer is selected from the group consisting of Adrenocortical carcinoma (ACC) tumors; Bladder Urothelial Carcinoma (BLCA) tumors; Breast invasive carcinoma (BRCA) tumors; BRCA basal tumors; BRCA non-basal tumors; Cervical squamous cell carcinoma and endocervical adenocarcinoma (CESC) tumors; Cholangiocarcinoma (CHOL) tumors; Colon adenocarcinoma (COAD) tumors; COAD CMS1 tumors; COAD CMS4 tumors; Lymphoid Neoplasm Diffuse Large B-cell Lymphoma (DLBC) tumors; Esophageal carcinoma (ESCA) tumors; Glioblastoma multiforme (GBM) tumors; Head and Neck squamous cell carcinoma (HNSC) tumors; Kidney Chromophobe (KICH) tumors; Kidney renal clear cell carcinoma (KIRC) tumors; Kidney renal papillary cell carcinoma (KIRP) tumors; Brain Lower Grade Glioma (LGG) tumors; Liver hepatocellular carcinoma (LIHC) tumors; LIHC S1 tumors; LIHC S2 tumors; LIHC S3 tumors; Lung adenocarcinoma (LUAD) tumors; Lung squamous cell carcinoma (LUSC) tumors; LUSC basal tumors; LUSC classical tumors; LUSC primitive tumors; LUSC secretory tumors; Malignant mesothelioma (MESO) tumors; Pancreatic adenocarcinoma (PAAD) tumors; Rectum adenocarcinoma (READ) tumors; READ CMS1 tumors; READ CMS2 tumors; is READ CMS4 tumors; Sarcoma (SARC) tumors; Skin Cutaneous Melanoma (SKCM) tumors; SKCM immune tumors; SKCM keratin tumors; STAD MSI tumors; STAD MSS_EMT tumors; Testicular Germ Cell Tumors (TGCT) tumors; Uterine Corpus Endometrial Carcinoma (UCEC) tumors; and Uterine Carcinosarcoma (UCS) tumors,
and
the method uses a biological sample of a tumor of said cancer, and
the method comprises at least the steps of:
a) measuring, in a biological sample of said tumor, the expression level of each gene of a set of genes associated with said tumor, the set of genes being selected from the group consisting of set a), set c), set d), set e), set g), set i), set j), set l), set m), set q), set r), t), set v), set x), set y), set aa), set bb), set dd), set ff), set gg), set ii), set kk), set mm), set rr), set ss), set uu), set vv), set xx), set zz), set kkk), set lll), set mmm), set nnn), set ppp), set rrr), set sss), set ttt), set uuu), set zzz), set bbbb), set hhhh), set kkkk), and set llll) of Table 1;
b) for each gene of said set of genes, converting the gene's expression level into a fractional rank by dividing the rank of the gene by the number of genes of said set of genes;
c) multiplying each fractional rank obtained at step b) by a coefficient associated with each gene of said set of genes, said coefficient for said gene and set of genes being shown in Table 2, to obtain a product (Pgeneᵢ) for each gene;
d) determining a discriminant score (DS) of said set of genes, the discriminant score (DS) being the sum of the products Pgeneᵢ obtained at step c) plus the constant coefficient of said set of genes shown in Table 2;
e) comparing, for said set of genes, the discriminant score (DS) obtained at step d) with a threshold associated with said set of genes, the threshold being shown in Table 3, to determine if the cancer is TEAD-active or TEAD-inactive.

A kit comprising a solid support comprising a panel of nucleic acid for determining the transcription of a set of genes according to the disclosure.

In some embodiments, the use or method may comprise detecting the RNA expression level of each of the genes in the panel.

In some embodiments, the use or method may comprise RNA sequencing.

In some embodiments, the disclosure relates to a kit comprising a solid support comprising a panel of nucleic acid biomarkers as disclosed herein.

In some embodiments, the disclosure relates to a kit comprising a solid support comprising a panel of nucleic acid for determining the transcription of a set of genes as disclosed herein.

### DETAILED DESCRIPTION

### Definitions

Signature, gene signature and transcriptional signature. Those terms and expression as used herein intend to refer to a pattern of transcription of a set of genes which can be associated with a TEAD-active cancer or tumor. A transcriptional signature is obtained by measure of the expression levels of the genes of the signature (or of the sets of genes of the signature) in a biological sample. In the biological sample, one may measure the expression levels of all or part of the genes susceptible to be expressed in the biological sample. When measuring only a part of the genes, the measure is necessarily carried out at least on all the genes of the set of genes of interest.

Transcriptome. The term “transcriptome” intends to refer to the set of all transcripts readouts (RNA), including coding and non-coding, in an individual or a population of cells. Within the disclosure, a transcriptome may be used in all or in part. When used in part, it necessarily comprises at least all the genes of the set of genes of interest.

Herein, “transcription” and “expression of a gene” are used interchangeably to refer to the production of RNA. The transcription or expression of a gene may increase or decrease resulting in an increase or decrease of the production of RNA.

Positive effector. A positive effector is a gene of which the transcript level correlates positively with TEAD activity. It increases when the TEAD pathway is somehow activated or decreases when the TEAD pathway is inhibited. A positive effector is not necessarily expected to respond in both directions of TEAD modulation. For example, a gene of which the normal expression in a tissue is null, may be seen increased upon TEAD activation but cannot be expected to decrease upon TEAD inhibition. Even at higher normal levels of expression, a positive effector gene may not necessarily be sensitive to both TEAD activation and inhibition.

Negative effector. A negative effector is a gene of which the transcript level correlates negatively with TEAD activity. It decreases upon TEAD activation or increases with TEAD inhibition. Like a positive effector, a negative effector is not necessarily expected to respond to TEAD modulation in both directions. For example, a gene of which the normal expression in a tissue is null, may be seen increased upon TEAD-pathway inhibition but cannot be expected to decrease upon TEAD activation. Even at higher normal levels of expression, a negative effector gene may be sensitive to TEAD activation, but immune to TEAD inhibition, or vice versa.

The TEAD-500 gene list. A list of 500, or less, most significant positive or negative effectors of TEAD modulation. These genes were selected by differential expression analyses of 18 published expression array datasets. Some of the 500 genes are direct targets of TEAD transcription factors, meaning that they present a TEAD-recognition motif in their promoter, but most of these 500 genes are subject to secondary or indirect regulation by the TEAD pathway. Due to technological evolution (from various commercial expression arrays to RNA sequencing techniques), but also to gene nomenclature inconsistencies, practically the panel does not contain 500 genes but now, 482 genes. Therefore, in the present disclosure it is referred to this panel as “TEAD-500” but this TEAD-500 panel consists of 482 genes to about 90% of the 482 genes. Dropouts up to at least 10% only negligibly influence its score. This is one of the reasons why TEAD-500 is highly robust.

TEAD signature. “TEAD signature” as used herein intends to refer to a transcriptional signature obtained by measure of the expression levels of the genes of a set of genes comprising any of 220 to 249 of positive effector genes and any of 210 to 233 of negative effector genes as listed herein. By extension, depending on the context, “TEAD signature” may refer to the associated set of genes.

Short TEAD signature. “Short TEAD signature” as used herein intends to refer to a transcriptional signature obtained by measure of the expression level of the genes of a set of genes selected from the group consisting of sets a) to mmmm) of Table 1. By extension, depending on the context, “short TEAD signature” may refer to the associated set of genes.

Scoring. TEAD-500 scoring (herein referred to as “TEAD score”) is a procedure of calculating the activity of the TEAD pathway in a sample, on a continuous scale of values. The score is based on the mean fractional ranks of the transcript levels of the positive and negative effectors. Depending on the available datasets, two types of scores may be calculated, referred to as dgR or deR. The two scores are not identical but are analogous. They both lead to the same conclusions, but their values are not comparable. They both represent the difference Rp-Rn, where Rp is the mean fractional rank of the positive effectors, and Rn, that of the negative effectors. They differ in the way by which the fractional ranks are defined and calculated.

Fractional rank. In an array of values such as the gene levels in a transcriptome, each value is replaced by its rank, with the smallest value taking the rank 1. The fractional rank of each gene equals its rank divided by the number of genes in the array (maximum rank).

Difference of genome-wide ranks (dgR). A TEAD pathway activity score calculated from whole transcriptomes. When entire transcriptomes are available, all transcript levels may be converted into fractional ranks (the level of a transcript divided by the level of the most abundant transcript observed in a sample), and the means ranks of positive and negative effectors are calculated from so transformed transcript levels.

Difference of effector ranks (deR). For very large cohorts, or when full transcriptomic data are not available, it may be convenient to interrogate only the genes of the TEAD-500 list. The levels of the 500 transcripts are transformed into fractional ranks, as for dgR above, but the ranks of 500 level-values are obviously not the same as the ranks of, say, 20000 values in the entire transcriptome. Therefore, deR differs from dgR but both scores can be used in the same way.

Calling (binning) the TEAD activity status. This is a binary transformation of the continuous deR or dgR scores of TEAD activity into two discrete status values: “active” or “inactive”. In some embodiments, we empirically set the deR threshold to 0.055. This value was chosen from an experiment with human mesothelioma cell lines. We observed that cell lines with deR score less than 0.055 did not respond to YAP1-siRNA treatment whereas cell lines with higher scores stopped growing when YAP1 was so knocked-out. The threshold of response may be different for different tissues or treatments. The score value of 0.055 approximately corresponds to the 88^{th} percentile of the scores observed in tumors of the TCGA cohort (all indications pooled). If the deR score of TEAD-500 is greater than 0.055, TEAD is called active, otherwise, inactive.

Discriminant function. Generally, this is a linear combination of predictors (in our case effector genes)—each accompanied by a coefficient—that separates two (in this case) or more classes of objects. Our objects are tumor samples, and the two classes are the “active” or “inactive” TEAD phenotypes as these are determined by TEAD activity calling based on the TEAD-500 transcriptomic signature. The resulting combination of non-redundant genes may be used as a linear classifier or for dimensionality reduction before later classification.

Discriminant score (S). The score of a sample for a discriminant function calculated as the sum of the products of predictor transcript levels (typically fractional ranks or other normalized metrics) multiplied by the corresponding predictor coefficients. For example, if a discriminant function includes the genes X, Y, and Z, with the coefficients x, y, and z, respectively, then, the discriminant score (S) of a sample i for that function will be:

$$Si = ( x * Xi ) + ( y * Yi ) + ( z * Zi )$$

where Xi, Yi, and Zi are the levels of transcripts X, Y, and Z respectively, in sample i.

Discriminant score (DS). The score of a sample for an optimal discriminant function calculated as the sum of the products of all the discriminant genes of the optimal discriminant function plus the constant coefficient of the optimal discriminant function. For example, if the optimal discriminant function for the indication in which the sample belongs has 10 genes, we multiply the fractional rank of each gene with the corresponding discriminant coefficient (known as the loading) of the gene, we add up the resulting products to the constant coefficient of the optimal discriminant function.

Discriminant score (DS)=Σ(fractional rank of Geneᵢ*coefficient for Geneᵢ)+discriminant constant, where Geneᵢ is a gene listed in an optimal discriminant function.

An optimal discriminant function is given for each of the set of genes a) to mmmm). To each of the set of genes is associated a specific cancer type.

Group centroid. Simply referred to as centroid, this is the mean score of a discriminant function for a group of samples. In some embodiments, to classify samples as TEAD-active or TEAD-inactive, one calculates the centroid of each group as the mean discriminant score within that group. The farther apart the means are, the less error there will be in classification. Every new sample is classified according to the distance of its discriminant score from the two centroids. If its distance from the TEAD-active centroid is shorter than its distance from the TEAD-inactive centroid, then the new sample is classified as TEAD-active, otherwise, as TEAD-inactive.

Optimal discriminant function. By default, all the significant predictors are used in a discriminant function. Using more than one alternative predictor increases the robustness of the model because it allows for dropouts without compromising the accuracy of prediction. Unless otherwise indicated as minimal, the general term discriminant function refers, herein, to an optimal solution.

Minimal discriminant function. For the sake of assay economy, it may be desirable to retain only the essential predictors for achieving 100% accuracy of classification. When an optimal discriminant function reaches 100% correct classification, it is possible to manually remove predictors and stop just before the accuracy falls below 100%. Minimal discriminant functions are relevant, and are reported herein, only when the optimal solution achieves 100% correct classification.

TEAD-pathway inhibitor. As used herein, a TEAD-pathway inhibitor is any small or large molecule compound that inhibits the HIPPO-YAP/WWTR1/TEAD pathway.

Expression level. As used herein, the “expression level” of a gene refers to the level of RNA expressed by any given gene.

### Gene Panels & Transcriptional Signatures

In some embodiments, a panel of N nucleic acid biomarkers is encompassed, wherein N is an integer of at least 160 and equal to or less than 482, and wherein the N biomarkers comprise nucleic acid capable of detecting any 80 to 249 of genes of a set of genes (1) (the positive effector genes): AASS, ABAT, ACAT2, ADAMTS1, ADM, ADRB2, AMOT, ANXA3, ARHGAP11A, ARHGDIB, AURKB, AVPI1, AXL, AZIN1, B4GALT4, BCAT1, BIRC5, BTG3, C4BPB, CAP2, CAV1, CAVIN1, CCBE1, CCDC80, CCN1, CCN2, CDC25A, CDC6, CDCA3, CDCA4, CDCA5, CDCA8, CDH4, CDK2, CDK6, CDV3, CENPA, CENPI, CENPM, CENPN, CHRNB1, CHST13, CKS2, CLDN1, CLIC3, CNN3, COBL, COL8A1, COTL1, CPA4, CRIM1, CRY1, CTH, CXCL1, CYTH3, DAPK1, DCLRE1B, DDAH1, DHCR7, DHFR, DIAPH3, DKK1, DLL1, DONSON, DUSP14, DUT, EBP, EIF2AK3, EMG1, EPHA2, EPS8L2, ESM1, ETS1, EXO1, EXOSC2, F3, FAHD2A, FAM83D, FANCA, FAT4, FDPS, FEN1, FMR1, FST, FSTL1, FSTL3, GADD45A, GADD45B, GINS1, GPC6, GPR176, GPRC5A, GPRC5B, GRAMD2B, HASPIN, HEG1, HEXB, HPS5, HSPB11, IDI1, IGFBP7, IKBIP, IL6, ITGB2, JDP2, JPH2, KPNA2, KRT8, KRT80, LCA5, LHFPL6, LMCD1, LMNB2, LRP8, LRRFIP2, LSM5, LYPD6, LYRM1, MAD2L1, MAP6D1, MATN2, MATN3, MCM10, MCM2, MCM5, MDC1, METRNL, MICB, MID1, MRPL33, MSRB3, MVD, MXRA7, NCAPD3, NEDD4, NEDD4L, NEK2, NEXN, NFIB, NNMT, NOC3L, NTN4, NUAK1, NUAK2, NUDCD1, NUP107, NUP37, OGFRL1, OLFML3, OLR1, OXCT1, PAK2, PCBD1, PCNA, PDLIM2, PDZD2, PEPD, PHLPP1, PKMYT1, PKP2, PKP4, PLCE1, PLEKHA7, PLK2, PLOD2, PPIH, PRPS1, PRPS2, PRSS23, PSG2, PSG6, PSG7, PSG9, PVR, PXMP2, QDPR, QKI, RAB11FIP1, RAB32, RACGAP1, RBM24, RBMS2, RCN2, RFC4, RND3, RNF144B, ROR1, RPS24, SCD5, SCML1, SDC2, SEC14L1, SGK1, SGMS2, SGTB, SH3RF1, SHCBP1, SKP2, SLC25A23, SLC25A3, SLC38A5, SLC3A2, SLC7A1, SLC7A5, SMPD4, SNAPC1, SNX24, SORT1, SPAG1, SPATA5, STK3, STX11, STXBP6, SUSD2, SUV39H1, SYDE2, TACC3, TAGLN, TEAD1, TEAD4, TENT5B, TGM2, THBS1, TK1, TMEM139, TMEM160, TNFAIP3, TNFRSF12A, TNNC1, TPM1, TPX2, TRIP13, TSPAN2, TTF2, TUBB6, TUFT1, TYMS, UAP1, UBE2C, UGCG, UHRF1, VKORC1L1, WWC1, WWC2, YAP1, ZBED2, ZDHHC18, ZNF488, and ZNF704 (the “positive effector genes”); and
any 80 to 233 of genes of a set of genes (2) (the negative effector genes): AASDH, ABCA1, ABCC5, ABI3BP, ABLIM3, ACADVL, ACOT11, ACOX2, ACSL5, ADAM28, AGL, AGPAT4, ALDH3A2, ANKRD12, ANKRD22, ANKRD29, ANKRD42, ANTXR2, APBB3, ARAP3, ARHGEF2, ASF1A, ATP7A, ATXN1, BCL11B, BHLHE41, BMF, CA2, CASP1, CBR3, CCNG2, CDC42EP4, CDK1, CEBPB, CELSR3, CLCN3, CLDN4, COL6A1, COL6A2, CPE, CRABP2, CROT, CSRNP2, CSTA, CTNNBIP1, CTSB, CTSK, CXXC5, CYP1B1, CYP27C1, DDR1, DEDD2, DHX32, DIAPH2, DSC2, DSG3, DUSP6, DYNC2LI1, ELN, EPS8L3, ERAP2, FAM102A, FAM117B, FAM83B, FAM89B, FERMT1, FKBP2, FOS, FTH1, FXYD3, GDPD1, GOLGA5, GOLPH3L, GPNMB, GPRC5C, GRB10, GSN, HAS3, HBP1, HDAC1, HDHD2, HEY1, HOXA5, IFI44, IGSF3, IGSF9, INTS3, IRAK2, IRF9, IRX5, ITGA2, KCNMA1, KCNMB3, KCNN4, KIFAP3, KLF10, KLF13, KLHL3, KLK11, KRCC1, KRIT1, KRTDAP, LMTK3, LRP10, LTBP4, LXN, LYPD3, MALL, MANSC1, MAPK13, MARCKSL1, MFSD1, MFSD5, MGST2, MGST3, MLLT11, MLPH, MMP13, MSX2, MTMR11, MTMR9, MTSS1, MYO1A, NAGK, NAPEPLD, NCOA3, NFIL3, NPAS2, NRIP1, OAS1, OAS2, OASL, OFD1, OSBPL7, OTUB2, OVOL1, PAG1, PAK1, PCDHB2, PCDHB9, PCGF3, PCMTD2, PERP, PHF21A, PIK3C2B, PIK3R1, PIK3R2, PIK3R3, PIP4P2, PJA2, PKIA, PLA2G4C, PNRC1, PPP1R11, PRRX2, PTPRE, PYGB, RAC2, RALGPS1, RAPGEFL1, RBM23, RBM45, RBM47, RBP1, REEP6, RGL2, RGS17, RHOC, S100A14, SAMD9, SEC14L2, SECISBP2, SH3PXD2A, SH3TC1, SHROOM2, SLC14A1, SLC1A2, SLC30A9, SLC35C1, SLC37A2, SLC39A11, SLFN5, SLITRK6, SLK, SMOC1, SNCG, SP1, SPIRE2, SPRY4, SQSTM1, SRD5A3, SSPN, STMN3, STX1A, TBX3, TCF25, TDO2, TET2, TFF1, TLR3, TMC4, TMC7, TMEM140, TMEM144, TMEM45B, TP53INP1, TP63, TPD52L1, TRAPPC6B, TRIB1, TRIB2, TRIM13, TRIM31, TRIM38, TRIOBP, TRIP11, TSC22D1, TSPAN1, TTC17, TTLL3, TUBB3, UBC, ULK1, VAMP8, VGF, VPS52, VSNL1, WDR13, ZCWPW1, ZNF292, ZNF467, ZNF75D, and ZSWIM7 (the “negative effector genes”).

In some embodiments, a panel of N nucleic acid biomarkers is encompassed, wherein the N biomarkers comprise nucleic acid capable of detecting at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, at least 233, at least 234, at least 235, at least 236, at least 237, at least 238, at least 239, at least 240, at least 241, at least 242, at least 243, at least 244, at least 245, at least 246, at least 247, at least 248, or all 249 of the genes of the set of genes (1) (the positive effector genes), and
comprises nucleic acid capable of detecting at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, at least 224, at least 225, at least 226, at least 227, at least 228, at least 229, at least 230, at least 231, at least 232, or all 233 of the genes of the set of genes (2) (the negative effector genes).

In some embodiments, a panel of nucleic acid biomarkers is encompassed, wherein N is an integer of at least 10 and equal to or less than 482, and wherein the N biomarkers comprise nucleic acid capable of detecting any 10 to 249, 20 to 249, 30 to 249, 40 to 249, 50 to 249, 60 to 249, 70 to 249, 80 to 249, 90 to 249, 100 to 249, 110 to 249, 120 to 249, 130 to 249, 140 to 249, 150 to 249, 160 to 249, 170 to 249, 180 to 249, 190 to 249, 200 to 249, 210 to 249, 220 to 249, 230 to 249, or 240 to 249 of the genes of the set of genes (1) (the positive effector genes); and
any 10 to 233, 20 to 233, 30 to 233, 40 to 233, 50 to 233, 60 to 233, 70 to 233, 80 to 233, 90 to 233, 100 to 233, 110 to 233, 120 to 233, 130 to 233, 140 to 233, 150 to 233, 160 to 233, 170 to 233, 180 to 233, 190 to 233, 200 to 233, 210 to 233, 220 to 233, or 230 to 233 of the genes of the set of genes (2) (the negative effector genes).

In some embodiments, the panel comprises nucleic acid biomarkers capable of detecting about 90% of the 482 the genes of the set of genes (1) and (2) (positive and negative effector genes). In some embodiments, panel comprises nucleic acid biomarkers capable of detecting 430, 431, 432, 433, 434, 436, or 437 genes.

In some embodiments, the panel comprises nucleic acid biomarkers capable of detecting expression levels of each gene within any one of the following sets of genes a) through mmmm).

In some embodiments, the panel comprises nucleic acid biomarkers capable of detecting expression levels of each gene within any one of the following sets of genes a) through mmmm), to the exception of sets of genes q1), kkk1), ppp1) and qqq1).

A transcriptional signature (“TEAD signature”) may be obtained by measure of the expression levels of genes of a set of genes comprising any 220 to 249 of the genes of the set of genes (1) (positive effector genes) and any 210 to 233 of the genes of the set of genes (2) (negative effector genes) above indicated.

A transcriptional signature (“TEAD signature”) may be obtained by measure of the expression levels of genes of a set of genes comprising any of 220 to 249 of the genes of the set of genes (1) (positive effector genes), or any of 225 to 249, or any of 230 to 249, or any of 235 to 249, or any of 240 to 249, or any of 245 to 249 of the genes of the set of genes (1) (the positive effector genes).

A transcriptional signature (“TEAD signature”) may be obtained by measure of the expression levels of genes of a set of genes comprising any of 210 to 233 of the genes of the set of genes (2) (negative effector genes), or any of 215 to 233, or any of 220 to 233, or any of 225 to 233, or any of 230 to 233 of the genes of the set of genes (2) (the negative effector genes).

A transcriptional signature (“TEAD signature”) may be obtained by measure of the expression levels of genes of a set of genes comprising any of 430 to 482 genes above indicated, or any of the 435 to 482, or any of 440 to 482, or any of 445 to 482, or any of 450 to 482, or any of 455 to 482, or any of 460 to 482, or any of 465 to 482, or any of 470 to 482, or any of 475 to 482, or any of 480 to 482 genes above indicated.

A transcriptional signature (“TEAD signature”) may be obtained by measure of the expression levels of genes of a set of genes comprising any of 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481 or 482 genes above indicated.

The transcriptional signature (“TEAD signature”) may be used to compute a TEAD-score according to Method 1 or Method 2 as above disclosed. According to the method used, a TEAD-score may be a dgR or a deR score. A dgR or a deR score greater than about 0.055 is indicative of a TEAD-active cancer. A dgR or a deR score of less than about 0.055 is indicative of a TEAD-inactive cancer.

In some embodiments, a transcriptional signature may be reduced to a shorter signature. Those shorter signature are named thereafter “short TEAD signature”.

A “short TEAD signature” is obtained by measure of expression levels of genes of a reduced number of genes and is specific to a given cancer type.

In some embodiments, the disclosure relates to a use of a transcriptional signature (“short TEAD signature”) for measuring a TEAD-activity of a cancer in a subject in need thereof, the transcriptional signature being obtained by measure of expression levels of genes of a set of genes, in a biological sample of a tumor of said cancer, the set of genes being selected from the groups of sets a) to mmmm) as disclosed in Tables 1 or 2 above.

Each set of genes is selective for analyzing a particular cancer type/subtype as described in Embodiments 3-59 of Tables 1 and 2.

A short TEAD signature may be used for computing a discriminant score (S) or (DS) according to Method 3 or Method 4.

In a Method 3, a discriminant score (S) is compared to the centroids of the TEAD-active (A) and TEAD-inactive (I) groups, which are provided for each cancer indication or subtype associated with a set of genes from the group of sets a) to mmmm) as shown Tables 1 and 2. If (S) is closer to (A) than it is to (I), then, the sample is classified as TEAD-active. Otherwise, the sample is classified as TEAD-inactive.

In some embodiments, in such method, the use of “short TEAD signature” does not include the use of sets of genes q1), kkk1), ppp1) and qqq1).

In a Method 4, a discriminant score (DS) is compared with a threshold from the thresholds shown in Table 6 and associated with the cancer indication or subtype associated with a set of genes of the group of sets a) to mmmm), to determine if the cancer is TEAD-active or TEAD-inactive.

### Methods and Uses

Methods and uses utilizing the panels are provided, including, measuring TEAD-dependent transcription levels, characterizing a subject as having a TEAD-active or TEAD-inactive cancer, and predicting whether a subject is likely to respond positively to a TEAD-pathway inhibitor. The methods comprise isolating a biological sample from a subject having or suspected of having cancer (solid or blood), detecting the expression level of each of the genes, and following the methods described herein to score the panel(s), wherein the methods differ depending on use of the TEAD-500 panel or the optimal/minimal discriminant functions for the TEAD-500 subset panels. In some embodiments, the methods/uses further comprise the active step of administering a TEAD-pathway inhibitor treatment to a subject identified by using the disclosed panel. In some embodiments, the step of isolating a biological sample is not part of the method and is carried out prior to the implementation of the method.

In some embodiments, a transcription signature (“TEAD signature” or “short TEAD signature”) may be for determining, measuring or characterizing the TEAD activity of tumor of a cancer. The transcriptional signatures may be useful in a variety of applications, including, predicting the likelihood that a subject will respond to a TEAD-pathway inhibitor treatment, selecting patients for clinical trials, assessing efficacy of TEAD-pathway inhibitor molecules, and prognosing survival, response to, and benefit from anti-TEAD pathway treatments.

In some embodiments, a transcription signature (“TEAD signature” or “short TEAD signature”) may be obtained by measuring the TEAD-dependent transcription levels of genes in a biological sample from a subject with or suspected of having cancer. The genes may be from the set of genes of the disclosures.

The use comprises detecting, or measuring, the expression level of each gene of the set of genes of the signature in a biological sample obtained from a subject in need thereof. The biological sample is isolated from a tumor present in said subject.

In some embodiments, a transcription signature (“TEAD signature” or “short TEAD signature”) may be for characterizing a subject as having a TEAD-active or TEAD-inactive cancer.

The use comprises detecting, or measuring, the expression level of each gene of the set of genes of the signature in a biological sample obtained from a subject with or suspected of having cancer.

A transcription signature may, according to the case, be used for computing a TEAD score dgR (Method 1) or deR (Method 2) or a discriminant score (S) (Method 3) or (DS) (Method 4).

The subject is deemed as having a TEAD-active cancer if the dgR or deR, obtained with a “TEAD signature” is greater than about 0.055.

The subject is deemed as having a TEAD-active cancer if the discriminant score (DS), obtained with a “short TEAD signature” is closer to the centroid of the TEAD-active (A) group than to the centroid of TEAD-inactive (I) group.

The subject is deemed as having a TEAD-active cancer if the discriminant score (S), obtained with a “short TEAD signature” is, depending on the case, above or below the threshold given in Table 3.

In some embodiments, a transcription signature (“TEAD signature” or “short TEAD signature”) may be for predicting whether a subject will respond positively to a TEAD-pathway inhibitor.

The subject is deemed likely to respond positively to a TEAD-pathway inhibitor if the dgR or deR, obtained with a “TEAD signature” is greater than about 0.055.

The subject is deemed likely to respond positively to a TEAD-pathway inhibitor if the discriminant score (DS), obtained with a “short TEAD signature” is closer to the centroid of the TEAD-active (A) group than to the centroid of TEAD-inactive (I) group.

The subject is deemed likely to respond positively to a TEAD-pathway inhibitor if the discriminant score (S), obtained with a “short TEAD signature” is, depending on the case, above or below the threshold given in Table 3.

In some embodiments comprising the use of a TEAD-500 panel, once the genes on the TEAD-500 panel are detected and the gene expression is calculated, a TEAD score is provided as described above in Method 1 or 2. The subject is said to have a TEAD-active cancer if the dgR or deR is greater than about 0.055, and a TEAD-inactive cancer if the dgR or deR is less than or equal to about 0.055. Likewise, a subject is likely to respond positively to a TEAD-pathway inhibitor if the dgR or deR is greater than about 0.055 and may not respond to a TEAD-pathway inhibitor if the dgR or deR is less than or equal to about 0.055.

In some embodiments, a panel for sets of genes a) to mmmm) is provided for use in any of the Methods 3 or 4 described herein, including measuring TEAD-dependent transcription levels, characterizing a subject as having a TEAD-active or TEAD-inactive cancer, or predicting whether a subject will respond positively to a TEAD-pathway inhibitor, where the panel comprises any one of the gene subsets of Embodiments 3 through 59 above.

Such Method 3 comprises the steps of i) determining the expression level of each gene in the set; ii) determining the fractional rank for each gene; iii) multiplying the fractional rank for each gene by the corresponding coefficient shown in parenthesis for each gene in each panel; iv) summing the products to give a discriminant score (S); v) comparing the discriminant score (S) to the centroids of the TEAD-active (A) and TEAD-inactive (I) groups; vi) determining if S is closer to (A) than it is to (I) according to the formula max(S,A)−min(S,A)<max(S,I)−min(S,I); and vii) characterizing the sample as TEAD-active if (S) is closer to (A) than it is to (I), and TEAD-inactive if (S) is closer to (I) than it is to (A).

In some embodiments, a Method 3 may comprise the steps of i) measuring, in a biological sample, the expression level of each gene of a set of genes of a “TEAD signature” (i.e., any of 220 to 249 of genes of a set of genes (1) (positive effector genes) and any of 210 to 233 of genes of a set of genes (2) (negative effector genes); ii) for each gene of a set of genes shown in Table 1, converting the gene's expression level obtained at step i) into a fractional rank by dividing the rank of said gene by the number of genes of said set of genes of said “TEAD signature”; iii) multiplying each fractional rank obtained at step iii) by a coefficient associated with each gene of said set of genes of Table 1, said coefficient for said gene and set of genes being shown in Table 2, to obtain a product for each gene; iv) summing the products to obtain a discriminant score (S); v) comparing the discriminant score (S) to the centroids of the TEAD-active (A) and TEAD-inactive (I) groups shown in Table 2 for said set of genes of Table 1; vi) determining if (S) is closer to (A) than it is to (I) according to the formula max(S,A)−min(S,A)<max(S,I)−min(S,I); and vii) characterizing the sample as TEAD-active if (S) is closer to (A) than it is to (I), and TEAD-inactive if (S) is closer to (I) than it is to (A).

In some embodiments, a Method 3 does not include the use of sets of genes q1), kkk1), ppp1) and qqq1).

The Method 4 comprises:
(i) determining the expression levels of the genes listed in the optimal discriminant function of sets a), set c), set d), set e), set g), set i), set j), set l), set m), set q), set r), t), set v), set x), set y), set aa), set bb), set dd), set ff), set gg), set ii), set kk), set mm), set rr), set ss), set uu), set vv), set xx), set zz), set kkk), set lll), set mmm), set nnn), set ppp), set rrr), set sss), set ttt), set uuu), set zzz), set bbbb), set hhhh), set kkkk), and set IIII) of Table 1 for the indication in which the tumor belongs (as indicated in Table 2). Expression levels may be determined by RNA sequencing, RT-qPCR, or any other reliable method;
(ii) for each gene of said set of genes, converting the gene's expression level into a fractional rank, which according to the definitions herein, is the rank of the gene (i.e., the expression of the gene relative to all the genes tested, where the gene with the lowest expression level is given the rank 1, the next highest level of expression a rank 2, and so on until the highest expressing gene is given the highest rank; equal expression levels are given the average rank, e.g. two genes with 0 expression are given the rank 1.5) divided by the number of genes of said set of genes;
(iii) multiplying each fractional rank obtained at step (ii) by a coefficient associated with each gene of said set of genes, said coefficient for said gene and set of genes being shown in Table 2, to obtain a product (Pgeneᵢ) for each gene,
(iv) computing a discriminant score (DS) of said set of genes of the tumor sample as the sum of the products of all the discriminant genes plus the constant coefficient of the discriminant function (i.e., constant coefficient for said set of genes shown in Table 2). For example, if the optimal discriminant function for the indication in which the sample belongs has 10 genes, we multiply the fractional rank of each gene with the corresponding discriminant coefficient (known as the loading) of the gene, we add up the resulting products to the constant coefficient of the optimal discriminant function.

$$Discriminant score = \sum ( fractional rank Genei * coefficient for Genei ) + discriminant constant$$

where Genei is a gene listed in a discriminant function;
and (v) determining the TEAD-activity status of the sample using the threshold for each indication as listed in Table 3.

In some embodiments, the Method 4 may:
apply when the cancer is elected from the group consisting of Adrenocortical carcinoma (ACC) tumors; Bladder Urothelial Carcinoma (BLCA) tumors; Breast invasive carcinoma (BRCA) tumors; BRCA basal tumors; BRCA non-basal tumors; Cervical squamous cell carcinoma and endocervical adenocarcinoma (CESC) tumors; Cholangiocarcinoma (CHOL) tumors; Colon adenocarcinoma (COAD) tumors; COAD CMS1 tumors; COAD CMS4 tumors; Lymphoid Neoplasm Diffuse Large B-cell Lymphoma (DLBC) tumors; Esophageal carcinoma (ESCA) tumors; Glioblastoma multiforme (GBM) tumors; Head and Neck squamous cell carcinoma (HNSC) tumors; Kidney Chromophobe (KICH) tumors; Kidney renal clear cell carcinoma (KIRC) tumors; Kidney renal papillary cell carcinoma (KIRP) tumors; Brain Lower Grade Glioma (LGG) tumors; Liver hepatocellular carcinoma (LIHC) tumors; LIHC S1 tumors; LIHC S2 tumors; LIHC S3 tumors; Lung adenocarcinoma (LUAD) tumors; Lung squamous cell carcinoma (LUSC) tumors; LUSC basal tumors; LUSC classical tumors; LUSC primitive tumors; LUSC secretory tumors; Malignant mesothelioma (MESO) tumors; Pancreatic adenocarcinoma (PAAD) tumors; Rectum adenocarcinoma (READ) tumors; READ CMS1 tumors; READ CMS2 tumors; is READ CMS4 tumors; Sarcoma (SARC) tumors; Skin Cutaneous Melanoma (SKCM) tumors; SKCM immune tumors; SKCM keratin tumors; STAD MSI tumors; STAD MSS_EMT tumors; Testicular Germ Cell Tumors (TGCT) tumors; Uterine Corpus Endometrial Carcinoma (UCEC) tumors; and Uterine Carcinosarcoma (UCS) tumors,
and
comprises:
a) measuring, in a biological sample of a tumor of said cancer, the expression level of each gene of a set of genes associated with said tumor, the set of genes being selected from the group consisting of set a), set c), set d), set e), set g), set i), set j), set l), set m), set q), set r), t), set v), set x), set y), set aa), set bb), set dd), set ff), set gg), set ii), set kk), set mm), set rr), set ss), set uu), set vv), set xx), set zz), set kkk), set lll), set mmm), set nnn), set ppp), set rrr), set sss), set ttt), set uuu), set zzz), set bbbb), set hhhh), set kkkk), and set IIII) of Table 1;
b) for each gene of said set of genes, converting the gene's expression level obtained at step a) into a fractional rank by dividing the rank of said gene by the number of genes of said set of genes;
c) multiplying each fractional rank obtained at step b) by a coefficient associated with each gene of said set of genes, said coefficient for said gene and set of genes being shown in Table 2, to obtain a product (Pgeneᵢ) for each gene;
d) determining a discriminant score (DS) of said set of genes, the discriminant score (DS) being the sum of the products Pgeneᵢ obtained at step c) plus the constant coefficient for said set of genes shown in Table 2,
e) comparing, for said set of genes, the discriminant score (DS) obtained at step d) with a threshold associated with said set of genes, the threshold being shown in Table 3, to determine if the cancer is TEAD-active or TEAD-inactive.

In some embodiments, methods and uses utilizing the TEAD-500 or any of the subset panels of Embodiments 3 through 59 (disclosed in Tables 1 and 2) are provided for monitoring the activity of the TEAD complex, evaluating the pharmacodynamics of inhibitors of the TEAD pathway in vitro or in vivo, diagnosing tumors of which the development or evolution is attributable to activation of the TEAD complex, recruiting patients with active TEAD to clinical trials designed to evaluate TEAD pathway inhibitors, prognosing survival, response to, and benefit from an anti-TEAD pathway treatment (e.g., treatment with a TEAD-pathway inhibitor), alone or in combination with other treatments, estimating the proportion of cases within cohorts where TEAD-dependent transcription may be responsible for tumor development or evolution to treatment resistance, or measuring TEAD-dependent transcription in any tissue, under any condition, pathological or not, in the laboratory or in the clinic.

In some embodiments, methods and uses of the disclosure do not include the use of sets of genes q1), kkk1), ppp1) and qqq1).

In some embodiments, the disclosure relates to a method for measuring a transcription signature (“TEAD signature” or “short TEAD signature”) in a biological sample from a subject with or suspected of having cancer, the method comprising the step of detecting, or measuring, the expression level of each gene of a set of genes of the transcription signature (“TEAD signature” or “short TEAD signature”) in the biological sample.

In some embodiments, the disclosure relates to a method for characterizing the TEAD-activity of a cancer in a subject in need thereof, the method using a biological sample of a tumor of said cancer and comprising at least the steps of
a) measuring, in a transcriptome obtained from said biological sample, the expression levels of genes,
b) for each gene of a set of genes of a “TEAD-signature”, i.e., any of 220 to 249 of genes of a set of genes (1) (positive effector genes) and any of 210 to 233 of genes of a set of genes (2) (negative effector genes), converting the gene's expression level obtained at step a) into a fractional rank by dividing the rank of said gene by the number of genes from said transcriptome,
c) isolating from the fractional ranks obtained at step b) the fractional rank obtained for the genes of the set of genes (1) and computing their mean fractional rank (MFR-positive),
d) isolating from the fractional ranks obtained at step b) the fractional rank obtained for the genes of the set of genes (2) and computing their mean fractional rank (MFR-negative), and
e) computing the dgR score as MFR-positive-MFR-negative;
or
a) measuring, in said biological sample, the expression level of each gene of a set of genes of a “TEAD-signature”, i.e., any of 220 to 249 of genes of a set of genes (1) (positive effector genes) and any of 210 to 233 of genes of a set of genes (2) (negative effector genes),
b) for each gene of said set of genes of a “TEAD-signature”, i.e., any of 220 to 249 of genes of a set of genes (1) (positive effector genes) and any of 210 to 233 of genes of a set of genes (2) (negative effector genes), converting the gene's expression level obtained at step a) into a fractional rank by dividing said rank of said gene by the number of genes from said set of genes,
c) isolating from the fractional ranks obtained at step b) the fractional rank obtained for the genes of the set of genes (1) and computing their mean fractional rank (MFR-positive),
d) isolating from the fractional ranks obtained at step b) the fractional rank obtained for the genes of the set of genes (2) and computing their mean fractional rank (MFR-negative),
e) computing the deR score as MFR-positive-MFR-negative;
wherein when the dgR or the deR score is greater than about 0.055, then the cancer is TEAD-active, when the dgR or deR score is less than or equal to about 0.055, then the cancer is TEAD-inactive.

In some embodiments, the disclosure relates to a method for characterizing the TEAD-activity of a cancer in a subject in need thereof, the method using a biological sample of a tumor of said cancer and comprising at least the steps of:
a) measuring, in said biological sample, the expression level of each gene of a set of genes of a “TEAD-signature”, i.e., any of 220 to 249 of genes of a set of genes (1) (positive effector genes) and any of 210 to 233 of genes of a set of genes (2) (negative effector genes),
b) for each gene of a set of genes shown in Table 1, converting the gene's expression level obtained at step a) into a fractional rank by dividing the rank of said gene by the number of genes of said set of genes of the “TEAD-signature”;
c) multiplying each fractional rank obtained at step b) by a coefficient associated with each gene of said set of genes of Table 1, said coefficient for said gene and set of genes being shown in Table 2, to obtain a product for each gene;
d) summing the products obtained at step c) to obtain a discriminant score (S);
e) comparing the discriminant score (S) obtained at step d) to the centroids of the TEAD-active (A) and TEAD-inactive (I) groups shown in Table 2 for said set of genes of Table 1;
f) determining if (S) is closer to (A) than it is to (I) according to the formula max(S,A)−min(S,A)<max(S,I)−min(S,I); and
g) characterizing the cancer as TEAD-active if (S) is closer to (A) than it is to (I).

In some embodiments, the disclosure relates to a method for measuring the TEAD-activity a cancer in a subject in need thereof, wherein
the cancer is a tumor selected from the group consisting of Adrenocortical carcinoma (ACC) tumors; Bladder Urothelial Carcinoma (BLCA) tumors; Breast invasive carcinoma (BRCA) tumors; BRCA basal tumors; BRCA non-basal tumors; Cervical squamous cell carcinoma and endocervical adenocarcinoma (CESC) tumors; Cholangiocarcinoma (CHOL) tumors; Colon adenocarcinoma (COAD) tumors; COAD CMS1 tumors; COAD CMS4 tumors; Lymphoid Neoplasm Diffuse Large B-cell Lymphoma (DLBC) tumors; Esophageal carcinoma (ESCA) tumors; Glioblastoma multiforme (GBM) tumors; Head and Neck squamous cell carcinoma (HNSC) tumors; Kidney Chromophobe (KICH) tumors; Kidney renal clear cell carcinoma (KIRC) tumors; Kidney renal papillary cell carcinoma (KIRP) tumors; Brain Lower Grade Glioma (LGG) tumors; Liver hepatocellular carcinoma (LIHC) tumors; LIHC S1 tumors; LIHC S2 tumors; LIHC S3 tumors; Lung adenocarcinoma (LUAD) tumors; Lung squamous cell carcinoma (LUSC) tumors; LUSC basal tumors; LUSC classical tumors; LUSC primitive tumors; LUSC secretory tumors; Malignant mesothelioma (MESO) tumors; Pancreatic adenocarcinoma (PAAD) tumors; Rectum adenocarcinoma (READ) tumors; READ CMS1 tumors; READ CMS2 tumors; is READ CMS4 tumors; Sarcoma (SARC) tumors; Skin Cutaneous Melanoma (SKCM) tumors; SKCM immune tumors; SKCM keratin tumors; STAD MSI tumors; STAD MSS_EMT tumors; Testicular Germ Cell Tumors (TGCT) tumors; Uterine Corpus Endometrial Carcinoma (UCEC) tumors; Uterine Carcinosarcoma (UCS) tumors,
and
the method uses a biological sample of a tumor of said cancer, and
the method comprises at least the steps of:
a) measuring, in said biological sample, the expression level of each gene of a set of genes associated with said tumor, the set of genes being selected from the group consisting of set a), set c), set d), set e), set g), set i), set j), set l), set m), set q), set r), t), set v), set x), set y), set aa), set bb), set dd), set ff), set gg), set ii), set kk), set mm), set rr), set ss), set uu), set vv), set xx), set zz), set kkk), set lll), set mmm), set nnn), set ppp), set rrr), set sss), set ttt), set uuu), set zzz), set bbbb), set hhhh), set kkkk), and set IIII) of Table 1;
b) for each gene of said set of genes, converting the gene's expression level into a fractional rank by dividing the rank of the gene by the number of genes of said set of genes;
c) multiplying each fractional rank obtained at step b) by a coefficient associated with each gene of said set of genes, said coefficient for said gene and set of genes being shown in Table 2, to obtain a product (Pgeneᵢ) for each gene;
d) determining a discriminant score (DS) of said set of genes, the discriminant score (DS) being the sum of the products Pgeneᵢ obtained at step c) plus the constant coefficient of said set of genes shown in Table 2;
e) comparing, for said set of genes, the discriminant score (DS) obtained at step d) with a threshold associated with said set of genes, the threshold being shown in Table 3, to determine if the cancer is TEAD-active or TEAD-inactive.

A method of the disclosure may be for characterizing a subject as having a TEAD-active or TEAD-inactive cancer.

In some embodiments, the cancer may comprise a solid tumor. In some embodiments, the solid tumor is in the lung, colon, ovary, cervix, uterus, peritoneum, testicles, penis, tongue, lymph node, pancreas bone, breast, prostate, soft tissue, connective tissue, kidney, liver, brain, thyroid, or skin. In some embodiments, the cancer may be adrenocortical carcinoma, bladder urothelial carcinoma, breast invasive carcinoma, cervical squamous cell carcinoma and endocervical adenocarcinoma, cholangiocarcinoma, consensus molecular subtypes 1 of colorectal cancer, consensus molecular subtypes 2 of colorectal cancer, consensus molecular subtypes 3 of colorectal cancer, consensus molecular subtypes 4 of colorectal cancer, colon adenocarcinoma, lymphoid neoplasm diffuse large b-cell lymphoma, esophageal carcinoma, glioblastoma multiforme, head and neck squamous cell carcinoma, kidney renal clear cell carcinoma, kidney renal papillary cell carcinoma, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, or uterine carcinosarcoma.

In some embodiments, the cancer is a blood cancer. In some embodiments, the blood cancer is leukemia, lymphoma, and myeloma.

A cancer may be selected from the group consisting of: Adrenocortical carcinoma (ACC) tumor; Bladder Urothelial Carcinoma (BLCA) tumor; Breast invasive carcinoma (BRCA); BRCA basal tumor; BRCA non-basal tumor; Cervical squamous cell carcinoma or endocervical adenocarcinoma (CESC) tumor; Cholangiocarcinoma (CHOL) tumor; Colon adenocarcinoma (COAD) tumor; COAD CMS1 tumor; COAD CMS2 tumor; COAD CMS4 tumor; Lymphoid Neoplasm Diffuse Large B-cell Lymphoma (DLBC) tumor; Esophageal carcinoma (ESCA) tumor; Glioblastoma multiforme (GBM) tumor; Head or Neck squamous cell carcinoma (HNSC) tumor; Kidney Chromophobe (KICH) tumor; Kidney renal clear cell carcinoma (KIRC) tumor; Kidney renal papillary cell carcinoma (KIRP) tumor; Brain Lower Grade Glioma (LGG) tumor; Liver hepatocellular carcinoma (LIHC) tumor; LIHC S1 tumor; LIHC S2 tumor; LIHC S3 tumor; Lung adenocarcinoma (LUAD) tumor; LUAD proximal-inflammatory tumor; LUAD proximal-proliferative tumor; Lung squamous cell carcinoma (LUSC) tumor; LUSC basal tumor; LUSC classical tumor; LUSC primitive tumor; LUSC secretory tumor; Malignant mesothelioma (MESO) tumor; Ovarian serous cystadenocarcinoma (OV) tumor; OV differentiated tumor; for OV immune-reactive tumor; OV mesenchymal tumor; OV proliferative tumor; Pancreatic adenocarcinoma (PAAD) tumor; Rectum adenocarcinoma (READ) tumor; READ CMS1 tumor; READ CMS2 tumor; READ CMS4 tumor; READ unclassifiable tumor; Sarcoma (SARC) tumor; Skin Cutaneous Melanoma (SKCM) tumor; SKCM immune tumor; SKCM keratin tumor; SKCM MITF-low tumor; Stomach adenocarcinoma (STAD) tumor; STAD MSI tumor; STAD MSS_EMT tumor; STAD MSS_TP53 tumor; STAD MSS_TP53+ tumor; Testicular Germ Cell Tumor (TGCT) tumor; Thyroid carcinoma (THYM) tumor; Uterine Corpus Endometrial Carcinoma (UCEC) tumor; and Uterine Carcinosarcoma (UCS) tumor.

In some embodiments the cancer is:
Adrenocortical carcinoma (ACC) tumor then the set of genes of the transcription signature is set of genes a) or b);
Bladder Urothelial Carcinoma (BLCA) tumor then the set of genes of the transcription signature is set of genes c);
Breast invasive carcinoma (BRCA) tumor then the set of genes of the transcription signature is set of genes d);
BRCA basal tumor then the set of genes of the transcription signature is set of genes e) or f);
BRCA non-basal tumor then the set of genes of the transcription signature is set of genes g) or h);
Cervical squamous cell carcinoma or endocervical adenocarcinoma (CESC) tumor then the set of genes of the transcription signature is set of genes i);
Cholangiocarcinoma (CHOL) tumor then the set of genes of the transcription signature is set of genes j) or k);
Colon adenocarcinoma (COAD) tumor then the set of genes of the transcription signature is set of genes l);
COAD CMS1 tumor then the set of genes of the transcription signature is set of genes m) or n);
COAD CMS2 tumor then the set of genes of the transcription signature is set of genes o) or p);
COAD CMS4 tumor then the set of genes of the transcription signature is set of genes q) or q1);
Lymphoid Neoplasm Diffuse Large B-cell Lymphoma (DLBC) tumor then the set of genes of the transcription signature is set of genes r) or s);
Esophageal carcinoma (ESCA) tumor then the set of genes of the transcription signature is set of genes t) or u);
Glioblastoma multiforme (GBM) tumor then the set of genes of the transcription signature is set of genes v) or w);
Head or Neck squamous cell carcinoma (HNSC) tumor then the set of genes of the transcription signature is set of genes x);
Kidney Chromophobe (KICH) tumor then the set of genes of the transcription signature is set of genes y) or z);
Kidney renal clear cell carcinoma (KIRC) tumor then the set of genes of the transcription signature is set of genes aa);
Kidney renal papillary cell carcinoma (KIRP) tumor then the set of genes of the transcription signature is set of genes bb) or cc);
Brain Lower Grade Glioma (LGG) tumor then the set of genes of the transcription signature is set of genes dd) or ee);
Liver hepatocellular carcinoma (LIHC) tumor then the set of genes of the transcription signature is set of genes ff);
LIHC S1 tumor then the set of genes of the transcription signature is set of genes gg) or hh);
LIHC S2 tumor then the set of genes of the transcription signature is set of genes ii) or jj);
LIHC S3 tumor then the set of genes of the transcription signature is set of genes kk) or II);
Lung adenocarcinoma (LUAD) tumor then the set of genes of the transcription signature is set of genes mm);
LUAD proximal-inflammatory tumor then the set of genes of the transcription signature is set of genes nn) or oo);
LUAD proximal-proliferative tumor then the set of genes of the transcription signature is set of genes pp) or qq);
Lung squamous cell carcinoma (LUSC) tumor then the set of genes of the transcription signature is set of genes rr);
LUSC basal tumor then the set of genes of the transcription signature is set of genes ss) or tt);
LUSC classical tumor then the set of genes of the transcription signature is set of genes uu);
LUSC primitive tumor then the set of genes of the transcription signature is set of genes vv) or ww);
LUSC secretory tumor then the set of genes of the transcription signature is set of genes xx) or yy);
Malignant mesothelioma (MESO) tumor then the set of genes of the transcription signature is set of genes zz) or aaa);
Ovarian serous cystadenocarcinoma (OV) tumor then the set of genes of the transcription signature is set of genes bbb);
OV differentiated tumor then the set of genes of the transcription signature is set of genes ccc) or ddd);
OV immune-reactive tumor then the set of genes of the transcription signature is set of genes eee) or fff);
OV mesenchymal tumor then the set of genes of the transcription signature is set of genes ggg) or hhh);
OV proliferative tumor then the set of genes of the transcription signature is set of genes iii) or jj);
Pancreatic adenocarcinoma (PAAD) tumor then the set of genes of the transcription signature is set of genes kkk) or kkk1);
Rectum adenocarcinoma (READ) tumor then the set of genes of the transcription signature is set of genes III);
READ CMS1 tumor then the set of genes of the transcription signature is set of genes mmm);
READ CMS2 tumor then the set of genes of the transcription signature is set of genes nnn) or ooo);
READ CMS4 tumor then the set of genes of the transcription signature is set of genes ppp) or ppp1);
READ unclassifiable tumor then the set of genes of the transcription signature is set of genes qqq) or qqq1);
Sarcoma (SARC) tumor then the set of genes of the transcription signature is set of genes rrr);
Skin Cutaneous Melanoma (SKCM) tumor then the set of genes of the transcription signature is set of genes sss);
SKCM immune tumor then the set of genes of the transcription signature is set of genes ttt);
SKCM keratin tumor then the set of genes of the transcription signature is set of genes uuu) or vvv);
SKCM MITF-low tumor then the set of genes of the transcription signature is set of genes www) or xxx);
Stomach adenocarcinoma (STAD) tumor then the set of genes of the transcription signature is set of genes yyy);
STAD MSI tumor then the set of genes of the transcription signature is set of genes zzz) or aaaa);
STAD MSS_EMT tumor then the set of genes of the transcription signature is set of genes bbbb) or cccc);
STAD MSS_TP53-tumor then the set of genes of the transcription signature is set of genes dddd) or eeee);
STAD MSS_TP53+ tumor then the set of genes of the transcription signature is set of genes ffff) or gggg);
Testicular Germ Cell Tumor (TGCT) tumor then the set of genes of the transcription signature is set of genes hhhh) or iiii);
Thyroid carcinoma (THYM) tumor then the set of genes of the transcription signature is set of genes jjjj);
Uterine Corpus Endometrial Carcinoma (UCEC) tumor then the set of genes of the transcription signature is set of genes kkkk); and
Uterine Carcinosarcoma (UCS) tumor then the set of genes of the transcription signature is set of genes IIII) or mmmm).

In some embodiments, the use of “short TEAD signature” does not include the use of sets of genes q1), kkk1), ppp1) and qqq1).

In some embodiments the cancer is selected from:
Adrenocortical carcinoma (ACC) tumors; Bladder Urothelial Carcinoma (BLCA) tumors; Breast invasive carcinoma (BRCA) tumors; BRCA basal tumors; BRCA non-basal tumors; Cervical squamous cell carcinoma and endocervical adenocarcinoma (CESC) tumors; Cholangiocarcinoma (CHOL) tumors; Colon adenocarcinoma (COAD) tumors; COAD CMS1 tumors; COAD CMS4 tumors; Lymphoid Neoplasm Diffuse Large B-cell Lymphoma (DLBC) tumors; Esophageal carcinoma (ESCA) tumors; Glioblastoma multiforme (GBM) tumors; Head and Neck squamous cell carcinoma (HNSC) tumors; Kidney Chromophobe (KICH) tumors; Kidney renal clear cell carcinoma (KIRC) tumors; Kidney renal papillary cell carcinoma (KIRP) tumors; Brain Lower Grade Glioma (LGG) tumors; Liver hepatocellular carcinoma (LIHC) tumors; LIHC S1 tumors; LIHC S2 tumors; LIHC S3 tumors; Lung adenocarcinoma (LUAD) tumors; Lung squamous cell carcinoma (LUSC) tumors; LUSC basal tumors; LUSC classical tumors; LUSC primitive tumors; LUSC secretory tumors; Malignant mesothelioma (MESO) tumors; Pancreatic adenocarcinoma (PAAD) tumors; Rectum adenocarcinoma (READ) tumors; READ CMS1 tumors; READ CMS2 tumors; is READ CMS4 tumors; Sarcoma (SARC) tumors; Skin Cutaneous Melanoma (SKCM) tumors; SKCM immune tumors; SKCM keratin tumors; STAD MSI tumors; STAD MSS_EMT tumors; Testicular Germ Cell Tumors (TGCT) tumors; Uterine Corpus Endometrial Carcinoma (UCEC) tumors; Uterine Carcinosarcoma (UCS) tumors,
and the set of genes for the transcriptional signature associated with said tumor is set a), set c), set d), set e), set g), set i), set j), set l), set m), set q), set r), t), set v), set x), set y), set aa), set bb), set dd), set ff), set gg), set ii), set kk), set mm), set rr), set ss), set uu), set vv), set xx), set zz), set kkk), set lll), set mmm), set nnn), set ppp), set rrr), set sss), set ttt), set uuu), set zzz), set bbbb), set hhhh), set kkkk), or set IIII).

### Assay/RNA Sequencing

In some embodiments, the expression level of a gene may be a nucleic acid expression level, such as an RNA expression level, an mRNA expression level, or a DNA expression level. Any suitable method of determining a nucleic acid expression level may be used. In some embodiments, the nucleic acid expression level is determined using RNA-seq. For example, the nucleic acid expression level could be determined using an RNA ACCESS protocol or TRUSEQ RIBO-ZERO00 protocol (ILLUMINA)), RT-qPCR, qPCR, multiplex qPCR or RT-qPCR, microarray analysis, SAGE, Mass-ARRAY techniques, or a combination thereof.

Methods for the evaluation of mRNAs in cells are well known and include, for example, RNA sequencing (RNA-seq), whole genome sequencing (WGS), serial analysis of gene expression (SAGE), and various nucleic acid amplification assays such as RT-PCR, using complementary primers specific for the predetermined set of genes. In some embodiments, qRT-PCR is used. In addition, such methods can include one or more steps that allow one to determine the levels of target mRNA in a biological sample, for example, by simultaneously examining the levels of a comparative control mRNA sequence of a “housekeeping” gene such as an actin family member. In some embodiments, the sequence of the amplified target cDNA can be determined. Optional methods include protocols which examine or detect mRNAs, such as target mRNAs, in a tissue or cell sample by microarray technologies. Using nucleic acid microarrays, test and control mRNA samples from test and control tissue samples are reverse transcribed and labeled to generate cDNA probes. The probes are then hybridized to an array of nucleic acids immobilized on a solid support. The array is configured such that the sequence and position of each member of the array is known. For example, any of the TEAD-500 genes or subsets of the Embodiments described herein may be arrayed on a solid support. Hybridization of a labeled probe with a particular array member indicates that the sample from which the probe was derived expresses that gene.

### Biological Samples

In some embodiments, the biological sample is a sample of blood, plasma, serum, cerebral spinal fluid (CSF), nasal aspirate, throat swab, vaginal or cervix swab, sputum, a tissue, a cell, or a tumor biopsy.

In some embodiments, any biological sample comprising one or more tumor cell(s) can be used in the methods disclosed herein. In some embodiments, the sample is selected from a tumor biopsy. In some embodiments, the sample obtained from the subject is a formalin-fixed tumor biopsy. In some embodiments, the sample obtained from the subject is a formalin-fixed paraffin-embedded tumor (FFPE) biopsy or a paraffin-embedded tumor biopsy. In some embodiments, the sample obtained from the subject is a fresh-frozen (FF) tumor biopsy. In some embodiments, the sample is a fresh tumor biopsy. In some embodiments, the sample is an archival tumor biopsy. In some embodiments, the sample is a frozen tumor biopsy.

In some instances of any of the methods and uses, the sample is obtained from the individual prior to administration of a TEAD-pathway inhibitor. In some embodiments, the sample is obtained minutes, hours, days, weeks, months, or years prior to administration of TEAD-pathway inhibitor as described herein. In other words, the sample may be a baseline sample. In some embodiments, the sample is obtained from the individual following administration of TEAD-pathway inhibitor as described herein. In some instances, the sample from the individual is obtained within thirty hours following administration of an endocrine therapy. In some embodiments, the sample is obtained minutes, hours, or days following administration of TEAD-pathway inhibitor as described herein. In some embodiments, multiple samples are obtained from the same individual at different time points, for example, prior to and following administration of TEAD-pathway inhibitor as described herein.

### TEAD-Pathway Inhibitors

In some embodiments, a TEAD-pathway inhibitor is administered to a subject identified as having a TEAD-active cancer, or as likely to respond to a TEAD-pathway inhibitor using the methods and panels described herein. Any TEAD-pathway inhibitor known in the art may be administered. For example, transcriptional enhanced associate domain (TEAD) proteins are transcription factors comprised of four family members (TEAD1-4) that function in modulating gene expression in response to the HIPPO pathway. TEAD proteins preferentially associate with transcription co-activators yes associated protein 1 (YAP1) or transcriptional co-activator with PDZ-binding motif (WWTR1, also known as TAZ). YAP1-TEAD or WWTR1-TEAD bind to DNA and initiate the transcription of multiple different genes involved in cell proliferation, survival, mobility, stemness, and differentiation (reviewed in Holden and Cunningham, Cancer 2018). YAP1/WWWTR1-TEAD activity is tightly controlled by the HIPPO pathway.

The HIPPO pathway was initially discovered in Drosophila melanogaster as a key regulator of tissue growth. It is an evolutionarily conserved signaling pathway regulating numerous biological processes, including cell growth and fate decision, organ size control, and regeneration. The core of the Hippo pathway in mammals consists of a cascade of kinases including MST1/2 and LATS1/2, their associated adaptor proteins SAV1 and MOB1, as well as upstream regulators, such as NF2, SCRIBBLE, CRUMBS, and multiple G protein-coupled receptors. The Hippo pathway is tightly regulated by both intrinsic and extrinsic signals, such as mechanical force, cell-cell contact, polarity, energy status, stress, as well as many diffusible hormonal factors (reviewed in Ma S et al. Annu Rev Biochem. 2019; 20; 88:577-604. Doi: 10.1146/annurev-biochem-013118-111829). Upon activation of Hippo pathway kinases (i.e. Hippo “on” state), cytosolic YAP1 and WWTR1 proteins are phosphorylated and therefore, remain inactive through sequestration in the cytoplasm and/or degradation by the proteasomal machinery. Upon inactivation of the Hippo pathway kinases (i.e. Hippo “off” state), cytosolic YAP1 and WWTR1 are not anymore phosphorylated and hence free to translocate into the cell nucleus, where they associate with TEAD transcription factors to bind DNA and regulate gene expression. Decreasing levels of pYAP1/YAP1 as well as increased expression of genes regulated by YAP1/WWTR1-TEAD activity and increased promoter activity at TEAD-regulated genes are general indicators of YAP1 activation (reviewed in Totaro A, et al. Nat Cell Biol. 2018; 20 (8): 888-899. Doi: 10.1038/s41556-018-0142-z).

The Hippo-YAP1/WWTR1/TEAD pathway and human cancer: In recent years, studies have demonstrated, that the deregulation of Hippo-YAP1/WWTR1-TEAD activity is at the origin of tumor progression and resistance to therapy in a number of different cancer indications and contexts. In mice, systematic genetic studies have clearly shown that either knocking out HIPPO pathway components (which are YAP1 inhibitors) or overexpressing YAP1 activators such as YAP1, WWTR1, TEAD lead to YAP1 activation and YAP1-TEAD-dependent tumor initiation and tumor progression (Zhang N et al. Dev Cell. 2010 Jul. 2; 19 (1:27-38. Do: 10.1016/j.devcel.2010.06.01; Lu L, et al. Proc Natl Acad Sci USA. 2010: 107 (4): 1437-42. Doi: 10.1073/pnas.0911427107; Nishio M. et al. Proc Natl Acad Sci USA. 2016: 113 (1): E71-80. Do: 10.1073/pnas. 151718811; Liu-Chittenden Y, et al. Genes Dev. 2012; 26 (12): 1300-5. Do i 10.1101/gad. 192856.112). In humans, genetic alterations in the pathway are most prevalent for NF2 (neurofibromin), an upstream regulator of the core Hippo pathway, that has been linked to a heritable cancer syndrome and that has been classified as a tumor suppressor gene. Hundreds of somatically acquired mutations have been reported in NF2, predominantly in meningiomas, mesotheliomas and peripheral nerve sheath tumors, but also in other cancer types (reviewed in Schroeder R D et al. Oncotarget. 2014; 5(1):67-77. Doi: 10.18632/oncotarget. 1557). Genetic alterations beyond NF2 and directly present within the core Hippo pathway are less frequently observed in patients and found at high prevalence only in certain indications such as, e.g., malignant mesothelioma. Malignant mesothelioma is a highly lethal cancer of serosal membranes and almost exclusively associated with asbestos exposure. It is a therapeutic indication that shows prevalent alterations in the HIPPO signaling pathway as well as high YAP1 activation and high dependence on YAP1-TEAD activity (reviewed in Sekido Y. Cancers (Basel). 2018 Mar. 22; 10 (4): 90. Doi. 10.3390/cancers10040090).

Increased YAP1 or YAP1-TEAD activity is not limited to genetic alterations in the HIPPO pathway and can also be the result of upregulation through multiple interconnected signals. Numerous pathways with critical n tumorigenesis feed into the HIPPO-YAP1/WWWTR1/TEAD1 cascade, well described examples include the RTK-RAS-RAF-MEK-ERK, WNT, TGF-beta, and AMPK pathways (reviewed in Han Y. J Transl Med. 2019; 17 (1): 116. Doi: 10.1186/s12967-019-1869-4). The number of tumor types that depend at least in part on YAP1-TEAD activation is hence tremendous and spans from breast, ovarian, uterine, and prostate cancers to lung, gastric, colorectal, bladder, pancreatic, and liver cancers, and further to sarcomas, esophageal, head and neck cancers, uveal melanoma, and glioma (reviewed in Zanconato F et al. Cancer Cell. 2016; 29 (6): 783-803. Doi: 10.1016/j.ccell.2016.05.005). Recent studies reveal an interplay between the HIPPO-YAP/WWTR1/TEAD pathway and the human immune response (reviewed in Yamauchi T. Moroishi T. Cells. 2019; 8 (5): 398. Doi: 10.3390/cells8050398).

YAP1 activation has been observed in the context of resistance to therapy and is recognized as a main mechanism of resistance and survival to anti-cancer treatment. In esophageal carcinoma, YAP1 is a positive regulator of EGFR (Epidermal Growth Factor Receptor) and the induction of YAP1 is associated with resistance to 5-FU and docetaxel. In the context of targeted therapies, YAP1 in BRAF-mutant tumors, acts as a parallel survival input to promote resistance to RAF and MEK inhibitor therapy in melanoma. Similarly, activation of YAP1 is a mechanism of survival to EGFR and MEK inhibitor treatment in the context of EGFR mutant lung cancer and multiple studies have identified YAP1 activation as one of the main bypass mechanisms to KRAS inhibition. In a hormone-dependent tumor context, WWTR1 inhibition was shown to restore sensitivity to tamoxifen in breast cancer. In prostate carcinoma cells, androgen deprivation therapy resistance was associated with increased YAP1 nuclear localization and activity (reviewed in Reggiani F et al. Biochim Biophys Acta Rev Cancer. 2020; 1873 (1): 188341. Doi: 10.1016/j.bbcan.2020.188341; Kurppa K J et al. Cancer Cell. 2020; 37 (1). 104-122.e12. doi. 10.1016/j ccell 2019.12.006).

Thus, the HIPPO-YAP/WWTR1/TEAD pathway is a key player in cancer development and tumor maintenance and targeting this pathway is key for cancer treatment, both in a first line therapy setting as well as in the context of overcoming drug resistance with multiple cancer indications.

As used herein, a “TEAD-pathway inhibitor” may be a small or large molecule that inhibits the HIPPO-YAP/WWTR1/TEAD pathway.

### Additional Anticancer Therapies

In some embodiments, the methods disclosed herein further comprise administering a TEAD-pathway inhibitor and an additional anticancer therapy. The additional anticancer therapy can comprise any therapy known in the art for the treatment of a tumor in a subject and/or any standard-of-care therapy. In some embodiments, the additional anticancer therapy comprises a surgery, a radiation therapy, a chemotherapy, an immunotherapy, a hormone therapy, or any combination thereof. In some embodiments, the additional anticancer therapy comprises a chemotherapy. In some embodiments, the additional anticancer therapy comprises an immunotherapy. In some embodiments, the additional anticancer therapy comprises a hormone therapy.

### Kits and Articles of Manufacture

In some embodiments, provided herein is a kit or an article of manufacture containing any one of the panels described herein, or materials useful in preparing such panels.

In some embodiments, the kit or article of manufacture may include one or more reagents for preparing a sample for RNA sequencing analysis.

In some embodiments, the kit or article of manufacture further includes one or more reagents for determining a TEAD-inactive or TEAD-active score from a sample.

In some embodiments, the kit or article of manufacture may include instructions to use the kit for any of the methods/uses as described herein.

In some embodiments, the kit or article of manufacture may include a container, a label on the container, and a composition contained within the container, wherein the composition includes one or more polynucleotides that hybridize to a complement of a gene listed herein under stringent conditions, and the label on the container indicates that the composition can be used to evaluate the activity of a set of genes listed herein, and wherein the kit includes instructions for using the polynucleotide(s) for evaluating the presence of the genes' RNA or DNA in a particular sample type.

In some embodiments, the kit or article of manufacture is oligonucleotide-based and may include, for example (1) an oligonucleotide, for example a detectably labeled oligonucleotide, which hybridizes to a nucleic acid sequence encoding a protein or (2) a pair of primers useful for amplifying a nucleic acid molecule. In some embodiments, the kit or article of manufacture may also include a buffering agent, a preservative, or a protein stabilizing agent. In some embodiments, the kit or article of manufacture may further include components necessary for detecting the detectable label, for example, an enzyme or a substrate. In some embodiments, the kit or article of manufacture may also contain a control sample or a series of control samples that can be assayed and compared to the test sample. In some embodiments, each component of the kit or article of manufacture can be enclosed within an individual container and all of the various containers can be within a single package, along with instructions for interpreting the results of the assays performed using the kit or article of manufacture.

### Example 1

### Materials and Methods

Key details of the materials and methods used are provided below.

### Compilation of the TEAD-500 Gene List.

We reviewed published datasets and selected genes that were differentially expressed at p-value<0.01 by analysis of variance compering treated samples with their corresponding controls. The resulting 37 gene lists were compiled into one. Genes that were seen modulated only once, and genes that were inconsistently modulated between experiments, were removed from the compilation. The remaining ˜700 genes, consistently modulated in at least 2 experiments were sorted by ascending p-value and the top 500 effectors were finally selected to be routinely interrogated in large cohorts.

### Calculation of the TEAD-500 Signature Score.

TEAD-dependent transcription was scored in single samples as the difference Rp-Rn, where Rp is the mean fractional rank of the positive TEAD-effectors, and Rn, that of the negative effectors.

We considered the most abundant transcript of a gene as the representative of the expression of the gene. We, therefore, retain the expression level of the major transcript (maximum level-value per gene and sample, level values may be expression-array signals, RNA sequencing counts, fragments per kilobase per million counts (FPKM), transcripts per million (TPM), or whatever other metric is conveniently used) and discarded the values of all other transcripts. The maximum expression levels of the genes were transformed into fractional ranks by dividing with the maximum expression value of all genes (most abundant gene) observed within a sample. No other normalization is required.

Due to technological evolution (from various commercial expression arrays to RNA sequencing techniques), but also to gene nomenclature inconsistencies, practically the panel does not typically contain all the 500 genes reported in the training datasets from TEAD-500. However, dropouts up to at least 10% only negligibly influence its score. This is one of the reasons why TEAD-500 is highly robust.

### Epidemiological and Experimental Test Datasets.

We scored and binned into “TEAD-active” and “TEAD-inactive” categories the TEAD-dependent transcription in the ˜9000 normal tissue samples of the Genotype-Tissue Expression project (GTEx Portal on Sep. 24, 2021; https://gtexportal.org/home/), in the primary, recurrent, or metastatic tumors of The Cancer Genome Atlas (TCGA; ˜11000 samples; https://www.cancer.gov/tcga). And in laboratory cell lines of the Cancer Cell Line Encyclopedia (CCLE; ˜1000 samples; https://sites.broadinstitute.org/ccle/) for selecting experimental models. We further applied the signature to follow the degree of TEAD inhibition in cell and tumor models either after genetic knockdown of YAP1 or TEAD1 or by pharmacological inhibition using the recently published (Kaneda A, et al. Am J Cancer Res. 2020; 10 (12): 4399-4415) TEAD palmitoylation inhibitor K-975 (Kyowa Kirin Co., Ltd., Shizuoka, Japan).

### Cells and Culture Conditions.

All cell lines were grown at 37° C. under five percent CO₂. MSTO-211H (#CRL-2081), NCI-H226 (#CRL-5826), NCI-H2052 (#CRL-5915), NCI-H28 (#CRL-5820), and HCT116 (#CCL-247) were purchased from the American Type Culture Collection (ATCC, Manassas, VA, USA) and cultured according to supplier's recommendation. ZL5 (No. 11120715), ZL34 (No. 11120713), SPC212 (No. 11120717), Mero-14 (No. 09100101), Mero-48a (No. 09100104), Mero-95 (No. 09100108), JU77 (No. 10092309), LO68 (No. 10092311), and ONE58 (No. 10092313) were purchased from the European Collection of Authenticated Cell Cultures (ECACC, Public Health England, Salisbury, UK) and cultured according to supplier's recommendation. A short tandem repeat assay authenticated all cell lines at the Microsynth AG (Balgach, Switzerland). PCR using the Venor™ GeM Mycoplasma Detection Kit (Biovalley, Nanterre, France) excluded mycoplasma infection.

### Cell Line Transfection and Constructs.

We generated cell lines stably expressing sh-YAP1, sh-Null, or TEAD2-DN, by transducing the MSTO-211H and HCT116 cells with PiggyBac transposon vectors using the Lipofectamine 3000 reagent protocol from Thermo Fisher™ (Wilmington, DE). Transduced cells were selected in media supplemented with 1 mg/ml puromycin. For the generation of PiggyBac constructs, DNA sequences of sh-YAP1 and sh-Null, were cloned into the pPiggyBac-KO-Tet-One-Puro DEST plasmid. The DNA sequences of human TEAD2-DN (Ser112-Asp447; Liu-Chittenden, Y., et al., Genes Dev, 2012. 26 (12): 1300-5) with a FLAG-tag at the N-terminus were cloned into pPiggyBac-A-Tet-One-puro DEST vector.

### Cell Growth and Apoptosis Assays.

Cells were seeded in medium supplemented or not with doxycycline (1 ug/ml) and were incubated for 96 hours at 37° C. and 5% CO₂. Cell growth was measured by the trypan blue dye exclusion method using Vi-CELL-XR Cell Viability Analyzer (Beckman Coulter). Caspase-3/7 activity was detected using the Cell-Event Caspase-3/7 Green Ready-Probes reagent (Molecular Probes) and measured by IncuCyte™ ZOOM live cell analysis system (Essen Bioscience) with scans every two hours for 72 h. The acquired fluorescent signal for activated caspase 3/7 was normalized with well confluency at each timepoint (=normalized apoptosis). Peak apoptosis was determined as the highest normalized caspase 3/7 activity value during the assay.

### Animals.

Female CB17/Icr-Prkdc^{scid}/IcrIcoCrl mice (6-8 weeks old) were bred at Charles River (Les Oncins, France), housed in Sanofi AAALAC accredited animal facilities, and were provided with irradiated food and filtered water ad libitum.

### In Vivo Xenograft Studies.

In vivo tumor growth of thirteen malignant mesothelioma cell lines (MSTO-211H; ZL5; NCI-H226; JU77; MERO 48A; MERO 95; H2052; ZL-34; SCP-212; MERO-14; Lo68; One58; H28) was evaluated after subcutaneous cell inoculation into the right flank of SCID mice at 3×10⁶ cells and 10×10⁶ cells. In vivo target validation studies were conducted using the MSTO-211H-TEAD2-DN, MSTO-211H-shYAP1, and HCT116-shYAP1 cell lines inoculated subcutaneously at 3×10⁶ cells mixed with Matrigel. Immediately after cell implantation, mice were divided into two groups. Each group received drinking water supplemented with either doxycycline and five percent glucose or with five percent glucose only.

In vivo efficacy studies of the K-975 compound were performed in SCID mice inoculated subcutaneously into the right flank with 3×10⁶ MSTO-211H or NCI-H226 cells. Mice bearing around 200 mm3 subcutaneous MSTO-211H tumors or 150 mm3 subcutaneous NCI-H226 tumors were randomly assigned to 5 groups of 8 mice/group and treated with either vehicle or K-975 compound twice daily at 30, 100, and 200 mg/kg for 18 consecutive days. Tumor perpendicular diameters were measured twice a week with a caliper, and tumor volume (V) was calculated according to the following equation: V (mm3)=(d2 (mm2)×D (mm))/2, where d is the smallest, and D the largest perpendicular tumor diameters.

### In Vitro Validation.

The effect of YAP1 downregulation on tumor growth was first tested in vitro using a genetic approach based on a TEAD dominant-negative (TEAD2-DN) construct. This construct was previously reported as an efficient inhibitor of YAP1 activity (Liu-Chittenden, Y., et al., Genes Dev, 2012. 26 (12): 1300-5) t is based on a truncated version of TEAD2, which lacks the DNA binding domain but retains its ability to associate with YAP1. It thereby acts as a non-functional competitor of endogenous TEAD for binding to YAP1.

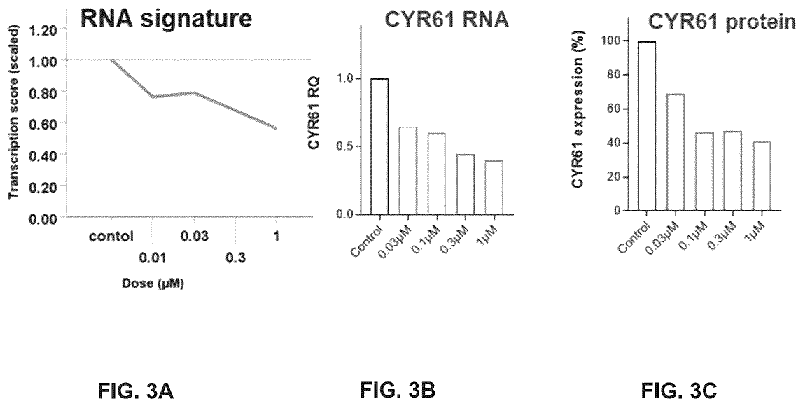

We engineered this construct behind a doxycycline-inducible promoter and used it for the stable transfection of MSTO-211H cells. Upon doxycycline addition, TEAD2-DN expression resulted in 66% inhibition of tumor cell growth in vitro 96 h post doxycycline induction, FIGS. 3A-3B, accompanied by a significant drop of the TEAD-500 score as well as of the classical TEAD-activity protein markers CYR61 and CTGF.

The latter protein markers were measured by ELISA assays. Cell lysates were diluted 1/500 in cell lysis buffer. A minimum of four biologic replicates were analyzed for each model. In the reading plate, standards and samples are run in duplicate. hCYR61 was assessed using the human Cyr61 Quantikine™ Colorimetric Sandwich ELISA Kit (R&D Systems, Minneapolis, MN). Diluted lysates were processed and analyzed as described in the manufacturer's instructions. Absorbance values were read using a microplate reader set to 450 nm.

### In Vivo Validation.

We generated cell lines stably expressing sh-YAP1, sh-Null, or TEAD2-DN, by transducing the MSTO-211H and HCT116 cells with piggyBac transposon vectors using the Lipofectamine 3000 reagent protocol from Thermo Fisher. Transduced cells were selected in media supplemented with 1 mg/ml puromycin. For the generation of piggyBac constructs, DNA sequences of sh-Yap1 and sh-Null, were cloned into the pPiggyBac-KO-Tet-One-Puro DEST plasmid. The DNA sequences of human TEAD2-DN (Ser112-Asp447; Liu-Chittenden, Y., et al., Genes Dev, 2012. 26 (12): 1300-5) with a FLAG-tag at the N-terminus were cloned into pPiggyBac-A-Tet-One-puro DEST vector. NCI-MSTO-211H malignant mesothelioma cells were transfected with a doxycycline-dependent dominant-negative TEAD1 shRNA (TEAD DN), implanted subcutaneously into SCID mice, and let grow for 26 days. The YAP1 shRNA was induced in established tumors and led to tumor regression in all mice tested.

### RNA-Sequencing.

Fresh tumors were preserved in RNAlater (Thermo Fisher) and frozen at −200° C. Copurification of miRNA and total RNA was performed using a miRNeasy Mini Kit (QIAGEN, 217004). The RNA concentration and purity were evaluated with an ND-1000 spectrophotometer (NanoDrop, Thermo Fisher). Quality metrics are the RNA Integrity Number (RIN) and DV200 value (% of RNA fragments with a length≥200 nt). 100 ng total RNA per sample was used as input material for the library preparation. The libraries were prepared with the KAPA HyperPrep Kit and KAPA Target Enrichment using the KAPA HyperCap V3.0 kit for Illumina following the manufacturer's recommendations (all kits purchased from Kapa Biosystems, Wilmington, MA). The libraries were sequenced using NextSeq 500 paired-end sequencing platform (Illumina Inc., San Diego, CA).

### Discriminant Functions.

To compute tissue-specific combinations of predictors of TEAD activity, a classical statistical method known as Fisher's stepwise linear discriminant function analysis was used. This algorithm builds a linear model in a stepwise manner. The most significant predictor (in this case, the most significant effector gene) enters the model first. The data are standardized for that predictor. At each step, the predictor that minimizes the overall Wilks' lambda is entered into the equation. All the predictors in the model are re-evaluated. Those that are no longer significant are removed from the model. Selection of predictors stops when all the predictors in the model are significant and all those remaining out are non-significant. The SPSS Statistic software (IBM, Armonk, NY) was used for these calculations.

### Results

### Robustness of the TEAD-500 Score.

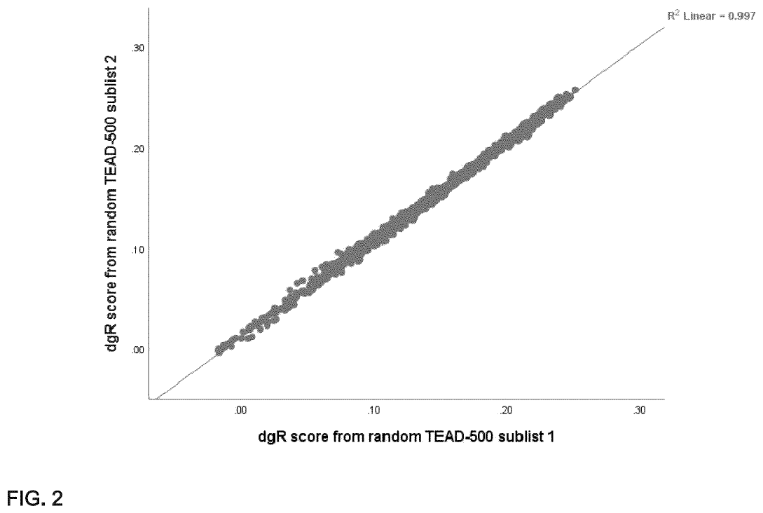

Due to the large number of genes in the TEAD-500 list and the high redundancy of this signature (correlations among genes), its scores are largely immune to gene-dropouts. We have simulated dropouts by producing two random sub-lists, each containing only 90% of the genes. A different 10% of the genes was omitted from each sub-list. Using these different sub-lists, we calculated dgR scores for 1112 experimental samples. These samples were cell lines with TEAD-500 scores at the upper extreme of the scale (TEAD-activated) but were grown under various TEAD-inhibitory conditions. As shown in FIG. 2, the correlation between the two randomly corrupted scores was almost perfect (R²=0.997).

### Validation of the TEAD-500 Score In Vitro.

When the malignant mesothelioma cell line NCI-H226 was treated with a benchmark TEAD-inhibitor (K-975; see, e.g., Kaneda A et al. Am J Cancer Res 2020; 10 (12) 4399-4415), for 24 hours, the TEAD-500 score regressed in a dose-dependent manner (FIGS. 3A-3B). So did the RNA and protein levels of the classical secreted marker of Hippo pathway activity, CYR61 (FIG. 3C).

### Validation of the TEAD-500 Score In Vivo.

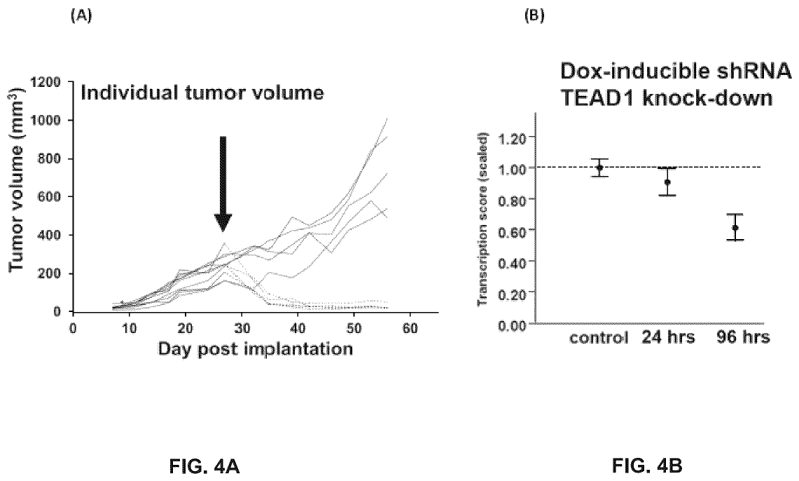

NCI-MSTO-211H malignant mesothelioma cells were transfected with a doxycycline-dependent dominant-negative TEAD1 shRNA, implanted subcutaneously into SCID mice, and let grow for 26 days. The YAP1 shRNA was induced in established tumors and led to tumor regression in all mice tested (FIG. 4A). Tumor regression came along with the significant decline of the TEAD-dependent transcription (TEAD-500 score) (FIG. 4B). TEAD-dependent transcription was, therefore, essential for tumor maintenance of malignant mesothelioma in vivo.

### TEAD-500 as a Pharmacodynamic Marker.

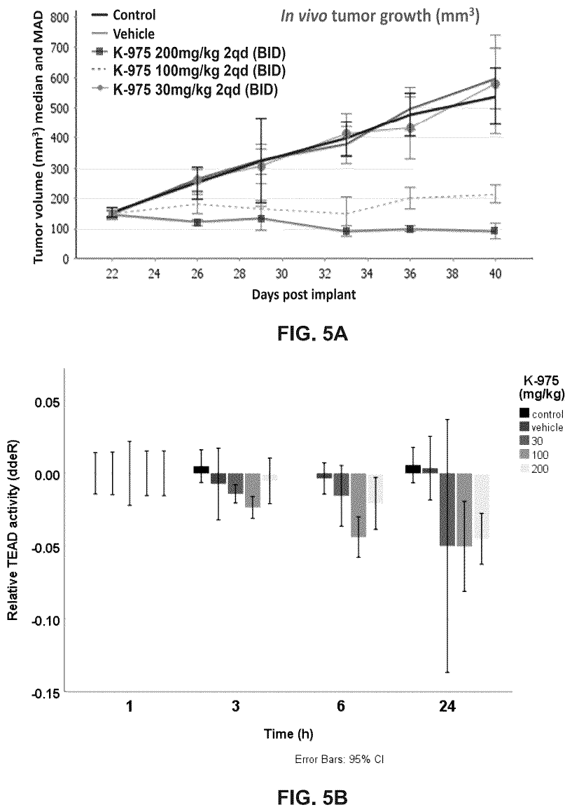

The same type of events was observed when SCID mice implanted with malignant mesothelioma parental NCI-H226 cells were treated with 200 mg/kg BID of the benchmark TEAD-inhibitor K-975 (Kaneda A et al. Am J Cancer Res. 2020; 10(12):4399-4415) for 17 consecutive days (FIG. 5). Approximately 30-40% inhibition of the TEAD-dependent transcription seems sufficient for tumor regression in this model. The dose of 100 mg/kg BID was still active with ΔT/ΔC=12 (almost tumor stasis), while the dose of 30 mg/kg BID was inactive. The TEAD-500 score correlates, therefore, with tumor behavior under treatment and can be used as a marker of the pharmacodynamics or efficacy of a drug.

### Epidemiology of the TEAD-500 Score.

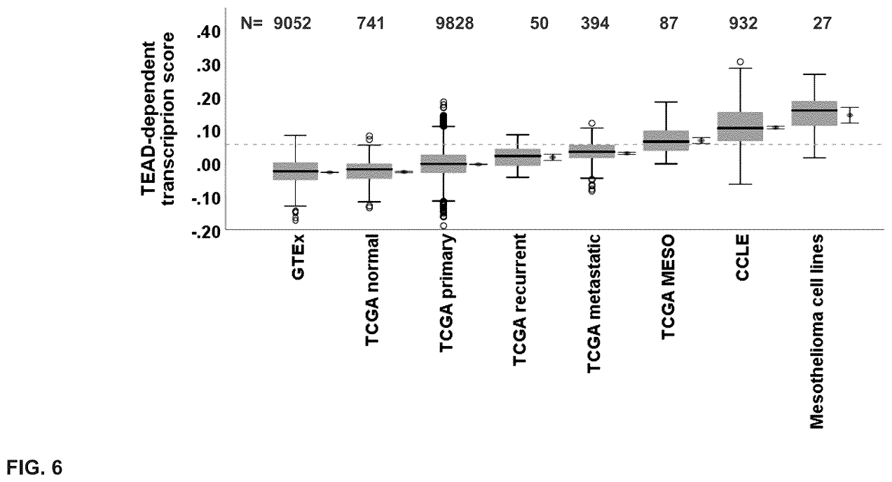

FIG. 6 shows that the TEAD-500 deR score distribution moves towards higher values in tumors (TCGA) compared to normal tissues (GTEx). Primary tumors tend to score higher than adjacent normal tissues. Recurrent and metastatic tumors score higher than primary tumors. Therefore, TEAD-dependent transcription increases with tumorigenesis and tumor progression, at least in some cases. This result is consistent with numerous observations that the TEAD pathway confers a mechanism of escape from treatment (Coggins G E et al. Cancer Res. 2019; 79 (24): 6204-6214. Doi: 10.1158/0008-5472.CAN-19-1415; Ansari D et al. Anticancer Res. 2019; 39 (7): 3317-3321 Doi: 10.21873/anticanres.13474; Kim C G et al. Cancer Immunol Res. 2018; 6 (3): 255-266. Doi: 10.1158/2326-6066.CIR-17-0320).

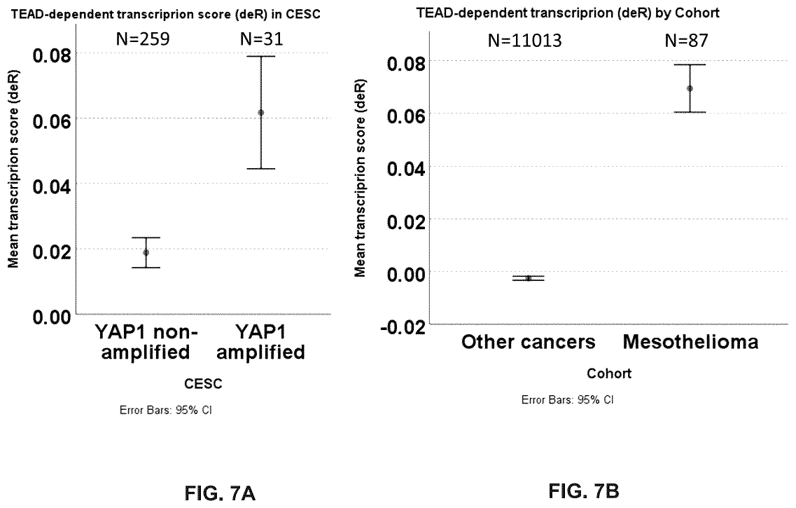

Primary malignant mesothelioma is an indication with very high TEAD activity among all TCGA cohorts tested. Its average TEAD-500 score is even higher than the average score in metastatic tumors. This confirms published observations that TEAD-dependent transcription increases in malignant mesothelioma (Mizuno, T., Murakami, H., Fujii, M. et al. Oncogene 31, 5117-5122 (2012). https://doi.org/10.1038/onc.2012.5) and justifies the choice of mesothelioma as the gateway indication for the proof-of-concept clinical trials of novel TEAD inhibitors (https://www.bolderscience.com/trial/nct04665206/). Cell lines (CCLE) attain much higher scores, presumably because they are clonal populations of advanced tumoral cells. The tested mesothelioma cell lines are among the top scorers. We also confirm published observations that TEAD-dependent transcription increases in in cervical cancers with YAP1 amplification (FIG. 7; Lorenzetto E, et al. Oncotarget. 2014; 5 (9): 2608-21; doi: 10.18632/oncotarget. 1844).

### Prognostic Value of the TEAD-500 Scores.

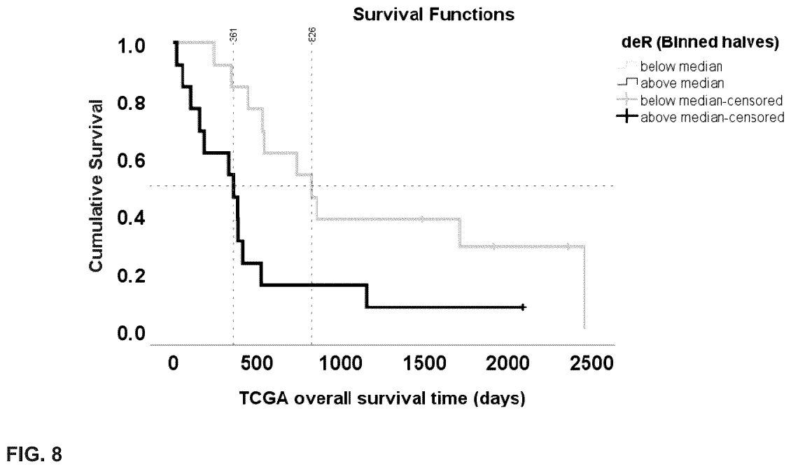

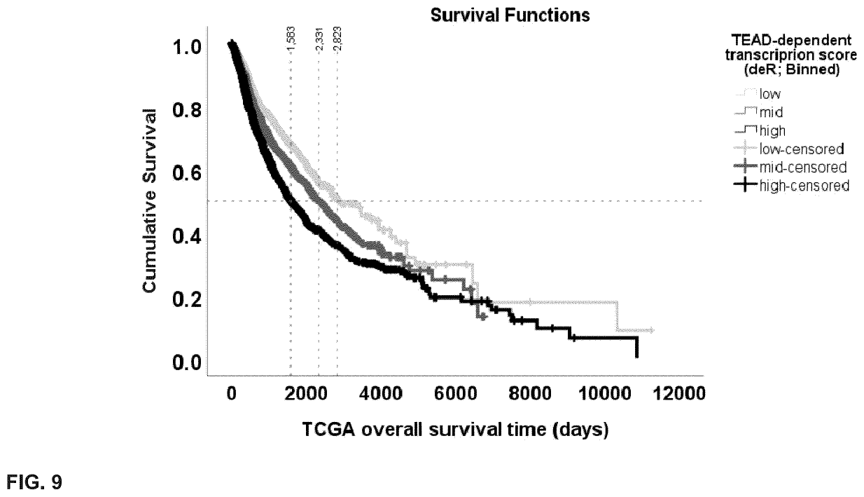

The mesothelioma cohort (TCGA MESO) was split into two equal groups according to TEAD-500 deR score of the tumor samples. Patients with low scores had a median overall survival of 826 days, which is about 2.3-fold longer than the median survival of 361 days for patients with high TEAD-500 scores (FIG. 8). The difference was significant by all, log rank (p=0.011), Breslow (p=0.004), and Tarone-Ware (p=0.006) tests. When all the TCGA indications were pooled and split into three equal groups according to the TEAD-500 score, we observed again a highly significant difference in overall survival among the three groups with p-values smaller than 1E-15 for all three statistical tests. The median survival was only 1583 days for the group with the highest scores, compared to 2331 days for the group with intermediate scores and 2823 for the lowest scorers (FIG. 9).

Independence of TEAD-500 from Classical Markers.

There is some significant but rather feeble (given the large sample sizes) correlation between the scores of the optimal discriminant functions (Table 4), or the minimal ones (Table 5), and the RNA levels of the two markers, CCN1 and CCN2, commonly used for measuring YAP1 or TEAD activity. Significant correlations are observed only in some cancer indications or subtypes. These correlations may be interpreted as yet another validation of our methodology. For the majority of cohorts, however, the discriminant scores seem to be independent predictors of TEAD-pathway activity, particularly in tissues where CCN1 and CCN2 biology is irrelevant.

TABLE 4
Significance of correlation between optimal discriminant
function scores and the RNA levels of the classical TEAD-
pathway activation markers CCN1 and CCN2 by cancer indication
and subtype (p-values < 0.05 are highlighted in bold)

| Tumor type | CCN1 | CCN2 |
|---|---|---|
| ACC | 2E−02 | 8E−01 |
| BLCA | 5E−05 | 2E−03 |
| BRCA | 9E−01 | 6E−01 |
| BRCA basal | 7E−03 | 2E−04 |
| BRCA non-Basal | 3E−01 | 8E−02 |
| CESC | 1E−14 | 4E−03 |
| CHOL | 6E−01 | 8E−01 |
| COAD | 5E−01 | 5E−01 |
| COAD CMS1 | 3E−02 | 4E−02 |
| COAD CMS2 | 8E−01 | 9E−01 |
| COAD CMS4 | 3E−01 | 3E−01 |
| DLBC | 7E−04 | 5E−06 |
| ESCA | 2E−01 | 9E−01 |
| GBM | 8E−01 | 4E−01 |
| HNSC | 2E−05 | 4E−01 |
| KICH | 6E−01 | 5E−01 |
| KIRC | 1E+00 | 6E−01 |
| KIRP | 1E−01 | 7E−01 |
| LGG | 2E−03 | 2E−02 |
| LIHC | 1E−02 | 1E−05 |
| LIHC S1 | 2E−01 | 6E−01 |
| LIHC S2 | 5E−02 | 9E−01 |
| LIHC S3 | 2E−01 | 8E−01 |
| LUAD | 7E−01 | 7E−01 |
| LUAD proximal-inflammatory | 1E−01 | 5E−01 |
| LUAD proximal-proliferative | 7E−02 | 7E−01 |
| LUSC | 2E−01 | 5E−02 |
| LUSC basal | 6E−01 | 3E−01 |
| LUSC classical | 2E−03 | 8E−03 |
| LUSC primitive | 2E−03 | 1E−03 |
| LUSC secretory | 7E−01 | 4E−01 |
| MESO | 2E−04 | 5E−02 |
| OV | 5E−03 | 5E−02 |
| OV differentiated | 2E−01 | 9E−01 |
| OV immune-reactive | 2E−02 | 3E−01 |
| OV mesenchymal | 4E−01 | 4E−01 |
| OV proliferative | 4E−02 | 1E−01 |
| PAAD | 5E−01 | 6E−01 |
| READ | 7E−02 | 3E−02 |
| READ CMS1 | 1E+00 | 6E−01 |
| READ CMS2 | 1E+00 | 9E−01 |
| READ CMS4 | 5E−01 | 4E−01 |
| READ unclassifiable | 9E−01 | 8E−01 |
| SARC | 3E−01 | 5E−01 |
| SKCM | 5E−03 | 1E−01 |
| SKCM immune | 4E−01 | 7E−01 |
| SKCM keratin | 5E−01 | 1E+00 |
| SKCM MITF-low | 2E−02 | 4E−01 |
| STAD MSI | 7E−03 | 2E−01 |
| STAD MSS_EMT | 9E−01 | 3E−01 |
| STAD MSS_TP53− | 1E−01 | 5E−01 |
| STAD MSS_TP53+ | 5E−01 | 7E−01 |
| TGCT | 7E−02 | 9E−03 |
| THYM | 9E−05 | 2E−04 |
| UCEC | 9E−02 | 1E−01 |
| UCS | 2E−01 | 3E−02 |

TABLE 5
Significance of correlation between minimal discriminant function
scores and the RNA levels of the classical TEAD-pathway activation
markers CCN1 and CCN2 by cancer indication and subtype.

| Tumor type | CCN1 | CCN2 |
|---|---|---|
| ACC | 2E−02 | 4E−01 |
| BRCA non-Basal | 8E−01 | 6E−01 |
| CHOL | 9E−02 | 4E−01 |
| COAD CMS1 | 3E−02 | 3E−02 |
| COAD CMS2 | 7E−01 | 8E−01 |
| COAD CMS4 | 1E−01 | 3E−01 |
| ESCA | 1E−01 | 8E−01 |
| GBM | 8E−01 | 4E−01 |
| KICH | 3E−01 | 4E−01 |
| LGG | 2E−03 | 1E−02 |
| LIHC S1 | 3E−01 | 8E−01 |
| LIHC S2 | 4E−02 | 8E−01 |
| LIHC S3 | 1E−01 | 8E−01 |
| LUAD proximal- | 3E−02 | 7E−01 |
| proliferative | | |
| LUSC basal | 5E−01 | 3E−01 |
| LUSC secretory | 7E−01 | 5E−01 |
| OV mesenchymal | 3E−01 | 3E−01 |
| OV proliferative | 5E−02 | 8E−02 |
| PAAD | 9E−01 | 8E−01 |
| READ CMS2 | 5E−01 | 6E−01 |
| READ CMS4 | 6E−01 | 5E−01 |
| READ unclassifiable | 7E−01 | 9E−01 |
| SKCM keratin | 6E−01 | 8E−01 |
| SKCM MITF-low | 2E−02 | 5E−01 |
| STAD MSS_TP53− | 1E−01 | 4E−01 |
| STAD MSS_TP53+ | 3E−01 | 7E−01 |

### Performance of Discriminant Functions as Classifiers.

Several metrics can be computed from the numbers of positive (P; TEAD-500>0.055) and negative (N; TEAD-500<0.055) samples and true positive (TP), true negative (TN), false positive (FP) and false negative (FN) predictions to evaluate the performance of classifiers. The positive predictions (PP) are the sum TP+FP. The negative predictions are the sum TN+FN. The prevalence of positive sample in a population (cohort) is the proportion of positives, P/(P+N). The accuracy of prediction is the proportion of correctly classified samples, (TP+TN)/(P+N). Precision, also known as positive predictive value (PPV), is the proportion of true positives among all the predicted positives, TP/PP. The false discovery rate (FDR) is the proportion of false positives among the predicted positives, FP/PP or 1-PPV. The false omission rate (FOR) is the proportion of predicted negatives that are true negatives, FN/PN or 1-NPV (negative predictive value). The negative predictive value (NPV) is the proportion of the predicted negatives that are true negatives, TN/PN, or 1-FOR. Sensitivity, also known as true positive rate (TPR), recall, or power of prediction, is the proportion of positives truly predicted as such, TP/P. Specificity, selectivity, or true negative rate (TNR), is the proportion of negatives that are truly predicted as such, TN/N. The false negative rate (FNR), or miss rate, is the proportion of positives that are falsely predicted to be negatives, FN/P.

The F-score, or F-measure, is a measure of a test's accuracy commonly used in statistical analysis of binary classification. This is calculated from the precision and sensitivity of the test as:

$$F score = ( 2 \times PPV \times TPR ) / ( PPV + TPR ) or 2 \times TP / ( 2 \times TP + FP + FN )$$

Perhaps the most frequently used single measure of performance of a classifier is informedness, or Youden's J statistic (also called Youden's index), defined in terms of sensitivity (true positive rate) and specificity (true negative rate) as TPR+TNR−1. This is a single statistic that captures the performance of a dichotomous diagnostic test.

The values of these metrics for all our discriminant classifiers, whether these are optimal or minimal, are shown in Table 6. Out of 57 disclosed classifiers, 40 achieve perfect discrimination (informedness=1) of tumors with active, against non-active TEAD-pathway, as these are defined by our generic TEAD-500 signature. Another 10 classifiers have excellent performance achieving informedness values equal or greater 0.9. Another 5 classifiers raise the probability of correct patient selection from 18% or less than 10% (prevalence or prior probability value) to the range of 85-90%. Even our most mediocre functions applied to THYM and READ cohorts provide for informed correct decisions at rates of 59% and 76%, instead of a 4% and 7% achievable by chance, respectively.

TABLE 6
Performance of the discriminant functions as classifiers of the
TEAD-pathway status in TCGA indications and tumor subtypes.

| | Total | | | | | | Miss | F- | |
|---|---|---|---|---|---|---|---|---|---|
| Cancer type | samples | Prevalence | Accuracy | Precision | Sensitivity | Specificity | rate | measure | Informedness |
| ACC | 79 | 0.08 | 1 | 1 | 1 | 1 | 0 | 1 | 1 |
| BLCA | 430 | 0.14 | 1 | 1 | 0.98 | 1 | 0.02 | 0.99 | 0.98 |
| BRCA | 1227 | 0.06 | 0.99 | 0.95 | 0.93 | 1 | 0.07 | 0.94 | 0.93 |
| BRCA basal | 212 | 0.28 | 1 | 1 | 1 | 1 | 0 | 1 | 1 |
| BRCA non-Basal | 758 | 0.01 | 1 | 1 | 1 | 1 | 0 | 1 | 1 |
| CESC | 309 | 0.20 | 0.99 | 1 | 0.97 | 1 | 0.03 | 0.98 | 0.97 |
| CHOL | 45 | 0.07 | 1 | 1 | 1 | 1 | 0 | 1 | 1 |
| COAD | 514 | 0.04 | 1 | 1 | 0.89 | 1 | 0.11 | 0.94 | 0.89 |
| COAD CMS1 | 53 | 0.09 | 1 | 1 | 1 | 1 | 0 | 1 | 1 |
| COAD CMS2 | 84 | 0.05 | 1 | 1 | 1 | 1 | 0 | 1 | 1 |
| COAD CMS4 | 65 | 0.02 | 1 | 1 | 1 | 1 | 0 | 1 | 1 |
| DLBC | 48 | 0.46 | 1 | 1 | 1 | 1 | 0 | 1 | 1 |
| ESCA | 198 | 0.08 | 1 | 1 | 1 | 1 | 0 | 1 | 1 |
| GBM | 191 | 0.15 | 1 | 1 | 1 | 1 | 0 | 1 | 1 |
| HNSC | 566 | 0.10 | 1 | 1 | 0.98 | 1 | 0.02 | 0.99 | 0.98 |
| KICH | 91 | 0.02 | 1 | 1 | 1 | 1 | 0 | 1 | 1 |
| KIRC | 609 | 0.02 | 1 | 1 | 0.87 | 1 | 0.13 | 0.93 | 0.87 |
| KIRP | 322 | 0.02 | 1 | 1 | 1 | 1 | 0 | 1 | 1 |
| LGG | 534 | 0.02 | 1 | 1 | 1 | 1 | 0 | 1 | 1 |
| LIHC | 424 | 0.08 | 1 | 0.97 | 1 | 1 | 0 | 0.99 | 1 |
| LIHC S1 | 76 | 0.01 | 1 | 1 | 1 | 1 | 0 | 1 | 1 |
| LIHC S2 | 135 | 0.17 | 1 | 1 | 1 | 1 | 0 | 1 | 1 |
| LIHC S3 | 141 | 0.06 | 1 | 1 | 1 | 1 | 0 | 1 | 1 |
| LUAD | 589 | 0.06 | 1 | 1 | 0.94 | 1 | 0.06 | 0.97 | 0.94 |
| LUAD proximal- | 139 | 0.11 | 1 | 1 | 1 | 1 | 0 | 1 | 1 |
| inflammatory | | | | | | | | | |
| LUAD proximal- | 182 | 0.11 | 1 | 1 | 1 | 1 | 0 | 1 | 1 |
| proliferative | | | | | | | | | |
| LUSC | 552 | 0.12 | 0.98 | 0.92 | 0.91 | 0.99 | 0.09 | 0.92 | 0.9 |
| LUSC basal | 103 | 0.01 | 1 | 1 | 1 | 1 | 0 | 1 | 1 |
| LUSC classical | 169 | 0.08 | 0.98 | 0.92 | 0.86 | 0.99 | 0.14 | 0.89 | 0.85 |
| LUSC primitive | 99 | 0.32 | 1 | 1 | 1 | 1 | 0 | 1 | 1 |
| LUSC secretory | 115 | 0.16 | 1 | 1 | 1 | 1 | 0 | 1 | 1 |
| MESO | 87 | 0.63 | 1 | 1 | 1 | 1 | 0 | 1 | 1 |
| OV | 430 | 0.29 | 0.98 | 0.98 | 0.95 | 0.99 | 0.05 | 0.96 | 0.94 |
| OV differentiated | 42 | 0.10 | 1 | 1 | 1 | 1 | 0 | 1 | 1 |
| OV immune- | 58 | 0.24 | 1 | 1 | 1 | 1 | 0 | 1 | 1 |
| reactive | | | | | | | | | |
| OV | 54 | 0.52 | 1 | 1 | 1 | 1 | 0 | 1 | 1 |
| mesenchymal | | | | | | | | | |
| OV proliferative | 53 | 0.38 | 1 | 1 | 1 | 1 | 0 | 1 | 1 |
| PAAD | 183 | 0.01 | 1 | 1 | 1 | 1 | 0 | 1 | 1 |
| READ | 177 | 0.07 | 0.98 | 0.91 | 0.77 | 0.99 | 0.23 | 0.83 | 0.76 |
| READ CMS1 | 4 | 0.50 | 1 | 1 | 1 | 1 | 0 | 1 | 1 |
| READ CMS2 | 32 | 0.13 | 1 | 1 | 1 | 1 | 0 | 1 | 1 |
| READ CMS4 | 27 | 0.11 | 1 | 1 | 1 | 1 | 0 | 1 | 1 |
| READ | 10 | 0.10 | 1 | 1 | 1 | 1 | 0 | 1 | 1 |
| unclassifiable | | | | | | | | | |
| SARC | 265 | 0.55 | 1 | 1 | 0.99 | 1 | 0.01 | 1 | 0.99 |
| SKCM | 472 | 0.30 | 0.97 | 0.94 | 0.95 | 0.97 | 0.05 | 0.94 | 0.92 |
| SKCM immune | 180 | 0.29 | 0.99 | 0.98 | 1 | 0.99 | 0 | 0.99 | 0.99 |
| SKCM keratin | 85 | 0.45 | 1 | 1 | 1 | 1 | 0 | 1 | 1 |
| SKCM MITF-low | 98 | 0.35 | 1 | 1 | 1 | 1 | 0 | 1 | 1 |
| STAD | 453 | 0.06 | 0.99 | 1 | 0.86 | 1 | 0.14 | 0.93 | 0.86 |
| STAD MSI | 101 | 0.13 | 1 | 1 | 1 | 1 | 0 | 1 | 1 |
| STAD MSS_EMT | 129 | 0.02 | 1 | 1 | 1 | 1 | 0 | 1 | 1 |
| STAD | 90 | 0.09 | 1 | 1 | 1 | 1 | 0 | 1 | 1 |
| MSS_TP53− | | | | | | | | | |
| STAD | 88 | 0.06 | 1 | 1 | 1 | 1 | 0 | 1 | 1 |
| MSS_TP53+ | | | | | | | | | |
| TGCT | 150 | 0.59 | 1 | 1 | 1 | 1 | 0 | 1 | 1 |
| THYM | 122 | 0.04 | 0.98 | 0.75 | 0.6 | 0.99 | 0.4 | 0.67 | 0.59 |
| UCEC | 586 | 0.18 | 0.97 | 0.96 | 0.88 | 0.99 | 0.12 | 0.92 | 0.87 |
| UCS | 57 | 0.53 | 1 | 1 | 1 | 1 | 0 | 1 | 1 |

### Example 2

### Mesothelioma—Comparison of the Optimal and Minimal Discriminant Function Scores to the TEAD-500 Score.

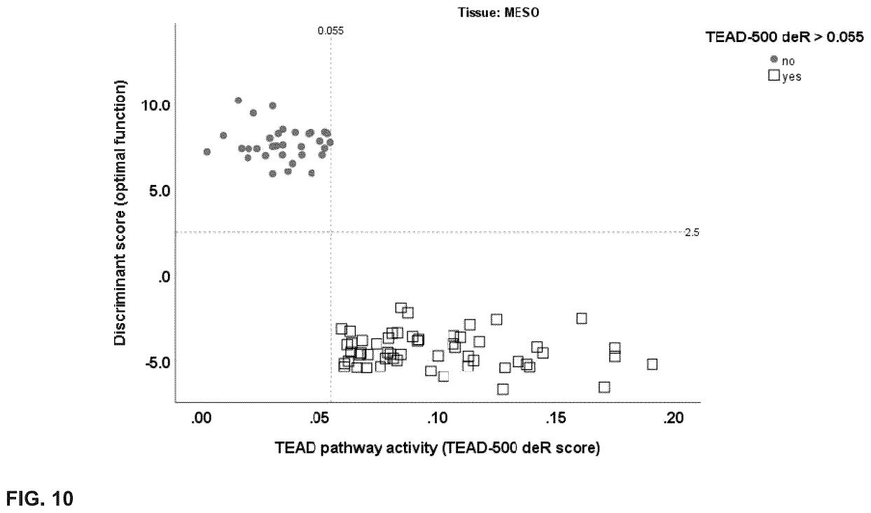

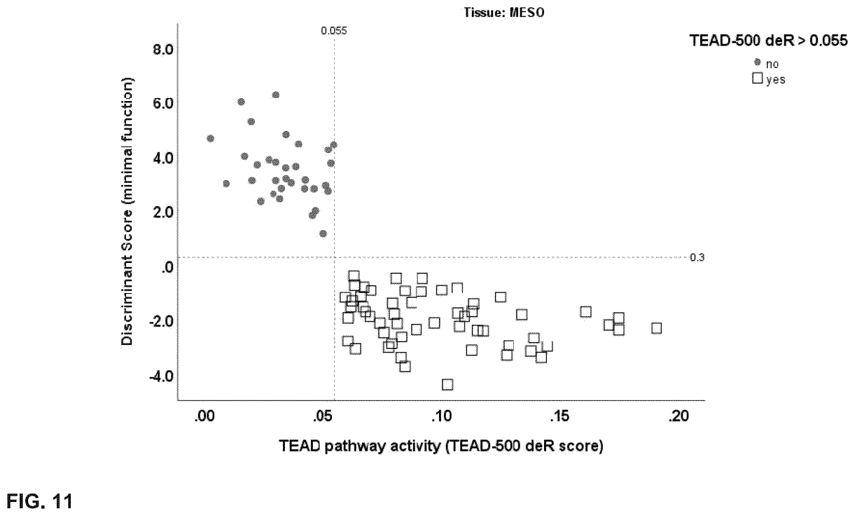

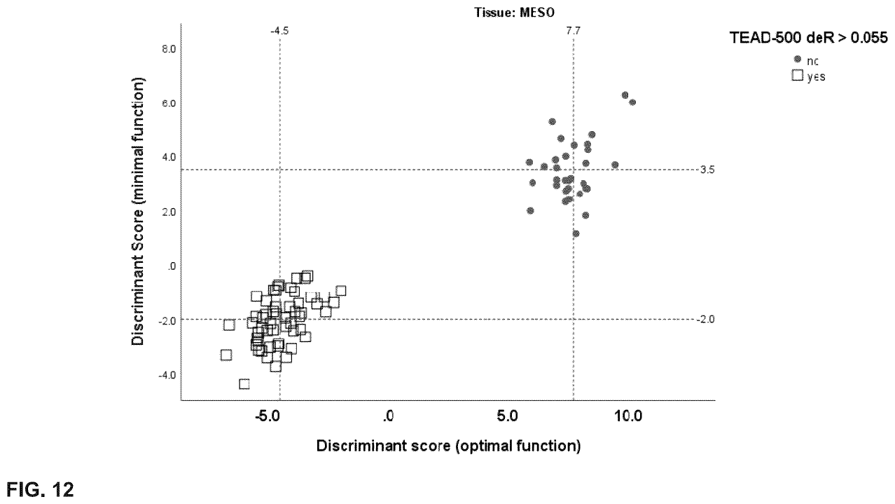

FIG. 10 and FIG. 11 show the relation of the scores of the optimal and minimal discriminant functions, respectively, with the gold standard measure of TEAD-pathway activity by TEAD-500 score. The TEAD-500 score varies continuously along the horizontal axis, but it is split into TEAD-active or TEAD-inactive groups by an arbitrary threshold based on a set of experimental observations (mesothelioma cell lines responding, or not, to an siRNA knockout of YAP1). The score of the optimal discriminant function, on the vertical axis (FIG. 10), comfortably separates the samples into two well distinct groups and predicts the response of mesothelioma tumors-assuming that the primary tumors are comparable to our cell lines-though using less predictor genes than TEAD-500. The scores of the minimal discriminant function for the same indication, based on even fewer predictors, still separate the two groups (FIG. 11). But the distance between the groups is lesser and the risk of misclassification of new samples is greater, particularly if the collected data are incomplete. The scores of the optimal and minimal discriminant functions are compared in FIG. 12, showing the coordinates of the centroids of each group for the two functions.

### Discriminant Score and Class Calculator.

Table 7 and Table 8 present a simulation of sample discrimination. Two mesothelioma samples are used as an example. Sample 1 has an active TEAD pathway whereas in Sample 2 the pathway is inactive according to the TEAD-500 score. The optimal set of predictors are listed in column A and their coefficients in column B. A subset of the predictors is used in the minimal discriminant function. Their coefficients are in column C. Columns D and E show the quantile-normalized expression data for these samples and genes. The fractional ranks of the RNA levels of the predictors, among the genes of the TEAD-500 list, are in columns F and G. Columns H and I contain the products of the fractional ranks of the genes in the two samples (columns F and G) multiplied with coefficients of the genes in the optimal function (column B). Similarly, columns J and K contain the products of the fractional ranks of the genes in the two samples (columns F and G) multiplied with coefficients of the genes in the minimal function (column C).

Row 29 contains the constants of the optimal and minimal functions, respectively in cells B29 and C29. These constants are added to the sum of products in columns H-K, accordingly. The total scores of the samples are shown in row 31. The distance of each score from each centroid is measured as:

$$Distance = max ( score , centroid ) - min ( score , centroid )$$

In this example, Sample-1 scores closer to centroid of the active group (2.76, using the optimal function, or 0.21, using the minimal function) than to the centroid of the inactive group (optimal distance=14.96; minimal distance=5.71). It is therefore, safely classified as active. Similarly, comparing the distances of the scores for Sample 2 (optimal in 131 and minimal in K31) with the centroid of the two functions for the two groups, we find both scores closer to the centroids of the inactive groups (compare 132 to 133, for the optimal function, or K32 to K33 for the minimal function). Sample 2 is, thus, classified as inactive.

TABLE 7
Calculator of discriminant scores.

| | Canonical coefficients | | Expression quantiles | | Fractional ranks | | Optimal Coef × Rank | | Minimal Coef × Rank | |
|---|---|---|---|---|---|---|---|---|---|---|
| Predictor | Optimal | Minimal | Sample 1 | Sample 2 | Sample 1 | Sample 2 | Sample 1 | Sample 2 | Sample 1 | Sample 2 |
| AASS | 9.1 | | −0.8 | −0.4 | 0.07 | 0.09 | 0.64 | 0.82 | 0.00 | 0.00 |
| ACOX2 | 13.8 | 6.7 | −0.7 | 3.2 | 0.08 | 0.48 | 1.10 | 6.62 | 0.54 | 3.22 |
| ADAMTS1 | −2.6 | | 5.4 | 1.5 | 0.82 | 0.24 | −2.13 | −0.62 | 0.00 | 0.00 |
| BCAT1 | −7 | −3.5 | 4.5 | 3.4 | 0.70 | 0.51 | −4.90 | −3.57 | −2.45 | −1.79 |
| CEBPB | −10.4 | | 4.9 | 7.0 | 0.76 | 0.94 | −7.90 | −9.78 | 0.00 | 0.00 |
| CHRNB1 | 22.6 | 8.3 | 2.6 | 2.4 | 0.38 | 0.36 | 8.59 | 8.14 | 3.15 | 2.99 |
| FXYD3 | 8.6 | 2.4 | −3.3 | −0.1 | 0.00 | 0.11 | 0.00 | 0.95 | 0.00 | 0.26 |
| IL6 | −5.5 | −1.3 | 5.1 | 1.0 | 0.78 | 0.20 | −4.29 | −1.10 | −1.01 | −0.26 |
| IRX5 | 10.3 | 6.1 | −0.1 | 3.2 | 0.11 | 0.49 | 1.13 | 5.05 | 0.67 | 2.99 |
| LYPD3 | 9.9 | | 0.3 | 0.0 | 0.13 | 0.11 | 1.29 | 1.09 | 0.00 | 0.00 |
| MALL | 4.3 | | 2.6 | 3.4 | 0.39 | 0.51 | 1.68 | 2.19 | 0.00 | 0.00 |
| NUAK1 | 8.8 | | 3.4 | 1.3 | 0.51 | 0.22 | 4.49 | 1.94 | 0.00 | 0.00 |
| PAK1 | −15.4 | −6 | 3.0 | 4.2 | 0.44 | 0.65 | −6.78 | −10.01 | −2.64 | −3.90 |
| PCNA | 17.8 | | 7.2 | 5.7 | 0.94 | 0.84 | 16.73 | 14.95 | 0.00 | 0.00 |
| PHF21A | 18.3 | 11.3 | 3.2 | 4.1 | 0.49 | 0.64 | 8.97 | 11.71 | 5.54 | 7.23 |
| PYGB | 9.6 | 7.4 | 5.8 | 5.4 | 0.86 | 0.82 | 8.26 | 7.87 | 6.36 | 6.07 |
| RACGAP1 | −9.6 | | 4.4 | 2.5 | 0.67 | 0.38 | −6.43 | −3.65 | 0.00 | 0.00 |
| RFC4 | −14.3 | | 4.0 | 3.7 | 0.62 | 0.57 | −8.87 | −8.15 | 0.00 | 0.00 |
| ROR1 | −4.3 | | 4.2 | 2.5 | 0.65 | 0.37 | −2.80 | −1.59 | 0.00 | 0.00 |
| RPS24 | 125.9 | | 9.8 | 11.0 | 0.99 | 1.00 | 124.64 | 125.90 | 0.00 | 0.00 |
| SMOC1 | 3.2 | | 2.5 | 1.6 | 0.37 | 0.25 | 1.18 | 0.80 | 0.00 | 0.00 |
| SORT1 | 8 | 4.7 | 2.5 | 1.2 | 0.38 | 0.21 | 3.04 | 1.68 | 1.79 | 0.99 |
| SUSD2 | −3.8 | | 5.2 | 5.2 | 0.79 | 0.80 | −3.00 | −3.04 | 0.00 | 0.00 |
| TEAD4 | −7.5 | | 4.1 | 4.5 | 0.64 | 0.70 | −4.80 | −5.25 | 0.00 | 0.00 |
| TMC4 | −4.9 | | 0.7 | 2.7 | 0.17 | 0.40 | −0.83 | −1.96 | 0.00 | 0.00 |
| TPM1 | −12 | −7.1 | 8.5 | 5.6 | 0.98 | 0.83 | −11.76 | −9.96 | −6.96 | −5.89 |
| Constant | −124.5 | 7.2 | | | | | −124.5 | −124.5 | −7.20 | −7.20 |

| | |
|---|---|
| optimal | minimal |

| | | | | | | | |
|---|---|---|---|---|---|---|---|
| Centroids: | | | Sum: | −7.26 | 6.53 | −2.21 | 4.71 |
| Inactive | 7.7 | 3.5 | distance from inactive centroid: | 14.96 | 1.17 | 5.71 | 1.21 |
| Active | −4.5 | −2 | distance from active centroid: | 2.76 | 11.03 | 0.21 | 6.71 |

TABLE 8

| | B | | C | | D | | E | | F | |
|---|---|---|---|---|---|---|---|---|---|---|
| A | Canonical coefficients | | Expression quantiles | | Fractional ranks | | Optimal Coef × Rank | | Minimal Coef × Rank | |
| Predictor | Optimal | Minimal | Sample 1 | Sample 2 | Sample 1 | Sample 2 | Sample 1 | Sample 2 | Sample 1 | Sample 2 |
| AASS | 9.1 | | −0.8 | −0.4 | 0.07 | 0.09 | 0.64 | 0.82 | 0.00 | 0.00 |
| ACOX2 | 13.8 | 6.7 | −0.7 | 3.2 | 0.08 | 0.48 | 1.10 | 6.62 | 0.54 | 3.22 |
| ADAMTS1 | −2.6 | | 5.4 | 1.5 | 0.82 | 0.24 | −2.13 | −0.62 | 0.00 | 0.00 |
| BCAT1 | −7 | −3.5 | 4.5 | 3.4 | 0.70 | 0.51 | −4.90 | −3.57 | −2.45 | −1.79 |
| CEBPB | −10.4 | | 4.9 | 7.0 | 0.76 | 0.94 | −7.90 | −9.78 | 0.00 | 0.00 |
| CHRNB1 | 22.6 | 8.3 | 2.6 | 2.4 | 0.38 | 0.36 | 8.59 | 8.14 | 3.15 | 2.99 |
| FXYD3 | 8.6 | 2.4 | −3.3 | −0.1 | 0.00 | 0.11 | 0.00 | 0.95 | 0.00 | 0.26 |
| IL6 | −5.5 | −1.3 | 5.1 | 1.0 | 0.78 | 0.20 | −4.29 | −1.10 | −1.01 | −0.26 |
| IRX5 | 10.3 | 6.1 | −0.1 | 3.2 | 0.11 | 0.49 | 1.13 | 5.05 | 0.67 | 2.99 |
| LYPD3 | 9.9 | | 0.3 | 0.0 | 0.13 | 0.11 | 1.29 | 1.09 | 0.00 | 0.00 |
| MALL | 4.3 | | 2.6 | 3.4 | 0.39 | 0.51 | 1.68 | 2.19 | 0.00 | 0.00 |
| NUAK1 | 8.8 | | 3.4 | 1.3 | 0.51 | 0.22 | 4.49 | 1.94 | 0.00 | 0.00 |
| PAK1 | −15.4 | −6 | 3.0 | 4.2 | 0.44 | 0.65 | −6.78 | −10.01 | −2.64 | −3.90 |
| PCNA | 17.8 | | 7.2 | 5.7 | 0.94 | 0.84 | 16.73 | 14.95 | 0.00 | 0.00 |
| PHF21A | 18.3 | 11.3 | 3.2 | 4.1 | 0.49 | 0.64 | 8.97 | 11.71 | 5.54 | 7.23 |
| PYGB | 9.6 | 7.4 | 5.8 | 5.4 | 0.86 | 0.82 | 8.26 | 7.87 | 6.36 | 6.07 |
| RACGAP1 | −9.6 | | 4.4 | 2.5 | 0.67 | 0.38 | −6.43 | −3.65 | 0.00 | 0.00 |
| RFC4 | −14.3 | | 4.0 | 3.7 | 0.62 | 0.57 | −8.87 | −8.15 | 0.00 | 0.00 |
| ROR1 | −4.3 | | 4.2 | 2.5 | 0.65 | 0.37 | −2.80 | −1.59 | 0.00 | 0.00 |
| RPS24 | 125.9 | | 9.8 | 11.0 | 0.99 | 1.00 | 124.64 | 125.90 | 0.00 | 0.00 |
| SMOC1 | 3.2 | | 2.5 | 1.6 | 0.37 | 0.25 | 1.18 | 0.80 | 0.00 | 0.00 |
| SORT1 | 8 | 4.7 | 2.5 | 1.2 | 0.38 | 0.21 | 3.04 | 1.68 | 1.79 | 0.99 |
| SUSD2 | −3.8 | | 5.2 | 5.2 | 0.79 | 0.80 | −3.00 | −3.04 | 0.00 | 0.00 |
| TEAD4 | −7.5 | | 4.1 | 4.5 | 0.64 | 0.70 | −4.80 | −5.25 | 0.00 | 0.00 |
| TMC4 | −4.9 | | 0.7 | 2.7 | 0.17 | 0.40 | −0.83 | −1.96 | 0.00 | 0.00 |
| TPM1 | −12 | −7.1 | 8.5 | 5.6 | 0.98 | 0.83 | −11.76 | −9.96 | −6.96 | −5.89 |
| Constant | −124.5 | −7.2 | | | | | −124.5 | −124.5 | −7.20 | −7.20 |

| | |
|---|---|
| optimal | minimal |

| | | | | | | | |
|---|---|---|---|---|---|---|---|
| Centroids: | | | Sum: | −7.26 | 6.53 | −2.21 | 4.71 |
| Inactive | 7.7 | 3.5 | distance from inactive centroid: | 14.96 | 1.17 | 5.71 | 1.21 |
| Active | −4.5 | −2 | distance from active centroid: | 2.76 | 11.03 | 0.21 | 6.71 |

### Example 3: Determination of a Discriminant Score (DS) of the Optimal Discriminant Function

### Determination of a (DS) Score

The dimensionality of testing was reduced from TEAD500 panel to subsets of the most informative genes applicable to specific cancer types. These panels (or sets) of fewer genes allow predicting the status of the TEAD-complex (active or inactive) in samples from a given cancer indication or subtype.

Using the Optimal discriminant function determined for the sets of genes a) to mmmm) it is possible to compute a discriminant score (DS) which is to be compared to a predetermined threshold for categorizing cancer or tumor sample as TEAD-active or TEAD-inactive cancer.

A (DS) score for a given cancer indication may be determined as follows:
1. Determination, in a tumor sample of an individual suffering from a cancer, of the expression levels of the genes listed in the optimal discriminant function of the set of genes associated with the specific tumor. The expression levels may be determined by RNA sequencing, NGS, RT-qPCR, or any other reliable method.
2. For each gene tested, converting the gene's expression level into a fractional rank, which according to the definitions herein, is the rank of the gene (i.e., the expression of the gene in relation with all the genes tested), where the gene with the lowest expression level is given the rank 1, the next highest level of expression a rank 2, and so on until the highest expressing gene is given the highest rank; equal expression levels are given the average rank, e.g. two genes with 0 expression are given the rank 1.5) divided by the number of genes tested.
3. Multiplying the fractional rank of each gene by the corresponding coefficient provided by the optimal discriminant function of the set of genes associated with the indication to obtain a product for each gene (Pgeneᵢ).
4. Computing the discriminant score (DS) of the tested set of genes in the tumor sample as the sum of the products Pgeneᵢ of all the discriminant genes plus the constant coefficient of the discriminant function, according to the formula:

(DS)=ΣPGeneᵢ+constant coefficient of the discriminant function of the tested set of genes,

where Geneᵢ is a gene listed in the discriminant function of the tested set of genes and Pgeneᵢ is equal to fractional rank of Geneᵢ*coefficient for Geneᵢ.

For example, if the optimal discriminant function for the indication in which the sample belongs has 10 genes, we multiply the fractional rank of each gene with the corresponding discriminant coefficient (known as the loading) of the gene, we add up the resulting products to the constant coefficient of the optimal discriminant function.
5. Determine the TEAD-activity status of the sample by comparing, for the tested set of genes, the (DS) with the threshold for each indication as listed in Table 3.

TABLE 3
Thresholds for determining the TEAD ‘active’ or TEAD ‘inactive’
status of a tumor per cohort (indication or sub-indication).

| Cancer indication - | Thresholds for TEAD-active status of a tumor | |
|---|---|---|
| Set of genes | less than | greater than |
| ACC | −156.02 | NA |
| set a) | | |
| BLCA | −1.76 | NA |
| set c) | | |
| BRCA | NA | 2.95 |
| set d) | | |
| BRCA_basal | NA | 5.81 |
| set e) | | |
| BRCA_non-basal | NA | 8.1 |
| set g) | | |
| CESC | −2.15 | NA |
| set i) | | |
| CHOL | NA | 63.98 |
| set j) | | |
| COAD | −3.37 | NA |
| set l) | | |
| COAD_CMS1 | −116.35 | NA |
| set m) | | |
| COAD_CMS4 | −556.79 | NA |
| set q) | | |
| DLBC | NA | −132.6 |
| set r) | | |
| ESCA | −5.52 | NA |
| set t) | | |
| GBM | −7.19 | NA |
| set v) | | |
| HNSC | −2.65 | NA |
| set x) | | |
| KICH | NA | 393.41 |
| set y) | | |
| KIRC | NA | 3.09 |
| set aa) | | |
| KIRP | NA | 12.65 |
| set bb) | | |
| LGG | NA | 7.77 |
| set dd) | | |
| LIHC | NA. | 2.73 |
| set ff) | | |
| LIHC_S1 | NA | 118.11 |
| set gg) | | |
| LIHC_S2 | −60.84 | NA |
| set ii) | | |
| LIHC_S3 | NA | 4.88 |
| set kk) | | |
| LUAD | NA | 4.15 |
| set mm) | | |
| LUSC | −1.86 | NA |
| set rr) | | |
| LUSC_basal | −455.53 | NA |
| set ss) | | |
| LUSC_classical | −3.44 | NA |
| set uu) | | |
| LUSC_primitive | NA | 60.27 |
| set vv) | | |
| LUSC_secretory | NA | 80.4 |
| set xx) | | |
| MESO | 2.05 | NA |
| set zz) | | |
| PAAD | −43 | NA |
| set kkk) | | |
| READ | −1.78 | NA |
| set lll) | | |
| READ_CMS1 | −44.65 | NA |
| set mmm) | | |
| READ_CMS2 | NA | 89.22 |
| set nnn) | | |
| READ_CMS4 | NA | −10.47 |
| set ppp) | | |
| SARC | 0.5 | NA |
| set rrr) | | |
| SKCM | NA | −0.85 |
| set sss) | | |
| SKCM_immune | −3.41 | NA |
| set ttt) | | |
| SKCM_keratin | NA | −30.14 |
| set uuu) | | |
| STAD_MSI | −74.44 | NA |
| set zzz) | | |
| STAD_MSS_EMT | −340.92 | NA |
| set bbbb) | | |
| TGCT | 11.89 | NA |
| set hhhh) | | |
| UCEC | −0.67 | NA |
| set kkkk) | | |
| UCS | NA | −49.52 |
| set llll) | | |

NA: Not applicable

Depending on the indication, the (DS) must be either lowed or greater than the given threshold to qualify a cancer as a TEAD-active cancer.

### Performance of Discriminant Scores (DS)

The performance of the discriminant score (DS) using the thresholds of Table 6 was evaluated from the confusion matrix (true positive, true negative, false positive and false negative counts) according to standard epidemiological metrics (https://en.wikipedia.org/wiki/Sensitivity_and_specificity) and presented in Table 9.

Several metrics can be computed from the numbers of positive (P) and negative (N) samples (determined with the comparison of DS to thresholds of Table 6) and true positive (TP), true negative (TN), false positive (FP) and false negative (FN) predictions to evaluate the performance of classifiers.

TP is a subject having a TEAD-active cancer correctly identified as having TEAD-active cancer; FP is a subject having a TEAD-inactive cancer incorrectly identified as having TEAD-active cancer; TN is a subject having a TEAD-inactive cancer correctly identified as having a TEAD-inactive cancer; and FN is a subject having a TEAD-active cancer incorrectly identified as having a TEAD-inactive cancer.

The positive predictions (PP) are the sum TP+FP. The negative predictions (NP) are the sum TN+FN. The prevalence of positive sample in a population (cohort) is the proportion of positives, P/(P+N). The accuracy of prediction is the proportion of correctly classified samples, (TP+TN)/(P+N). Precision, also known as positive predictive value (PPV), is the proportion of true positives among all the predicted positives, TP/PP. The false discovery rate (FDR) is the proportion of false positives among the predicted positives, FP/PP or 1−PPV. The false omission rate (FOR) is the proportion of predicted negatives that are true negatives, FN/PN or 1−NPV (negative predictive value). The negative predictive value (NPV) is the proportion of the predicted negatives that are true negatives, TN/PN, or 1−FOR. Sensitivity, also known as true positive rate (TPR), recall, or power of prediction, is the proportion of positives truly predicted as such, TP/P. Specificity, selectivity, or true negative rate (TNR), is the proportion of negatives that are truly predicted as such, TN/N. The false negative rate (FNR), or miss rate, is the proportion of positives that are falsely predicted to be negatives, FN/P.

The “F1-score” in Table 9 is a measure of a tests accuracy. This is calculated from the precision and sensitivity of the test as:

$$F 1 - score = ( 2 \times PPV \times TPR ) / ( PPV + TPR ) or 2 \times TP / ( 2 \times TP + FP + FN )$$

The “informesdness” in Table 9 is a measure of the performance of the method for each cancer type, defined in terms of sensitivity (true positive rate) and specificity (true negative rate) as TPR+TNR−1.

The values of these metrics for all the discriminant score (DS) are shown in Table 8. Out of 43 disclosed (DS), 20 achieve perfect discrimination (informedness=1) of tumors with active TEAD-pathway. Another 10 (DS) have excellent performance achieving informedness values equal or greater than 0.9. Another 5 (DS) have correct performance patient achieving informedness values in the range of 85-90%, and 8 (DS) have a moderate performance in the range of 0.6 to less than 0.85, and only one (DS) has performance slightly below 0.5 (0.46).

TABLE 9
Performance of the discriminant scores when only the optimal relevant genes
are interrogated following Method 3B per cancer types or sub-types

| Cancer | Total | | | | | miss | fall- | F1 | |
|---|---|---|---|---|---|---|---|---|---|
| type | sample | accuracy | precision | sensitivity | specificity | rate | out | score | informedness |
| ACC | 79 | 1 | 1 | 1 | 1 | 0 | 0 | 1 | 1 |
| BLCA | 430 | 0.99 | 0.95 | 0.97 | 0.99 | 0.03 | 0.01 | 0.96 | 0.96 |
| BRCA | 1227 | 0.99 | 0.92 | 0.91 | 0.99 | 0.09 | 0.01 | 0.91 | 0.9 |
| BRCA_basal | 212 | 0.98 | 0.95 | 0.97 | 0.98 | 0.03 | 0.02 | 0.96 | 0.95 |
| BRCA_non_{—} | 758 | 1 | 1 | 1 | 1 | 0 | 0 | 1 | 1 |
| basal | | | | | | | | | |
| CESC | 309 | 0.97 | 0.93 | 0.9 | 0.98 | 0.1 | 0.02 | 0.92 | 0.89 |
| CHOL | 45 | 1 | 1 | 1 | 1 | 0 | 0 | 1 | 1 |
| COAD | 514 | 1 | 1 | 1 | 1 | 0 | 0 | 1 | 1 |
| COAD_{—} | 53 | 1 | 1 | 1 | 1 | 0 | 0 | 1 | 1 |
| CMS1 | | | | | | | | | |
| COAD_{—} | 65 | 1 | 1 | 1 | 1 | 0 | 0 | 1 | 1 |
| CMS4 | | | | | | | | | |
| DLBC | 48 | 0.83 | 0.79 | 0.86 | 0.81 | 0.14 | 0.19 | 0.83 | 0.67 |
| ESCA | 198 | 1 | 1 | 1 | 1 | 0 | 0 | 1 | 1 |
| GBM | 191 | 0.97 | 0.93 | 0.86 | 0.99 | 0.14 | 0.01 | 0.89 | 0.85 |
| HNSC | 566 | 0.98 | 0.88 | 0.95 | 0.98 | 0.05 | 0.02 | 0.91 | 0.93 |
| KICH | 91 | 1 | 1 | 1 | 1 | 0 | 0 | 1 | 1 |
| KIRC | 609 | 1 | 1 | 1 | 1 | 0 | 0 | 1 | 1 |
| KIRP | 322 | 1 | 1 | 1 | 1 | 0 | 0 | 1 | 1 |
| LGG | 534 | 1 | 1 | 1 | 1 | 0 | 0 | 1 | 1 |
| LIHC | 424 | 0.98 | 0.89 | 0.91 | 0.99 | 0.09 | 0.01 | 0.9 | 0.9 |
| LIHC_S1 | 76 | 1 | 1 | 1 | 1 | 0 | 0 | 1 | 1 |
| LIHC_S2 | 135 | 1 | 1 | 1 | 1 | 0 | 0 | 1 | 1 |
| LIHC_S3 | 141 | 0.98 | 0.8 | 0.89 | 0.98 | 0.11 | 0.02 | 0.84 | 0.87 |
| LUAD | 589 | 0.99 | 0.97 | 0.92 | 1 | 0.08 | 0 | 0.94 | 0.91 |
| LUSC | 552 | 0.97 | 0.92 | 0.85 | 0.99 | 0.15 | 0.01 | 0.88 | 0.84 |
| LUSC_basal | 103 | 1 | 1 | 1 | 1 | 0 | 0 | 1 | 1 |
| LUSC_{—} | 169 | 0.85 | 0.32 | 0.79 | 0.85 | 0.21 | 0.15 | 0.46 | 0.64 |
| classical | | | | | | | | | |
| LUSC_{—} | 99 | 1 | 1 | 1 | 1 | 0 | 0 | 1 | 1 |
| primitive | | | | | | | | | |
| LUSC_{—} | 115 | 1 | 1 | 1 | 1 | 0 | 0 | 1 | 1 |
| secretory | | | | | | | | | |
| MESO | 87 | 0.98 | 0.98 | 0.98 | 0.97 | 0.02 | 0.03 | 0.98 | 0.95 |
| PAAD | 183 | 1 | 1 | 1 | 1 | 0 | 0 | 1 | 1 |
| READ | 177 | 0.95 | 0.69 | 0.69 | 0.98 | 0.31 | 0.02 | 0.69 | 0.67 |
| READ_{—} | 4 | 1 | 1 | 1 | 1 | 0 | 0 | 1 | 1 |
| CMS1 | | | | | | | | | |
| READ_{—} | 32 | 0.91 | 0.67 | 0.5 | 0.96 | 0.5 | 0.04 | 0.57 | 0.46 |
| CMS2 | | | | | | | | | |
| READ_{—} | 27 | 0.93 | 0.67 | 0.67 | 0.96 | 0.33 | 0.04 | 0.67 | 0.63 |
| CMS4 | | | | | | | | | |
| SARC | 265 | 0.98 | 0.99 | 0.97 | 0.98 | 0.03 | 0.02 | 0.98 | 0.96 |
| SKCM | 472 | 0.93 | 0.88 | 0.9 | 0.95 | 0.1 | 0.05 | 0.89 | 0.84 |
| SKCM_{—} | 180 | 0.95 | 0.96 | 0.87 | 0.98 | 0.13 | 0.02 | 0.91 | 0.85 |
| immune | | | | | | | | | |
| SKCM_{—} | 85 | 0.88 | 0.85 | 0.89 | 0.87 | 0.11 | 0.13 | 0.87 | 0.77 |
| keratin | | | | | | | | | |
| STAD_MSI | 101 | 1 | 1 | 1 | 1 | 0 | 0 | 1 | 1 |
| STAD_MSS_{—} | 129 | 1 | 1 | 1 | 1 | 0 | 0 | 1 | 1 |
| EMT | | | | | | | | | |
| TGCT | 150 | 0.96 | 0.96 | 0.98 | 0.93 | 0.02 | 0.07 | 0.97 | 0.91 |
| UCEC | 586 | 0.94 | 0.78 | 0.93 | 0.94 | 0.07 | 0.06 | 0.85 | 0.88 |
| UCS | 57 | 0.96 | 0.97 | 0.97 | 0.96 | 0.03 | 0.04 | 0.97 | 0.93 |

The results show that the Discriminant Score (DS) obtained with the Optimal Discriminant Function defined for the sets of genes a) to mmmm) and the associated cancer indication is a useful and efficient tool for categorizing the cancer of a patient as being TEAD-active or TEAD-inactive and managing the treatment of the patient.

1: A method for characterizing a TEAD-activity of a cancer in a subject in need thereof, comprising obtaining a transcriptional signature by measuring expression levels of genes of a set of genes in a biological sample of a tumor of said cancer, and using the transcriptional signature for characterizing the TEAD-activity of the cancer in the subject, wherein the set of genes comprises:
any of 220 to 249 of genes of a set of genes (1): AASS, ABAT, ACAT2, ADAMTS1, ADM, ADRB2, AMOT, ANXA3, ARHGAP11A, ARHGDIB, AURKB, AVPI1, AXL, AZIN1, B4GALT4, BCAT1, BIRC5, BTG3, C4BPB, CAP2, CAV1, CAVIN1, CCBE1, CCDC80, CCN1, CCN2, CDC25A, CDC6, CDCA3, CDCA4, CDCA5, CDCA8, CDH4, CDK2, CDK6, CDV3, CENPA, CENPI, CENPM, CENPN, CHRNB1, CHST13, CKS2, CLDN1, CLIC3, CNN3, COBL, COL8A1, COTL1, CPA4, CRIM1, CRY1, CTH, CXCL1, CYTH3, DAPK1, DCLRE1B, DDAH1, DHCR7, DHFR, DIAPH3, DKK1, DLL1, DONSON, DUSP14, DUT, EBP, EIF2AK3, EMG1, EPHA2, EPS8L2, ESM1, ETS1, EXO1, EXOSC2, F3, FAHD2A, FAM83D, FANCA, FAT4, FDPS, FEN1, FMR1, FST, FSTL1, FSTL3, GADD45A, GADD45B, GINS1, GPC6, GPR176, GPRC5A, GPRC5B, GRAMD2B, HASPIN, HEG1, HEXB, HPS5, HSPB11, IDI1, IGFBP7, IKBIP, IL6, ITGB2, JDP2, JPH2, KPNA2, KRT8, KRT80, LCA5, LHFPL6, LMCD1, LMNB2, LRP8, LRRFIP2, LSM5, LYPD6, LYRM1, MAD2L1, MAP6D1, MATN2, MATN3, MCM10, MCM2, MCM5, MDC1, METRNL, MICB, MID1, MRPL33, MSRB3, MVD, MXRA7, NCAPD3, NEDD4, NEDD4L, NEK2, NEXN, NFIB, NNMT, NOC3L, NTN4, NUAK1, NUAK2, NUDCD1, NUP107, NUP37, OGFRL1, OLFML3, OLR1, OXCT1, PAK2, PCBD1, PCNA, PDLIM2, PDZD2, PEPD, PHLPP1, PKMYT1, PKP2, PKP4, PLCE1, PLEKHA7, PLK2, PLOD2, PPIH, PRPS1, PRPS2, PRSS23, PSG2, PSG6, PSG7, PSG9, PVR, PXMP2, QDPR, QKI, RAB11FIP1, RAB32, RACGAP1, RBM24, RBMS2, RCN2, RFC4, RND3, RNF144B, ROR1, RPS24, SCD5, SCML1, SDC2, SEC14L1, SGK1, SGMS2, SGTB, SH3RF1, SHCBP1, SKP2, SLC25A23, SLC25A3, SLC38A5, SLC3A2, SLC7A1, SLC7A5, SMPD4, SNAPC1, SNX24, SORT1, SPAG1, SPATA5, STK3, STX11, STXBP6, SUSD2, SUV39H1, SYDE2, TACC3, TAGLN, TEAD1, TEAD4, TENT5B, TGM2, THBS1, TK1, TMEM139, TMEM160, TNFAIP3, TNFRSF12A, TNNC1, TPM1, TPX2, TRIP13, TSPAN2, TTF2, TUBB6, TUFT1, TYMS, UAP1, UBE2C, UGCG, UHRF1, VKORC1L1, WWC1, WWC2, YAP1, ZBED2, ZDHHC18, ZNF488, and ZNF704;
and
any of 210 to 233 of genes of a set of genes (2): AASDH, ABCA1, ABCC5, ABI3BP, ABLIM3, ACADVL, ACOT11, ACOX2, ACSL5, ADAM28, AGL, AGPAT4, ALDH3A2, ANKRD12, ANKRD22, ANKRD29, ANKRD42, ANTXR2, APBB3, ARAP3, ARHGEF2, ASFIA, ATP7A, ATXN1, BCL11B, BHLHE41, BMF, CA2, CASP1, CBR3, CCNG2, CDC42EP4, CDK1, CEBPB, CELSR3, CLCN3, CLDN4, COL6A1, COL6A2, CPE, CRABP2, CROT, CSRNP2, CSTA, CTNNBIP1, CTSB, CTSK, CXXC5, CYP1B1, CYP27C1, DDR1, DEDD2, DHX32, DIAPH2, DSC2, DSG3, DUSP6, DYNC2LI1, ELN, EPS8L3, ERAP2, FAM102A, FAM117B, FAM83B, FAM89B, FERMT1, FKBP2, FOS, FTH1, FXYD3, GDPD1, GOLGA5, GOLPH3L, GPNMB, GPRC5C, GRB10, GSN, HAS3, HBP1, HDAC1, HDHD2, HEY1, HOXA5, IFI44, IGSF3, IGSF9, INTS3, IRAK2, IRF9, IRX5, ITGA2, KCNMA1, KCNMB3, KCNN4, KIFAP3, KLF10, KLF13, KLHL3, KLK11, KRCC1, KRIT1, KRTDAP, LMTK3, LRP10, LTBP4, LXN, LYPD3, MALL, MANSC1, MAPK13, MARCKSL1, MFSD1, MFSD5, MGST2, MGST3, MLLT11, MLPH, MMP13, MSX2, MTMR11, MTMR9, MTSS1, MYO1A, NAGK, NAPEPLD, NCOA3, NFIL3, NPAS2, NRIP1, OAS1, OAS2, OASL, OFD1, OSBPL7, OTUB2, OVOL1, PAG1, PAK1, PCDHB2, PCDHB9, PCGF3, PCMTD2, PERP, PHF21A, PIK3C2B, PIK3R1, PIK3R2, PIK3R3, PIP4P2, PJA2, PKIA, PLA2G4C, PNRC1, PPP1R11, PRRX2, PTPRE, PYGB, RAC2, RALGPS1, RAPGEFL1, RBM23, RBM45, RBM47, RBP1, REEP6, RGL2, RGS17, RHOC, S100A14, SAMD9, SEC14L2, SECISBP2, SH3PXD2A, SH3TC1, SHROOM2, SLC14A1, SLCIA2, SLC30A9, SLC35C1, SLC37A2, SLC39A11, SLFN5, SLITRK6, SLK, SMOC1, SNCG, SP1, SPIRE2, SPRY4, SQSTMI, SRD5A3, SSPN, STMN3, STX1A, TBX3, TCF25, TDO2, TET2, TFF1, TLR3, TMC4, TMC7, TMEM140, TMEM144, TMEM45B, TP53INP1, TP63, TPD52L1, TRAPPC6B, TRIB1, TRIB2, TRIM13, TRIM31, TRIM38, TRIOBP, TRIP11, TSC22D1, TSPAN1, TTC17, TTLL3, TUBB3, UBC, ULK1, VAMP8, VGF, VPS52, VSNL1, WDR13, ZCWPW1, ZNF292, ZNF467, ZNF75D, and ZSWIM7.

2: The method of claim 1, wherein the set of genes comprises 430, 431, 432, 433, 434, 436, 437, or 482 genes.

3: The method of claim 1, wherein the cancer is a cancer comprising a solid tumor.

4: The method of claim 3, wherein the solid tumor is in the lung, colon, ovary, cervix, uterus, peritoneum, testicles, penis, tongue, lymph node, pancreas bone, breast, prostate, soft tissue, connective tissue, kidney, liver, brain, thyroid, or skin.

5: The method of claim 1, wherein the cancer is selected from the group consisting of adrenocortical carcinoma, bladder urothelial carcinoma, breast invasive carcinoma, cervical squamous cell carcinoma and endocervical adenocarcinoma, cholangiocarcinoma, consensus molecular subtypes 1 of colorectal cancer, consensus molecular subtypes 2 of colorectal cancer, consensus molecular subtypes 3 of colorectal cancer, consensus molecular subtypes 4 of colorectal cancer, colon adenocarcinoma, lymphoid neoplasm diffuse large b-cell lymphoma, esophageal carcinoma, glioblastoma multiforme, head and neck squamous cell carcinoma, kidney renal clear cell carcinoma, kidney renal papillary cell carcinoma, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma and paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine corpus endometrial carcinoma, and uterine carcinosarcoma.

6: The method of claim 1, comprising:
I. (a) measuring, in a transcriptome obtained from said biological sample, the expression levels of genes,
(b) for each gene of said set of genes, converting the gene's expression level obtained at step (a) into a fractional rank by dividing the rank of said gene by the number of genes from said transcriptome,
(c) isolating from the fractional ranks obtained at step (b) the fractional rank obtained for the genes of the set of genes (1) and computing their mean fractional rank (MFR-positive),
(d) isolating from the fractional ranks obtained at step (b) the fractional rank obtained for the genes of the set of genes (2) and computing their mean fractional rank (MFR-negative), and
(e) computing a dgR score as MFR-positive-MFR-negative;
or
II. (a) measuring, in said biological sample, the expression level of each gene of said set of genes,
(b) for each gene of said set of genes, converting the gene's expression level obtained at step (a) into a fractional rank by dividing the rank of said gene by the number of genes from said set of genes,
(c) isolating from the fractional ranks obtained at step (b) the fractional rank obtained for the genes of the set of genes (1) and computing their mean fractional rank (MFR-positive),
(d) isolating from the fractional ranks obtained at step (b) the fractional rank obtained for the genes of the set of genes (2) and computing their mean fractional rank (MFR-negative), and
(e) computing a deR score as MFR-positive-MFR-negative;
wherein when the dgR or the deR score is greater than about 0.055, then the cancer is TEAD-active, and when the dgR or deR score is less than or equal to about 0.055, then the cancer is TEAD-inactive.

7: A method for characterizing a TEAD-activity of a cancer in a subject in need thereof, comprising obtaining a transcriptional signature by measuring expression levels of genes of a set of genes in a biological sample of a tumor of said cancer, and using the transcriptional signature for characterizing the TEAD-activity of the cancer in the subject, wherein the set of genes is selected from the group consisting of sets (a) to (mmmm) of Table 1.

8: The method of claim 7, wherein when the cancer is a
Adrenocortical carcinoma (ACC) tumor, then the set of genes of the transcriptional signature is set of genes (a) or (b);
Bladder Urothelial Carcinoma (BLCA) tumor, then the set of genes of the transcriptional signature is set of genes (c);
Breast invasive carcinoma (BRCA) tumor, then the set of genes of the transcriptional signature is set of genes (d);
BRCA basal tumor, then the set of genes of the transcriptional signature is set of genes (e) or (f);
BRCA non-basal tumor, then the set of genes of the transcriptional signature is set of genes (g) or (h);
Cervical squamous cell carcinoma or endocervical adenocarcinoma (CESC) tumor, then the set of genes of the transcriptional signature is set of genes (i);
Cholangiocarcinoma (CHOL) tumor, then the set of genes of the transcriptional signature is set of genes (j) or (k);
Colon adenocarcinoma (COAD) tumor, then the set of genes of the transcriptional signature is set of genes (1);
COAD CMS1 tumor, then the set of genes of the transcriptional signature is set of genes (m) or (n);
COAD CMS2 tumor, then the set of genes of the transcriptional signature is set of genes (o) or (p);
COAD CMS4 tumor, then the set of genes of the transcriptional signature is set of genes (q) or (q1);
Lymphoid Neoplasm Diffuse Large B-cell Lymphoma (DLBC) tumor, then the set of genes of the transcriptional signature is set of genes (r) or(s);
Esophageal carcinoma (ESCA) tumor, then the set of genes of the transcriptional signature is set of genes (t) or (u);
Glioblastoma multiforme (GBM) tumor, then the set of genes of the transcriptional signature is set of genes (v) or (w);
Head or Neck squamous cell carcinoma (HNSC) tumor, then the set of genes of the transcriptional signature is set of genes (x);
Kidney Chromophobe (KICH) tumor, then the set of genes of the transcriptional signature is set of genes (y) or (z);
Kidney renal clear cell carcinoma (KIRC) tumor, then the set of genes of the transcriptional signature is set of genes (aa);
Kidney renal papillary cell carcinoma (KIRP) tumor, then the set of genes of the transcriptional signature is set of genes (bb) or (cc);
Brain Lower Grade Glioma (LGG) tumor, then the set of genes of the transcriptional signature is set of genes (dd) or (ee);
Liver hepatocellular carcinoma (LIHC) tumor, then the set of genes of the transcriptional signature is set of genes (ff);
LIHC S1 tumor, then the set of genes of the transcriptional signature is set of genes (gg) or (hh);
LIHC S2 tumor, then the set of genes of the transcriptional signature is set of genes (ii) or (jj);
LIHC S3 tumor, then the set of genes of the transcriptional signature is set of genes (kk) or (ll);
Lung adenocarcinoma (LUAD) tumor, then the set of genes of the transcriptional signature is set of genes (mm);
LUAD proximal-inflammatory tumor, then the set of genes of the transcriptional signature is set of genes (nn) or (oo);
LUAD proximal-proliferative tumor, then the set of genes of the transcriptional signature is set of genes (pp) or (qq);
Lung squamous cell carcinoma (LUSC) tumor, then the set of genes of the transcriptional signature is set of genes (rr);
LUSC basal tumor, then the set of genes of the transcriptional signature is set of genes (ss) or (tt);
LUSC classical tumor, then the set of genes of the transcriptional signature is set of genes (uu);
LUSC primitive tumor, then the set of genes of the transcriptional signature is set of genes (vv) or (ww);
LUSC secretory tumor, then the set of genes of the transcriptional signature is set of genes (xx) or (yy);
Malignant mesothelioma (MESO) tumor, then the set of genes of the transcriptional signature is set of genes (zz) or (aaa);
Ovarian serous cystadenocarcinoma (OV) tumor, then the set of genes of the transcriptional signature is set of genes (bbb);
OV differentiated tumor, then the set of genes of the transcriptional signature is set of genes (ccc) or (ddd);
OV immune-reactive tumor, then the set of genes of the transcriptional signature is set of genes (eee) or (fff);
OV mesenchymal tumor, then the set of genes of the transcriptional signature is set of genes (ggg) or (hhh);
OV proliferative tumor, then the set of genes of the transcriptional signature is set of genes (iii) or (jjj);
Pancreatic adenocarcinoma (PAAD) tumor, then the set of genes of the transcriptional signature is set of genes (kkk) or (kkk1);
Rectum adenocarcinoma (READ) tumor, then the set of genes of the transcriptional signature is set of genes (lll);
READ CMS1 tumor, then the set of genes of the transcriptional signature is set of genes (mmm);
READ CMS2 tumor, then the set of genes of the transcriptional signature is set of genes (nnn) or (ooo);
READ CMS4 tumor, then the set of genes of the transcriptional signature is set of genes (ppp) or (ppp1);
READ unclassifiable tumor, then the set of genes of the transcriptional signature is set of genes (qqq) or (qqq1);
Sarcoma (SARC) tumor, then the set of genes of the transcriptional signature is set of genes (rrr);
Skin Cutaneous Melanoma (SKCM) tumor, then the set of genes of the transcriptional signature is set of genes (sss);
SKCM immune tumor, then the set of genes of the transcriptional signature is set of genes (ttt);
SKCM keratin tumor, then the set of genes of the transcriptional signature is set of genes (uuu) or (vvv);
SKCM MITF-low tumor, then the set of genes of the transcriptional signature is set of genes (www) or (xxx);
Stomach adenocarcinoma (STAD) tumor, then the set of genes of the transcriptional signature is set of genes (yyy);
STAD MSI tumor, then the set of genes of the transcriptional signature is set of genes (zzz) or (aaaa);
STAD MSS_EMT tumor, then the set of genes of the transcriptional signature is set of genes (bbbb) or (cccc);
STAD MSS_TP53-tumor, then the set of genes of the transcriptional signature is set of genes (dddd) or (eeee);
STAD MSS_TP53+ tumor, then the set of genes of the transcriptional signature is set of genes (ffff) or (gggg);
Testicular Germ Cell Tumor (TGCT) tumor, then the set of genes of the transcriptional signature is set of genes (hhhh) or (iiii);
Thyroid carcinoma (THYM) tumor, then the set of genes of the transcriptional signature is set of genes (jjjj);
Uterine Corpus Endometrial Carcinoma (UCEC) tumor, then the set of genes of the transcriptional signature is set of genes (kkkk); and
Uterine Carcinosarcoma (UCS) tumor, then the set of genes of the transcriptional signature is set of genes (llll) or (mmmm).

9: The method of claim 7, comprising:
(a) measuring, in said biological sample, the expression level of each gene of a set of genes, wherein the set of genes comprises:
any of 220 to 249 of genes of a set of genes (1): AASS, ABAT, ACAT2, ADAMTS1, ADM, ADRB2, AMOT, ANXA3, ARHGAP11A, ARHGDIB, AURKB, AVPI1, AXL, AZIN1, B4GALT4, BCAT1, BIRC5, BTG3, C4BPB, CAP2, CAVI, CAVIN1, CCBE1, CCDC80, CCN1, CCN2, CDC25A, CDC6, CDCA3, CDCA4, CDCA5, CDCA8, CDH4, CDK2, CDK6, CDV3, CENPA, CENPI, CENPM, CENPN, CHRNB1, CHST13, CKS2, CLDN1, CLIC3, CNN3, COBL, COL8A1, COTL1, CPA4, CRIM1, CRY1, CTH, CXCL1, CYTH3, DAPK1, DCLRE1B, DDAH1, DHCR7, DHFR, DIAPH3, DKK1, DLL1, DONSON, DUSP14, DUT, EBP, EIF2AK3, EMG1, EPHA2, EPS8L2, ESM1, ETS1, EXO1, EXOSC2, F3, FAHD2A, FAM83D, FANCA, FAT4, FDPS, FEN1, FMR1, FST, FSTL1, FSTL3, GADD45A, GADD45B, GINS1, GPC6, GPR176, GPRC5A, GPRC5B, GRAMD2B, HASPIN, HEG1, HEXB, HPS5, HSPB11, IDI1, IGFBP7, IKBIP, IL6, ITGB2, JDP2, JPH2, KPNA2, KRT8, KRT80, LCA5, LHFPL6, LMCD1, LMNB2, LRP8, LRRFIP2, LSM5, LYPD6, LYRM1, MAD2L1, MAP6D1, MATN2, MATN3, MCM10, MCM2, MCM5, MDC1, METRNL, MICB, MID1, MRPL33, MSRB3, MVD, MXRA7, NCAPD3, NEDD4, NEDD4L, NEK2, NEXN, NFIB, NNMT, NOC3L, NTN4, NUAK1, NUAK2, NUDCD1, NUP107, NUP37, OGFRL1, OLFML3, OLR1, OXCT1, PAK2, PCBD1, PCNA, PDLIM2, PDZD2, PEPD, PHLPP1, PKMYT1, PKP2, PKP4, PLCE1, PLEKHA7, PLK2, PLOD2, PPIH, PRPS1, PRPS2, PRSS23, PSG2, PSG6, PSG7, PSG9, PVR, PXMP2, QDPR, QKI, RAB11FIP1, RAB32, RACGAP1, RBM24, RBMS2, RCN2, RFC4, RND3, RNF144B, ROR1, RPS24, SCD5, SCML1, SDC2, SEC14L1, SGK1, SGMS2, SGTB, SH3RF1, SHCBP1, SKP2, SLC25A23, SLC25A3, SLC38A5, SLC3A2, SLC7A1, SLC7A5, SMPD4, SNAPC1, SNX24, SORT1, SPAG1, SPATA5, STK3, STX11, STXBP6, SUSD2, SUV39H1, SYDE2, TACC3, TAGLN, TEAD1, TEAD4, TENT5B, TGM2, THBS1, TK1, TMEM139, TMEM160, TNFAIP3, TNFRSF12A, TNNC1, TPM1, TPX2, TRIP13, TSPAN2, TTF2, TUBB6, TUFT1, TYMS, UAP1, UBE2C, UGCG, UHRF1, VKORC1L1, WWC1, WWC2, YAP1, ZBED2, ZDHHC18, ZNF488, and ZNF704;
and
any of 210 to 233 of genes of a set of genes (2): AASDH, ABCA1, ABCC5, ABI3BP, ABLIM3, ACADVL, ACOT11, ACOX2, ACSL5, ADAM28, AGL, AGPAT4, ALDH3A2, ANKRD12, ANKRD22, ANKRD29, ANKRD42, ANTXR2, APBB3, ARAP3, ARHGEF2, ASF1A, ATP7A, ATXN1, BCL11B, BHLHE41, BMF, CA2, CASPI, CBR3, CCNG2, CDC42EP4, CDK1, CEBPB, CELSR3, CLCN3, CLDN4, COL6A1, COL6A2, CPE, CRABP2, CROT, CSRNP2, CSTA, CTNNBIP1, CTSB, CTSK, CXXC5, CYP1B1, CYP27C1, DDR1, DEDD2, DHX32, DIAPH2, DSC2, DSG3, DUSP6, DYNC2LI1, ELN, EPS8L3, ERAP2, FAM102A, FAM117B, FAM83B, FAM89B, FERMT1, FKBP2, FOS, FTH1, FXYD3, GDPD1, GOLGA5, GOLPH3L, GPNMB, GPRC5C, GRB10, GSN, HAS3, HBP1, HDAC1, HDHD2, HEY1, HOXA5, IFI44, IGSF3, IGSF9, INTS3, IRAK2, IRF9, IRX5, ITGA2, KCNMA1, KCNMB3, KCNN4, KIFAP3, KLF10, KLF13, KLHL3, KLK11, KRCC1, KRIT1, KRTDAP LMTK3, LRP10, LTBP4, LXN, LYPD3, MALL, MANSC1, MAPK13, MARCKSL1, MFSD1, MFSD5, MGST2, MGST3, MLLT11, MLPH, MMP13, MSX2, MTMR11, MTMR9, MTSS1, MYO1A, NAGK, NAPEPLD, NCOA3, NFIL3, NPAS2, NRIP1, OAS1, OAS2, OASL, OFD1, OSBPL7, OTUB2, OVOL1, PAG1, PAK1, PCDHB2, PCDHB9, PCGF3, PCMTD2, PERP, PHF21A, PIK3C2B, PIK3R1, PIK3R2, PIK3R3, PIP4P2, PJA2, PKIA, PLA2G4C, PNRC1, PPP1R11, PRRX2, PTPRE, PYGB, RAC2, RALGPS1, RAPGEFL1, RBM23, RBM45, RBM47, RBP1, REEP6, RGL2, RGS17, RHOC, S100A14, SAMD9, SEC14L2, SECISBP2, SH3PXD2A, SH3TC1, SHROOM2, SLC14A1, SLC1A2, SLC30A9, SLC35C1, SLC37A2, SLC39A11, SLFN5, SLITRK6, SLK, SMOC1, SNCG, SP1, SPIRE2, SPRY4, SQSTMI, SRD5A3, SSPN, STMN3, STX1A, TBX3, TCF25, TDO2, TET2, TFF1, TLR3, TMC4, TMC7, TMEM140, TMEM144, TMEM45B, TP53INP1, TP63, TPD52L1, TRAPPC6B, TRIB1, TRIB2, TRIM13, TRIM31, TRIM38, TRIOBP, TRIP11, TSC22D1, TSPAN1, TTC17, TTLL3, TUBB3, UBC, ULK1, VAMP8, VGF, VPS52, VSNL1, WDR13, ZCWPW1, ZNF292, ZNF467, ZNF75D, and ZSWIM7,
(b) for each gene of a set of genes shown in Table 1, converting the gene's expression level obtained at step (a) into a fractional rank by dividing the rank of said gene by the number of genes of said set of genes according to step (a);
(c) multiplying each fractional rank obtained at step (b) by a coefficient associated with each gene of said set of genes of Table 1, wherein said coefficient for said gene and set of genes is shown in Table 2, to obtain a product for each gene;
(d) summing the products obtained at step (c) to obtain a discriminant score (S);
(e) comparing the discriminant score (S) obtained at step (d) to the centroids of the TEAD-active (A) and TEAD-inactive (I) groups shown in Table 2 for said set of genes of Table 1;
(f) determining if (S) is closer to (A) than it is to (I) according to the formula max(S,A)−min(S,A)<max(S,I)−min(S,I); and
(g) characterizing the sample as TEAD-active if (S) is closer to (A) than it is to (I).

10: The method of claim 7, wherein
the cancer is selected from the group consisting of Adrenocortical carcinoma (ACC) tumors; Bladder Urothelial Carcinoma (BLCA) tumors; Breast invasive carcinoma (BRCA) tumors; BRCA basal tumors; BRCA non-basal tumors; Cervical squamous cell carcinoma and endocervical adenocarcinoma (CESC) tumors; Cholangiocarcinoma (CHOL) tumors; Colon adenocarcinoma (COAD) tumors; COAD CMS1 tumors; COAD CMS4 tumors; Lymphoid Neoplasm Diffuse Large B-cell Lymphoma (DLBC) tumors; Esophageal carcinoma (ESCA) tumors; Glioblastoma multiforme (GBM) tumors; Head and Neck squamous cell carcinoma (HNSC) tumors; Kidney Chromophobe (KICH) tumors; Kidney renal clear cell carcinoma (KIRC) tumors; Kidney renal papillary cell carcinoma (KIRP) tumors; Brain Lower Grade Glioma (LGG) tumors; Liver hepatocellular carcinoma (LIHC) tumors; LIHC S1 tumors; LIHC S2 tumors; LIHC S3 tumors; Lung adenocarcinoma (LUAD) tumors; Lung squamous cell carcinoma (LUSC) tumors; LUSC basal tumors; LUSC classical tumors; LUSC primitive tumors; LUSC secretory tumors; Malignant mesothelioma (MESO) tumors; Pancreatic adenocarcinoma (PAAD) tumors; Rectum adenocarcinoma (READ) tumors; READ CMS1 tumors; READ CMS2 tumors; is READ CMS4 tumors; Sarcoma (SARC) tumors; Skin Cutaneous Melanoma (SKCM) tumors; SKCM immune tumors; SKCM keratin tumors; STAD MSI tumors; STAD MSS_EMT tumors; Testicular Germ Cell Tumors (TGCT) tumors; Uterine Corpus Endometrial Carcinoma (UCEC) tumors; Uterine Carcinosarcoma (UCS) tumors,
and wherein
the method comprises:
(a) measuring, in said biological sample, the expression level of each gene of a set of genes associated with said tumor, the set of genes being selected from the group consisting of set (a), set (c), set (d), set (e), set (g), set (i), set (j), set (l), set (m), set (q), set (r), (t), set (v), set (x), set (y), set (aa), set (bb), set (dd), set (ff), set (gg), set (ii), set (kk), set (mm), set (rr), set (ss), set (uu), set (vv), set (xx), set (zz), set (kkk), set (lll), set (mmm), set (nnn), set (ppp), set (rrr), set (sss), set (ttt), set (uuu), set (zzz), set (bbbb), set (hhhh), set (kkkk), and set (llll) of Table 1;
(b) for each gene of said set of genes, converting the gene's expression level obtained at step (a) into a fractional rank by dividing the rank of said gene by the number of genes of said set of genes;
(c) multiplying each fractional rank obtained at step (b) by a coefficient associated with each gene of said set of genes, wherein said coefficient for said gene and set of genes is shown in Table 2, to obtain a product (Pgeneᵢ) for each gene;
(d) determining a discriminant score (DS) of said set of genes, wherein the discriminant score (DS) is the sum of the products Pgeneᵢ obtained at step (c) plus the constant coefficient for said set of genes shown in Table 2, and
(e) comparing, for said set of genes, the discriminant score (DS) obtained at step (d) with a threshold associated with said set of genes, wherein the threshold is shown in Table 3, to determine if the cancer is TEAD-active or TEAD-inactive.

11: The method of claim 1, wherein the biological sample is a sample from blood, plasma, serum, cerebral spinal fluid (CSF), a tissue, a cell, or a tumor biopsy.

12: The method of claim 1, wherein the subject has a TEAD-active or TEAD-inactive cancer.

13: The method of claim 1, comprising predicting whether a subject will respond positively to a TEAD-pathway inhibitor.

14: The method of claim 12, wherein the subject is administered a TEAD-pathway inhibitor if the subject is characterized as having a TEAD-active cancer or if the subject is predicted to respond positively to a TEAD-pathway inhibitor.

15: A method for characterizing the TEAD-activity of a cancer in a subject in need thereof, wherein the method uses a biological sample of a tumor of said cancer and comprises at least the steps of:
(a) measuring, in a transcriptome obtained from said biological sample, the expression levels of genes,
(b) for each gene of a set of genes according to claim 1, converting the gene's expression level obtained at step (a) into a fractional rank by dividing the rank of said gene by the number of genes from said transcriptome,
(c) isolating from the fractional ranks obtained at step (b) the fractional rank obtained for the genes of the set of genes (1) and computing their mean fractional rank (MFR-positive),
(d) isolating from the fractional ranks obtained at step (b) the fractional rank obtained for the genes of the set of genes (2) and computing their mean fractional rank (MFR-negative), and
(e) computing the dgR score as MFR-positive-MFR-negative;
or
II. (a) measuring, in said biological sample, the expression level of each gene of a set of genes according to claim 1,
(b) for each gene of said set of genes according to claim 1, converting the gene's expression level obtained at step (a) into a fractional rank by dividing said rank of said gene by the number of genes from said set of genes,
(c) isolating from the fractional ranks obtained at step (b) the fractional rank obtained for the genes of the set of genes (1) and computing their mean fractional rank (MFR-positive),
(d) isolating from the fractional ranks obtained at step (b) the fractional rank obtained for the genes of the set of genes (2) and computing their mean fractional rank (MFR-negative), and
(e) computing the deR score as MFR-positive-MFR-negative;
wherein when the dgR or the deR score is greater than about 0.055, then the cancer is TEAD-active, and when the dgR or deR score is less than or equal to about 0.055, then the cancer is TEAD-inactive.

16: A method for characterizing the TEAD-activity of a cancer in a subject in need thereof, wherein the method uses a biological sample of a tumor of said cancer and comprises at least the steps of:
(a) measuring, in said biological sample, the expression level of each gene of a set of genes according to claim 1,
(b) for each gene of a set of genes shown in Table 1, converting the gene's expression level obtained at step (a) into a fractional rank by dividing the rank of said gene by the number of genes of said set of genes according to claim 1;
(c) multiplying each fractional rank obtained at step (b) by a coefficient associated with each gene of said set of genes of Table 1, wherein said coefficient for said gene and set of genes is shown in Table 2, to obtain a product for each gene;
(d) summing the products obtained at step (c) to obtain a discriminant score (S);
(e) comparing the discriminant score (S) obtained at step (d) to the centroids of the TEAD-active (A) and TEAD-inactive (I) groups shown in Table 2 for said set of genes of Table 1;
(f) determining if (S) is closer to (A) than it is to (I) according to the formula max(S,A)−min(S,A)<max(S,I)−min(S,I); and
(g) characterizing the cancer as TEAD-active if (S) is closer to (A) than it is to (I).

17: A method for characterizing the TEAD-activity of a cancer in a subject in need thereof, wherein:
the cancer is selected from the group consisting of Adrenocortical carcinoma (ACC) tumors; Bladder Urothelial Carcinoma (BLCA) tumors; Breast invasive carcinoma (BRCA) tumors; BRCA basal tumors; BRCA non-basal tumors; Cervical squamous cell carcinoma and endocervical adenocarcinoma (CESC) tumors; Cholangiocarcinoma (CHOL) tumors; Colon adenocarcinoma (COAD) tumors; COAD CMS1 tumors; COAD CMS4 tumors; Lymphoid Neoplasm Diffuse Large B-cell Lymphoma (DLBC) tumors; Esophageal carcinoma (ESCA) tumors; Glioblastoma multiforme (GBM) tumors; Head and Neck squamous cell carcinoma (HNSC) tumors; Kidney Chromophobe (KICH) tumors; Kidney renal clear cell carcinoma (KIRC) tumors; Kidney renal papillary cell carcinoma (KIRP) tumors; Brain Lower Grade Glioma (LGG) tumors; Liver hepatocellular carcinoma (LIHC) tumors; LIHC S1 tumors; LIHC S2 tumors; LIHC S3 tumors; Lung adenocarcinoma (LUAD) tumors; Lung squamous cell carcinoma (LUSC) tumors; LUSC basal tumors; LUSC classical tumors; LUSC primitive tumors; LUSC secretory tumors; Malignant mesothelioma (MESO) tumors; Pancreatic adenocarcinoma (PAAD) tumors; Rectum adenocarcinoma (READ) tumors; READ CMS1 tumors; READ CMS2 tumors; is READ CMS4 tumors; Sarcoma (SARC) tumors; Skin Cutaneous Melanoma (SKCM) tumors; SKCM immune tumors; SKCM keratin tumors; STAD MSI tumors; STAD MSS_EMT tumors; Testicular Germ Cell Tumors (TGCT) tumors; Uterine Corpus Endometrial Carcinoma (UCEC) tumors; and Uterine Carcinosarcoma (UCS) tumors,
and
the method uses a biological sample of a tumor of said cancer, and
the method comprises at least the steps of:
(a) measuring, in said biological sample, the expression level of each gene of a set of genes associated with said tumor, wherein the set of genes is selected from the group consisting of set (a), set (c), set (d), set (e), set (g), set (i), set (j), set (l), set (m), set (q), set (r), (t), set (v), set (x), set (y), set (aa), set (bb), set (dd), set (ff), set (gg), set (ii), set (kk), set (mm), set (rr), set (ss), set (uu), set (vv), set (xx), set (zz), set (kkk), set (lll), set (mmm), set (nnn), set (ppp), set (rrr), set (sss), set (ttt), set (uuu), set (zzz), set (bbbb), set (hhhh), set k (kkk), and set (llll) of Table 1;
(b) for each gene of said set of genes, converting the gene's expression level into a fractional rank by dividing the rank of the gene by the number of genes of said set of genes;
(c) multiplying each fractional rank obtained at step (b) by a coefficient associated with each gene of said set of genes, wherein said coefficient for said gene and set of genes is shown in Table 2, to obtain a product (Pgeneᵢ) for each gene;
(d) determining a discriminant score (DS) of said set of genes, wherein the discriminant score (DS) is the sum of the products Pgeneᵢ obtained at step (c) plus the constant coefficient of said set of genes shown in Table 2;
(e) comparing, for said set of genes, the discriminant score (DS) obtained at step (d) with a threshold associated with said set of genes, wherein the threshold is shown in Table 3, to determine if the cancer is TEAD-active or TEAD-inactive.

18: A kit comprising a solid support comprising a panel of nucleic acid for determining the transcription of a set of genes according to claim 1.
